(19) United States
(12) Patent Application Publication
(10) Pub. No.: US 2024/0060091 A1
(43) Pub. Date: Feb. 22, 2024

(54) METHOD FOR PROGRAMMABLE CONTROL OF RNA TRANSCRIPT LEVELS WITH AUTOREGULATED CRISPR-CAS13D

(71) Applicant: University of Florida Research Foundation Research, Gainesville, FL (US)

(72) Inventors: Eric Tzy-Shi Wang, Gainesville, FL (US); Chase P. Kelley, Gainesville, FL (US)

(73) Assignee: University of Florida Research Foundation, incorporation, Gainesville, FL (US)

(21) Appl. No.: 18/267,144

(22) PCT Filed: Dec. 17, 2021
(86) PCT No.: PCT/US2021/064232

§ 371 Date: Jun. 14, 2023

Related U.S. Application Data
(60) Provisional application No. 63/127,110, filed on Dec. 17, 2020.

(51) Int. Cl. C12N 15/90; C12N 15/86; C12N 15/11; C12N 9/22

(52) U.S. Cl. CPC C12N 15/907; C12N 15/86; C12N 15/11; C12N 15/111; C12N 9/22; C12N 2310/20; C12N 2800/80; C12N 2750/14143; C12N 2820/002; C07K 2319/81; A61K 38/00

(57) ABSTRACT

Autoregulatory systems that have been implemented to control the expression and activity of Cast 3d, including the activity of Cast 3d in vivo, thereby reducing collateral damage in cells at off-target sites (e.g., non-target messenger RNA transcripts. The autoregulatory system is in the form of a nucleic acid molecule comprising a zinc finger binding site, a promoter, and a nucleotide sequence encoding a Cast 3d fusion protein, and a transcriptional repressor domain, and wherein the Cast 3d fusion protein binds to the zinc finger binding site and represses transcription of the nucleotide sequence encoding the Cast 3d fusion protein.

### RELATED APPLICATIONS

This application claims the benefit under 35 U.S.C 119(e) of the filing date of U.S. Provisional Application Ser. No. 63/127,110, filed Dec. 17, 2020, the entire contents of which are incorporated herein by reference.

### GOVERNMENT SUPPORT

This invention was made with government support under Grant No. R01 AG058636, awarded by the National Institutes of Health. In addition, this invention was made with whole or in part with support under Application Number: 1000259801 awarded by The NSF Graduate Research Fellowship Program. The government has certain rights in this invention.

### BACKGROUND

CRISPR-Cas13 is a recently discovered family of RNA-guided RNA endonucleases capable of sequence-specific binding to and cleavage of target RNA transcripts. Cas13 is a Type VI CRISPR system having tremendous potential as a biochemical tool and as an agent of gene therapy. Type VI editors, such as Cas13, are CRISPR systems that are capable of RNA cleavage and editing. The ability to cleave RNA makes Type VI systems particularly applicable in RNA mediated diseases, such as repeat expansion diseases (e.g., myotonic dystrophy type 1 disease).

CRISPR-Cas13 editors typically comprise two basic components, namely, a Cas13 protein and a guide RNA. The Cas13 protein comprises a nuclease domain and a guide RNA binding domain. The guide RNA comprises a spacer sequence that is complementary to a target polynucleotide (e.g., RNA transcript) and a direct repeat sequence that binds with the Cas13 protein. The Cas13 protein and guide RNA form a complex which is directed to the target polynucleotide based on the sequence of the guide RNA spacer. Once localized and bound, the Cas13 nuclease cleaves the target RNA, which results in knockdown of gene expression.

Cas13 has four major isoforms Cas13a-Cas13d. Of therapeutic interest is Cas13d, because it is compact (190 to 300 amino acids smaller than Cas13a-Cas13c), which makes Cas13d particularly suitable for in vivo delivery. (Yan et al. Molecular cell 70.2 (2018): 327-339). Additionally, there are conflicting reports that other isoforms of Cas13 (e.g., Cas13a) have non-specific RNase activity that is capable of cleaving bystander RNAs in trans (i.e., “collateral damage”). (Wang et al. Advanced Science 6.20 (2019): 1901299; Lin, Ping, et al. Molecular cell 78.5 (2020): 850-861; Abudayyeh et al. “Nature 550.7675 (2017): 280-284; Aman etl al. Genome biology 19.1 (2018): 1-9.). However, previous work has suggested that Cas13d does not have collateral activity in mammalian cells or plant cells (Konermann Cell 173.3 (2018): 665-676; Mahas et al. Genome biology 20.1 (2019): 1-16). That Cas13 is associated with

The development of programmable editors that are capable of sequence-specific binding to and cleavage of target RNA transcripts while eliminating or mitigating collateral damage would significantly advance the art.

### SUMMARY

This disclosure relates in part to the surprising finding that Cas13d causes collateral damage in mammalian cells. These findings are surprising because previous studies have reported that Cas13d does not cause collateral damage in mammalian cells mammalian cells or plant cells (Konermann Cell 173.3 (2018): 665-676; Mahas et al. Genome biology 20.1 (2019): 1-16.). Collateral damage occurs when the Cas13d nuclease targets and cleaves a nucleotide that is not specified by the guide RNA.

To reduce Cas13d collateral damage, the present disclosure provides autoregulatory systems that have been implemented to control the expression and activity of Cas13d, including the activity of Cas13d in vivo. The results described herein demonstrate that these autoregulatory systems are sufficient to reduce collateral damage while still maintaining robust degradation (or knockdown) of a specific target transcript specified by a Cas13d guide RNA.

Accordingly, in one aspect, the present disclosure provides autoregulatory systems that have been implemented to control the expression and activity of Cas13d, including the activity of Cas13d in vivo.

In some embodiments, the present disclosure provides an autoregulatory system in the form of a nucleic acid molecule comprising a zinc finger binding site, a promoter, and a nucleotide sequence encoding a Cas13d fusion protein, wherein the Cas13d fusion protein comprises a Cas13d domain, a zinc finger (ZnF) domain, and a transcriptional repressor domain, and wherein the Cas13d fusion protein is capable of binding to the zinc finger binding site thereby repressing transcription of the nucleotide sequence encoding the Cas13d fusion protein. Without being bound to theory, as the Cas13d fusion protein concentration increases due to expression, the Cas13d fusion protein binds to the zinc finger domain upstream of the fusion protein gene. The transcription repressor domain (e.g., Kruppel associate box (KRAB) domain) then acts to repress transcription of the Cas13d fusion protein transcript, thereby decreasing the expression of the Cas13d fusion protein.

In another embodiment, the present disclosure provides an autoregulatory system in the form of a nucleic acid molecule comprising in a nucleotide sequence encoding a Cas13d domain and a Cas13d processing sequence (e.g., a pre-guide RNA) on a single Cas13d autoregulation transcript. Without being bound by theory, Cas13d autoregulation occurs when the HEPN-2 domain of the Cas13d cleaves the Cas13d processing sequence from the Cas13d autoregulation transcript. This cleavage exposes the transcript to RNA exonuclease degradation. Hence as Cas13d domain concentration increases in the cell, Cas13d autoregulation transcript abundance is decreased and in turn decreases Cas13d collateral damage as compared to Cas13d with no autoregulation.

In other aspects, the disclosure provides a method of transcript target knockdown using an Cas13d fusion protein, comprising expressing a nucleic acid molecule comprising a zinc finger binding site, a promoter, and a nucleotide sequence encoding a Cas13d fusion protein, wherein the Cas13d fusion protein comprises a Cas13d domain, a zinc finger (ZnF) domain, and a transcriptional repressor domain, and wherein the Cas13d fusion protein binds to the zinc finger binding site and represses transcription of the nucleotide sequence encoding the Cas13d fusion protein.

In still another aspect, the disclosure provides a method of target transcript knockdown using an autoregulated Cas13d domain, comprising expression of a nucleic acid molecule comprising a nucleotide sequence encoding a Cas13d domain and Cas13d processing sequence (e.g., pre-guide RNA) on a single Cas13d-gRNA transcript.

In various other aspects, the present disclosure provides viral vectors comprising the herein disclosed nucleic acid molecules comprising the Cas13d domain autoregulatory systems. In still other aspects, the present disclosure provides plasmids comprising the herein disclosed nucleic acid molecules comprising the Cas13d domain autoregulatory systems. In other aspects, the disclosure provides compositions comprising a nucleic acid molecule, nucleic acid plasmid, or viral vector described herein, and a pharmaceutically acceptable excipient. In still other aspects, the disclosure provides cells comprising the nucleic acid molecules, plasmids, or vectors described herein. In still other aspects, the disclosure provides kits comprising the nucleic acid molecules, plasmids, or vectors described herein.

In various embodiments, the zinc finger binding site used in the present disclosure comprises an amino acid sequence of any one of SEQ ID NO: 9 (HLTR3 ZnF), SEQ ID NO: 11 (HLTR1 ZnF), SEQ ID NO: 13 (HLTR6 ZnF), and SEQ ID NO: 21 (hCCR5 ZnF), or an amino acid sequence having at least 80%, 85%, 90%, 95%, or 99% or more sequence identity with any one of SEQ ID NOs: 9, 11, 13, or 21.

In other embodiments, the nucleic acid molecule encoding the Cas13d fusion protein is operably linked to a promoter. In some embodiments, the promoter can be an inducible promoter or a constitutive promoter. The promoter can also be from any organism, such as a eukaryotic or a prokaryotic organism.

In various embodiments, the eukaryotic promoter can be a CMV promoter, a EF1a promoter, a CAG promoter, a PGK promoter, a TRE promoter, a U6 promoter, a UAS promoter, a SK448 promoter, a SPc5-12 promoter, a MCK promoter, a DES promoter, a skeletal actin promoter, a heart alpha actin promoter, a myosin promoter, an RSV promoter, a C5-12 promoter, a ESYN promoter, a MSCV promoter, a CaMKII alpha promoter, a MHCK7 promoter, or a SK-CRM4-Des promoter. Such promoters are well-known in the art and readily obtainable by one of ordinary skill in the art.

In various other embodiments, the prokaryotic promoter can be a T7 promoter, a T71ac promoter, a Sp6 promoter, a lac promoter, an araBad promoter, a trp promoter, a Ptac promoter, a P1 promoter, a T3 promoter, or a Ptet promoter.

In various embodiments, the Cas13d fusion protein comprises an amino acid sequence of any one of SEQ ID NO: 1 (AdmCas13d domain), SEQ ID NO: 2 (EsCas13d domain), SEQ ID NO: 3 (P1E0Cas13d domain), SEQ ID NO: 4 (RaCas13d domain), SEQ ID NO: 5 (RffCas13d domain), SEQ ID NO: 6 (RfxCas13d domain), SEQ ID NO: 7 (UrCas13d domain), or an amino acid sequence having at least 80%, 85%, 90%, 95%, or 99% or more sequence identity with any one of SEQ ID NOs: 1-7.

In various embodiments, the Cas13d fusion protein comprises a zinc finger domain comprising an amino acid sequence of any one of SEQ ID NO: 8 (HLTR3 ZnF), SEQ ID NO: 10 (HLTR1 ZnF), SEQ ID NO: 12 (HLTR6 ZnF), and SEQ ID NO: 20 (hCCR5 ZnF), or an amino acid sequence having at least 80%, 85%, 90%, 95%, or 99% or more sequence identity with any one of SEQ ID NOs: 8, 10, 12, or 20.

In various embodiments, the Cas13d fusion protein comprises a transcriptional repressor domain comprises an amino acid sequence of any one of SEQ ID NO: 22 (SID), SEQ ID NO: 23 (SID4X), SEQ ID NO: 24 (KRAB-A), or an amino acid sequence having at least 80%, 85%, 90%, 95%, or 99% or more sequence identity with any one of SEQ ID NOs: 22-24.

In various other embodiments, the transcriptional repressor domain can comprise an amino acid sequence of any one of SEQ ID NOs: 22-23, or an amino acid sequence having at least 90% sequence identity with any of SEQ ID NOs: 22-23. In some embodiments, the transcriptional repressor domain comprises SID (SEQ ID NO:22) or SIDX4 (SEQ ID NO:23). In some embodiments, the transcriptional repressor domain consists of SID (SEQ ID NO:22) or SIDX4 (SEQ ID NO:23).

In certain embodiments, the transcriptional repressor domain is a KRAB domain comprising an amino acid sequence of any one of SEQ ID NOs: 24, or an amino acid sequence having at least 90% sequence identity with any of SEQ ID NOs: 24.

In various embodiments, the Cas13d fusion protein can comprise one or more linkers. The one or more linkers can have an amino acid sequence selected from the group consisting of any of SEQ ID NOs: 25-35, or an amino acid sequence having at least 80%, 85%, 90%, 95%, or 99% or more sequence identity with any one of SEQ ID NOs: 25-35.

In various embodiments, the linker joins a Cas13d domain and a zinc finger (ZnF) domain. In other embodiments, the linker joins a zinc finger (ZnF) domain and a transcriptional repressor domain, e.g., a KRAB domain.

The Cas13d fusion proteins described herein can also comprise one or more nuclear localization signal (NLS) sequences.

The Cas13d fusion proteins described herein can also comprise one or more mitochondrial localization signals (MLS).

In various embodiments, the Cas13d domain may be fused to an NLS at the C- or the N-terminus of the Cas13d domain. In various other embodiments, the zinc finger binding domain may be fused to an NLS at the C- or the N-terminus of the zinc finger binding domain. In still other embodiments, the transcriptional repressor (e.g., KRAB domain) may be fused to an NLS at the C- or N-terminus of the transcriptional repressor domain.

In various other embodiments, the Cas13d fusion proteins described herein, or any one or more components of said Cas13d fusion proteins (e.g., the Cas13d domain, transcriptional repressor domain, or the zinc finger binding domain) may further comprise a peptide tag, e.g., such as a histidine tag or other purification, detection, or separation tag.

In some embodiments, the nucleic acid molecule described herein, further comprises a peptide tag, e.g., a His-tag (SEQ ID NO: 51), HA-tag (SEQ ID NO: 49), Flag-tag (SEQ ID NO: 50), a Myc tag (SEQ ID NO: 52), a V5 tag (SEQ ID NO: 53), or an AviTag-PT-6 (SEQ ID NO: 54).

In various embodiments, the viral vectors described herein may be an adenovirus vector, an adeno-associated virus vector, or a lentivirus vector, or other suitable virus vectors. In the case of an adeno-associated virus vector, the vector can be an AAV1, AAV2, AAV3, AAV4, AAV5, AAV6, AAV7, AAV8, AAV9 or chimera thereof (e.g., AAV1/2 chimera, AAV3/4 chimera, or an AAV5/6 chimera, and the like). These and other aspects are described in the following drawings, examples, and claims.

In some aspects, the present disclosure describes a nucleic acid molecule comprising a zinc finger binding site, and a nucleotide sequence encoding a Cas13d fusion protein, wherein the Cas13d fusion protein comprises a Cas13d domain, a zinc finger (ZnF) domain, and a transcriptional repressor domain, and wherein the Cas13d fusion protein is capable of binding to the zinc finger binding site and to repress transcription of the nucleotide sequence encoding the Cas13d fusion protein, In some embodiments, the zinc finger binding site is 5′ to the Cas13d fusion protein.

In some embodiments, the zinc finger binding site comprises a nucleotide sequence of any one of SEQ ID NOs: 9, 11, 13, 15, 17, 19, or 21.

In some embodiments, the nucleic acid molecule further comprising a promoter.

In some embodiments, the promoter is an inducible promoter

In some embodiments, the promoter is a constitutive promoter.

In some embodiments, the promoter is a eukaryotic promoter.

In some embodiments, the eukaryotic promoter is a CMV promoter, a EF1a promoter, a CAG promoter, a PGK promoter, a TRE promoter, a U6 promoter, or a UAS promoter.

In some embodiments, the promoter is a bacterial promoter.

In some embodiments, the bacterial promoter is a T7 promoter, a T71ac promoter, a Sp6 promoter, a lac promoter, an araBad promoter, a trp promoter, a Ptac promoter, a P1 promoter, a T3 promoter, or a Ptet promoter.

In some embodiments, the Cas13d domain comprises an amino acid sequence of any one of SEQ ID NOs: 1-7, or an amino acid sequence having at least 90% sequence identity with any of SEQ ID NOs: 1-7.

In some embodiments, the zinc finger (ZnF) domain comprises an amino acid sequence of any one of SEQ ID NOs: 8, 10, 12, 14, 16, 18, or 20, or an amino acid sequence having at least 90% sequence identity with any of SEQ ID NOs: 8, 10, 12, 14, 16, 18, or 20.

In some embodiments, the transcriptional repressor domain comprises an amino acid sequence of any one of SEQ ID NOs: 22-24, or an amino acid sequence having at least 90% sequence identity with any of SEQ ID NOs: 22-24.

In some embodiments, the transcriptional repressor domain is a KRAB domain comprising an amino acid sequence of SEQ ID NOs: 24, or an amino acid sequence having at least 90% sequence identity with SEQ ID NOs: 24.

In some embodiments, the Cas13d fusion protein comprises one or more linkers.

In some embodiments, the one or more linkers have an amino acid sequence selected from the group consisting of any of SEQ ID NOs: 25-35, or an amino acid sequence having at least 90% sequence identity with any one of SEQ ID NOs: 25-35.

In some embodiments, the Cas13d fusion protein comprises a linker joining the Cas13d domain and the zinc finger (ZnF) domain.

In some embodiments, the Cas13d fusion protein comprises a linker joining the zinc finger (ZnF) domain and the transcriptional repressor domain.

In some embodiments, the nucleic acid molecule further comprises a first nuclear localization signal (NLS) sequence.

In some embodiments, the nucleic acid molecule further comprises a second nuclear localization signal (NLS) sequence.

In some embodiments, the first NLS is fused to the N-terminus of the Cas13d domain.

In some embodiments, the second NLS is fused to the C-terminus of the Cas13d domain.

In some embodiments, the nucleic acid molecule further comprises a peptide tag.

In some embodiments, the peptide tag is selecting from the group consisting of a His-tag (SEQ ID NO: 51), HA-tag (SEQ ID NO: 49), Flag-tag (SEQ ID NO: 50), a Myc tag (SEQ ID NO: 52), a V5 tag (SEQ ID NO: 53), or an AviTag-PT-6 (SEQ ID NO: 54).

In some embodiments, the nucleic acid molecule comprises SEQ ID NO: 55.

A nucleic acid plasmid comprising the nucleic acid molecule as described above.

A viral vector comprising the nucleic acid molecule as described above.

In some embodiments, the viral vector is an adenovirus vector, an adeno-associated virus vector, or a lentivirus vector.

In some embodiments, the adeno-associated virus vector is an AAV1, AAV2, AAV3, AAV4, AAV5, AAV6, AAV7, AAV8, AAV9 or chimera thereof.

In some embodiments, the nucleic acid plasmid described above, or the viral vector described aboved, further comprises a nucleotide sequence encoding one or more guide RNAs which are capable of targeting the Cas13d domain to a transcript target.

In some embodiments, the transcript target comprises CUG repeat expansions (CUGexp).

In some embodiments, the transcript target encodes a myotonic dystrophy protein kinase comprising CUGexp.

In some embodiments, one or more of the one or more guide RNAs comprise a nucleotide sequence complementary to any one of SEQ ID NO:58-77.

In some embodiments, the present disclosure describes a composition comprising one or more of the nucleic acid molecules as described above, a nucleic acid plasmid as described above, or a viral vector as described above, and a pharmaceutically acceptable excipient.

In some embodiments, the present application describes a composition comprising one or more of a nucleic acid molecule as described above, a nucleic acid plasmid as described above, or a viral vector as described above. In some embodiments, the composition further comprises a nucleic acid molecule encoding one or more guide RNAs which are capable of targeting the Cas13d domain to a transcript target and a pharmaceutically acceptable excipient.

In some embodiments, the present disclosure describes a cell comprising any one of a nucleic acid molecule as described above, a nucleic acid plasmid as described above, a viral vector as described above, and a compositions as described above.

In some embodiments, the present disclosure describes a kit comprising one or more of a nucleic acid molecule as described above, a nucleic acid plasmid as described above, a viral vector as described above, and a composition as described above.

In some embodiments, the present disclosure describes a method of transcript target knockdown using an autoregulated Cas13d domain, comprising expression of any one of a nucleic acid molecule as described above, a nucleic acid plasmid as described above, a viral vector as described above, and a compositions as described above.

In some embodiments, the present disclosure describes a method of transcript target knockdown using an autoregulated Cas13d domain, comprising expression of a nucleic acid molecule comprising in the 5′ to 3′ direction a zinc finger binding site, a promoter, and a nucleotide sequence encoding a Cas13d fusion protein, wherein the Cas13d fusion protein comprises a Cas13d domain, a zinc finger (ZnF) domain, and a transcriptional repressor domain, and wherein the Cas13d fusion protein binds to the zinc finger binding site and represses transcription of the nucleotide sequence encoding the Cas13d fusion protein.

In some embodiments, autoregulation of the Cas13d domain reduces collateral damage.

In some embodiments, the transcript target is knocked down by a greater amount than can be achieved using RNA interference or CRISPR (Cas9) interference.

In some embodiments, the means for autoregulation of the Cas13d domain comprises binding of the Cas13d fusion protein ZnF domain to the ZnF sequence motif, thereby causing the transcriptional repressor domain to inhibit transcription of the sequence encoding the Cas13d fusion protein.

In some embodiments, the present disclosure describes a nucleic acid molecule comprising a nucleotide sequence encoding a Cas13d domain and a guide RNA (gRNA) on a single Cas13d autoregulation transcript.

In some embodiments, the Cas13d domain and the guide RNA are at least 20 base pairs apart.

In some embodiments, the guide RNA is 5′ of the Cas13d domain.

In some embodiments, the gRNA is inserted into an intron of the Cas13d domain.

In some embodiments, the gRNA is inserted into the Cas13d domain.

In some embodiments, the gRNA is inserted into the Cas13d domain at position 143 of SEQ ID NO 57.

In some embodiments, the guide RNA of the nucleic acid encoding the Cas13d autoregulation transcript is located within the Cas13d domain 5′ untranslated region or the Cas13d domain 3′ untranslated region.

In some embodiments, a nucleic acid molecule described above, further comprises a promoter.

In some embodiments, the promoter is an inducible promoter.

In some embodiments, the promoter is a constitutive promoter.

In some embodiments, the promoter is a eukaryotic promoter.

In some embodiments, the eukaryotic promoter is a CMV promoter, a EF1a promoter, a CAG promoter, a PGK promoter, a TRE promoter, a U6 promoter, or a UAS promoter.

In some embodiments, the promoter is a bacterial promoter.

In some embodiments, the bacterial promoter is a T7 promoter, a T71ac promoter, a Sp6 promoter, a lac promoter, an araBad promoter, a trp promoter, a Ptac promoter, a P1 promoter, a T3 promoter, or a Ptet promoter.

In some embodiments, the Cas13d domain comprises an amino acid sequence of any one of SEQ ID NOs: 1-7, or an amino acid sequence having at least 90% sequence identity with any of SEQ ID NOs: 1-7.

In some embodiments, a nucleic acid molecule as described above further comprises a first nuclear localization signal (NLS) sequence.

In some embodiments, a nucleic acid molecule as described above further comprises a second nuclear localization signal (NLS) sequence.

In some embodiments, the first NLS is fused to the n-terminus of the cas13d domain.

In some embodiments, the second NLS is fused to the c-terminus of the cas13d domain.

In some embodiments, a nucleic acid molecule as described above, further comprises a peptide tag.

In some embodiments, the peptide tag is selecting from the group consisting of a His-tag (SEQ ID NO: 51), HA-tag (SEQ ID NO: 49), Flag-tag (SEQ ID NO: 50), a Myc tag (SEQ ID NO: 52), a V5 tag (SEQ ID NO: 53), or an AviTag-PT-6 (SEQ ID NO: 54).

In some embodiments, a nucleic acid molecule as described above, comprises SEQ ID NO: 2.

In some embodiments, a nucleic acid molecule as described above, further comprises a nucleotide sequence encoding one or more additional guide RNAs, wherein the guide RNAs encoded in the nucleic acid molecule are capable of targeting the Cas13d domain to a transcript target.

In some embodiments, the present disclosure describes a nucleic acid plasmid comprising the nucleic acid molecule as described above.

In some embodiments, the present disclosure describes a viral vector comprising a nucleic acid molecule as described above.

In some embodiments, the viral vector is an adenovirus vector, an adeno-associated virus vector, or a lentivirus vector.

In some embodiments, the adeno-associated virus vector is an AAV1, AAV2, AAV3, AAV4, AAV5, AAV6, AAV7, AAV8, AAV9 or chimera thereof.

In some embodiments, the present disclosure describes a nucleic acid molecule of claim as described above, a plasmid as described above, or a vector as described above, wherein the transcript target comprises CUG repeat expansions (CUGexp).

In some embodiments, the present disclosure describes a nucleic acid molecule as described above, a plasmid as described above, or a vector as described above, wherein the transcript target encodes myotonic dystrophy protein kinase and comprises CUG repeat expansions (CUGexp).

In some embodiments, the present disclosure describes a nucleic acid molecule as described above, a plasmid as described above, or a vector as described above, wherein at least one of the one or more guide RNAs comprise a nucleotide sequence capable of targeting of any one of SEQ ID NO: 58-77.

In some embodiments, the present disclosure describes a nucleic acid molecule as described above, a plasmid as described above, or a vector as described above, wherein all the guide RNAs are pre-guide RNAs, each pre-guide RNA comprises a spacer flanked by direct repeat regions.

In some embodiments, the present disclosure describes a composition comprising one or more of a nucleic acid molecules as described above, a nucleic acid plasmid as described above, or a viral vector as described above, and a pharmaceutically acceptable excipient.

In some embodiments, the present disclosure describes a composition comprising one or more of a nucleic acid molecule as described above, a nucleic acid plasmid as described above, or a viral vector as described above. In some embodiments, the composition further comprises a nucleic acid molecule encoding one or more guide RNAs which are capable of targeting the Cas13d domain to a transcript target, and a pharmaceutically acceptable excipient.

In some embodiments, the present disclosure describes a cell comprising any one of a nucleic acid molecule as described above, a nucleic acid plasmid as described above, or the viral vector as described above, and a composition as described above.

In some embodiments, the present disclosure describes a kit comprising one or more of a nucleic acid molecule as described above, a nucleic acid plasmid as described above, or a viral vector as described above, and a compositions as described above.

In some embodiments, the present disclosure describes a method of target transcript knockdown using an autoregulated Cas13d domain, comprising expression of any one of a nucleic acid molecule as described above, a nucleic acid plasmid as described above, a viral vector as described above, and a composition as described above.

In some embodiments, the present disclosure describes a method of target transcript knockdown using an autoregulated Cas13d domain, comprising expression of a nucleic acid molecule comprising in the 5′ to 3′ direction a nucleotide sequence encoding a Cas13d domain and a guide RNA (gRNA) on a single Cas13d autoregulation transcript.

In some embodiments, autoregulation of the Cas13d domain reduces collateral damage.

In some embodiments, the target transcript is knocked down by a greater amount than can be achieved using RNA interference or CRISPR (Cas9) interference.

In some embodiments, the means for autoregulation of Cas13d expression comprises RNA exonuclease degradation of the Cas13d autoregulation transcript following Cas13d protein-dependent cleavage of the sequence encoding the guide RNA.

In some embodiments, the present disclosure describes a method of administering a therapeutically effective amount of a compositions as described above to a subject in need thereof.

In some embodiments, the subject has a disease associated with transcriptional dysregulation.

In some embodiments, the disease is associated with mRNA aggregation.

In some embodiments, the disease is Spinocerebellar ataxia type 12, Fragile X-associated tremor/ataxia syndrome, Neuronal intranuclear inclusion disease, C9ORF72 amyotrophic lateral sclerosis/frontotemporal dementia, Benign adult familial myoclonic epilepsy (familial adult myoclonic epilepsy 1), Cerebellar ataxia, neuropathy, vestibular areflexia syndrome, Myotonic dystrophy type 2, Fuchs endothelial corneal dystrophy, Spinocerebellar ataxia type 10, Spinocerebellar ataxia type 31, Spinocerebellar ataxia type 36 (Asidan, Costa da Morte ataxia), Spinocerebellar ataxia type 37, Dentatorubral-pallidoluysian atrophy (Haw River syndrome, Naito-Oyanagi disease), Huntington's disease, Spinal-bulbar muscular atrophy, Spinocerebellar ataxia type 1, Spinocerebellar ataxia type 2, Spinocerebellar ataxia type 3 (Machado-Joseph disease), Spinocerebellar ataxia type 6, Spinocerebellar ataxia type 7, Spinocerebellar ataxia type 8, Spinocerebellar ataxia type 17, Myotonic dystrophy type 1, Huntington's disease-like 2, Blepharophimosis syndrome, Cleidocranial dysplasia, Congenital central hypoventilation syndrome, Hand-foot-genital syndrome, Holoprosencephaly, Oculopharyngeal muscular dystrophy, Synpolydactyly syndrome, X-linked mental retardation and abnormal genitalia, X-linked mental retardation, X-linked mental retardation and growth hormone deficit, Pseudoachondroplasia and multiple epiphyseal dysplasia, Familial adult myoclonic epilepsy 2, Familial adult myoclonic epilepsy 3, Familial adult myoclonic epilepsy 4, Familial adult myoclonic epilepsy 6, Familial adult myoclonic epilepsy 7, Oculopharyngeal myopathy with leukoencephalopathy, Oculopharyngodistal myopathy 1, or Oculopharyngodistal myopathy 2.

In some embodiments, the disease is muscular dystrophy.

In some embodiments, a viral vector as described above comprises a nucleic acid sequence of any one of SEQ ID NOs: 178-179.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. It is to be understood that the data illustrated in the drawings in no way limit the scope of the disclosure.

FIGS. 1A-1F. Cas13d destroys CUG^{exp} RNA foci and restores MBNL1-mediated alternative splicing in cell models of myotonic dystrophy. FIG. 1A shows an experimental workflow schematic. Plasmids were co-transfected into HeLa and HEK293 cells, incubated for 2-3 days, and analyzed for on- and off-target Cas13d activity. FIG. 1B shows the structure and sequence of target and guide RNAs. Three gRNAs were tested, each of which aligns in a different register to the CUG^{exp} target RNA in DMPK pre-mRNA. SEQ ID NOs: 185 (CUG^{exp} target), 182 (CUG-1), 183 (CUG-2), and 184 (CUG-3) are shown. FIG. 1C depicts images obtained by fluorescence in situ hybridization (FISH) of the CUG^{exp} RNA in HEK293 cells in the presence or absence of CRISPR effector, guide RNA, and a DMPK minigene containing 480 CTG repeats. FIG. 1D shows the effect of different CRISPR approaches on MBNL-mediated splicing in HeLa cells. Exon inclusion (percent spliced in, T) of a minigene reporter was measured by RT-PCR and capillary electrophoresis. FIG. 1E shows off-target specificity of repeat-targeting approaches determined by RNA-seq. Genes were grouped across the transcriptome by the length of their longest CUG repeat, and median differential expression of genes as a function of maximum CUG tract length is plotted. FIG. 1F shows median knockdown of genes with CUG repeat tracts at or longer than the spacer sequence length (22 nt).

FIGS. 2A-2B. Cas13d exhibits collateral damage in human cells. FIG. 2A depicts images showing that reduction of Cas13d expression is observed when transfected with CUG-1 gRNA and CUG^{exp} target in HeLa cells. 10× fluorescence images indicate expression of EGFP, a marker co-translated with Cas13d. FIG. 2B shows measurement of mCherry collateral damage reporter in individual cells by 20× widefield fluorescence microscopy. 61% reduction of mCherry expression is observed when all Cas13 targeting components are present.

FIGS. 3A-3E. Negative autoregulation reduces collateral damage while maintaining on-target efficacy. FIG. 3A shows schematic designs of negative autoregulatory approaches to minimize Cas13d expression. FIG. 3B depicts immunofluorescence images of HA-Cas13d showing reduction of Cas13d expression by Approach 1. FIG. 3C depicts 10× widefield microscopy images of co-translated EGFP marker showing reduction of Cas13 expression by Approach 2. FIG. 3D shows an assessment of on-target efficacy in rescuing MBNL activity in HeLa cells expressing 480 CTG repeats by RT-PCR of MBNL splicing minigene and capillary electrophoresis. FIG. 3E shows 10× widefield microscopy images of mCherry collateral damage reporter.

FIGS. 4A-4F. Cas13d reduces accumulation of (CUG), repeat RNA and rescues MBNL-dependent alternative splicing in a HeLa cell culture model of DM1. FIG. 4A Diagram of cell culture cotransfection experiments to evaluate Cas13d on-target activity and MBNL1-dependent splicing. Plasmids encoding Cas13d and EGFP, gRNA, (CUG)₄₈₀ target RNA, and an MBNL1 exon 5 splicing minigene are transfected into HeLa cells and allowed to express before (CUG). RNA FISH and RT-PCR. FIG. 4B Illustration of gRNAs evaluated in this study, including three gRNAs targeting the (CUG). RNA and a non-targeting (NT) control. SEQ ID NOs: 185 (CUG^{exp} target), 182 (CUG-1), 183 (CUG-2), 184 (CUG-3), and 186 (NT) are shown. FIG. 4C Representative FISH images for (CUG)ₙ RNA in targeting and non-targeting conditions. Cells are stained with DAPI (nuclei) and CellMask (plasma membrane) for segmentation. Scale bars 10 μm. FIG. 4D Median (CUG). FISH signal per nucleus, relative to NT. Error bars indicate 95% CI, estimated by bootstrapping. n>7 nuclei per condition. *p<0.05 (CI overlap). FIG. 4E Diagram of splicing minigene assay. Sequestration of MBNL by (CUG)ₙ RNA increases MBNL1 exon 5 inclusion ratio (ψ). Effective targeting by Cas13d aims to rescue ψ to unperturbed levels. FIG. 4F MBNL1 exon 5ψ for targeting and non-targeting conditions, measured by RT-PCR and capillary electrophoresis. Transcriptional inhibition by deactivated SpCas9 (dCas9) with matching spacer sequences is also shown. Error bars indicate standard deviation. n=3, all conditions. *p<0.05, **p<0.01, ***p<0.001 (two-tailed Student's t test).

FIGS. 5A-5I. Activation of Cas13d reduces expression of orthogonal reporter genes in eukaryotic cells. FIG. 5A Diagram of fluorescence assays to detect collateral activity of Cas13d. HeLa cells are transfected with plasmids encoding Cas13d and EGFP, gRNA, target RNA, and a constitutively expressed orthogonal mCherry reporter. After 20 hr, mCherry fluorescence is measured by plate reader or epifluorescence microscopy. FIG. 5B Interpretation of fluorescence assays. If collateral activity is weak, expression of EGFP and mCherry reporter genes are unaffected by Cas13d activation. If collateral activity is strong, both mCherry and EGFP gene products are depleted upon activation. FIG. 5C Bulk quantitation of mCherry fluorescence by plate reader in (CUG)ₙ-targeting and non-targeting conditions, relative to NT. Error bars indicate standard deviation. n=5, all conditions. ***p<0.001 (two-tailed Student's t test). FIG. 5D Representative images from epifluorescence microscopy of mCherry and EGFP reporter genes in (CUG)ₙ-targeting and non-targeting conditions. Nuclei are stained with DAPI. Scale bars 10 μm. FIG. 5E Quantitation of mCherry fluorescence by epifluorescence microscopy. Measurements from individual cells are shown (grey dots) along with the mean (black line). Error bars indicate SEM. *p<0.05, **p<0.01 (two-sided Mann-Whitney U test). FIG. 5F Bulk mCherry measurement when targeting (CUG)₄₈₀ RNA with Cas13d in HEK293 and Neuro2a cell lines. ***p<0.001 (two-tailed Student's t test). FIG. 5G Bulk mCherry measurement when targeting a transgenic transcript containing 24×MS2 hairpin sequences with Cas13d using MS2-targeting gRNAs. A control plasmid (pUC19) that does not express the MS2 target is also included. ***p<0.001 (two-tailed Student's t test). FIG. 5H Bulk mCherry measurement when targeting a transgenic transcript encoding puromycin acetyltransferase (pac) with Cas13d at unique sites using pac-targeting gRNAs. ***p<0.001 (two-tailed Student's t test). FIG. 5I Relationship between on-target efficacy (rescue of MBNL1 exon 5ψ) and mCherry inhibition for gRNAs targeting unique DMPK sequences present in the (CUG)₄₈₀ transcript. Grey line indicates ordinary least squares linear regression (Pearson r=0.81).

FIGS. 6A-6C. Targeting endogenous mRNAs with Cas13d activates collateral RNAse activity. FIG. 6A Description of the collateral activity RNA assay. The HeLa-tet:Cas13d-mCherry cell line expresses mCherry constitutively and Cas13d under a doxycycline-inducible Tet-On promoter. As collateral activity likely depletes transgenic reporter and endogenous RNAs, non-transgenic HeLa cells are spiked in at a 4:1 ratio prior to transfection of gRNA plasmid and induction of Cas13d expression by doxycycline. After 44 hr, RNA is extracted, and mCherry and control (GAPDH) mRNA expression is measured by RT-qPCR. FIG. 6B Assay validation using (CUG)ₙ-targeting gRNAs and transfected CTG₄₈₀ target plasmid. n=3 biological replicates per condition, averaged across two technical qPCR replicates. Error bars indicate SEM of ΔC_{q} propagated to the plotted ratio. *p<0.05, **p<0.01, ***p<0.001 (one-tailed Student's t test of ΔC_{q}). FIG. 6C Ratio of mCherry to GAPDH mRNA expression observed when targeting endogenous genes. Error bars indicate SEM of ΔC_{q} propagated to the plotted ratio. Grey line depicts a logistic function fit to the data using non-linear least squares regression.

FIGS. 7A-7H. Negative autoregulation by gRNA excision reduces Cas13d collateral activity and retains on-target activity at (CUG)ₙ target RNA. FIG. 7A Diagram describing rationale for negative autoregulation of Cas13d. Overexpression likely produces many copies of Cas13d:gRNA binary complex for each CUGₙ target RNA, activating many molecules of Cas13d for trans cleavage. Reducing Cas13d expression may reduce collateral activity dramatically with minimal loss of on-target activity. FIG. 7B Description of gRNA excision for negative-autoregulatory optimization (GENO) approach to control Cas13d expression. In GENO, the pre-crRNA is located within the UTR of the Cas13d transcript, leading to cleavage and degradation of Cas13d mRNA during crRNA processing. FIG. 7C Simulation of steady-state concentration of Cas13d:gRNA binary complex in autoregulated (solid line) and unregulated (dashed lines) conditions as a function of Cas13d transcription rate (rₜ, horizontal axis) and gRNA transcription rate in the unregulated design (r_{t.gRNA}, isolines). The autoregulation efficiency η_{GENO} is also annotated. Translation rate constant k_{T}=8.3×10⁻³; protein mRNA⁻¹, crRNA processing rate constant k_{proc}=1.7×10⁻⁷ cell molec⁻¹s⁻¹. FIG. 7D Simulation of Cas13d:gRNA complex concentration as a function of translation rate constant (k_{T}, horizontal axis) and crRNA processing rate constant (k_{proc}, isolines). Cas13d transcription rate rₜ=0.02 mRNA s⁻¹, gRNA transcription rate in unregulated design r_{t.gRNA}=0.96 RNA s⁻¹. FIG. 7E Fluorescent Western blot comparing Cas13d expression from unregulated and GENO plasmids transfected into HeLa cells. Cas13d is visualized with anti-HA antibody (green), and HSP70 is stained as a loading control (pink). Protein ladder is shown (L). FIG. 7F Quantification of Cas13d Western blot. Error bars indicate standard deviation. n=3 transfections per condition, ***p<0.001 (two-tailed Student's t test). FIG. 7G mCherry fluorescence 20 hr after transfection of HeLa cells with unregulated (purple) and GENO (green) Cas13d and CUG-1 gRNA plasmids, CUG₄₈₀ target, mCherry, and pUC19 filler plasmid as a function of mass of Cas13d plasmid transfected. Measurements relative to NT gRNA. Error bars indicate standard deviation. n=3 transfections per condition. Logistic functions fit to the data are shown as solid lines. FIG. 7H Splicing inclusion of the MBNL1 exon 5 minigene measured by RT-PCR and capillary electrophoresis. The mass of Cas13d plasmid transfected matches the condition highlighted in (G) (box with dashed lines). Error bars indicate standard deviation. n=3, all conditions. *p<0.05, **p<0.01, ***p<0.001 (two-tailed Student's t test).

FIGS. 8A-8C. gRNA only controls for splicing assay and dCas13d/MBNL1 competition. FIG. 8A MBNL1 exon 5 minigene splicing assay after transfection of HeLa cells with gRNA and target plasmids, in the absence of Cas13d. n=3 transfections per condition. n.s.: not significant, p>0.05, two-tailed Student's t test. FIG. 8B Simultaneous FISH/IF of transfected HeLa cells for CUG₄₈₀ RNA (CAG₁₀ FISH probe, red), dCas13d (anti-HA IF, green), and MBNL1 (anti-MBNL1 IF, blue). Nuclei stained with DAPI (white). Scale bars 10 μm. FIG. 8C Quantification of colocalization of dCas13d and MBNL1 IF signal with nuclear CUG₄₈₀ RNA foci in FISH/IF experiment. n=4 nuclei per condition. *p<0.05, two-sided Mann-Whitney U test.

FIGS. 9A-9C. CUG-targeted Cas13d suppresses EGFP expression and reduces cell viability. FIG. 9A Visualization of EGFP marker on Cas13d plasmid 20 hr after transfection with Cas13d, gRNA, and CUG₄₈₀ target plasmids. PC: phase contrast. Scale bars 20 μm. FIG. 9B Quantification of EGFP expression by plate reader 20 hr after transfection. n=3 transfections per condition. Error bars indicate standard deviation. *p<0.05, two-tailed Student's t test. FIG. 9C Resazurin cell viability assay performed 20 hr and 44 hr after transfection with Cas13d, gRNA, and CUG₄₈₀ target plasmids. n=5 transfections per condition. Error bars indicate standard deviation. *p<0.05, **p<0.01, ***p<0.001, two-tailed Student's t test.

FIGS. 10A-10H. CUG-targeted Cas13d upregulates stress response and apoptosis pathways in HeLa. FIG. 10A Description of RNA-seq experiment to assess transcriptomic changes induced by Cas13d. HeLa cells were transfected with Cas13d, dCas9, or shRNA in CUG-targeting or non-targeting conditions and incubated for 3 days prior to RNA extraction, library preparation, and sequencing. n=3 transfections per condition. Data were processed using kallisto, HISAT2, and DESeq2 for alignment and differential expression (DE) analysis. FIG. 10B Heatmap of correlation coefficients of log 10 TPM between sequencing libraries. FIG. 10C Volcano plots of DESeq2 false discovery rate (FDR)-corrected q-value vs. fold change in targeting and non-targeting conditions. DE genes (FDR q<0.05) are highlighted as red (downregulated) or blue (upregulated). FIG. 10D Plot of median fold change of transcripts in targeting and non-targeting conditions, binned by maximum CUG repeat length within the transcript in the human reference genome. FIG. 10E Median knockdown between targeting and non-targeting conditions of all transcripts containing a CUG repeat longer than the length of the Cas13d spacer (22 nt). FIG. 10F PANTHER gene ontology (GO) analysis of biological processes enriched in the DE genes between CUG-targeting and non-targeting Cas13d conditions. Enriched processes are defined as processes with a ratio of observed to expected genes >5 and FDR q<0.05. For each process, FDR q is plotted on the vertical axis and enrichment is indicated by circle area. Color indicates classification into functional categories. FIG. 10G-H PANTHER GO analysis of processes enriched in DE genes between CUG-targeting and non-targeting shRNA conditions.

FIGS. 11A-11E. Development of HeLa-tet:Cas13d-mCherry cell line and cell viability upon targeting endogenous genes. FIG. 11A Fluorescent Western blot of protein extracted from clonal HeLa cell lines after treatment with lentivirus encoding Cas13d-T2A-EGFP under the constitutive EF1a promoter. Blot stained with anti-HA (green) and anti-HSP70 (red) primary antibodies. Expected MW of Cas13d is 117 kDa, lower bands indicate truncations of Cas13d that retained expression of the downstream EGFP marker. L: protein ladder, pXR001: transient transfection of Cas13d plasmid in HeLa. FIG. 11B Fluorescent Western blot of protein extracted from clonal HeLa cell lines after integration of constitutive mCherry and tetracycline-inducible Cas13d-T2A-EGFP. Expression induced with 2 uM doxycycline for 44 hr prior to protein extraction. Blot stained with anti-HA (green) and anti-HSP70 (red) primary antibodies. ‡ indicates the clone used in subsequent experiments. L: protein ladder, pXR001: transient transfection of Cas13d plasmid in HeLa. FIG. 11C Visualization of EGFP and mCherry before and after 44 hr doxycycline treatment by fluorescence microscopy. PC: phase contrast. Scale bars 20 μm. FIG. 11D Resazurin cell viability assay of HeLa-tet:Cas13d-mCherry cells transfected with plasmids encoding gRNAs targeting endogenous genes and induced with 2 uM doxycycline for 44 hr. n=5 transfections per condition. Error bars indicate standard deviation. *p<0.05, **p<0.01, ***p<0.001, two-tailed Student's t test. FIG. 11E Comparison of cell viability measured 44 hr after Cas13d expression with gRNA targeting endogenous genes vs. depletion of the same genes in a CRISPR essentiality screen in HeLa [2015 Hart]. Pearson correlation coefficient is shown (p>0.05, beta distribution c.d.f.).

FIGS. 12A-12F. Prediction of Cas13d binary complex concentration and autoregulation efficiency from simulation of GENO dynamics. FIG. 12A Equilibrium Cas13d binary complex concentration as a function of transcription rate and crRNA processing rate, for high (left) and low (right) translation rate. FIG. 12B Equilibrium binary complex concentration as a function of translation rate and transcription rate, for high (left) and low (right) crRNA processing rate. FIG. 12C Equilibrium binary complex concentration as a function of crRNA processing rate and translation rate, for high (left) and low (right) transcription rate. FIG. 12D Autoregulation efficiency (ηGENO, defined in Supplemental Note) as a function of transcription rate and crRNA processing rate, for high (left) and low (right) translation rate. FIG. 12E ηGENO as a function of translation rate and transcription rate, for high (left) and low (right) crRNA processing rate. FIG. 12F ηGENO as a function of crRNA processing rate and translation rate, for high (left) and low (right) transcription rate.

FIGS. 13A-13G describe the kinetics and equilibria of a Cas13d negative autoregulation strategy mediated by gRNA excision (GENO) and a corresponding reference model (REF) that does not have Cas13 autoregulation. FIGS. 13A-13C are the equations forming the GENO model. FIGS. 13C-13G are the equations forming the REF model.

FIGS. 14A-14Y show the equilibrium mathematical analysis of the GENO model (FIGS. 14A-14L) and the REF model (FIGS. 14M-14Y).

FIGS. 15A-15B show the mathematical theorem that at equilibrium, negative autoregulation by gRNA excision reduces the expression of the Cas13d mRNA compared to the reference model.

FIGS. 16A-16D shows the mathematical theorem that negative autoregulation by gRNA excision reduces the concentration of active Cas13d:gRNA binary complex compared to the reference model when the GRNA is highly expressed in the reference model and is present in excess and Cas13d protein translation is faster than mRNA degradation.

FIGS. 17A-17G AAV-delivered autoregulated Cas13d reduces CUGn RNA accumulation in human DM1 myoblasts. FIG. 17A Fluorescent Western blot comparing Cas13d protein expression from AAV transduction of patient-derived DM1 myoblasts. Cas13d is visualized with anti-HA antibody, and HSP70 is stained as a loading control. Protein ladder is shown (L). FIG. 17B Representative images from confocal microscopy of AAV-treated DM1 myoblasts stained for Cas13d mRNA (HCR FISH) and protein (α-HA IF, yellow). DAPI was used to visualize the nucleus. Images taken at 20× magnification. FIG. 17C Mean nuclear intensity of Cas13d HCR FISH across nuclei in unregulated and GENO-regulated non-targeting conditions. Dots represent individual nuclei, black line indicates median. n>33 nuclei per condition, >3 images per condition. ***p<0.001, two-sided Mann-Whitney U test. Grey line indicates mean baseline nuclear FISH signal in PBS-treated myoblasts and grey shaded region indicates standard deviation, n=26 nuclei, 3 images. FIG. 17D Mean nuclear intensity of α-HA IF across nuclei in unregulated and GENO-regulated non-targeting conditions. Dots represent individual nuclei, black line indicates median. n>33 nuclei per condition, >3 images per condition. ***p<0.001, two-sided Mann-Whitney U test. Grey line indicates mean baseline nuclear FISH signal in PBS-treated myoblasts and grey shaded region indicates standard deviation. n=26 nuclei, 3 images. FIG. 17E Representative images from confocal microscopy of AAV-treated DM1 myoblasts stained for Cas13d mRNA (HCR FISH, magenta) and CUGn RNA (CAG10-AlexaFluor 594 FISH, greyscale). DAPI was used to visualize the nucleus (cyan). Images taken at 40× magnification. FIG. 17F Mean nuclear intensity of CUG FISH across nuclei in GENO-regulated targeting and non-targeting conditions. Dots represent individual nuclei, black line indicates median. n>43 nuclei per condition, 21 images per condition. *p<0.05, one-sided Mann-Whitney U test. n.s.: not significant (p>0.05). FIG. 17G Cumulative distribution function (c.d.f.) plot of the diffraction-limited FISH spot intensity of individual CUGn RNA foci in GENO-regulated targeting (magenta, solid) and non-targeting (grey, solid) conditions. PBS-treated condition is also shown (grey, dashed). ***p<0.001, two-sided Mann-Whitney U test. n.s.: not significant (p>0.05).

### DETAILED DESCRIPTION

The present disclosure provides autoregulatory systems that have been implemented to control the expression and activity of Cas13d, including the activity of Cas13d in vivo, thereby reducing collateral damage in cells at off-target sites (e.g., non-target messenger RNA transcripts). The disclosure relates in part to the surprising finding that Cas13d causes collateral damage in vivo. These findings are surprising because multiple previous studies have reported that Cas13d does not cause collateral damage in mammalian and plant cells (Konermann Cell 173.3 (2018): 665-676; Mahas et al. Genome biology 20.1 (2019): 1-16). Collateral damage occurs when the Cas13d nuclease targets and cleaves a nucleotide that is not specified by the guide RNA. The results described herein demonstrate that these autoregulatory systems are sufficient to reduce collateral damage while still maintaining robust degradation (or knockdown) of a specific target transcript specified by a Cas13d guide RNA.

In one aspect, the present disclosure provides autoregulatory systems that have been implemented to control the expression and activity of Cas13d, including the activity of Cas13d in vivo. In another aspect, the present disclosure provides an autoregulatory system in the form of a nucleic acid molecule comprising in the 5′ to 3′ direction a zinc finger binding site, a promoter, and a nucleotide sequence encoding a Cas13d fusion protein, wherein the Cas13d fusion protein comprises a Cas13d domain, a zinc finger (ZnF) domain, and a transcriptional repressor domain, and wherein the Cas13d fusion protein binds to the zinc finger binding site and represses transcription of the nucleotide sequence encoding the Cas13d fusion protein. In still another aspect, the present disclosure provides an autoregulatory system in the form of a nucleic acid molecule comprising in the 5′ to 3′ direction a nucleotide sequence encoding a Cas13d domain and a Cas13d processing sequence on a single Cas13d autoregulation transcript.

In other aspects, the disclosure provides a method of transcript target knockdown using an autoregulated Cas13d domain, comprising expression of a nucleic acid molecule comprising in the 5′ to 3′ direction a zinc finger binding site, a promoter, and a nucleotide sequence encoding a Cas13d fusion protein, wherein the Cas13d fusion protein comprises a Cas13d domain, a zinc finger (ZnF) domain, and a transcriptional repressor domain, and wherein the Cas13d fusion protein binds to the zinc finger binding site and represses transcription of the nucleotide sequence encoding the Cas13d fusion protein. In still another aspect, the disclosure provides a method of target transcript knockdown using an autoregulated Cas13d domain, comprising expression of a nucleic acid molecule comprising in the 5′ to 3′ direction a nucleotide sequence encoding a Cas13d domain and a Cas13d processing sequence on a single Cas13d autoregulation transcript.

In various other aspects, the present disclosure provides viral vectors comprising the herein disclosed nucleic acid molecules comprising the Cas13d domain autoregulatory systems. In still other aspects, the present disclosure provides plasmids comprising the herein disclosed nucleic acid molecules comprising the Cas13d domain autoregulatory systems. In other aspects, the disclosure provides compositions comprising nucleic acid molecules, nucleic acid plasmids, or viral vectors described herein, and a pharmaceutically acceptable excipient. In still other aspects, the disclosure provides cells comprising the Cas13d domain autoregulatory systems nucleic acid molecules, plasmids, or vectors described herein. In still other aspects, the disclosure provides kits comprising the Cas13d domain autoregulatory systems nucleic acid molecules, plasmids, or vectors described herein.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

### Adeno-Associated Virus (AAV)

In some embodiments, a Cas13d autoregulating construct (e.g., GENO or a Cas13d fusion protein) is delivered to a target cell using an adeno-associated virus vector. The term, “Adeno-associated virus (AAV), as used herein refers to an adeno-associated virus that is used for delivery of a nucleic acid molecule to a cell. In some embodiments, the AAV may refer to any AAV serotype known in the art for delivering a nucleic acid sequence to a target cell. In some embodiments, the AAV serotype is selected from the group consisting of AAV1, AAV2, AAV3, AAV4, AAV5, AAV6, AAV7, AAV8, or AAV9. In some embodiments, the AAV is a chimeric AAV (i.e., chimera). The term, “Chimeric AAV” refers to an AAV that has a genome from at least two different AAVs serotypes.

### Autoregulating

The term “autoregulating” or“autoregulation” refers to the ability of a protein or Cas13d fusion protein (e.g., Cas13d or Cas13d fusion protein) to control the expression level or activity of itself. The level of autoregulation is determined by comparing the activity or concentration of a protein or Cas13d fusion protein engineered to autoregulate (e.g., anyone of the nucleic acid molecules, constructs, plasmids or vectors herein classified to autoregulate Cas13d protein or Cas13d fusion protein expression) with the activity or concentration of a protein or Cas13d fusion protein that is not engineered to autoregulate. Concentration can be determined directly by measuring the concentration of the Cas13d protein or Cas13d fusion protein or indirectly by measuring the transcript levels associate with the Cas13d protein or the Cas13d fusion protein using tools that are wells known in the art (e.g., fluorescent reporters, immunofluorescence, western blot, qPCR, etc.). In a nonlimiting example, this can occur through negative feedback mechanisms where increases in Cas13d protein or Cas13d fusion protein concentration result in decreased expression (transcription or translation) of the Cas13d or Cas13d fusion protein transcript.

In some embodiments, the level of autoregulation of Cas13 protein or Cas13d fusion protein is a decrease in Cas13d protein or Cas13d fusion protein expression compared to a Cas13d protein or Cas13d fusion protein not capable of autoregulation by at least 10% (e.g., at least 10%, at least 20%, at least 30%, at least 40%, at least 50%, at least 60%, at least 70%, at least 80%, or at least 90%). In some embodiments, the level of autoregulation of Cas13 protein or Cas13d fusion protein is a decrease in Cas13d protein or Cas13d fusion protein expression compared to a Cas13d protein or Cas13d fusion protein not capable of autoregulation by 10% (e.g., 10%, 20%, 30%, 40%, 50%, 60%, 70%, 80%, or 90%). In some embodiments, the level of autoregulation of Cas13 protein or Cas13d fusion protein is a decrease in Cas13d protein or Cas13d fusion protein expression compared to a Cas13d protein or Cas13d fusion protein not capable of autoregulation by 10-30%, 30-50%, 50-70%, or 70-90%.

### Cas13

Cas13 is a class II type VI CRISPR-Cas system that is RNA-guided and comprises RNA-targeting machineries that provide prokaryote immunity against RNA elements, such as RNA phages. All type VI CRISPR-Cas systems have a single effector protein known as the Cas13 effector, previously known as the C2c2 CRISPR-Cas system. To date, four Cas13 protein families have been identified, including Cas13a, Cas13b, Cas13c, and Cas13d. Cas13 effectors possess an RNase activity provided by the two higher eukaryotic and prokaryotic nucleotide-binding domains (HEPN), which is required for target RNA degradation and the processing and maturation of the pre-CRISPR RNA (crRNA) into mature crRNA (64-66-nt in length) to ensure target specificity. By recognizing a short hairpin in the crRNA, the Cas13 protein forms complexes with the guide RNA. Target specificity is determined by a 28-30 nt spacer complementary to the target region via the protospacer-flanking site (PFS) sequence.

### Cas13d

Cas13d is a recently discovered type VI CRISPR RNA editing enzyme (Cas13d (W. X. Yan, “,” Molecular Cell, Apr. 19, 2018, Vol. 70, pp. 327-339, which is incorporated herein by reference). CRISPR is a family of DNA sequences (i.e., CRISPR clusters) in bacteria and archaea that represent snippets of prior infections by a virus that have invaded the prokaryote. The snippets of DNA are used by the prokaryotic cell to detect and destroy DNA from subsequent attacks by similar viruses and effectively compose, along with an array of CRISPR-associated proteins. Cas13d CRISPR comprises two HEPN-1 domains and one HEPN-2 domain, which are responsible for the ribonuclease activity of the enzyme. Cas13d is capable of processing pre-gRNA sequences containing spacers and direct repeat regions without the assistance of helper proteins using the HEPN-2 domain. The HEPN-1 domains are responsible for RNA cleavage. As with other Cas13 family proteins, Cas13d forms a complex with a guide RNA (processed spacer and direct repeat) (also referred a crRNA) and the guide RNA provides specificity to the Cas13d for a target RNA. Upon binding, the Cas13d nuclease cleaves the target RNA resulting in knockdown of gene expression. In some embodiments, Cas13d can be any naturally occurring Cas13d from any organism. In other embodiments, Cas13d can be a variant of any naturally occurring Cas13d from any organism, wherein a variant re one of homologues Eubacterium siraeum DSM15702 (EsCas13d), Anaerobic digester metagenome 15706 (AdmCas13d), Gut metagenome assembly P1E0-k21 (P1E0Cas13d), Ruminococcus albus (RaCas13d), Ruminococcus flavefaciens FD1 (RffCas13d), Ruminococcus flavefaciens XPD3002 (RfxCas13d), Uncultured Ruminococcus species (UrCas13d) and Rumninococcus sp (RxCas13d). In various embodiments, the Cas13d domain comprises an amino acid sequence of any one of SEQ ID NO: 1 (AdmCas13d domain), SEQ ID NO: 2 (EsCas13d domain), SEQ ID NO: 3 (P1E0Cas13d domain), SEQ ID NO: 4 (RaCas13d domain), SEQ ID NO: 5 (RffCas13d domain), SEQ ID NO: 6 (RfxCas13d domain), SEQ ID NO: 7 (UrCas13d domain), or an amino acid sequence having at least 80%, 85%, 90%, 95%, or 99% or more sequence identity with any one of SEQ ID NOs: 1-7.

In various embodiments, the Cas13d domain comprises a nucleic acid sequence of any one of SEQ ID NO: 78 (AdmCas13d domain), SEQ ID NO: 79 (EsCas13d domain), SEQ ID NO: 80 (P1E0Cas13d domain), SEQ ID NO: 81 (RaCas13d domain), SEQ ID NO: 82 (RffCas13d domain), SEQ ID NO: 83 (RfxCas13d domain), SEQ ID NO: 84 (UrCas13d domain), or an amino acid sequence having at least 80%, 85%, 90%, 95%, or 99% or more sequence identity with any one of SEQ ID NOs: 78-84. In various embodiments, the Cas13d domain consists of a nucleic acid sequence of any one of SEQ ID NO: 78 (AdmCas13d domain), SEQ ID NO: 79 (EsCas13d domain), SEQ ID NO: 80 (P1E0Cas13d domain), SEQ ID NO: 81 (RaCas13d domain), SEQ ID NO: 82 (RffCas13d domain), SEQ ID NO: 83 (RfxCas13d domain), SEQ ID NO: 84 (UrCas13d domain).

In some embodiments the Cas13d protein comprises an amino acid sequence that has 85%, 86%, 87%, 88%, 89%, 90%, 91%, 92%, 93%, 94%, 95%, 96%, 97%, 98%, 99%, 99.5%, or 100% sequence identity to any one of SEQ ID NO: 1-7. In some embodiments the Cas13d protein comprises an amino acid sequence that has at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or 100% sequence identity to any one of SEQ ID NO: 1-7. In some embodiments, the Cas13d protein comprises any one of Seq ID NO: 1-7. In some embodiments, the Cas13d protein consists of any one of SEQ ID NO: 1-7.

In some embodiments, the concentration of Cas13d domain is quantified using a fluorescent reporter that is fused to the Cas13d domain. In some embodiments, the concentration of Cas13d protein is quantified using an antibody that is specific for the Cas13d domain, the Cas13d fusion protein, or a peptide tag fused to the Cas13d domain or the Cas13d fusion protein. In some embodiments, the activity of Cas13d protein is quantified using a fluorescent reporter of target transcript knockdown (e.g., targeting a fluorescent protein with Cas13d). In some embodiments, the expression of Cas13d protein is quantified using immunofluorescence.

It is to be understood that the term Cas13d may refer to the Cas13d protein or Cas13d domain found in any species.

### Cas13d Autoregulatory Transcript

The term “Cas13d autoregulatory transcript”, refers to an RNA transcript comprising a nucleic sequence encoding for a Cas13d protein as described herein and a nucleic acid sequence encoding a Cas13d processing sequence as described herein.

### Cas13d Processing Sequence

The term “Cas13d processing sequence” as used herein refers to an RNA sequence that can be bound by a Cas13d protein independent of the guide RNA spacer sequence. In other words, Cas13d does not need to be bound to a guide RNA in order to bind a Cas13d processing sequence. After binding, the Cas13 either cleaves the Cas13d processing sequence or cleaves upstream and/or downstream of the Cas13d processing sequence. In some embodiments, the Cas13d cleaves within 50 nucleotides of the 5′ end of the Cas13 processing sequence. In some embodiments, the Cas13d cleaves within 20 nucleotides of the 5′ end of the Cas13 processing sequence. In some embodiments, the Cas13d cleaves within 5 nucleotides of the 5′ end of the Cas13 processing sequence. In some embodiments, the Cas13d cleaves within 100 nucleotides of the 3′ end of the Cas13 processing sequence. In some embodiments, the Cas13d cleaves within 50 nucleotides of the 3′ end of the Cas13 processing sequence. In some embodiments, the Cas13d cleaves within 20 nucleotides of the 3′ end of the Cas13 processing sequence. In some embodiments, the Cas13d processing sequence comprises a nucleic acid sequence that can be bound by HEPN (a nuclease domain in Cas13d).

In some embodiments, the Cas13d processing sequence comprises a direct repeat. It is known in the art that direct repeat regions form stem loops that are bound by Cas13d (Konermann et al. Cell 173.3 (2018): 665-676 and Yan et al. “Molecular cell 70.2 (2018): 327-339, both of which are incorporated by reference in their entirety). The Cas13d may bind the DR and cleave immediately 5′ of the direct repeat. Cas13d direct repeat sequences are well known in the art, are described in Konermann et al. Cell 173.3 (2018): 665-676, and include SEQ ID NOs: 186-192. In some embodiments, the Cas13d processing sequence comprises a direct repeat of any one of SEQ ID NOs: 186-192, or a variant thereof. In some embodiments, the Cas13d processing sequence comprises a direct repeat having at least at least 80%, 85%, 90%, 95%, or 99% or more sequence identity with any one of SEQ ID NOs: 186-192. In some embodiments, the Cas13 processing sequence comprises a pre-guide RNA. A pre-guide RNA comprises a spacer sequence flanked by two direct repeats (DR) (5′ DR 1-Spacer-DR2 3′). It is known in the art that Cas13d binds to pre-mRNAs independent of being bound to a guide RNA (Konermann et al. Cell 173.3 (2018): 665-676). After binding the pre-guide RNA, the Cas13d HEPN nuclease domains cleave the immediately 5′ of DR1 and between the spacer and DR2 (Konermann et al. Cell 173.3 (2018): 665-676). In some embodiments, the Cas13d binding sequence comprises a pre-guide RNA array that comprises 1, 2, 3, 4, 5, 6, 7, 8, 9, 10, 11, 12, 13, 14, 15, 16, 17, 18, 19, 20 or more pre-guide RNAs.

### Cas13d Fusion Protein

The term “Cas13d fusion protein” as used herein refers to a hybrid polypeptide which comprises a Cas13d protein and at least one protein domain from a protein other than Cas13d. The Cas13d protein or the protein domain may be located at the amino-terminal (N-terminal) portion of the Cas13d fusion protein or at the carboxy-terminal (C-terminal) protein thus forming an “amino-terminal Cas13d fusion protein” or a “carboxy-terminal Cas13d fusion protein,” respectively. In some embodiments, the Cas13d fusion protein comprises a Cas13d protein, a ZnF domain and a KRAB domain. In some embodiments, the Cas13d fusion protein comprises the amino acid sequence of SEQ ID NO: 55. In some embodiments, the Cas13d fusion protein consists of the amino acid sequence of SEQ ID NO: 55. In some embodiments, the nucleic acid sequence encoding the Cas13d fusion protein comprises SEQ ID NO: 56. In some embodiments, the nucleic acid sequence encoding the Cas13d fusion protein consists of SEQ ID NO: 56.

In some embodiments, the concentration of Cas13d fusion protein is quantified using a fluorescent reporter that is fused to the Cas13d fusion protein. In some embodiments, the concentration of Cas13d domain is quantified using an antibody that is specific for the Cas13d domain, the Cas13d fusion protein, or a peptide tag fused to the Cas13d domain or the Cas13d fusion protein. In some embodiments, the concentration of the Cas13d fusion protein is quantified using immunofluorescence. In some embodiments, the activity of Cas13d fusion protein is quantified using a fluorescent reporter of the target transcript (e.g., targeting a fluorescent protein with Cas13d fusion protein). In some embodiments, the activity of Cas13d fusion protein is quantified by measuring the concentration of the target transcript (e.g., qPCR or RNASeq).

### Collateral Damage

As used herein, “collateral damage” refers to Cas13d cleavage of a nucleotide (e.g., RNA or a transcript) that is not specified by the guide RNA. In other words, collateral damage occurs when the Cas-guide RNA complex binds to and cleaves a nucleotide that is not fully complementary (not specified) to the guide RNA. In a non-limiting example of collateral damage, Cas13d targeted to CUG repeat expansions also decreased the expression of a fluorescent marker despite the guide RNA lacking complementarity with the marker. In some embodiments, collateral damage is measured by quantifying off-target knockdown effects on a reporter gene (e.g., a fluorescent reporter) using techniques that are well known in the art (e.g., microscopy or flow cytometry). In some embodiments, Cas13d protein or Cas13d fusion protein collateral damage is quantified by directly measuring off-target effects on mRNA levels using techniques that are well known in the art (RNA sequence, qPCR or Norther blot) and comparing those effects to a catalytically dead Cas13d (dCas13d). In some embodiments Cas13d collateral damage is reduced by autoregulating the expression or activity of the Cas13d protein or the Cas13d fusion protein (i.e., a Cas13d fusion protein comprising a Cas13d domain). In some embodiments, Cas13d autoregulation results in less collateral damage than is observed when using RNAi. In some embodiments, reduction of collateral damage caused by Cas13d protein or a Cas13d fusion protein comprising a Cas13d domain is quantified by measuring the collateral damage with and without autoregulation of Cas13d protein or Cas13d fusion protein expression or activity.

In some embodiments, the level of collateral damage of autoregulated Cas13d protein or a Cas13d fusion protein comprising a Cas13d domain is decreased compared to a Cas13d protein or Cas13d fusion protein not capable of autoregulation by at least 10% (e.g., at least 10%, at least 20%, at least 30%, at least 40%, at least 50%, at least 60%, at least 70%, at least 80%, or at least 90%). In some embodiments, the level of collateral damage associated with autoregulated Cas13d protein or a Cas13d fusion protein comprising a Cas13d domain is a decreased compared to a Cas13d protein or Cas13d fusion protein not capable of autoregulation by 10% (e.g., 10%, 20%, 30%, 40%, 50%, 60%, 70%, 80%, or 90%). In some embodiments, the level of collateral damage of autoregulated Cas13d protein or Cas13d fusion protein is a decreased compared to a Cas13d protein or Cas13d fusion protein not capable of autoregulation by 10-30%, 30-50%, 50-70%, or 70-90%.

### Complementary

As used herein, “complementary” refers to a nucleotide base pairing interaction between two nucleic acid sequences such that guanine is paired with cytosine, and adenosine is paired with thymine or uracil. Unless specified otherwise, complementary refers to nucleic acids having 100% pairing between base pairs (e.g., a guide RNA being 100% complementary to a target transcript). Complementary less than 100% is specifically specified by including a modifier, for example 90% complementary. In some embodiments complementary nucleic acids have at least 50%, at least 60%, at least 70%, at least 80%, at least 90%, at least 95%, or at least 99% pairing between base pairs. In some embodiments complementary nucleic acids have 50%, 60%, 70%, 80%, 90%, 95%, or 99% pairing between base pairs. In some embodiments, the guide RNA spacer region is complementary to a specific transcript. In some embodiments the guide RNA spacer region is at least 50%, at least 60%, at least 70%, at least 80%, at least 90%, at least 95%, or at least 99% complementary with a specific RNA transcript. In some embodiments the guide RNA spacer region is 50%, 60%, 70%, 80%, 90%, 95%, or 99% complementary with a specific RNA transcript. In some embodiments the guide RNA spacer region is 100% complementary with a specific RNA transcript.

### Construct

As used herein, the term “construct” refers to an artificially composed nucleic acid molecule capable of expressing a transcript within a cell. In some embodiment the construct is any one of a plasmid, a vector, a viral vector, e.g., an AAV.

### CUG Repeat Expansions

As used herein, CUG repeat expansions (CUG^{exp}) are CUG repeat mutations which are transcribed with a given gene transcript. The CUG^{exp} can result in aggregation of the gene transcript. CUG^{exp} aggregations can result in numerous diseases that are well known to those of ordinary skill in the art and include myotonic dystrophy, amyotrophic later sclerosis/frontotemporal dementia, Huntington disease and numerous other polyglutamine disorders including Friedreich ataxia and Fragile X syndrome. In some embodiments a construct comprising a Cas13d protein and a means for autoregulating the expression of the Cas13d protein is used to decrease the expression of CUG repeat transcripts associated with the above-mentioned diseases. In some embodiments a construct comprising a Cas13d protein and a means for autoregulating the expression of the Cas13d protein is used to treat the above-mentioned diseases.

### Delivery

In some aspects, the invention provides methods comprising delivering one or more polynucleotides, such as or one or more vectors as described herein, one or more transcripts thereof, and/or one or proteins transcribed therefrom, to a host cell. In some aspects, the invention further provides cells produced by such methods, and organisms (such as animals, plants, or fungi) comprising or produced from such cells. In some embodiments, an autoregulated Cas13d as described herein in combination with (and optionally complexed with) a guide sequence is delivered to a cell.

In some embodiments, the method of delivery provided comprises nucleofection, microinjection, biolistics, virosomes, liposomes, immunoliposomes, polycation or lipid:nucleic acid conjugates, naked DNA, artificial virions, and agent-enhanced uptake of DNA.

Exemplary methods of delivery of nucleic acids include lipofection, nucleofection, electoporation, stable genome integration (e.g., piggybac), microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™, Lipofectin™ and SF Cell Line 4D-Nucleofector X Kit™ (Lonza)). Cationic and neutral lipids that are suitable for efficient receptor-recognition lipofection of polynucleotides include those of Feigner, WO 91/17424; WO 91/16024. Delivery may be to cells (e.g., in vitro or ex vivo administration) or target tissues (e.g., in vivo administration). Delivery may be achieved through the use of RNP complexes.

The preparation of lipid:nucleic acid complexes, including targeted liposomes such as immunolipid complexes, is well known to one of skill in the art (see. e.g., Crystal, Science 270:404-410 (1995); Blaese et al., Cancer Gene Ther. 2:291-297 (1995); Behr et al., Bioconjugate Chem. 5:382-389 (1994): Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

Additional methods for the delivery of nucleic acids to cells are known to those skilled in the art. See, for example, US 2003/0087817, incorporated herein by reference.

Other aspects of the present disclosure provide methods of delivering the Cas13d autoregulatory constructs into a cell. For example, in some embodiments, a cell is contacted with a construct, plasmid or vector described herein (e.g., constructs comprising Cas13d autoregulatory constructs or AAV particles containing nucleic acid vectors comprising such nucleotide sequences). In some embodiments, the contacting results in the delivery of such nucleotide sequences into a cell.

It should be appreciated that any rAAV particle, nucleic acid molecule or composition provided herein may be introduced into the cell in any suitable way, either stably or transiently. In some embodiments, the disclosed proteins may be transfected into the cell. In some embodiments, the cell may be transduced or transfected with a nucleic acid molecule. For example, a cell may be transduced (e.g., with a virus encoding a Cas13d autoregulatory construct), or transfected (e.g., with a plasmid encoding a Cas13d autoregulatory construct) with a nucleic acid molecule, or an rAAV particle containing a viral genome encoding one or more nucleic acid molecules. Such transduction may be a stable or transient transduction. In some embodiments, cells expressing a split protein or containing a split protein may be transduced or transfected with one or more guide RNA sequences. In some embodiments, a plasmid expressing a Cas13d autoregulatory construct may be introduced into cells through electroporation, transient (e.g., lipofection) and stable genome integration (e.g., piggybac) and viral transduction or other methods known to those of skill in the art.

In certain embodiments, the compositions provided herein comprise a lipid and/or polymer. In certain embodiments, the lipid and/or polymer is cationic. The preparation of such lipid particles is well known. See, e.g., U.S. Pat. Nos. 4,880,635; 4,906,477; 4,911,928; 4,917,951; 4,920,016; 4,921,757; and 9,737,604, each of which is incorporated herein by reference.

The compositions of this disclosure may be administered or packaged as a unit dose, for example. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent, i.e., a carrier or vehicle.

Treatment of a disease or disorder includes delaying the development or progression of the disease, or reducing disease severity. Treating the disease does not necessarily require curative results.

As used therein, “delaying” the development of a disease means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset.

As used herein “onset” or “occurrence” of a disease includes initial onset and/or recurrence. Conventional methods, known to those of ordinary skill in the art of medicine, can be used to administer the isolated polypeptide or pharmaceutical composition to the subject, depending upon the type of disease to be treated or the site of the disease.

### Downstream

As used herein, the terms “upstream” and “downstream” are terms of relativity that define the linear position of at least two elements located in a nucleic acid molecule (whether single or double-stranded) that is orientated in a 5′-to-3′ direction. In particular, a first element is upstream of a second element in a nucleic acid molecule where the first element is positioned somewhere that is 5′ to the second element. RNA (double or single stranded), or a hybrid of DNA and RNA. The analysis is the same for single strand nucleic acid molecule and a double strand molecule since the terms upstream and downstream are in reference to only a single strand of a nucleic acid molecule, except that one needs to select which strand of the double stranded molecule is being considered. Often, the strand of a double stranded DNA which can be used to determine the positional relativity of at least two elements is the “sense” or “coding” strand. In genetics, a “sense” strand is the segment within double-stranded DNA that runs from 5′ to 3′, and which is complementary to the antisense strand of DNA, or template strand, which runs from 3′ to 5′.

### Effective Amount

The term “effective amount,” as used herein, refers to an amount of a biologically active agent that is sufficient to elicit a desired biological response. For example, an effective amount of a construct encoding the machinery for cas13d capable of autoregulation would be the amount of construct sufficient to induce knockdown of the target transcript. As will be appreciated by the skilled artisan, the effective amount of an agent, e.g., a Cas13d fusion protein, a nuclease, a hybrid protein, a protein dimer, a complex of a protein (or protein dimer) and a polynucleotide, or a polynucleotide, may vary depending on various factors as, for example, on the desired biological response, e.g., on the specific allele, genome, or target RNA to be degraded, or the cell or tissue being targeted, and/or the agent being used.

### Fluorescent Reporter

As used herein a fluorescent reporter refers to a fluorescent molecule that is expressed in response to a biological event. The fluorescent reporter may be any one of green fluorescent protein (GFP), enhanced green fluorescent protein (eGFP), yellow fluorescent protein (YFP), red fluorescent protein (RPF), cyan fluorescent protein (CFP), Venus, Emerald, Strawberry, mCherry, Alexa or any other fluorescent reporter that is well known to one of ordinary skill in the art. In some embodiments a Cas13d fusion protein comprises a fluorescent protein. In some embodiments cas13d is fused to a fluorescent protein. In some embodiments a fluorescent reporter is used to quantify the expression level of cas13d in a cell. In some embodiments a fluorescent reporter is used to quantify collateral damage.

### Genetic Element

Nucleic acids of the present disclosure may include one or more genetic elements. A “genetic element” refers to a particular nucleotide sequence that has a role in nucleic acid expression (e.g., promoter, enhancer, terminator) or encodes a discrete product of an engineered nucleic acid (e.g., a nucleotide sequence encoding a Cas13d processing sequence, a pre-guide RNA, a guide RNA, a protein and/or an RNA interference molecule).

### GENO

As used here, the term “gRNA excision for negative-autoregulatory optimization (GENO)” refers to a method of autoregulating Cas13d expression. In GENO, a Cas13d protein and a Cas13d processing sequence (e.g., a pre-guide RNA) are encoded in the same transcript. The transcript is capable of being translated to produce Cas13d protein. Without being bound to theory, the Cas13d protein can cleave at or near the Cas13d processing sequence, which exposes the transcript to exonuclease degradation. This decreases the concentration of the transcript and in turn decreases expression of Cas13d protein. In this way Cas13d autoregulates its own expression. The skilled person would understand that a Cas13d protein does not necessarily cleave the transcript from which it was translated, but rather can cleave any transcript comprising the Cas13d processing sequence.

Guide RNA (Pre-Guide RNA, Flanked, Spacer, Direct Repeat, gRNA)

As used herein, the term “guide RNA” is a particular type of guide nucleic acid which is commonly associated with a Cas protein of a CRISPR-Cas13d and which associates with Cas13d, directing the Cas13d protein to a specific sequence in a DNA molecule that includes complementarity to spacer sequence of the guide RNA. A guide RNA can be synthetically designed or naturally occurring. The term guide RNA also embraces the equivalent guide nucleic acid molecules that associate with Cas13d equivalents, homologs, orthologs, or paralogs, whether naturally occurring or non-naturally occurring (e.g., engineered or recombinant), and which otherwise program the Cas13d equivalent to localize to a specific target nucleotide sequence. The Cas13 equivalents may include other type VI CRISPR systems including Cas13a, Cas13b, and Cas13c.

In some embodiments, the construct, vector or plasmid as described herein may comprise 1, 2, 3, 4, 5, 6, 7, 8, 9, 10, 11, 12, 13, 14, 15, 16, 17, 18, 19, or more guide RNAs (i.e., a guide RNA array). In some embodiments, each guide RNA in the guide RNA array targets a different gene. In some embodiments, at least two guide RNAs in the guide RNA array target the same gene.

Guide RNAs may comprise various structural elements that include, but are not limited to:

Spacer sequence—the sequence in the guide RNA which binds to the target nucleic acid sequence (e.g., a target transcript). In some embodiments, a spacer is not naturally occurring. In some embodiments, a spacer and Cas13d do not naturally occur on the same transcript.

Direct repeats (DR)—refers to the sequences flanking the spacer region. The direct repeat may be about 30 nt in length. In some embodiments, the direct repeat comprises a sequence having at least 80%, 85%, 90%, 95%, or 99% or more sequence identity with any one of SEQ ID NOs: 186-192. In some embodiments, the direct repeat comprises a sequence of any one of SEQ ID NOs: 186-192, or a variant thereof. In some embodiments, the direct repeat consists of a sequence of any one of SEQ ID NOs: 186-192.

crRNA—refers to the complex formed when the mature direct repeat and spacer regions bind together. This is also referred to as a guide RNA.

### Host Cell

The term “host cell,” as used herein, refers to a cell that can host, replicate, and express a vector described herein. A host cell may also refer to a cell that is capable of hosting, replicating, and expressing a construct or plasmid described herein.

### Knockdown

As used herein, “knockdown” refers to a decrease in expression of an RNA transcript. The RNA transcript can be a coding or non-coding RNA. Knockdown can occur by decreasing or inhibiting the transcription of DNA into RNA, by degradation of a RNA transcript, or by stopping a transcript from being translated into a protein. Knockdown can be measured by quantifying RNA levels or protein levels using methods that are well known in the art. The term “knockdown” can be used interchangeably with terms well known in the art that to have similar meaning such as “decrease(d) expression”. It is to be understood that one of ordinary skill in the art would readily understand what is mean by expression of a transcript target is knocked down. Knocking down expression of a target transcript may also be referred to as knocking down target gene expression, knocking down target protein expression, decreasing target transcript abundance, decreasing target protein expression, decreasing target gene expression, specific gene knockdown, decreasing expression of a specific gene, decreasing specific gene transcript abundance, gene expression knockdown, transcript expression knockdown, protein expression knockdown or any other synonymous term that is well known in the art.

### Krüppel-Associated Box (KRAB) Domain.

As used herein, “Krüppel-associated box (KRAB) domain” refers to a category of transcriptional repressors domains that are typically associate with ZnF domains. KRAB domains are typically 75 amino acids in length with a minimal repression module being about 45 amino acids. In some embodiments the KRAB domain comprises SEQ ID NO: 24 (KRAB-A).

### Linker

The term “linker” as used herein, refers to a molecule linking two other molecules or moieties. In some embodiments, the linker can be an amino acid sequence in the case of a linker joining proteins or domains within a Cas13d fusion protein. In a non-limiting example, Cas13d can be fused to a ZnF domain or a KRAB domain using a polypeptide linker. In some embodiments, the linker can also be a nucleotide sequence in the case of joining two nucleotide sequences together. In other embodiments, the linker is an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated. In some embodiments the polypeptide linker comprises the sequence (GGGGS)₃ (SEQ ID NO: 27). In some embodiments, the linker comprises a sequence of any one of SEQ ID NOs: 25-35.

### Mitochondrial Localization Sequence

As used herein, “Mitochondrial localization sequence (MLS)” refers to an amino acid sequence that “tags” a protein for import into the mitochondria. In some embodiments, the MLS may be any one of COX4 (SEQ ID NO: 46), TOM7 (SEQ ID NO: 47) or ActA (SEQ ID NO: 48) or an amino acid sequence having at least 80%, 85%, 90%, 95%, or 99% or more sequence identity with any one of SEQ ID NOs: 46-48.

### Muscular Dystrophy

As used herein, “Muscular dystrophy” refers to a group of inherited diseases that damage a subjects muscles overtime. Types of Muscular dystrophy include Duchenne muscular dystrophy, Becker muscular dystrophy, Congenital muscular dystrophy, Myotonic dystrophy, Facioscapulohumeral muscular dystrophy (FSHD), Limb-girdle muscular dystrophy, Oculopharyngeal muscular dystrophy (OPMD), Distal muscular dystrophy, and Emery-Dreifuss muscular dystrophy. Muscular dystrophy can be caused by transcript mutations that add CUG repeat expansions to mRNAs associate with these diseases. In a nonlimiting example, myotonic dystrophy protein kinase contains CUG repeat expansions, which result in protein aggregation. In some embodiments, a Cas13d capable of autoregulation is used to knockdown the expression of a transcript associated with Muscular dystrophy. In some embodiments, a Cas13d capable of autoregulation is used to knockdown the expression of a CUG containing transcript associate with Muscular dystrophy. In some embodiments, a Cas13d capable of autoregulation is used to treat muscular dystrophy.

### Nuclear Localization Signal (NLS)

A “nuclear localization signal” or “NLS” refers to as an amino acid sequence that “tags” a protein for import into the cell nucleus by nuclear transport. Typically, this signal consists of one or more short sequences of positively charged lysines or arginines exposed on the protein surface. One or more NLS may be added to the N- or C-terminus of a protein, or internally (e.g., between two protein domains). For example, one or more NLS may be added to the N- or C-terminus of a Cas13d or a Cas13d fusion protein. In some embodiments, 1, 2, 3, 4, 5, or more NLS may be added. NLS sequences are known in the art and would be apparent to the skilled artisan. For example, NLS sequences are described in Plank et al., PCT/EP2000/011690, filed Nov. 23, 2000, the contents of which are incorporated herein by reference for its disclosure of exemplary nuclear localization sequences. In some embodiments, a NLS comprises a bipartite nuclear localization signal comprising an amino acid sequence selected from the group consisting of KRTADGSEFEPKKKRKV (SEQ ID NO: 36), KRPAATKKAGQAKKKK (SEQ ID NO: 37), KKTELQTTNAENKTKKL (SEQ ID NO: 38), KRGINDRNFWRGENGRKTR (SEQ ID NO: 39), RKSGKIAAIVVKRPRK (SEQ ID NO: 40), PKKKRKV (SEQ ID NO: 41). MSRRRKANPTKLSENAKKLAKEVEN (SEQ ID NO: 42) or MDSLLMNRRKFLYQFKNVRWAKGRRETYLC (SEQ ID NO: 43). In some embodiments, a linker is inserted between the Cas13d protein and the ZnF domain of the Cas13d fusion protein. In certain embodiments, the NLS comprises the amino acid sequence of any one of SEQ ID NO: 36-45 or 85. In some embodiments, the NLS comprises the amino acid sequence of SEQ ID NO: 36-45 or 85.

An NLS can be classified as monopartite or bipartite. A non-limiting example of a monopartite NLS is the sequence PKKKRKV (SEQ ID NO: 41) in the SV40 Large T-antigen. A “bipartite” NLS typically contains two clusters of basic amino acids, separated by a spacer of about 10 amino acids. One non-limiting example of a bipartite NLS is the NLS of nucleoplasmin, KRPAATKKAGQAKKKK (SEQ ID NO: 37). In some embodiments, the NLS used in accordance with the present disclosure is the NLS of nucleoplasmin comprising the amino acid sequence of KRPAATKKAGQAKKKK (SEQ ID NO: 37). Other bipartite NLSs that may be used in accordance with the present disclosure include, without limitation: SV40 bipartite NLS (KRTADGSEFESPKKKRKV (SEQ ID NO: 85), e.g., as described in Hodel et al., J Biol Chem. 2001 Jan. 12; 276(2):1317-25, incorporated herein by reference); Kanadaptin bipartite NLS (KKTELQTTNAENKTKKL (SEQ ID NO: 38), e.g., as described in Hubner et al., Biochem J. 2002 Jan. 15; 361(Pt 2):287-96, incorporated herein by reference); influenza A nucleoprotein bipartite NLS (KRGINDRNFWRGENGRKTR (SEQ ID NO: 39), e.g., as described in Ketha et al., BMC Cell Biology. 2008; 9:22, incorporated herein by reference); and ZO-2 bipartite NLS (RKSGKIAAIVVKRPRK (SEQ ID NO: 40), e.g., as described in Quiros et al., Nusrat A, ed. Molecular Biology of the Cell. 2013; 24(16):2528-2543, incorporated herein by reference).

The nucleotide sequence encoding an NLS is “operably linked” to the nucleotide sequence encoding a protein to which the NLS is fused (e.g., a Cas13d or the Cas13d fusion protein) when two coding sequences are “in-frame with each other” and are translated as a single polypeptide fusing two sequences.

### Nucleic Acid Sequence

The terms “polynucleotide”, “nucleotide sequence”, “nucleic acid”, “nucleic acid molecule”, “nucleic acid sequence”, and “oligonucleotide” refer to a series of nucleotide bases (also called “nucleotides”) in DNA and RNA, and mean any chain of two or more nucleotides. The polynucleotides can be chimeric mixtures or derivatives or modified versions thereof, single-stranded or double-stranded. The oligonucleotide can be modified at the base moiety, sugar moiety, or phosphate backbone, for example, to improve stability of the molecule, its hybridization parameters, etc. The antisense oligonuculeotide may comprise a modified base moiety which is selected from the group including, but not limited to, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid, 5-methyl-2-thiouracil, 3-(3-amino-3-N-2-carboxypropyl) uracil, a thio-guanine, and 2,6-diaminopurine. A nucleotide sequence typically carries genetic information, including the information used by cellular machinery to make proteins and enzymes. These terms include double- or single-stranded genomic and cDNA, RNA, any synthetic and genetically manipulated polynucleotide, and both sense and antisense polynucleotides. This includes single- and double-stranded molecules, i.e., DNA-DNA, DNA-RNA and RNA-RNA hybrids, as well as “protein nucleic acids” (PNAs) formed by conjugating bases to an amino acid backbone. This also includes nucleic acids containing carbohydrate or lipids. Exemplary DNAs include single-stranded DNA (ssDNA), double-stranded DNA (dsDNA), plasmid DNA (pDNA), genomic DNA (gDNA), complementary DNA (cDNA), antisense DNA, chloroplast DNA (ctDNA or cpDNA), microsatellite DNA, mitochondrial DNA (mtDNA or mDNA), kinetoplast DNA (kDNA), provirus, lysogen, repetitive DNA, satellite DNA, and viral DNA. Exemplary RNAs include single-stranded RNA (ssRNA), double-stranded RNA (dsRNA), small interfering RNA (siRNA), messenger RNA (mRNA), precursor messenger RNA (pre-mRNA), small hairpin RNA or short hairpin RNA (shRNA), microRNA (miRNA), guide RNA (gRNA), transfer RNA (tRNA), antisense RNA (asRNA), heterogeneous nuclear RNA (hnRNA), coding RNA, non-coding RNA (ncRNA), long non-coding RNA (long ncRNA or lncRNA), satellite RNA, viral satellite RNA, signal recognition particle RNA, small cytoplasmic RNA, small nuclear RNA (snRNA), ribosomal RNA (rRNA), Piwi-interacting RNA (piRNA), polyinosinic acid, ribozyme, flexizyme, small nucleolar RNA (snoRNA), spliced leader RNA, viral RNA, and viral satellite RNA.

### Peptide Tag

The term “peptide tag (PT)” refers to a peptide amino acid sequence that is genetically fused to a protein sequence to impart one or more functions onto the proteins that facilitate the manipulation of the protein for various purposes, such as, visualization, purification, solubilization, and separation, etc. In some embodiments, peptide tags can include various types of tags categorized by purpose or function, which may include “affinity tags” (to facilitate protein purification), “solubilization tags” (to assist in proper folding of proteins), “chromatography tags” (to alter chromatographic properties of proteins), “epitope tags” (to bind to high affinity antibodies), “fluorescence tags” (to facilitate visualization of proteins in a cell or in vitro).

### Pre-Guide RNA

Use herein a pre-guide RNA refers to a spacer (S) element fused to direct repeats (DR) on both the 3′ and 5′ termini. A pre-gRNA can be both naturally occurring or synthetically produced. Multiple pre-guide RNAs can be bound together in a repeating pattern as follows DR-S-DR-S-DR-S . . . -DR-S (i.e., pre-guide RNA array). In some embodiments, the pre-guide RNA array comprises 1, 2, 3, 4, 5, 6, 7, 8, 9, 10, 11, 12, 13, 14, 15, 16, 17, 18, 19, 20 or more spacers. In some embodiments, each spacer in the pre-guide RNA array targets a different gene. In some embodiments, at least two spacers in the pre-guide RNA array target the same gene. Cas13d is capable of separating spacers and direct repeats by process the pre-gRNA with a nuclease domain. The separated spacer and direct repeat can then form a mature crRNA or a guide RNA, which is capable of binding to the Cas13d protein. In some embodiments, the pre-guide RNA is a Cas13d processing sequence.

### Promoter

A “promoter” refers to a control region of a nucleic acid sequence at which initiation and rate of transcription of the remainder of a nucleic acid sequence are controlled. A promoter may also contain sub-regions at which regulatory proteins and molecules may bind, such as RNA polymerase and other transcription factors. Promoters may be constitutive, inducible, activatable, repressible, tissue-specific, or any combination thereof. A promoter drives expression or drives transcription of the nucleic acid sequence that it regulates. Herein, a promoter is considered to be “operably linked” when it is in a correct functional location and orientation in relation to a nucleic acid sequence it regulates to control (“drive”) transcriptional initiation and/or expression of that sequence.

A promoter may be one naturally associated with a gene or sequence, as may be obtained by isolating the 5′ non-coding sequences located upstream of the coding segment of a given gene or sequence. Such a promoter is referred to as an “endogenous promoter.” In some embodiments, a coding nucleic acid sequence may be positioned under the control of a recombinant or heterologous promoter, which refers to a promoter that is not normally associated with the encoded sequence in its natural environment. Such promoters may include promoters of other genes; promoters isolated from any other cell; and synthetic promoters or enhancers that are not “naturally occurring” such as, for example, those that contain different elements of different transcriptional regulatory regions and/or mutations that alter expression through methods of genetic engineering that are known in the art. In addition to producing nucleic acid sequences of promoters and enhancers synthetically, sequences may be produced using recombinant cloning and/or nucleic acid amplification technology, including polymerase chain reaction (PCR).

In some embodiments, promoters used in accordance with the present disclosure are “inducible promoters,” which are promoters that are characterized by regulating (e.g., initiating or activating) transcriptional activity when in the presence of, influenced by or contacted by an inducer signal. An inducer signal may be endogenous or a normally exogenous condition (e.g., light), compound (e.g., chemical or non-chemical compound) or protein that contacts an inducible promoter in such a way as to be active in regulating transcriptional activity from the inducible promoter. Thus, a “signal that regulates transcription” of a nucleic acid refers to an inducer signal that acts on an inducible promoter. A signal that regulates transcription may activate or inactivate transcription, depending on the regulatory system used. Activation of transcription may involve directly acting on a promoter to drive transcription or indirectly acting on a promoter by inactivation a repressor that is preventing the promoter from driving transcription. Conversely, deactivation of transcription may involve directly acting on a promoter to prevent transcription or indirectly acting on a promoter by activating a repressor that then acts on the promoter. In some embodiments, the promotor is a synthetic promoter, for example as described in Edelman, Gerald M., et al. “PNAS 97.7 (2000): 3038-3043, which is incorporated by reference in its entirety.

A promoter can be constitutively active, meaning that the promoter is always active in a given cellular context, or conditionally active, meaning that the promoter is only active in the presence of a specific condition. For example, a conditional promoter may only be active in the presence of a specific protein that connects a protein associated with a regulatory element in the promoter to the basic transcriptional machinery, or only in the absence of an inhibitory molecule. A subclass of conditionally active promoters are inducible promoters that require the presence of a small molecule “inducer” for activity. Examples of inducible promoters include, but are not limited to, arabinose-inducible promoters, Tet-on promoters, and tamoxifen-inducible promoters. A variety of constitutive, conditional, and inducible promoters are well known to the skilled artisan, and the skilled artisan will be able to ascertain a variety of such promoters useful in carrying out the instant invention, which is not limited in this respect.

### Protein, Peptide, and Polypeptide:

The terms “protein,” “peptide,” and “polypeptide” are used interchangeably herein, and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof. Any of the proteins provided herein may be produced by any method known in the art. For example, the proteins provided herein may be produced via recombinant protein expression and purification, which is especially suited for Cas13d fusion proteins comprising a peptide linker. Methods for recombinant protein expression and purification are well known, and include those described by Green and Sambrook, Molecular Cloning: A Laboratory Manual (4th ed., Cold Spring Harbor Laboratory Press, Cold Spring Harbor. N.Y. (2012)), the entire contents of which are incorporated herein by reference.

### RNA Interference (RNAi)

The term “RNA interference”, refers to a process of RNA degradation that a double stranded RNA, miRNA, siRNA, shRNA or any other type of RNA compatible for targeting the RISC complex to a target RNA. In some embodiments cas13d mediated gene knockdown causes a greater decrease in transcript abundance than RNAi knockdown using shRNA. In some embodiments, cas13d knockdown decreases target gene transcript abundance by at least 10% (e.g., at least 10%, at least 20%, at least 30%, at least 40%, at least 50%, at least 60%, at least 70%, at least 80%, at least 90%, at least 95%, at least 99%). In some embodiments cas13d mediated gene knockdown results in less collateral damage than RNAi gene knockdown using shRNA.

### Subject

The term “subject,” as used herein, refers to an individual organism, for example, an individual mammal. In some embodiments, the subject is a human. In some embodiments, the subject is a non-human mammal. In some embodiments, the subject is a non-human primate. In some embodiments, the subject is a rodent. In some embodiments, the subject is a sheep, a goat, a cow, a cat, or a dog. In some embodiments, the subject is a vertebrate, an amphibian, a reptile, a fish, an insect, a fly, or a nematode. In some embodiments, the subject is a research animal. In some embodiments, the subject is genetically engineered, e.g., a genetically engineered non-human subject. The subject may be of either sex and at any stage of development.

### Target Site

The term “target site” refers to a sequence within a nucleic acid molecule that is edited or cleaved by the Cas13d or Cas13d fusion protein disclosed herein. The target site further refers to the sequence within a nucleic acid molecule (e.g., RNA) to which the Cas13d-gRNA or Cas13d fusion protein-gRNA complex binds. In some embodiments, the target site is any one of SEQ ID NO: 58-77.

### Terminator

A “transcriptional terminator” or “terminator” is a nucleic acid sequence that causes transcription to stop. A transcriptional terminator may be unidirectional or bidirectional. It is comprised of a DNA sequence involved in specific termination of an RNA transcript by an RNA polymerase. A transcriptional terminator sequence prevents transcriptional activation of downstream nucleic acid sequences by upstream promoters. A transcriptional terminator may be necessary in vivo to achieve desirable expression levels or to avoid transcription of certain sequences. A transcriptional terminator is considered to be “operably linked to” a nucleotide sequence when it is able to terminate the transcription of the sequence it is linked to.

The most commonly used type of terminator is a forward terminator. When placed downstream of a nucleic acid sequence that is usually transcribed, a forward transcriptional terminator will cause transcription to abort. In some embodiments, bidirectional transcriptional terminators are provided, which usually cause transcription to terminate on both the forward and reverse strand. In some embodiments, reverse transcriptional terminators are provided, which usually terminate transcription on the reverse strand only.

In prokaryotic systems, terminators usually fall into two categories (1) rho-independent terminators and (2) rho-dependent terminators. Rho-independent terminators are generally composed of palindromic sequence that forms a stem loop rich in G-C base pairs followed by several T bases. Without wishing to be bound by theory, the conventional model of transcriptional termination is that the stem loop causes RNA polymerase to pause, and transcription of the poly-A tail causes the RNA:DNA duplex to unwind and dissociate from RNA polymerase.

In eukaryotic systems, the terminator region may comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in some embodiments involving eukaryotes, a terminator may comprise a signal for the cleavage of the RNA. In some embodiments, the terminator signal promotes polyadenylation of the message. The terminator and/or polyadenylation site elements may serve to enhance output nucleic acid levels and/or to minimize read through between nucleic acids.

Terminators for use in accordance with the present disclosure include any terminator of transcription described herein or known to one of ordinary skill in the art. Examples of terminators include, without limitation, the termination sequences of genes such as, for example, the bovine growth hormone terminator, and viral termination sequences such as, for example, the SV40 terminator, spy, yejM, secG-leuU, thrLABC, rrnB T1, hisLGDCBHAFI, metZWV, rrnC, xapR, aspA and arcA terminator. In some embodiments, the termination signal may be a sequence that cannot be transcribed or translated, such as those resulting from a sequence truncation.

### Therapeutically Effective Amount

“A therapeutically effective amount” as used herein refers to the amount of each therapeutic agent (e.g., a construct encoding a Cas13D capable of autoregulation, or an AAV encoding said construct) described in the present disclosure required to confer therapeutic effect on the subject, either alone or in combination with one or more other therapeutic agents. Effective amounts vary, as recognized by those skilled in the art, depending on the particular condition being treated, the severity of the condition, the individual subject parameters including age, physical condition, size, gender, and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a subject may insist upon a lower dose or tolerable dose for medical reasons, psychological reasons or for virtually any other reasons. Empirical considerations, such as the half-life, generally will contribute to the determination of the dosage. For example, therapeutic agents that are compatible with the human immune system, such as polypeptides comprising regions from humanized antibodies or fully human antibodies, may be used to prolong half-life of the polypeptide and to prevent the polypeptide being attacked by the host's immune system.

### Transcript

As described herein a “transcript” refers to an RNA molecule that has been transcribed from DNA. In some embodiments a transcript is anyone of a messenger RNA (mRNA), a ribosomal RNA (rRNA), a transfer RNA (tRNA), a transfer RNA (tRNA), a small nuclear RNA (snRNA), a non-coding RNA, a long non-coding RNA (lncRNA), a regulatory RNA such as a silencing RNA (siRNA) or a microRNA (miRNA). In some embodiments the transcript comprises introns and exons. In some embodiments the transcript does not comprise an exon. In some embodiments the transcript comprises one or more exon. In some embodiments the transcript comprises spliceosome sites. In some embodiments the transcript is double stranded RNA.

### Treat

The terms “treatment,” “treat,” and “treating,” refer to a clinical intervention aimed to reverse, alleviate, delay the onset of, or inhibit the progress of a disease or disorder, or one or more symptoms thereof, as described herein. As used herein, the terms “treatment,” “treat,” and “treating” refer to a clinical intervention aimed to reverse, alleviate, delay the onset of, or inhibit the progress of a disease or disorder, or one or more symptoms thereof, as described herein. In some embodiments, treatment may be administered after one or more symptoms have developed and/or after a disease has been diagnosed. In other embodiments, treatment may be administered in the absence of symptoms, e.g., to prevent or delay onset of a symptom or inhibit onset or progression of a disease. For example, treatment may be administered to a susceptible individual prior to the onset of symptoms (e.g., in light of a history of symptoms and/or in light of genetic or other susceptibility factors). Treatment may also be continued after symptoms have resolved, for example, to prevent or delay their recurrence.

### Upstream

As used herein, the terms “upstream” and “downstream” are terms of relativity that define the linear position of at least two elements located in a nucleic acid molecule (whether single or double-stranded) that is orientated in a 5′-to-3′ direction. In particular, a first element is upstream of a second element in a nucleic acid molecule where the first element is positioned somewhere that is 5′ to the second element. RNA (double or single stranded), or a hybrid of DNA and RNA. The analysis is the same for single strand nucleic acid molecule and a double strand molecule since the terms upstream and downstream are in reference to only a single strand of a nucleic acid molecule, except that one needs to select which strand of the double stranded molecule is being considered. Often, the strand of a double stranded DNA which can be used to determine the positional relativity of at least two elements is the “sense” or “coding” strand. In genetics, a “sense” strand is the segment within double-stranded DNA that runs from 5′ to 3′, and which is complementary to the antisense strand of DNA, or template strand, which runs from 3′ to 5′.

### Wild Type, Mutant, and Variant

As used herein the term “wild type” is a term of the art understood by skilled persons and means the typical form of an organism, strain, gene or characteristic as it occurs in nature as distinguished from mutant or variant forms. A “mutant” refers to a version of a wild type protein or nucleic acid molecule in which one or more amino acid residues or nucleotide base pairs, as the case may be, has been changed. The changed one or more amino acid residues or nucleotide base pairs, as the case may be, are referred to as “mutations.” Mutations may include point mutations or “polymorphisms” (including substitutions, insertions, or deletions), chromosomal mutations (e.g., inversions, deletion of a region, duplications, and translocations), and copy number variations (e.g., gene amplifications, expanding trinucleotide repeats). The process of introducing one or more mutations typically involves introducing changes to the genetic information at the nucleic acid level and can result from any naturally occurring process (e.g., exposure to mutagens, during replication of DNA, or exposure to ultraviolet light or radiation) or experimentally by using one or more laboratory procedures (e.g., directed mutagenesis, site-directed mutagenesis, PCR-based mutagenesis, insertional mutagenesis, transposon-based mutagenesis, mutational evolutional processes. The following references relating to mutagenesis are incorporated herein by reference: McCullum et al., “Random mutagenesis by error-prone PCR,” Methods Mol Biol., 2010, 634: 103-9; Bose et al., “Chemical and UV mutagenesis,” Methods Mol Biol., 2016, 1373: 111-115: Cadwell et al., Randomization of genes by PCR mutagenesis,” PCR Methods Appl., 1992, 2(1): 28-33; and Malinovska et al., “Step-by-step in vitro mutagenesis; Lessons from Fucose-Binding Lectin PA-IIL,” Method Mol Biol, 2017; 1498: 399-419. The terms “mutant” and “variant” may be used interchangeably with respect to a protein sequence, however, the term “variant” refers more commonly to protein variants or “isoforms” that arise with some frequency in a population as a result of a mutation occurring in a gene or gene family, or another genetic difference, such as alternative splicings, alternative promoter usage, or other post-transcriptional modifications, or post-translational modifications (PTMs). PTMs are chemical modifications that play a key role in functional proteomic because they regulate activity, localization, and interaction with other cellular molecules such as proteins, nucleic acids, lipids and cofactors. PTMs occur at distinct amino acid side chains or peptide linkages, and they are most often mediated by enzymatic activity. It is estimated that 5% of the proteome comprises enzymes that perform more than 200 types of post-translational modifications. These enzymes include kinases, phosphatases, transferases and ligases, which add or remove functional groups, proteins, lipids or sugars to or from amino acid side chains; and proteases, which cleave peptide bonds to remove specific sequences or regulatory subunits. Many proteins can also modify themselves using autocatalytic domains, such as autokinase and autoproteolytic domains. Post-translational modification can occur at any step in the “life cycle” of a protein. For example, many proteins are modified shortly after translation is completed to mediate proper protein folding or stability or to direct the nascent protein to distinct cellular compartments (e.g., nucleus, membrane). Other modifications occur after folding and localization are completed to activate or inactivate catalytic activity or to otherwise influence the biological activity of the protein. Proteins are also covalently linked to tags that target a protein for degradation. Besides single modifications, proteins are often modified through a combination of post-translational cleavage and the addition of functional groups through a step-wise mechanism of protein maturation or activation.

### Zinc Finger (Domain and Binding Sequence)

As used herein, the term “zinc finger,” “zinc finger domain,” or “ZnF or ZnF domain,” refers to zinc finger polypeptides that bind to DNA. These zinc finger domains are well known to those or ordinary skill in the art. In some embodiments a ZnF domain in the Cas13d fusion protein is selected from, but not limited to, any one of the following HLTR3, HLTR1, HLTR6, mTYR, mCFTR, hDMPK, and hCCR5. In some embodiments a ZnF domain in the Cas13d fusion protein is selected from any one SEQ ID NO: 8, 10, 12, 14, 16, 18, or 20. The following references relating to and disclosing zinc finger proteins are incorporated herein by reference in their entireties: (1) Ecco G, Imbeault M, Trono D. KRAB zinc finger proteins. Development. 2017 Aug. 1; 144(15):2719-2729. doi: 10.1242/dev.132605. PMID: 28765213: PMCID: PMC7117961; (2) Laity J H, Lee B M, Wright P E. Zinc finger proteins: new insights into structural and functional diversity. Curr Opin Struct Biol. 2001 February; 11(1):39-46. doi: 10.1016/s0959-440x(00)00167-6. PMID: 11179890; (3) Eom K S, Cheong J S, Lee S J. Structural Analyses of Zinc Finger Domains for Specific Interactions with DNA. J Microbiol Biotechnol. 2016 Dec. 28; 26(12):2019-2029. doi: 10.4014/jmb.1609.09021. PMID: 27713215; (4) Wolfe S A, Nekludova L, Pabo C O. DNA recognition by Cys2His2 zinc finger proteins. Annu Rev Biophys Biomol Struct. 2000; 29:183-212. doi: 10.1146/annurev.biophys.29.1.183. PMID: 10940247; (5) Wang J, Wang J, Tian C Y. Evolution of KRAB-containing zinc finger proteins and their roles in species evolution. Yi Chuan. 2016 Nov. 20; 38(11):971-978. doi: 10.16288/j.yczz.16-056. PMID: 27867147; (6) Papworth M, Kolasinska P, Minczuk M. Designer zinc-finger proteins and their applications. Gene. 2006 Jan. 17; 366(1):27-38. doi: 10.1016/j.gene.2005.09.011. Epub 2005 Nov. 17. PMID: 16298089; (7) Negi S, Imanishi M. Matsumoto M, Sugiura Y. New redesigned zinc-finger proteins: design strategy and its application. Chemistry. 2008; 14(11):3236-49. doi: 10.1002/chem.200701320. PMID: 18236477; (8) Urrutia R. KRAB-containing zinc-finger repressor proteins. Genome Biol. 2003; 4(10):231. doi: 10.1186/gb-2003-4-10-231. Epub 2003 Sep. 23. PMID: 14519192, PMCID: PMC328446; and (9) Klug A. The discovery of zinc fingers and their applications in gene regulation and genome manipulation. Annu Rev Biochem. 2010; 79:213-31. Doi: 10.1146/annurev-biochem-010909-095056. PMID: 20192761.

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Cas13d Autoregulating Constructs

In one aspect, the present disclosure provides Cas13d autoregulating constructs which generally refer to recombinant nucleic acid molecules which comprise a nucleotide sequence encoding a Cas13d domain and one or more regulatory elements which result in the autoregulation of the expression and/or activity of the Cas13d domain. Without being bound by theory, the autoregulation of Cas13d reduces or eliminates the collateral damage associated with Cas13d activity in a cell, which prior to the present disclosure, was an unrecognized property of Cas13d. The present disclosure contemplates any suitable means to autoregulate the expression and/or activity of Cas13d.

In some embodiments the Cas13d domain being autoregulated comprises any one of Eubacterium siraeum DSM15702 (EsCas13d), Anaerobic digester metagenome 15706 (AdmCas13d), Gut metagenome assembly P1E0-k21 (P1E0Cas13d), Ruminococcus albus (RaCas13d), Rumninococcus flavefaciens FD1 (RffCas13d), Ruminococcus flavefaciens XPD3002 (RfxCas13d), Uncultured Ruminococcus species (UrCas13d) and Ruminococcus sp (RxCas13d). In some embodiments the Cas13d domain being autoregulated comprises any one of SEQ ID NO: 1-7. In some embodiments, the Cas13d domain being autoregulated comprises an amino acid sequence that has 85%, 86%, 87%, 88%, 89%, 90%, 91%, 92%, 93%, 94%, 95%, 96%, 97%, 98%, 99%, 99.5%, or 100% sequence identity to any one of SEQ ID NO: 1-7. In some embodiments, the Cas13d domain being autoregulated comprises an amino acid sequence that has at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5%, sequence identity to any one of SEQ ID NO: 1-7. In some embodiments, the Cas13d domain being autoregulated comprises an amino acid sequence of any one of SEQ ID NO: 1-7. In some embodiments, the Cas13d domain being autoregulated consists of an amino acid sequence of any one of SEQ ID NO: 1-7. In some embodiments, the Cas13d being autoregulated is RfxCas13d (SEQ ID NO: 6).

In some embodiments, the concentration of the Cas13d domain is quantified using a fluorescent reporter that is fused to the Cas13d domain. In some embodiments, the concentration of Cas13d domain is quantified using an antibody that is specific for the Cas13d domain, the Cas13d fusion protein, or a peptide tag fused to the Cas13d domain or the Cas13d fusion protein. In some embodiments, the activity of Cas13d domain is quantified using a fluorescent reporter of target transcript knockdown (e.g., targeting a fluorescent protein with Cas13d). In some embodiments, the expression of Cas13d protein is quantified using immunofluorescence.

In some embodiments, the activity of Cas13d domain is quantified using a fluorescent reporter of target transcript knockdown (e.g., targeting a fluorescent protein transcript with Cas13d). In some embodiments, the expression of Cas13d protein is quantified using immunofluorescence. However, any suitable method known to those of ordinary skill in the art can be used to evaluate, measure, or detect the activity of Cas13d or Cas13d fusion protein described herein.

In some embodiments, the nucleic acid encoding a Cas13d domain or a Cas13d fusion protein may comprise a linker, a peptide tag or a nuclear localization sequence. In some embodiments, the linker can be an amino acid sequence in the case of a linker joining proteins or domains within a Cas13d fusion protein. In a non-limiting example, Cas13d can be fused to a ZnF domain or a KRAB domain using a polypeptide linker. In some embodiments, the linker can also be a nucleotide sequence in the case of joining two nucleotide sequences together. In other embodiments, the linker is an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated. In some embodiments the polypeptide linker comprises the sequence (GGGGS)₃ (SEQ ID NO: 27).

In some embodiments, peptide tags can include various types of tags categorized by purpose or function, which may include “affinity tags” (to facilitate protein purification), “solubilization tags” (to assist in proper folding of proteins), “chromatography tags” (to alter chromatographic properties of proteins), “epitope tags” (to bind to high affinity antibodies), “fluorescence tags” (to facilitate visualization of proteins in a cell or in vitro).

In some embodiments, one or more NLS may be added to the N- or C-terminus of a protein, or internally (e.g., between two protein domains). In a non-limiting example, one or more NLS may be added to the N- or C-terminus of a Cas13d domain or the Cas13d fusion protein. In some embodiments, 1, 2, 3, 4, 5, or more NLS may be added. In some embodiments, a NLS comprises a bipartite nuclear localization signal comprising an amino acid sequence selected from the group consisting of SEQ ID NO: 36-45.

In some embodiments, one or more MLS may be added to the N- or C-terminus of a protein, or internally (e.g., between two protein domains). In a non-limiting example, one or more MLS may be added to the N- or C-terminus of the Cas13d domain, the ZnF domain, the transcriptional repressor domain, or the Cas13d fusion protein. In some embodiments, 1, 2, 3, 4, 5, or more MLS may be added. In some embodiments, a MLS comprises an amino acid sequence selected from the group consisting of SEQ ID NO: 46-48.

In some embodiments, a Cas13d domain or ta Cas13d fusion protein may comprise a peptide tag. In some embodiments, peptide tags can include various types of tags categorized by purpose or function, which may include “affinity tags” (to facilitate protein purification), “solubilization tags” (to assist in proper folding of proteins), “chromatography tags” (to alter chromatographic properties of proteins), “epitope tags” (to bind to high affinity antibodies), “fluorescence tags” (to facilitate visualization of proteins in a cell or in vitro). In some embodiments, the peptide tag may be a HA tag, a FLAG tag, a His tag, a Myc tag, a V5 tag, or a AviTag. In some embodiments, the Cas13d domain or the Cas13d fusion protein comprises a peptide tag, wherein the peptide tag comprises any one of a His-tag (SEQ ID NO: 51), HA-tag (SEQ ID NO: 49), Flag-tag (SEQ ID NO: 50), a Myc tag (SEQ ID NO: 52), a V5 tag (SEQ ID NO: 53), or an AviTag-PT-6 (SEQ ID NO: 54). In some embodiments, the Cas13d domain or the Cas13d fusion protein comprises a peptide tag, wherein the peptide tag consists of any one of a His-tag (SEQ ID NO: 51), HA-tag (SEQ ID NO: 49), Flag-tag (SEQ ID NO: 50), a Myc tag (SEQ ID NO: 52), a V5 tag (SEQ ID NO: 53), or an AviTag-PT-6 (SEQ ID NO: 54).

### Cas13d Fusion Proteins

In some embodiments, the present disclosure relates to Cas13d fusion proteins that comprise a Cas13d protein and a DNA binding domain. For example, the autoregulatory system may be in the form of a nucleic acid molecule comprising a zinc finger binding site, a promoter, and a nucleotide sequence encoding a Cas13d fusion protein, wherein the Cas13d fusion protein comprises a Cas13d domain, a zinc finger (ZnF) domain, and a transcriptional repressor domain, and wherein the Cas13d fusion protein binds to the zinc finger binding site and represses transcription of the nucleotide sequence encoding the Cas13d fusion protein. For example, the autoregulatory system may be in the form of a nucleic acid molecule comprising in the 5′ to 3′ direction a zinc finger binding site, a promoter, and a nucleotide sequence encoding a Cas13d fusion protein, wherein the Cas13d fusion protein comprises a Cas13d domain, a zinc finger (ZnF) domain, and a transcriptional repressor domain, and wherein the Cas13d fusion protein binds to the zinc finger binding site and represses transcription of the nucleotide sequence encoding the Cas13d fusion protein.

In some embodiments, the Cas13d fusion protein comprises an amino acid sequence that has 85%, 86%, 87%, 88%, 89%, 90%, 91%, 92%, 93%, 94%, 95%, 96%, 97%, 98%, 99%, 99.5%, or 100% sequence identity to SEQ ID NO: 56. In some embodiments, the Cas13d fusion protein comprises an amino acid sequence that has at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5%, sequence identity to any one of SEQ ID NO:56. In some embodiments, the Cas13d fusion protein comprises an amino acid sequence of any one of SEQ ID NO: 56. In some embodiments, the Cas13d fusion protein consists of an amino acid sequence of any one of SEQ ID NO: 56. In some embodiments, the nucleic acid molecule encoding Cas13d fusion protein comprises a nucleic acid sequence that has 85%, 86%, 87%, 88%, 89% 0.90%, 91%, 92%, 93%, 94%, 95%, 96%, 97%, 98%, 99%, 99.5%, or 100% sequence identity to SEQ ID NO: 55. In some embodiments, the nucleic acid molecule encoding Cas13d fusion protein comprises an a nucleotide sequence that has at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5%, sequence identity to any one of SEQ ID NO: 55. In some embodiments, the nucleic acid molecule encoding Cas13d fusion protein comprises a nucleic acid sequence of SEQ ID NO: 56. In some embodiments, the nucleic acid molecule encoding Cas13d fusion protein comprises a nucleic acid sequence of any one of SEQ ID NO: 55.

In some embodiments, the zinc finger domain of the Cas13d fusion protein comprises any one of SEQ ID NO: 8, 10, 12, 14, 16, 18, and 20. In various embodiments, the zinc finger domain comprises an amino acid sequence of any one of SEQ ID NO: 8 (HLTR3 ZnF), SEQ ID NO: 10 (HLTR1 ZnF), SEQ ID NO: 12 (HLTR6 ZnF), SEQ ID NO: 16 (mCFTR ZnF), SEQ ID NO: 18 (hDMPK ZnF), and SEQ ID NO: 20 (hCCR5 ZnF), or an amino acid sequence having at least 80%, 85%, 90%, 95%, or 99% or more sequence identity with any one of SEQ ID NOs: 8, 10, 12, 14, 16, 18 or 20. In some embodiments, the ZnF domain of the Cas13d fusion protein is selected from any one of ZnF domains that are well known in the art.

In some embodiments the zinc finger domain of the Cas13d fusion protein comprises an amino acid sequence that has 85%, 86%, 87%, 88%, 89%, 90%, 91%, 92%, 93%, 94%, 95%, 96%, 97%, 98%, 99%, 99.5%, or 100% sequence identity to any one of SEQ ID NO: 8, 10, 12, 14, 16, 18 or 20. In some embodiments the zinc finger domain of the Cas13d fusion protein comprises an amino acid sequence that has at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or at least 100% sequence identity to any one of SEQ ID NO: 8, 10, 12, 14, 16, 18 or 20. In some embodiments the zinc finger domain of the Cas13d fusion protein comprises an amino acid sequence of any one of SEQ ID NO: 8, 10, 12, 14, 16, 18 or 20. In some embodiments the zinc finger domain of the Cas13d fusion protein consists of an amino acid sequence of any one of SEQ ID NO: 8, 10, 12, 14, 16, 18 or 20. In some embodiments the zinc finger domain of the Cas13d fusion protein is synthetically modified to alter binding to DNA motifs.

In some embodiments, a linker is inserted between the Cas13d protein and the ZnF domain of the Cas13d fusion protein. In certain embodiments, the linker comprises the amino acid sequence of any one of SEQ ID NO: 25-35.

In certain other embodiments, an alternative domain to the zinc finger binding domain may be used to achieve the same effect. For example, any suitable DNA binding domain that is capable of recognizing and binding to a specific recognition site including engineered Transcription Activator-Like Effectors (TALEs) and repurposed CRISPR systems, including CRISPR Cas9 systems. TALEs and CRISPR Cas9 systems are known in the art.

Other suitable DNA binding proteins and associated binding sites that could be used in the autoregulatory constructs provided herein can include, but are not limited to:

TABLE 1

| | | | SEQ ID NO |
|---|---|---|---|
| | | | (Binding |
| iGem Name | Domain Description | Binding sequence | Sequence) |
| BBa_K105004 | cI-repressor from | 1. taacaccgtg cgtgttgact | 86 |
| | E.coli phage lambda | attttacctc tggcggtgat aatggttgc | |
| | | 2. tttactatca ccgccagagg taacgta | 180 |
| BBa_K105007 | Gal4-DNA binding | tttaccggag gacagtactc cgacgta | 87 |
| | domain | | |
| BBa_K747000 | TAL- | TAA NN NN NN NN NN T | 88 |
| | Protein_AA1_DiRepeat | | |
| BBa_K747016 | TAL- | TNN AA NN NN NN NN T | 89 |
| | Protein_AA2_Direpeat | | |
| BBa_K747032 | TAL- | T NN NN AA NN NN NN T | 90 |
| | Protein_AA3_Direpeat | | |
| BBa_K747048 | TAL- | T NN NN NN AA NN NN T | 91 |
| | Protein_AA4_Direpeat | | |
| BBa_K747064 | TAL- | TNN NN NN NN AA NN T | 92 |
| | Protein_AA5_Direpeat | | |
| BBa_K747080 | TAL- | T NN NN NN NN NN AA T | 93 |
| | Protein_AA6_Direpeat | | |
| BBa_K747017 | TAL- | TNN AC NN NN NN NN T | 94 |
| | Protein_AC2_Direpeat | | |
| BBa_K747033 | TAL- | T NN NN AC NN NN NN T | 95 |
| | Protein_AC3_Direpeat | | |
| BBa_K747049 | TAL | TNNNN NN ACNN NN T | 96 |
| | Protein_AC4_Direpeat | | |
| BBa_K747065 | TAL- | T NN NN NN NN ACNN T | 97 |
| | Protein_AC5_Direpeat | | |
| BBa_K747081 | TAL- | T NN NN NN NN NN AC T | 98 |
| | Protein_AC6_Direpeat | | |
| BBa_K747002 | TAL- | T NN AG NN NN NN NN T | 99 |
| | Protein_AG1_Direpeat | | |
| BBa_K747018 | TAL- | TNN NN AG NN NN NN T | 100 |
| | Protein_AG2_Direpeat | | |
| BBa_K747034 | TAL- | T NN NN NN AG NN NN T | 101 |
| | Protein_AG3_Direpeat | | |
| BBa_K747050 | TAL- | T NN NN NN NN AG NN T | 102 |
| | Protein_AG4_Direpeat | | |
| BBa_K747082 | TAL- | T NN NN NN NN NN AG T | 103 |
| | Protein_AG6_Direpea | | |
| BBa_K747019 | TAL- | TNN AT NN NN NN NN T | 104 |
| | Protein_AT2_Direpeat | | |
| BBa_K747035 | TAL- | TNN NN AT NN NN NN T | 105 |
| | Protein_AT3_Direpeat | | |
| BBa_K747051 | TAL | TNN NN NN AT NN NN T | 106 |
| | Protein_AT4_Direpeat | | |
| BBa_K747067 | TAL- | TNN NN NN NN AT NN T | 107 |
| | Protein_AT5_Direpeat | | |
| BBa_K747083 | TAL- | T NN NN NN NN NN ATT | 108 |
| | Protein_AT6_Direpeat | | |
| BBa_K747020 | TAL- | T NN CA NN NN NN NN T | 109 |
| | Protein_CA2_Direpeat | | |
| BBa_K747068 | TAL | T NN NN NN NN CA NN T | 110 |
| | Protein_CA5_Direpeat | | |
| BBa_K747084 | TAL | TNN NN NN NN NN CAT | 111 |
| | Protein_CA6_Direpeat | | |
| BBa_K747005 | TAL- | T CC NN NN NN NN NN T | 112 |
| | Protein_CC1_Direpeat | | |
| BBa_K747021 | TAL- | T NN CC NN NN NN NN T | 113 |
| | Protein_CC2_Direpeat | | |
| BBa_K747037 | TAL- | T NN NN CC NN NN NN T | 114 |
| | Protein_CC3_Direpeat | | |
| BBa_K747053 | TAL- | T NN NN NN CC NN NN T | 115 |
| | Protein_CC4_Direpeat | | |
| BBa_K747069 | TAL- | T NN NN NN NN CC NN T | 116 |
| | Protein_CC5_Direpeat | | |
| BBa_K747085 | TAL- | T NN NN NN NN NN CC T | 117 |
| | Protein_CC6_Direpeat | | |
| BBa_K747006 | TAL- | T CG NN NN NN NN NN T | 118 |
| | Protein_CG1_Direpeat | | |
| BBa_K747022 | TAL- | T NN CG NN NN NN NN T | 119 |
| | Protein_CG2_Direpeat | | |
| BBa_K747038 | TAL- | TNN NN CG NN NN NN T | 120 |
| | Protein_CG3_Direpeat | | |
| BBa_K747054 | TAL- | T NN NN NN CG NN NN T | 121 |
| | Protein_CG4_Direpeat | | |
| BBa_K747070 | TAL | T NN NN NN NN CG NN T | 122 |
| | Protein_CG5_Direpeat | | |
| BBa_K747086 | TAL- | T NN NN NN NN NN CG T | 123 |
| | Protein_CG6_Direpeat | | |
| BBa_K747039 | TAL- | T NN NN CT NN NN NN T | 124 |
| | Protein_CT3_Direpeat | | |
| BBa_K747055 | TAL- | T NN NN NN CT NN NN T | 125 |
| | Protein CT4_Direpeat | | |
| BBa_K747071 | TAL- | T NN NN NN NN CT NN T | 126 |
| | Protein_CT5_Direpeat | | |
| BBa_K747087 | TAL- | T NN NN NN NN NN CTT | 127 |
| | Protein_CT6_Direpeat | | |
| BBa_K747008 | TAL- | T GA NN NN NN NN NN T | 128 |
| | Protein_GA1_Direpeat | | |
| BBa_K747024 | TAL- | T NN GA NN NN NN NN T | 129 |
| | Protein_GA2_Direpeat | | |
| BBa_K747040 | TAL- | T NN NN GA NN NN NN T | 130 |
| | Protein_GA3_Direpeat | | |
| BBa_K747056 | TAL- | T NN NN NN GA NN NN T | 131 |
| | Protein_GA4_Direpeat | | |
| BBa_K747072 | TAL- | T NN NN NN NN GA NN T | 132 |
| | Protein_GA5_Direpeat | | |
| BBa_K747088 | TAL- | T NN NN NN NN NN GA T | 133 |
| | Protein_GA6_Direpeat | | |
| BBa_K747009 | TAL- | T GC NN NN NN NN NN T | 134 |
| | Protein_GC1_Direpeat | | |
| BBa_K747025 | TAL- | T NN GC NN NN NN NN T | 135 |
| | Protein_GC2_Direpeat | | |
| BBa_K747041 | TAL- | T NN NN GC NN NN NN T | 136 |
| | Protein_GC3_Direpeat | | |
| BBa_K747057 | TAL- | T NN NN NN GC NN NN T | 137 |
| | Protein_GC4_Direpeat | | |
| BBa_K747073 | TAL- | T NN NN NN NN GC NN T | 138 |
| | Protein_GC5_Direpeat | | |
| BBa_K747089 | TAL- | T NN NN NN NN NN GC T | 139 |
| | Protein_GC6_Direpeat | | |
| BBa_K747010 | TAL- | T GG NN NN NN NN NN T | 140 |
| | Protein_GG1_Direpeat | | |
| BBa_K747026 | TAL- | T NN GG NN NN NN NN T | 141 |
| | Protein_GG2_Direpeat | | |
| BBa_K747042 | TAL- | T NN NN GG NN NN NN T | 142 |
| | Protein_GG3_Direpeat | | |
| BBa_K747058 | TAL- | TNN NN NN GG NN NN T | 143 |
| | Protein_GG4_Direpeat | | |
| BBa_K747074 | TAL- | T NN NN NN NN GG NN T | 144 |
| | Protein_GG5_Direpeat | | |
| BBa_K747090 | TAL- | T NN NN NN NN NN GG T | 145 |
| | Protein_GG6_Direpeat | | |
| BBa_K747011 | TAL- | T GT NN NN NN NN NN T | 146 |
| | Protein_GT1_Direpeat | | |
| BBa_K747027 | TAL- | T NN GT NN NN NN NN T | 147 |
| | Protein_GT2_Direpeat | | |
| BBa_K747043 | TAL- | TNN NN GT NN NN NN T | 148 |
| | Protein_GT3_Direpeat | | |
| BBa_K747059 | TAL- | T NN NN NN GT NN NN T | 149 |
| | Protein_GT4_Direpeat | | |
| BBa_K747075 | TAL- | T NN NN NN NN GT NN T | 150 |
| | Protein_GT5_Direpeat | | |
| BBa_K747091 | TAL- | T NN NN NN NN NN GT T | 151 |
| | Protein_GT6_Direpeat | | |
| BBa_K747012 | TAL | TTA NN NN NN NN NN T | 152 |
| | Protein_TA1_Direpeat | | |
| BBa_K747028 | TAL- | T NN TA NN NN NN NN T | 153 |
| | Protein_TA2_Direpeat | | |
| BBa_K747044 | TAL- | T NN NN TA NN NN NN T | 154 |
| | Protein_TA3_Direpeat | | |
| BBa_K747060 | TAL- | T NN NN NN TA NN NN T | 155 |
| | Protein_TA4_Direpeat | | |
| BBa_K747076 | TAL- | T NN NN NN NN TA NN T | 156 |
| | Protein_TA5_Direpeat | | |
| BBa_K747092 | TAL- | T NN NN NN NN NN TA T | 157 |
| | Protein_TA6_Direpeat | | |
| BBa_K747013 | TAL- | TTC NN NN NN NN NN T | 158 |
| | Protein_TC1_Direpeat | | |
| BBa_K747029 | TAL- | T NN TC NN NN NN NN T | 159 |
| | Protein_TC2_Direpeat | | |
| BBa_K747045 | TAL- | T NN NN TC NN NN NN T | 160 |
| | Protein_TC3_Direpeat | | |
| BBa_K747061 | TAL- | T NN NN NN TC NN NN T | 161 |
| | Protein_TC4_Direpeat | | |
| BBa_K747077 | TAL- | T NN NN NN NN TC NN T | 162 |
| | Protein_TC5_Direpeat | | |
| BBa_K747093 | TAL- | TNN NN NN NN NN TC T | 163 |
| | Protein_TC6_Direpeat | | |
| BBa_K747014 | TAL- | TTG NN NN NN NN NN T | 164 |
| | Protein_TG1_Direpeat | | |
| BBa_K747030 | TAL- | T NN TG NN NN NN NN T | 165 |
| | Protein_TG2_Direpeat | | |
| BBa_K747046 | TAL- | T NN NN TG NN NN NN T | 166 |
| | Protein_TG3_Direpeat | | |
| BBa_K747062 | TAL- | TNN NN NN TG NN NN T | 167 |
| | Protein_TG4_Direpeat | | |
| BBa_K747078 | TAL- | T NN NN NN NN TG NN T | 168 |
| | Protein_TG5_Direpeat | | |
| BBa_K747094 | TAL- | T NN NN NN NN NN TG T | 169 |
| | Protein_TG6_Direpeat | | |
| BBa_K747015 | TAL- | TTT NN NN NN NN NN T | 170 |
| | Protein_TT1_Direpeat | | |
| BBa_K747031 | TAL- | T NN TT NN NN NN NN T | 171 |
| | Protein_TT2_Direpeat | | |
| BBa_K747047 | TAL- | TNN NN TT NN NN NN T | 172 |
| | Protein TT3_Direpeat | | |
| BBa_K747063 | TAL- | T NN NN NN TT NN NN T | 173 |
| | Protein_TT4_Direpeat | | |
| BBa_K747079 | TAL- | T NN NN NN NN TT NN T | 174 |
| | Protein_TT5_Direpeat | | |
| BBa_K747095 | TAL- | T NN NN NN NN NN TT T | 175 |
| | Protein_TT6_Direpeat | | |
| BBa_K165007 | Gli-1 DNA-binding | GAC CAC CCA AGA CGA | 176 |
| | domain | | |
| BBa_K105003 | tetR-DNA binding | 1. tccctatcag tgatagagat | 177 |
| | domain | tgacatccct atcagtgata gagatactga | |
| | | gcac | |
| | | 2. tttactccct atcagtgata | 181 |
| | | gagaacgta | |

The sequences of these DNA binding proteins are incorporated by reference from: parts.igem.org/cgi/partsdb/pgroup.cgi?pgroup=DNA binding protein coding domain&show=1

In some embodiments the transcriptional repressor domain of the Cas13d fusion protein comprises any one of SID (SEQ ID NO: 22) or SIDX4 (SEQ ID NO: 23). In some embodiments, the transcriptional repressor domain of the Cas13d fusion protein is a KRAB domain. In some embodiments, the transcriptional repressor domain of the Cas13d fusion protein is a KRAB-A domain. In some embodiments the KRAB-A domain comprises an amino acid sequence that has 85%, 86%, 87%, 88%, 89% 0.90%, 91%, 92%, 93%, 94%, 95%, 96%, 97%, 98%, 99%, 99.5%, or 100% sequence identity to SEQ ID NO: 24. In some embodiments the KRAB-A domain comprises an amino acid sequence that has at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or at least 100% sequence identity to SEQ ID NO: 24. In some embodiments the KRAB-A domain comprises an amino acid sequence that comprises SEQ ID NO: 24. In some embodiments the KRAB-A domain comprises an amino acid sequence that consists of SEQ ID NO: 24.

In some embodiments, the zinc finger binding site may be located within the promoter of the nucleic acid molecule. In some embodiments, the zinc finger binding site may be inserted into the promoter of the nucleic acid molecule. In certain embodiments, the direction of elements on nucleic acid molecule may be in a different arrangement, order, or sequence. For example, the following constructs are contemplated, but any suitable arrangement can be used herein instead:
Construct embodiment 1: 5′-[zinc finger binding site]-[promoter]-[Cas13d domain]-[zinc finger binding domain]-[transcriptional repressor domain]
Alternative embodiment 2: [zinc finger binding site]-[promoter]-[Cas13d domain]-[transcriptional repressor domain]-[zinc finger binding domain]
Alternative embodiment 3: [zinc finger binding site]-[promoter]-[transcriptional repressor domain]-[Cas13d domain]-[zinc finger binding domain]
Alternative embodiment 4: [zinc finger binding site]-[promoter]-[transcriptional repressor domain]-[zinc finger binding domain]-[Cas13d domain]
Alternative embodiment 5: [promoter]-[zinc finger binding site]-[Cas13d domain]-[transcriptional repressor domain]-[zinc finger binding domain]
Alternative embodiment 6: [promoter]-[zinc finger binding site]-[transcriptional repressor domain]-[Cas13d domain]-[zinc finger binding domain]
Alternative embodiment 7: [promoter]-[zinc finger binding site]-[transcriptional repressor domain]-[zinc finger binding domain]-[Cas13d domain]
Construct embodiment 8: 5′-[hCCR5 binding motif]-[promoter]-[Cas13d domain]-[hCCR5 ZnF Domain]-[transcriptional repressor domain]
Alternative embodiment 9: [hCCR5 binding motif]-[promoter]-[Cas13d domain]-[transcriptional repressor domain]-[hCCR5 ZnF Domain]
Alternative embodiment 10: [hCCR5 binding motif]-[promoter]-[transcriptional repressor domain]-[Cas13d domain]-[hCCR5 ZnF Domain]
Alternative embodiment 11: [hCCR5 binding motif]-[promoter]-[transcriptional repressor domain]-[hCCR5 ZnF Domain]-[Cas13d domain]
Alternative embodiment 12: [promoter]-[hCCR5 binding motif]-[Cas13d domain]-[transcriptional repressor domain]-[hCCR5 ZnF Domain]
Alternative embodiment 13: [promoter]-[hCCR5 binding motif]-[transcriptional repressor domain]-[Cas13d domain]-[hCCR5 ZnF Domain]
Alternative embodiment 14: [promoter]-[hCCR5 binding motif]-[transcriptional repressor domain]-[hCCR5 ZnF Domain]-[Cas13d domain]
Alternative embodiment 15: 5′-[hCCR5 binding motif]-[promoter]-[RfxCas13d]-[hCCR5 ZnF Domain]-[transcriptional repressor domain]
Alternative embodiment 16: [hCCR5 binding motif]-[promoter]-[RfxCas13d]-[transcriptional repressor domain]-[hCCR5 ZnF Domain]
Alternative embodiment 17: [hCCR5 binding motif]-[promoter]-[transcriptional repressor domain]-[RfxCas13d]-[hCCR5 ZnF Domain]
Alternative embodiment 18: [hCCR5 binding motif]-[promoter]-[transcriptional repressor domain]-[hCCR5 ZnF Domain]-[RfxCas13d]
Alternative embodiment 19: [promoter]-[hCCR5 binding motif]-[RfxCas13d]-[transcriptional repressor domain]-[hCCR5 ZnF Domain]
Alternative embodiment 20: [promoter]-[hCCR5 binding motif]-[transcriptional repressor domain]-[RfxCas13d]-[hCCR5 ZnF Domain]
Alternative embodiment 21: [promoter]-[hCCR5 binding motif]-[transcriptional repressor domain]-[hCCR5 ZnF Domain]-[RfxCas13d]
Alternative embodiment 22: 5′-[zinc finger binding site]-[promoter]-[RfxCas13d]-[zinc finger binding domain]-[transcriptional repressor domain]
Alternative embodiment 23: [zinc finger binding site]-[promoter]-[RfxCas13d]-[transcriptional repressor domain]-[zinc finger binding domain]
Alternative embodiment 24: [zinc finger binding site]-[promoter]-[transcriptional repressor domain]-[RfxCas13d]-[zinc finger binding domain]
Alternative embodiment 25: [zinc finger binding site]-[promoter]-[transcriptional repressor domain]-[zinc finger binding domain]-[RfxCas13d]
Alternative embodiment 26: [promoter]-[zinc finger binding site]-[RfxCas13d]-[transcriptional repressor domain]-[zinc finger binding domain]
Alternative embodiment 27: [promoter]-[zinc finger binding site]-[transcriptional repressor domain]-[RfxCas13d]-[zinc finger binding domain]
Alternative embodiment 28: [promoter]-[zinc finger binding site]-[transcriptional repressor domain]-[zinc finger binding domain]-[RfxCas13d]

gRNA Excision for Negative-Autoregulatory Optimization (GENO) Constructs

In some embodiments, Cas13d autoregulation is achieved using gRNA excision for negative-autoregulatory optimization (GENO). In some embodiments, Cas13d autoregulation using GENO is achieved by encoding the Cas13d domain and a Cas13d processing sequence (e.g., a pre-guide RNA) on a nucleic acid molecule in such a way that Cas13d and the Cas13d processing sequence are transcribed on the same transcript, which results in Cas13d mediated degradation or Cas13d autoregulation of the Cas13d autoregulation transcript. Without being bound by theory, Cas13d autoregulation occurs when the HEPN-2 domain of the Cas13d cleaves the Cas13d processing sequence from the Cas13d autoregulation transcript containing the Cas13d processing sequence and the Cas13d domain. This cleavage exposes the transcript to RNA exonuclease degradation. Hence as Cas13d domain concentration increases in the cell, Cas13d autoregulation transcript abundance is decreased and in turn decreases Cas13d collateral damage as compared to Cas13d with no autoregulation.

In some embodiments, the nucleic acid molecule comprises in the 5′ to 3′ direction a nucleotide sequence encoding a Cas13d domain and a Cas13d processing sequence on a single Cas13d autoregulation transcript. In some embodiments, the nucleic acid molecule comprises in the 5′ to 3′ direction a nucleotide sequence encoding a Cas13d processing sequence and a Cas13d domain on a single Cas13d autoregulation transcript.

In some embodiments, the Cas13d processing sequence (e.g. the pre-guide RNA) and Cas13d domain of the nucleic acid molecule encoding the Cas13d autoregulation transcript are separated by at least 10 basepairs (e.g. at least 10 basepairs, at least 20 basepairs, at least 30 basepairs, at least 40 basepairs, at least 50 basepairs, at least 60 basepairs, at least 70 basepairs, at least 80 basepairs, at least 90 basepairs, at least 100 basepairs, at least 200 basepairs, at least 400 basepairs, or at least 800 basepairs). In some embodiments, the Cas13d processing sequence and Cas13d domain of the nucleic acid molecule encoding the Cas13d autoregulation transcript are separated by 10 basepairs, 20 basepairs, 30 basepairs, 40 basepairs, 50 basepairs, 60 basepairs, 70 basepairs, 80 basepairs, 90 basepairs, 100 basepairs, 200 basepairs, 400 basepairs, or 800 basepairs. In some embodiments, the Cas13d processing sequence and Cas13d domain of the nucleic acid molecule encoding the Cas13d autoregulation transcript are separated by 0-10 basepairs, 10-20 basepairs, 20-30 basepairs, 30-40 basepairs, 40-50 basepairs, 50-60 basepairs, 60-70 basepairs, 70-80 basepairs, 80-90 basepairs, 90-100 basepairs, 100-200 basepairs, 200-400 basepairs, 400-800 basepairs, 800-1600 basepairs or 1600-320 basepairs. In some embodiments, the Cast 3d processing sequence and Cas13d domain of the nucleic acid molecule encoding the Cas13d autoregulation transcript are separated by 10-100 basepairs, 20-90 basepairs, 30-80 basepairs, or 40-70 basepairs.

In some embodiments, the Cas13d domain of the nucleic acid encoding the Cas13d autoregulation transcript comprises an intron region. In some embodiments, the intron in the Cas13d domain of the nucleic acid comprises Cas13d processing sequence (e.g., the pre-guide RNA). In some embodiments, the Cas13d processing sequence of the nucleic acid encoding the Cas13d autoregulation transcript is located within the Cas13d domain 5′ untranslated region. In some embodiments, the Cas13d processing sequence of the nucleic acid encoding the Cas13d autoregulation transcript is located within the Cas13d domain 3′ untranslated region.

In some embodiments, the Cas13d processing sequence is inserted into the Cas13d domain sequence. In some embodiments, the Cas13d processing sequence is inserted into the Cas13d domain at position 138, 139, 140, 141, 142, 143, 144, 145, or 146 of SEQ ID NO: 57.

In some embodiments, the nucleic acid molecule comprising the nucleotide sequence encoding the Cas13d autoregulation transcript further comprise 1, 2, 3, 4, 5, 6, 7, 8, 9, or 10 pre-guide RNAs. In some embodiments, the pre-guide RNAs encoded on the nucleic acid molecule encoding the Cas13d domain target the same transcript target. In some embodiments, the pre-guide RNAs encoded on the nucleic acid molecule target 1, 2, 3, 4, 5, 6, 7, 8, 9, or 10 different transcript targets. In some embodiments, the pre-guide RNAs encoded on the nucleic acid molecule comprise spacers [S] and direct repeats [DR]. In some embodiments, the guide RNAs encoded on the nucleic acid molecule may be arranged in a repeating as follows [DR][S][DR][S][DR][S][DR] up to comprising about 10 spacers. In some embodiments, the nucleic acid molecule may comprise 10-50 spacers.

In some embodiments, the GENO construct comprises a rfxCas13d and pre-guide RNA. In some embodiments, the Geno construct comprises a rfxCas13d and 1, 2, 3, 4, 5, 6, 7, 8, 9, 10 or more pre-guide RNAs. In some embodiments, the Geno construct comprises a rfxCas13d and a guide RNA that targets a CUG repeat. In some embodiments, the Geno construct comprises a rfxCas13d and a pre-guide RNA that targets any one of SEQ ID NOs: 58-77.

In some embodiments, the GENO construct comprises a nucleic acid molecule comprising in the 5′ to 3′ direction a nucleotide sequence encoding a pre-guide RNA and a rfxCas13d domain on a single Cas13d autoregulation transcript. In some embodiments, the pre-guide RNA is encoded within a UTR of the rfxCas13d transcript. In some embodiments, the pre-guide RNA is encoded within a 5′ UTR of the rfxCas13d transcript. In some embodiments, the pre-guide RNA is encoded within a 3′ UTR of the rfxCas13d transcript.

In some embodiments, the GENO construct comprises a nucleic acid molecule comprising in the 5′ to 3′ direction a nucleotide sequence encoding a pre-guide RNA targeting a CUG repeat and a rfxCas13d domain on a single Cas13d autoregulation transcript. In some embodiments, the pre-guide RNA targeting a CUG repeat is encoded within a UTR of the rfxCas13d transcript. In some embodiments, the pre-guide RNA targeting a CUG repeat is encoded within a 5′ UTR of the rfxCas13d transcript. In some embodiments, the pre-guide RNA targeting a CUG repeat is encoded within a 3′ UTR of the rfxCas13d transcript.

In some embodiments, the GENO construct comprises a nucleic acid molecule comprising in the 5′ to 3′ direction a nucleotide sequence encoding a pre-guide RNA that targets any one of SEQ ID NOs: 58-77 and a rfxCas13d domain on a single Cas13d autoregulation transcript. In some embodiments, the pre-guide RNA that targets any one of SEQ ID NOs: 58-77 is encoded within a UTR of the rfxCas13d transcript. In some embodiments, the pre-guide RNA that targets any one of SEQ ID NOs: 58-77 is encoded within a 5′ UTR of the rfxCas13d transcript. In some embodiments, the pre-guide RNA that targets any one of SEQ ID NOs: 58-77 is encoded within a 3′ UTR of the rfxCas13d transcript.

In some embodiments, a Cas13 autoregulating construct comprises a nucleic acid molecule encoding a Cas13a domain, a Cas13b domain, a Cas13c domain, or a Cas13d domain and one or more guide RNAs on a single Cas13-gRNA transcript.

### Methods of Target Transcript Knockdown

In some aspects, the present disclosure provides methods for knocking down gene expression using autoregulated Cas13d domains. In some embodiments, Cas13d autoregulation may result in a reduction in Cas13d collateral damage as compared to Cas13d collateral damage without autoregulation. In some embodiments, knocking down gene expression in accomplished using any one of the autoregulated Cas13d nucleic acid molecules or constructs described herein (e.g., Cas13 autoregulation using a Zinc Finger domain or GENO).

It is to be understood that one of ordinary skill in the art would readily understand what is mean by expression of a transcript target is knocked down. Knocking down expression of a target transcript may also be referred to as knocking down target gene expression, knocking down target protein expression, decreasing target transcript abundance, decreasing target protein expression, decreasing target gene expression, specific gene knockdown, decreasing expression of a specific gene, decreasing specific gene transcript abundance, gene expression knockdown, transcript expression knockdown, protein expression knockdown or any other synonymous term that is well known in the art.

In some embodiments, expression of a specific transcript is knocked down using an autoregulated Cas13d fusion protein, wherein autoregulation of the Cas13d fusion protein reduces Cas13d collateral damage.

In some embodiments, Cas13d collateral damage is reduced by autoregulating the expression or activity of the Cas13d domain or the Cas13d fusion protein (i.e., a Cas13d fusion protein comprising a Cas13d domain). In some embodiments, Cas13d autoregulation results in less collateral damage than is observed when using RNAi to knockdown a target transcript. In some embodiments, Cas13d autoregulation results in less collateral damage than is observed when using RNAi to knockdown a target transcript. In some embodiments, collateral damage associated with knocking down the expression of a target transcript using an autoregulated Cas13d domain or the Cas13d fusion protein (i.e., a Cas13d fusion protein comprising a Cas13d domain) is less than the collateral damage when using RNAi by at least (e.g., at least 10%, at least 20%, at least 30%, at least 40%, at least 50%, at least 60%, at least 70%, at least 80%, or at least 90%). In some embodiments, collateral damage associated with knocking down the expression of a target transcript using an autoregulated Cas13d domain or the Cas13d fusion protein (i.e., a Cas13d fusion protein comprising a Cas13d domain) is less than the collateral damage when using a RNAi by 10%-20%, 20%-30%, 30%-40%, 40%-50%, 50%-60%, 60%-70%, 70%-80%, or 80%-90%.

In some embodiments, Cas13d autoregulation results in less collateral damage than is observed when using a Cas13d domain or the Cas13d fusion protein (i.e., a Cas13d fusion protein comprising a Cas13d domain) that is not autoregulated. In some embodiments, collateral damage associated with knocking down the expression of a target transcript using an autoregulated Cas13d domain or the Cas13d fusion protein (i.e., a Cas13d fusion protein comprising a Cas13d domain) is less than the collateral damage when using a not autoregulated Cas13d domain by at least (e.g., at least 10%, at least 20%, at least 30%, at least 40%, at least 50%, at least 60%, at least 70%, at least 80%, or at least 90%). In some embodiments, collateral damage associated with knocking down the expression of a target transcript using an autoregulated Cas13d domain or the Cas13d fusion protein (i.e., a Cas13d fusion protein comprising a Cas13d domain) is less than the collateral damage when using a not autoregulated Cas13d domain by 10%-20%, 20%-30%, 30%-40%, 40%-50%, 50%-60%, 60%-70%, 70%-80%, or 80%-90%.

### Method of Transcript Knockdown Using Transcript Knockdown: Cas13 Autoregulation Using a Zinc Finger Domain

In some embodiments, expression of a transcript target is knocked down using an autoregulated Cas13d domain, comprising expression of a nucleic acid molecule comprising in the 5′ to 3′ direction a zinc finger binding site, a promoter, and a nucleotide sequence encoding a Cas13d fusion protein, wherein the Cas13d fusion protein comprises a Cas13d domain, a zinc finger (ZnF) domain, and a transcriptional repressor domain, and wherein the Cas13d fusion protein binds to the zinc finger binding site and represses transcription of the nucleotide sequence encoding the Cas13d fusion protein.

In some embodiments, expression of a transcript target is knocked down using an autoregulated Cas13d domain, comprising expression of a nucleic acid molecule comprising in the 5′ to 3′ direction a zinc finger binding site, a promoter, and a nucleotide sequence encoding a Cas13d fusion protein, wherein the Cas13d fusion protein comprises a Cas13d domain, a zinc finger (ZnF) domain, and a transcriptional repressor domain, and wherein the Cas 13d fusion protein binds to the zinc finger binding site and represses transcription of the nucleotide sequence encoding the Cas13d fusion protein.

In some embodiments, expression of a specific transcript is knocked down using an autoregulated Cas13d fusion protein comprising a nucleic acid molecule encoding a promoter, a Cas13d protein, a zinc finger (ZnF) binding sequence motif, a ZnF domain, and a Krüppel-associated box (KRAB) domain, wherein the Cas13d protein, the ZnF domain and the KRAB domain comprise a Cas13d fusion protein that is operably linked to the promoter, wherein the ZnF binding sequence motif is upstream of the promoter, wherein the construct comprises a means of autoregulation of the of expression the Cas13d fusion protein.

In some embodiments, the means for autoregulation of transcription of the Cas13d fusion protein comprises expressing the Cas13d fusion protein comprising the ZnF domain and KRAB domain, wherein the ZnF domain binds to the zinc finger sequence motif, thereby causing the KRAB domain to inhibit transcription of the sequence encoding the Cas13d fusion protein.

In some embodiments, expression of a specific transcript is knocked down using an autoregulated Cas13d fusion protein comprising a nucleic acid molecule encoding a promoter, a Cas13d protein, a zinc finger (ZnF) binding sequence motif, a ZnF domain, and a Krüppel-associated box (KRAB) domain, wherein the Cas13d protein, the ZnF domain and the KRAB domain comprise a Cas13d fusion protein that is operably linked to the promoter, wherein the ZnF binding sequence motif is upstream of the promoter, and the nucleic acid molecule further comprises at least 1 guide RNA (e.g. at least 1, at least 2, at least 3, at least 4, at least 5, at least 6, at least 7, at least 8, at least 9, at least 10 guide RNAs).

In some embodiments, expression of a transcript target is knocked down using an autoregulated Cas13d domain, wherein transcript target knocked down is achieved by administering a nucleic acid sequence encoding a guide RNA that targets the nucleic acid sequence of any one of SEQ ID NO: 58-77.

In some embodiments, the ZnF sequence motif and the ZnF domain of the Cas13d fusion protein may be replaced with any one of the DNA binding proteins and corresponding sequence motifs described herein (e.g., an engineered Talen, and modified CRISPR Cas system) or that is well known in the art. In some embodiments, the KRAB domain of the Cas13d fusion protein may be replaced with an alternative transcriptional repressor domain as described herein or well known in the art.

Method of Transcript Knockdown Using gRNA Excision for Negative-Autoregulatory Optimization (GENO)

In some embodiments, the expression of a specific transcript is knocked down using a GENO construct, as described above. In some embodiments, the expression of a specific transcript is knocked down using a GENO construct comprising a nucleic acid sequence encoding a Cas13d protein and a pre-guide RNA. In some embodiments, the pre-guide RNA comprises a spacer sequence that targets a gene associated with a disease or disorder listed herein. In some embodiments, the pre-guide RNA comprises a spacer sequence that targets a CUG expansion. In some embodiments, the pre-guide RNA targets the nucleic acid sequence of any one of SEQ ID NO: 58-77. In some embodiments, the expression of a specific transcript is knocked down using a GENO construct comprising a nucleic acid molecule comprising in the 5′ to 3′ direction a nucleotide sequence encoding a Cas13d domain and a pre-guide RNA on a single Cas13d autoregulation transcript. In some embodiments, the expression of a specific transcript is knocked down using a GENO construct comprising a nucleic acid molecule comprising in the 5′ to 3′ direction a nucleotide sequence encoding a pre-guide RNA and a Cas13d domain on a single Cas13d autoregulation transcript. In some embodiments, the pre-guide RNA is encoded within a UTR of the Cas13d transcript. In some embodiments, the pre-guide RNA is encoded within a 5′ UTR of the Cas13d transcript. In some embodiments, the pre-guide RNA is encoded within a 3′ UTR of the Cas13d transcript.

In some embodiments, the expression of a specific transcript is knocked down using a GENO construct comprising a rfxCas13d and pre-guide RNA. In some embodiments, the Geno construct comprises a rfxCas13d and 1, 2, 3, 4, 5, 6, 7, 8, 9, 10 or more pre-guide RNAs. In some embodiments, the Geno construct comprises a rfxCas13d and a guide RNA that targets a CUG repeat. In some embodiments, the Geno construct comprises a rfxCas13d and a pre-guide RNA that targets any one of SEQ ID NOs: 58-77.

In some embodiments, the expression of a specific transcript is knocked down using a GENO construct comprising a nucleic acid molecule comprising in the 5′ to 3′ direction a nucleotide sequence encoding a pre-guide RNA and a rfxCas13d domain on a single Cas13d autoregulation transcript. In some embodiments, the pre-guide RNA is encoded within a UTR of the rfxCas13d transcript. In some embodiments, the pre-guide RNA is encoded within a 5′ UTR of the rfxCas13d transcript. In some embodiments, the pre-guide RNA is encoded within a 3′ UTR of the rfxCas13d transcript.

In some embodiments, the expression of a specific transcript is knocked down using a GENO construct comprising a nucleic acid molecule comprising in the 5′ to 3′ direction a nucleotide sequence encoding a pre-guide RNA targeting a CUG repeat and a rfxCas13d domain on a single Cas13d autoregulation transcript. In some embodiments, the pre-guide RNA targeting a CUG repeat is encoded within a UTR of the rfxCas13d transcript. In some embodiments, the pre-guide RNA targeting a CUG repeat is encoded within a 5′ UTR of the rfxCas13d transcript. In some embodiments, the pre-guide RNA targeting a CUG repeat is encoded within a 3′ UTR of the rfxCas13d transcript.

In some embodiments, the expression of a specific transcript is knocked down using a GENO construct comprising a nucleic acid molecule comprising in the 5′ to 3′ direction a nucleotide sequence encoding a pre-guide RNA that targets any one of SEQ ID NOs: 58-77 and a rfxCas13d domain on a single Cas13d autoregulation transcript. In some embodiments, the pre-guide RNA that targets any one of SEQ ID NOs: 58-77 is encoded within a UTR of the rfxCas13d transcript. In some embodiments, the pre-guide RNA that targets any one of SEQ ID NOs: 58-77 is encoded within a 5′ UTR of the rfxCas13d transcript. In some embodiments, the pre-guide RNA that targets any one of SEQ ID NOs: 58-77 is encoded within a 3′ UTR of the rfxCas13d transcript.

### Methods of Treating Disease

The instant disclosure provides methods for the treatment of a subject diagnosed with a disease associated with or caused by transcriptional dysregulation, protein expression dysregulation, RNA aggregation, genetic repeat expansions (e.g., CUG expansions), or dominant negative mutant alleles, each of which may be corrected by one or more of the Cas13d autoregulatory constructs provided herein (e.g., a Cas13d fusion protein or GENO) and/or a method of transcript knockdown described herein. For example, in some embodiments, a method is provided that comprises administering to a subject having such a disease, e.g., a muscular dystrophy associated CUG expansion and RNA aggregation as described above, an effective amount of Cas13d autoregulatory construct that decreases the expression of the disease-associated gene transcript target as mediated by Cas13d cleavage of the transcript target. In some embodiments, the disease is a proliferative disease. In some embodiments, the disease is a genetic disease. In some embodiments, the disease is a neoplastic disease. In some embodiments, the disease is a metabolic disease. In some embodiments, the disease is a lysosomal storage disease. Other diseases that can be treated by decreasing the expression of a target transcript will be known to those of skill in the art, and the disclosure is not limited in this respect.

The instant disclosure provides methods for the treatment of additional diseases or disorders, e.g., diseases or disorders that are associated or caused by expression of a gene, expression of a gene mutant, or increased expression of any gene corrected by autoregulated Cas13d knockdown of the gene transcript target. Some such diseases are described herein, and additional suitable diseases that can be treated with the strategies and by Cas3d autoregulatory construct provided herein will be apparent to those of skill in the art based on the instant disclosure. Exemplary suitable diseases and disorders are listed below.

The instant disclosure provides methods for the treatment of diseases using Cas13d autoregulatory constructs. In some embodiments, treatment of disease with an using Cas13d autoregulatory constructs reduces collateral damage as compared to treatment of a disease using a Cas13d nucleic acid without autoregulation.

Treatment of the following exemplary diseases and disorders associated with expanded DNA or RNA repeats have been contemplated for treatment using RNA transcript knockdown: Spinocerebellar ataxia type 12, Fragile X-associated tremor/ataxia syndrome, Neuronal intranuclear inclusion disease, C9ORF72 amyotrophic lateral sclerosis/frontotemporal dementia, Benign adult familial myoclonic epilepsy (familial adult myoclonic epilepsy 1), Cerebellar ataxia, neuropathy, vestibular areflexia syndrome, Myotonic dystrophy type 2, Fuchs endothelial corneal dystrophy, Spinocerebellar ataxia type 10, Spinocerebellar ataxia type 31, Spinocerebellar ataxia type 36 (Asidan, Costa da Morte ataxia), Spinocerebellar ataxia type 37, Dentatorubral-pallidoluysian atrophy (Haw River syndrome, Naito-Oyanagi disease), Huntington's disease, Spinal-bulbar muscular atrophy, Spinocerebellar ataxia type 1, Spinocerebellar ataxia type 2, Spinocerebellar ataxia type 3 (Machado-Joseph disease), Spinocerebellar ataxia type 6, Spinocerebellar ataxia type 7, Spinocerebellar ataxia type 8, Spinocerebellar ataxia type 17, Myotonic dystrophy type 1, Huntington's disease-like 2, Blepharophimosis syndrome, Cleidocranial dysplasia, Congenital central hypoventilation syndrome, Hand-foot-genital syndrome, Holoprosencephaly, Oculopharyngeal muscular dystrophy, Synpolydactyly syndrome, X-linked mental retardation and abnormal genitalia, X-linked mental retardation, X-linked mental retardation and growth hormone deficit, Pseudoachondroplasia and multiple epiphyseal dysplasia, Familial adult myoclonic epilepsy 2, Familial adult myoclonic epilepsy 3, Familial adult myoclonic epilepsy 4, Familial adult myoclonic epilepsy 6, Familial adult myoclonic epilepsy 7, Oculopharyngeal myopathy with leukoencephalopathy, Oculopharyngodistal myopathy 1, Oculopharyngodistal myopathy 2.

Treatment of the following exemplary diseases and disorders has been contemplated to the extent possible that these diseases could be corrected by RNA transcript knockdown. Exemplary suitable diseases and disorders include, without limitation: 2-methyl-3-hydroxybutyric aciduria; 3 beta-Hydroxysteroid dehydrogenase deficiency; 3-Methylglutaconic aciduria; 3-Oxo-5 alpha-steroid delta 4-dehydrogenase deficiency; 46,XY sex reversal, type 1, 3, and 5; 5-Oxoprolinase deficiency; 6-pyruvoyl-tetrahydropterin synthase deficiency; Aarskog syndrome; Aase syndrome; Achondrogenesis type 2; Achromatopsia 2 and 7; Acquired long QT syndrome; Acrocallosal syndrome, Schinzel type; Acrocapitofemoral dysplasia; Acrodysostosis 2, with or without hormone resistance; Acroerythrokeratoderma; Acromicric dysplasia; Acth-independent macronodular adrenal hyperplasia 2; Activated P13K-delta syndrome; Acute intermittent porphyria; deficiency of Acyl-CoA dehydrogenase family, member 9; Adams-Oliver syndrome 5 and 6; Adenine phosphoribosyltransferase deficiency; Adenylate kinase deficiency; hemolytic anemia due to Adenylosuccinate lyase deficiency; Adolescent nephronophthisis; Renal-hepatic-pancreatic dysplasia; Meckel syndrome type 7;

Adrenoleukodystrophy; Adult junctional epidermolysis bullosa; Epidermolysis bullosa, junctional, localisata variant; Adult neuronal ceroid lipofuscinosis; Adult neuronal ceroid lipofuscinosis; Adult onset ataxia with oculomotor apraxia; ADULT syndrome; Afibrinogenemia and congenital Afibrinogenemia; autosomal recessive Agammaglobulinemia 2; Age-related macular degeneration 3, 6, 11, and 12; Aicardi Goutieres syndromes 1, 4, and 5; Chilbain lupus 1; Alagille syndromes 1 and 2; Alexander disease; Alkaptonuria; Allan-Herndon-Dudley syndrome; Alopecia universalis congenital; Alpers encephalopathy; Alpha-1-antitrypsin deficiency; autosomal dominant, autosomal recessive, and X-linked recessive Alport syndromes; Alzheimer disease, familial, 3, with spastic paraparesis and apraxia; Alzheimer disease, types, 1, 3, and 4; hypocalcification type and hypomaturation type, IIA1 Amelogenesis imperfecta; Aminoacylase I deficiency; Amish infantile epilepsy syndrome; Amyloidogenic transthyretin amyloidosis; Amyloid Cardiomyopathy, Transthyretin-related;

Cardiomyopathy; Amyotrophic lateral sclerosis types 1, 6, 15 (with or without frontotemporal dementia), 22 (with or without frontotemporal dementia), and 10; Frontotemporal dementia with TDP43 inclusions, TARDBP-related; Andermann syndrome; Andersen Tawil syndrome; Congenital long QT syndrome; Anemia, nonspherocytic hemolytic, due to G6PD deficiency; Angelman syndrome; Severe neonatal-onset encephalopathy with microcephaly; susceptibility to Autism, X-linked 3; Angiopathy, hereditary, with nephropathy, aneurysms, and muscle cramps; Angiotensin i-converting enzyme, benign serum increase; Aniridia, cerebellar ataxia, and mental retardation; Anonychia; Antithrombin III deficiency; Antley-Bixler syndrome with genital anomalies and disordered steroidogenesis; Aortic aneurysm, familial thoracic 4, 6, and 9; Thoracic aortic aneurysms and aortic dissections;

Multisystemic smooth muscle dysfunction syndrome; Moyamoya disease 5; Aplastic anemia; Apparent mineralocorticoid excess; Arginase deficiency; Argininosuccinate lyase deficiency; Aromatase deficiency; Arrhythmogenic right ventricular cardiomyopathy types 5, 8, and 10; Primary familial hypertrophic cardiomyopathy; Arthrogryposis multiplex congenita, distal, X-linked; Arthrogryposis renal dysfunction cholestasis syndrome; Arthrogryposis, renal dysfunction, and cholestasis 2; Asparagine synthetase deficiency; Abnormality of neuronal migration; Ataxia with vitamin E deficiency; Ataxia, sensory, autosomal dominant; Ataxia-telangiectasia syndrome; Hereditary cancer-predisposing syndrome; Atransferrinemia; Atrial fibrillation, familial, 11, 12, 13, and 16; Atrial septal defects 2, 4, and 7 (with or without atrioventricular conduction defects); Atrial standstill 2; Atrioventricular septal defect 4; Atrophia bulborum hereditaria; ATR-X syndrome; Auriculocondylar syndrome 2; Autoimmune disease, multisystem, infantile-onset; Autoimmune lymphoproliferative syndrome, type 1a; Autosomal dominant hypohidrotic ectodermal dysplasia; Autosomal dominant progressive external ophthalmoplegia with mitochondrial DNA deletions 1 and 3;

Autosomal dominant torsion dystonia 4; Autosomal recessive centronuclear myopathy; Autosomal recessive congenital ichthyosis 1, 2, 3, 4A, and 4B; Autosomal recessive cutis laxa type IA and 1B; Autosomal recessive hypohidrotic ectodermal dysplasia syndrome; Ectodermal dysplasia 11b; hypohidrotic/hair/tooth type, autosomal recessive; Autosomal recessive hypophosphatemic bone disease; Axenfeld-Rieger syndrome type 3; Bainbridge-Ropers syndrome; Bannayan-Riley-Ruvalcaba syndrome; PTEN hamartoma tumor syndrome; Baraitser-Winter syndromes 1 and 2; Barakat syndrome; Bardet-Biedl syndromes 1, 11, 16, and 19; Bare lymphocyte syndrome type 2, complementation group E; Bartter syndrome antenatal type 2; Bartter syndrome types 3, 3 with hypocalciuria, and 4; Basal ganglia calcification, idiopathic, 4; Beaded hair; Benign familial hematuria; Benign familial neonatal seizures 1 and 2; Seizures, benign familial neonatal, 1, and/or myokymia; Seizures,

Early infantile epileptic encephalopathy 7; Benign familial neonatal-infantile seizures; Benign hereditary chorea; Benign scapuloperoneal muscular dystrophy with cardiomyopathy; Bernard-Soulier syndrome, types A1 and A2 (autosomal dominant); Bestrophinopathy, autosomal recessive; beta Thalassemia; Bethlem myopathy and Bethlem myopathy 2; Bietti crystalline corneoretinal dystrophy; Bile acid synthesis defect, congenital, 2; Biotinidase deficiency; Birk Barel mental retardation dysmorphism syndrome; Blepharophimosis, ptosis, and epicanthus inversus; Bloom syndrome; Borjeson-Forssman-Lehmann syndrome; Boucher Neuhauser syndrome; Brachydactyly types A1 and A2; Brachydactyly with hypertension; Brain small vessel disease with hemorrhage; Branched-chain ketoacid dehydrogenase kinase deficiency; Branchiootic syndromes 2 and 3; Breast cancer, early-onset; Breast-ovarian cancer, familial 1, 2, and 4; Brittle cornea syndrome 2; Brody myopathy; Bronchiectasis with or without elevated sweat chloride 3; Brown-Vialetto-Van laere syndrome and Brown-Vialetto-Van Laere syndrome 2; Brugada syndrome; Brugada syndrome 1;

Ventricular fibrillation; Paroxysmal familial ventricular fibrillation; Brugada syndrome and Brugada syndrome 4; Long QT syndrome; Sudden cardiac death; Bull eye macular dystrophy; Stargardt disease 4; Cone-rod dystrophy 12; Bullous ichthyosiform erythroderma; Burn-Mckeown syndrome; Candidiasis, familial, 2, 5, 6, and 8; Carbohydrate-deficient glycoprotein syndrome type I and II; Carbonic anhydrase VA deficiency, hyperammonemia due to: Carcinoma of colon; Cardiac arrhythmia; Long QT syndrome, LQT1 subtype; Cardioencephalomyopathy, fatal infantile, due to cytochrome c oxidase deficiency; Cardiofaciocutaneous syndrome; Cardiomyopathy; Danon disease; Hypertrophic cardiomyopathy; Left ventricular noncompaction cardiomyopathy; Carnevale syndrome; Carney complex, type 1; Carnitine acylcarnitine translocase deficiency; Carnitine palmitoykransferase I, II, II (late onset), and II (infantile) deficiency; Cataract 1, 4, autosomal dominant, autosomal dominant, multiple types, with microcornea, coppock-like, juvenile, with microcornea and glucosuria, and nuclear diffuse nonprogressive; Catecholaminergic polymorphic ventricular tachycardia; Caudal regression syndrome; Cd8 deficiency, familial;

Central core disease; Centromeric instability of chromosomes 1,9 and 16 and immunodeficiency; Cerebellar ataxia infantile with progressive external ophthalmoplegi and Cerebellar ataxia, mental retardation, and dysequilibrium syndrome 2; Cerebral amyloid angiopathy, APP-related; Cerebral autosomal dominant and recessive arteriopathy with subcortical infarcts and leukoencephalopathy; Cerebral cavernous malformations 2; Cerebrooculofacioskeletal syndrome 2; Cerebro-oculo-facio-skeletal syndrome; Cerebroretinal microangiopathy with calcifications and cysts; Ceroid lipofuscinosis neuronal 2, 6, 7, and 10; Ch\xc3\xa9diak-Higashi syndrome, Chediak-Higashi syndrome, adult type; Charcot-Marie-Tooth disease types 1B, 2B2, 2C, 2F, 2I, 2U (axonal), 1C (demyelinating), dominant intermediate C, recessive intermediate A, 2A2, 4C, 4D, 4H, IF, IVF, and X;

Scapuloperoneal spinal muscular atrophy; Distal spinal muscular atrophy, congenital nonprogressive; Spinal muscular atrophy, distal, autosomal recessive, 5; CHARGE association; Childhood hypophosphatasia; Adult hypophosphatasia; Cholecystitis; Progressive familial intrahepatic cholestasis 3; Cholestasis, intrahepatic, of pregnancy 3; Cholestanol storage disease; Cholesterol monooxygenase (side-chain cleaving) deficiency; Chondrodysplasia Blomstrand type; Chondrodysplasia punctata 1, X-linked recessive and 2 X-linked dominant; CHOPS syndrome; Chronic granulomatous disease, autosomal recessive cytochrome b-positive, types 1 and 2; Chudley-McCullough syndrome; Ciliary dyskinesia, primary, 7, 11, 15, 20 and 22; Citrullinemia type I; Citrullinemia type I and II; Cleidocranial dysostosis; C-like syndrome; Cockayne syndrome type A; Coenzyme Q10 deficiency, primary 1, 4, and 7; Coffin Siris/Intellectual Disability; Coffin-Lowry syndrome;

Cohen syndrome; Cold-induced sweating syndrome 1; COLE-CARPENTER SYNDROME 2; Combined cellular and humoral immune defects with granulomas; Combined d-2- and 1-2-hydroxyglutaric aciduria; Combined malonic and methylmalonic aciduria; Combined oxidative phosphorylation deficiencies 1, 3, 4, 12, 15, and 25; Combined partial and complete 17-alpha-hydroxylase/17,20-lyase deficiency; Common variable immunodeficiency 9; Complement component 4, partial deficiency of, due to dysfunctional c1 inhibitor; Complement factor B deficiency; Cone monochromatism; Cone-rod dystrophy 2 and 6; Cone-rod dystrophy amelogenesis imperfecta; Congenital adrenal hyperplasia and Congenital adrenal hypoplasia, X-linked; Congenital amegakaryocytic thrombocytopenia;

Congenital aniridia; Congenital central hypoventilation; Hirschsprung disease 3; Congenital contractual arachnodactyly; Congenital contractures of the limbs and face, hypotonia, and developmental delay; Congenital disorder of glycosylation types 1B, 1D, 1G, 1H, 1J, 1K, 1N, 1P, 2C, 2J, 2K, Ilm; Congenital dyserythropoietic anemia, type I and II; Congenital ectodermal dysplasia of face; Congenital erythropoietic porphyria; Congenital generalized lipodystrophy type 2; Congenital heart disease, multiple types, 2; Congenital heart disease; Interrupted aortic arch; Congenital lipomatous overgrowth, vascular malformations, and epidermal nevi; Non-small cell lung cancer; Neoplasm of ovary; Cardiac conduction defect, nonspecific; Congenital microvillous atrophy; Congenital muscular dystrophy; Congenital muscular dystrophy due to partial LAMA2 deficiency;
Congenital muscular dystrophy-dystroglycanopathy with brain and eye anomalies, types A2, A7, A8, A11, and A14; Congenital muscular dystrophy-dystroglycanopathy with mental retardation, types B2, B3, B5, and B15; Congenital muscular dystrophy-dystroglycanopathy without mental retardation, type B5; Congenital muscular hypertrophy-cerebral syndrome; Congenital myasthenic syndrome, acetazolamide-responsive; Congenital myopathy with fiber type disproportion; Congenital ocular coloboma; Congenital stationary night blindness, type 1 A, 1B, 1C, 1E, 1F, and 2A; Coproporphyria; Cornea plana 2; Corneal dystrophy, Fuchs endothelial, 4; Corneal endothelial dystrophy type 2; Corneal fragility keratoglobus, blue sclerae and joint hypermobility; Cornelia de Lange syndromes 1 and 5; Coronary artery disease, autosomal dominant 2; Coronary heart disease; Hyperalphalipoproteinemia 2; Cortical dysplasia, complex, with other brain malformations 5 and 6; Cortical malformations, occipital; Corticosteroid-binding globulin deficiency; Corticosterone methyloxidase type 2 deficiency; Costello syndrome; Cowden syndrome 1; Coxa plana; Craniodiaphyseal dysplasia, autosomal dominant; Craniosynostosis 1 and 4; Craniosynostosis and dental anomalies; Creatine deficiency, X-linked; Crouzon syndrome; Cryptophthalmos syndrome;

Cryptorchidism, unilateral or bilateral; Cushing symphalangism; Cutaneous malignant melanoma 1; Cutis laxa with osteodystrophy and with severe pulmonary, gastrointestinal, and urinary abnormalities; Cyanosis, transient neonatal and atypical nephropathic; Cystic fibrosis; Cystinuria; Cytochrome c oxidase i deficiency; Cytochrome-c oxidase deficiency; D-2-hydroxyglutaric aciduria 2; Darier disease, segmental; Deafness with labyrinthine aplasia microtia and microdontia (LAMM); Deafness, autosomal dominant 3a, 4, 12, 13, 15, autosomal dominant nonsyndromic sensorineural 17, 20, and 65; Deafness, autosomal recessive 1 A, 2, 3, 6, 8, 9, 12, 15, 16, 18b, 22, 28, 31, 44, 49, 63, 77, 86, and 89; Deafness, cochlear, with myopia and intellectual impairment, without vestibular involvement, autosomal dominant, X-linked 2; Deficiency of 2-methylbutyryl-CoA dehydrogenase; Deficiency of 3-hydroxyacyl-CoA dehydrogenase; Deficiency of alpha-mannosidase; Deficiency of aromatic-L-amino-acid decarboxylase; Deficiency of bisphosphoglycerate mutase; Deficiency of butyryl-CoA dehydrogenase; Deficiency of ferroxidase; Deficiency of galactokinase; Deficiency of guanidinoacetate methyltransferase; Deficiency of hyaluronoglucosaminidase; Deficiency of ribose-5-phosphate isomerase; Deficiency of steroid 11-beta-monooxygenase; Deficiency of UDPglucose-hexose-1-phosphate uridylyltransferase;

Deficiency of xanthine oxidase; Dejerine-Sottas disease; Charcot-Marie-Tooth disease, types ID and IVF; Dejerine-Sottas syndrome, autosomal dominant; Dendritic cell, monocyte, B lymphocyte, and natural killer lymphocyte deficiency; Desbuquois dysplasia 2; Desbuquois syndrome; DFNA 2 Nonsyndromic Hearing Loss; Diabetes mellitus and insipidus with optic atrophy and deafness; Diabetes mellitus, type 2, and insulin-dependent, 20; Diamond-Blackfan anemia 1, 5, 8, and 10; Diarrhea 3 (secretory sodium, congenital, syndromic) and 5 (with tufting enteropathy, congenital); Dicarboxylic aminoaciduria; Diffuse palmoplantar keratoderma, Bothnian type; Digitorenocerebral syndrome; Dihydropteridine reductase deficiency; Dilated cardiomyopathy 1A, 1AA, 1C, 1G, 1BB, 1DD, 1FF, 1HH, 1I, 1KK, 1N, 1S, 1Y, and 3B; Left ventricular noncompaction 3; Disordered steroidogenesis due to cytochrome p450 oxidoreductase deficiency; Distal arthrogryposis type 2B; Distal hereditary motor neuronopathy type 2B; Distal myopathy Markesbery-Griggs type; Distal spinal muscular atrophy, X-linked 3; Distichiasis-lymphedema syndrome; Dominant dystrophic epidermolysis bullosa with absence of skin; Dominant hereditary optic atrophy; Donnai Barrow syndrome; Dopamine beta hydroxylase deficiency; Dopamine receptor d2, reduced brain density of; Dowling-degos disease 4; Doyne honeycomb retinal dystrophy; Malattia leventinese;

Duane syndrome type 2; Dubin-Johnson syndrome; Duchenne muscular dystrophy; Becker muscular dystrophy; Dysfibrinogenemia; Dyskeratosis congenita autosomal dominant and autosomal dominant, 3; Dyskeratosis congenita, autosomal recessive, 1, 3, 4, and 5; Dyskeratosis congenita X-linked; Dyskinesia, familial, with facial myokymia; Dysplasminogenemia; Dystonia 2 (torsion, autosomal recessive), 3 (torsion, X-linked), 5 (Dopa-responsive type), 10, 12, 16, 25, 26 (Myoclonic); Seizures, benign familial infantile, 2; Early infantile epileptic encephalopathy 2, 4, 7, 9, 10, 11, 13, and 14; Atypical Rett syndrome; Early T cell progenitor acute lymphoblastic leukemia; Ectodermal dysplasia skin fragility syndrome; Ectodermal dysplasia-syndactyly syndrome 1; Ectopia lentis, isolated autosomal recessive and dominant; Ectrodactyly, ectodermal dysplasia, and cleft lip/palate syndrome 3; Ehlers-Danlos syndrome type 7 (autosomal recessive), classic type, type 2 (progeroid), hydroxylysine-deficient, type 4, type 4 variant, and due to tenascin-X deficiency; Eichsfeld type congenital muscular dystrophy; Endocrine-cerebroosteodysplasia; Enhanced s-cone syndrome; Enlarged vestibular aqueduct syndrome; Enterokinase deficiency; Epidermodysplasia verruciformis; Epidermolysa bullosa simplex and limb girdle muscular dystrophy, simplex with mottled pigmentation, simplex with pyloric atresia, simplex, autosomal recessive, and with pyloric atresia; Epidermolytic palmoplantar keratoderma; Familial febrile seizures 8; Epilepsy, childhood absence 2, 12 (idiopathic generalized, susceptibility to) 5 (nocturnal frontal lobe), nocturnal frontal lobe type 1, partial, with variable foci, progressive myoclonic 3, and X-linked, with variable learning disabilities and behavior disorders; Epileptic encephalopathy, childhood-onset, early infantile, 1, 19, 23, 25, 30, and 32; Epiphyseal dysplasia, multiple, with myopia and conductive deafness; Episodic ataxia type 2; Episodic pain syndrome, familial, 3; Epstein syndrome; Fechtner syndrome; Erythropoietic protoporphyria; Estrogen resistance; Exudative vitreoretinopathy 6; Fabry disease and Fabry disease, cardiac variant; Factor H, VII, X, v and factor viii, combined deficiency of 2, xiii, a subunit, deficiency; Familial adenomatous polyposis 1 and 3; Familial amyloid nephropathy with urticaria and deafness; Familial cold urticarial; Familial aplasia of the vermis; Familial benign pemphigus; Familial cancer of breast; Breast cancer, susceptibility to; Osteosarcoma; Pancreatic cancer 3; Familial cardiomyopathy; Familial cold autoinflammatory syndrome 2; Familial colorectal cancer; Familial exudative vitreoretinopathy, X-linked; Familial hemiplegic migraine types 1 and 2; Familial hypercholesterolemia; Familial hypertrophic cardiomyopathy 1, 2, 3, 4, 7, 10, 23 and 24; Familial hypokalemia-hypomagnesemia; Familial hypoplastic, glomerulocystic kidney; Familial infantile myasthenia; Familial juvenile gout; Familial Mediterranean fever and Familial mediterranean fever, autosomal dominant; Familial porencephaly; Familial porphyria cutanea tarda; Familial pulmonary capillary hemangiomatosis; Familial renal glucosuria; Familial renal hypouricemia; Familial restrictive cardiomyopathy 1; Familial type 1 and 3 hyperlipoproteinemia; Fanconi anemia, complementation group E, I, N, and O; Fanconi-Bickel syndrome; Favism, susceptibility to: Febrile seizures, familial, 11; Feingold syndrome 1; Fetal hemoglobin quantitative trait locus 1; FG syndrome and FG syndrome 4; Fibrosis of extraocular muscles, congenital, 1, 2, 3a (with or without extraocular involvement), 3b; Fish-eye disease; Fleck corneal dystrophy; Floating-Harbor syndrome; Focal epilepsy with speech disorder with or without mental retardation; Focal segmental glomerulosclerosis 5; Forebrain defects; Frank Ter Haar syndrome; Borrone Di Rocco Crovato syndrome; Frasier syndrome; Wilms tumor 1; Freeman-Sheldon syndrome; Frontometaphyseal dysplasia land 3; Frontotemporal dementia; Frontotemporal dementia and/or amyotrophic lateral sclerosis 3 and 4; Frontotemporal Dementia Chromosome 3-Linked and Frontotemporal dementia ubiquitin-positive; Fructose-biphosphatase deficiency; Fuhrmann syndrome; Gamma-aminobutyric acid transaminase deficiency; Gamstorp-Wohlfart syndrome; Gaucher disease type 1 and Subacute neuronopathic; Gaze palsy, familial horizontal, with progressive scoliosis; Generalized dominant dystrophic epidermolysis bullosa; Generalized epilepsy with febrile seizures plus 3, type 1, type 2; Epileptic encephalopathy Lennox-Gastaut type; Giant axonal neuropathy; Glanzmann thrombasthenia; Glaucoma 1, open angle, e, F, and G; Glaucoma 3, primary congenital, d; Glaucoma, congenital and Glaucoma, congenital, Coloboma; Glaucoma, primary open angle, juvenile-onset; Glioma susceptibility 1; Glucose transporter type 1 deficiency syndrome; Glucose-6-phosphate transport defect; GLUT1 deficiency syndrome 2; Epilepsy, idiopathic generalized, susceptibility to, 12; Glutamate formiminotransferase deficiency; Glutaric acidemia IIA and IIB; Glutaric aciduria, type 1; Gluthathione synthetase deficiency; Glycogen storage disease 0 (muscle), II (adult form), IXa2, IXc, type 1A; type II, type IV, IV (combined hepatic and myopathic), type V, and type VI; Goldmann-Favre syndrome; Gordon syndrome; Gorlin syndrome; Holoprosencephaly sequence; Holoprosencephaly 7; Granulomatous disease, chronic, X-linked, variant; Granulosa cell tumor of the ovary; Gray platelet syndrome; Griscelli syndrome type 3; Groenouw corneal dystrophy type I; Growth and mental retardation, mandibulofacial dysostosis, microcephaly, and cleft palate; Growth hormone deficiency with pituitary anomalies; Growth hormone insensitivity with immunodeficiency; GTP cyclohydrolase I deficiency; Hajdu-Cheney syndrome; Hand foot uterus syndrome; Hearing impairment; Hemangioma, capillary infantile; Hematologic neoplasm; Hemochromatosis type 1, 2B, and 3; Microvascular complications of diabetes 7; Transferrin serum level quantitative trait locus 2; Hemoglobin H disease, nondeletional; Hemolytic anemia, nonspherocytic, due to glucose phosphate isomerase deficiency; Hemophagocytic lymphohistiocytosis, familial, 2; Hemophagocytic lymphohistiocytosis, familial, 3; Heparin cofactor II deficiency; Hereditary acrodermatitis enteropathica; Hereditary breast and ovarian cancer syndrome; Ataxia-telangiectasia-like disorder; Hereditary diffuse gastric cancer; Hereditary diffuse leukoencephalopathy with spheroids; Hereditary factors II, IX, VIII deficiency disease; Hereditary hemorrhagic telangiectasia type 2; Hereditary insensitivity to pain with anhidrosis; Hereditary lymphedema type I; Hereditary motor and sensory neuropathy with optic atrophy; Hereditary myopathy with early respiratory failure; Hereditary neuralgic amyotrophy; Hereditary Nonpolyposis Colorectal Neoplasms; Lynch syndrome I and II; Hereditary pancreatitis; Pancreatitis, chronic, susceptibility to; Hereditary sensory and autonomic neuropathy type IIB amd IIA; Hereditary sideroblastic anemia; Hermansky-Pudlak syndrome 1, 3, 4, and 6; Heterotaxy, visceral, 2, 4, and 6, autosomal; Heterotaxy, visceral, X-linked; Heterotopia; Histiocytic medullary reticulosis; Histiocytosis-lymphadenopathy plus syndrome; Holocarboxylase synthetase deficiency; Holoprosencephaly 2, 3,7, and 9; Holt-Oram syndrome; Homocysteinemia due to MTHFR deficiency, CBS deficiency, and Homocystinuria, pyridoxine-responsive; Homocystinuria-Megaloblastic anemia due to defect in cobalamin metabolism, cblE complementation type; Howel-Evans syndrome; Hurler syndrome; Hutchinson-Gilford syndrome; Hydrocephalus; Hyperammonemia, type III; Hypercholesterolaemia and Hypercholesterolemia, autosomal recessive; Hyperekplexia 2 and Hyperekplexia hereditary; Hyperferritinemia cataract syndrome; Hyperglycinuria; Hyperimmunoglobulin D with periodic fever; Mevalonic aciduria; Hyperimmunoglobulin E syndrome; Hyperinsulinemic hypoglycemia familial 3, 4, and 5; Hyperinsulinism-hyperammonemia syndrome; Hyperlysinemia; Hypermanganesemia with dystonia, polycythemia and cirrhosis; Hyperornithinemia-hyperammonemia-homocitrullinuria syndrome; Hyperparathyroidism 1 and 2; Hyperparathyroidism, neonatal severe; Hyperphenylalaninemia, bh4-deficient, a, due to partial pts deficiency, BH4-deficient, D, and non-pku; Hyperphosphatasia with mental retardation syndrome 2, 3, and 4; Hypertrichotic osteochondrodysplasia; Hypobetalipoproteinemia, familial, associated with apob32; Hypocalcemia, autosomal dominant 1; Hypocalciuric hypercalcemia, familial, types 1 and 3; Hypochondrogenesis; Hypochromic microcytic anemia with iron overload; Hypoglycemia with deficiency of glycogen synthetase in the liver; Hypogonadotropic hypogonadism 11 with or without anosmia; Hypohidrotic ectodermal dysplasia with immune deficiency; Hypohidrotic X-linked ectodermal dysplasia; Hypokalemic periodic paralysis 1 and 2; Hypomagnesemia 1, intestinal; Hypomagnesemia, seizures, and mental retardation; Hypomyelinating leukodystrophy 7; Hypoplastic left heart syndrome; Atrioventricular septal defect and common atrioventricular junction; Hypospadias 1 and 2, X-linked; Hypothyroidism, congenital, nongoitrous, 1; Hypotrichosis 8 and 12; Hypotrichosis-lymphedema-telangiectasia syndrome; I blood group system; Ichthyosis bullosa of Siemens; Ichthyosis exfoliativa; Ichthyosis prematurity syndrome; Idiopathic basal ganglia calcification 5; Idiopathic fibrosing alveolitis, chronic form; Dyskeratosis congenita, autosomal dominant, 2 and 5; Idiopathic hypercalcemia of infancy; Immune dysfunction with T-cell inactivation due to calcium entry defect 2; Immunodeficiency 15, 16, 19, 30, 31C, 38, 40, 8, due to defect in cd3-zeta, with hyper IgM type 1 and 2, and X-Linked, with magnesium defect, Epstein-Barr virus infection, and neoplasia; Immunodeficiency-centromeric instability-facial anomalies syndrome 2; Inclusion body myopathy 2 and 3; Nonaka myopathy; Infantile convulsions and paroxysmal choreoathetosis, familial; Infantile cortical hyperostosis; Infantile GM1 gangliosidosis; Infantile hypophosphatasia; Infantile nephronophthisis; Infantile nystagmus, X-linked; Infantile Parkinsonism-dystonia; Infertility associated with multi-tailed spermatozoa and excessive DNA; Insulin resistance; Insulin-resistant diabetes mellitus and acanthosis nigricans; Insulin-dependent diabetes mellitus secretory diarrhea syndrome; Interstitial nephritis, karyomegalic; Intrauterine growth retardation, metaphyseal dysplasia, adrenal hypoplasia congenita, and genital anomalies; Iodotyrosyl coupling defect; IRAK4 deficiency; Iridogoniodysgenesis dominant type and type 1; Iron accumulation in brain; Ischiopatellar dysplasia; Islet cell hyperplasia; Isolated 17,20-lyase deficiency; Isolated lutropin deficiency; Isovaleryl-CoA dehydrogenase deficiency; Jankovic Rivera syndrome; Jervell and Lange-Nielsen syndrome 2; Joubert syndrome 1, 6, 7, 9/15 (digenic), 14, 16, and 17, and Orofaciodigital syndrome xiv; Junctional epidermolysis bullosa gravis of Herlitz; Juvenile GM>1<gangliosidosis; Juvenile polyposis syndrome; Juvenile polyposis/hereditary hemorrhagic telangiectasia syndrome; Juvenile retinoschisis; Kabuki make-up syndrome; Kallmann syndrome 1, 2, and 6; Delayed puberty; Kanzaki disease; Karak syndrome; Kartagener syndrome; Kenny-Caffey syndrome type 2; Keppen-Lubinsky syndrome; Keratoconus 1; Keratosis follicularis; Keratosis palmoplantaris striata 1; Kindler syndrome; L-2-hydroxyglutaric aciduria; Larsen syndrome, dominant type; Lattice corneal dystrophy Type III; Leber amaurosis; Zellweger syndrome; Peroxisome biogenesis disorders; Zellweger syndrome spectrum; Leber congenital amaurosis 11, 12, 13, 16, 4, 7, and 9; Leber optic atrophy; Aminoglycoside-induced deafness; Deafness, nonsyndromic sensorineural, mitochondrial; Left ventricular noncompaction 5; Left-right axis malformations; Leigh disease; Mitochondrial short-chain Enoyl-CoA Hydratase 1 deficiency; Leigh syndrome due to mitochondrial complex I deficiency; Leiner disease; Leri Weill dyschondrosteosis; Lethal congenital contracture syndrome 6; Leukocyte adhesion deficiency type I and III; Leukodystrophy, Hypomyelinating, 11 and 6; Leukoencephalopathy with ataxia, with Brainstem and Spinal Cord Involvement and Lactate Elevation, with vanishing white matter, and progressive, with ovarian failure; Leukonychia totalis; Lewy body dementia; Lichtenstein-Knorr Syndrome; Li-Fraumeni syndrome 1; Lig4 syndrome; Limb-girdle muscular dystrophy, type 1B, 2A, 2B, 2D, C1, C5, C9, C14; Congenital muscular dystrophy-dystroglycanopathy with brain and eye anomalies, type A14 and B14; Lipase deficiency combined; Lipid proteinosis; Lipodystrophy, familial partial, type 2 and 3; Lissencephaly 1, 2 (X-linked), 3, 6 (with microcephaly), X-linked; Subcortical laminar heterotopia, X-linked; Liver failure acute infantile; Loeys-Dietz syndrome 1, 2, 3; Long QT syndrome 1, 2, 2/9, 2/5, (digenic), 3, 5 and 5, acquired, susceptibility to; Lung cancer; Lymphedema, hereditary, id; Lymphedema, primary, with myelodysplasia; Lymphoproliferative syndrome 1, 1 (X-linked), and 2; Lysosomal acid lipase deficiency; Macrocephaly, macrosomia, facial dysmorphism syndrome; Macular dystrophy, vitelliform, adult-onset; Malignant hyperthermia susceptibility type 1; Malignant lymphoma, non-Hodgkin; Malignant melanoma; Malignant tumor of prostate; Mandibuloacral dysostosis; Mandibuloacral dysplasia with type A or B lipodystrophy, atypical; Mandibulofacial dysostosis, Treacher Collins type, autosomal recessive; Mannose-binding protein deficiency; Maple syrup urine disease type 1A and type 3; Marden Walker like syndrome; Marfan syndrome; Marinesco-Sj\xc3\xb6gren syndrome; Martsolf syndrome; Maturity-onset diabetes of the young, type 1, type 2, type 11, type 3, and type 9; May-Hegglin anomaly; MYH9 related disorders; Sebastian syndrome; McCune-Albright syndrome; Somatotroph adenoma; Sex cord-stromal tumor; Cushing syndrome; McKusick Kaufman syndrome; McLeod neuroacanthocytosis syndrome; Meckel-Gruber syndrome; Medium-chain acyl-coenzyme A dehydrogenase deficiency; Medulloblastoma; Megalencephalic leukoencephalopathy with subcortical cysts land 2a; Megalencephaly cutis marmorata telangiectatica congenital; PIK3CA Related Overgrowth Spectrum; Megalencephaly-polymicrogyria-polydactyly-hydrocephalus syndrome 2; Megaloblastic anemia, thiamine-responsive, with diabetes mellitus and sensorineural deafness; Meier-Gorlin syndromes land 4; Melnick-Needles syndrome; Meningioma; Mental retardation, X-linked, 3, 21, 30, and 72; Mental retardation and microcephaly with pontine and cerebellar hypoplasia; Mental retardation X-linked syndromic 5; Mental retardation, anterior maxillary protrusion, and strabismus; Mental retardation, autosomal dominant 12, 13, 15, 24, 3, 30, 4, 5, 6, and 9; Mental retardation, autosomal recessive 15, 44, 46, and 5; Mental retardation, stereotypic movements, epilepsy, and/or cerebral malformations; Mental retardation, syndromic, Claes-Jensen type, X-linked; Mental retardation, X-linked, nonspecific, syndromic, Hedera type, and syndromic, wu type; Merosin deficient congenital muscular dystrophy; Metachromatic leukodystrophy juvenile, late infantile, and adult types; Metachromatic leukodystrophy; Metatrophic dysplasia; Methemoglobinemia types 1 and 2; Methionine adenosyltransferase deficiency, autosomal dominant; Methylmalonic acidemia with homocystinuria; Methylmalonic aciduria cblB type; Methylmalonic aciduria due to methylmalonyl-CoA mutase deficiency; METHYLMALONIC ACIDURIA, mut(0) TYPE; Microcephalic osteodysplastic primordial dwarfism type 2; Microcephaly with or without chorioretinopathy, lymphedema, or mental retardation; Microcephaly, hiatal hernia and nephrotic syndrome; Microcephaly; Hypoplasia of the corpus callosum; Spastic paraplegia 50, autosomal recessive; Global developmental delay; CNS hypomyelination; Brain atrophy; Microcephaly, normal intelligence and immunodeficiency; Microcephaly-capillary malformation syndrome; Microcytic anemia; Microphthalmia syndromic 5, 7, and 9; Microphthalmia, isolated 3, 5, 6, 8, and with coloboma 6; Microspherophakia; Migraine, familial basilar; Miller syndrome; Minicore myopathy with external ophthalmoplegia; Myopathy, congenital with cores; Mitchell-Riley syndrome; mitochondrial 3-hydroxy-3-methylglutaryl-CoA synthase deficiency; Mitochondrial complex I, II, III, III (nuclear type 2, 4, or 8) deficiency; Mitochondrial DNA depletion syndrome 11, 12 (cardiomyopathic type), 2, 4B (MNGIE type), 8B (MNGIE type); Mitochondrial DNA-depletion syndrome 3 and 7, hepatocerebral types, and 13 (encephalomyopathic type); Mitochondrial phosphate carrier and pyruvate carrier deficiency; Mitochondrial trifunctional protein deficiency; Long-chain 3-hydroxyacyl-CoA dehydrogenase deficiency; Miyoshi muscular dystrophy 1; Myopathy, distal, with anterior tibial onset; Mohr-Tranebjaerg syndrome; Molybdenum cofactor deficiency, complementation group A; Mowat-Wilson syndrome; Mucolipidosis III Gamma; Mucopolysaccharidosis type VI, type VI (severe), and type VII; Mucopolysaccharidosis, MPS-I-H/S, MPS-II, MPS-III-A, MPS-III-B, MPS-III-C, MPS-IV-A, MPS-IV-B; Retinitis Pigmentosa 73; Gangliosidosis GM1 type1 (with cardiac involvement) 3; Multicentric osteolysis nephropathy; Multicentric osteolysis, nodulosis and arthropathy; Multiple congenital anomalies; Atrial septal defect 2; Multiple congenital anomalies-hypotonia-seizures syndrome 3; Multiple Cutaneous and Mucosal Venous Malformations; Multiple endocrine neoplasia, types land 4; Multiple epiphyseal dysplasia 5 or Dominant; Multiple gastrointestinal atresias; Multiple pterygium syndrome Escobar type; Multiple sulfatase deficiency; Multiple synostoses syndrome 3; Muscle AMP guanine oxidase deficiency; Muscle eye brain disease; Muscular dystrophy, congenital, megaconial type; Myasthenia, familial infantile, 1; Myasthenic Syndrome, Congenital, 11, associated with acetylcholine receptor deficiency; Myasthenic Syndrome, Congenital, 17, 2A (slow-channel), 4B (fast-channel), and without tubular aggregates; Myeloperoxidase deficiency; MYH-associated polyposis; Endometrial carcinoma; Myocardial infarction 1; Myoclonic dystonia; Myoclonic-Atonic Epilepsy; Myoclonus with epilepsy with ragged red fibers; Myofibrillar myopathy 1 and ZASP-related; Myoglobinuria, acute recurrent, autosomal recessive; Myoneural gastrointestinal encephalopathy syndrome; Cerebellar ataxia infantile with progressive external ophthalmoplegia; Mitochondrial DNA depletion syndrome 4B, MNGIE type; Myopathy, centronuclear, 1, congenital, with excess of muscle spindles, distal, 1, lactic acidosis, and sideroblastic anemia 1, mitochondrial progressive with congenital cataract, hearing loss, and developmental delay, and tubular aggregate, 2; Myopia 6; Myosclerosis, autosomal recessive; Myotonia congenital; Congenital myotonia, autosomal dominant and recessive forms; Nail-patella syndrome; Nance-Horan syndrome; Nanophthalmos 2; Navajo neurohepatopathy; Nemaline myopathy 3 and 9; Neonatal hypotonia; Intellectual disability; Seizures; Delayed speech and language development; Mental retardation, autosomal dominant 31; Neonatal intrahepatic cholestasis caused by citrin deficiency; Nephrogenic diabetes insipidus, Nephrogenic diabetes insipidus, X-linked; Nephrolithiasis/osteoporosis, hypophosphatemic, 2; Nephronophthisis 13, 15 and 4; Infertility; Cerebello-oculo-renal syndrome (nephronophthisis, oculomotor apraxia and cerebellar abnormalities); Nephrotic syndrome, type 3, type 5, with or without ocular abnormalities, type 7, and type 9; Nestor-Guillermo progeria syndrome; Neu-Laxova syndrome 1; Neurodegeneration with brain iron accumulation 4 and 6; Neuroferritinopathy; Neurofibromatosis, type land type 2; Neurofibrosarcoma; Neurohypophyseal diabetes insipidus; Neuropathy, Hereditary Sensory, Type IC; Neutral 1 amino acid transport defect; Neutral lipid storage disease with myopathy; Neutrophil immunodeficiency syndrome; Nicolaides-Baraitser syndrome; Niemann-Pick disease type C1, C2, type A, and type C1, adult form; Non-ketotic hyperglycinemia; Noonan syndrome 1 and 4, LEOPARD syndrome 1; Noonan syndrome-like disorder with or without juvenile myelomonocytic leukemia; Normokalemic periodic paralysis, potassium-sensitive; Norum disease; Epilepsy, Hearing Loss. And Mental Retardation Syndrome; Mental Retardation, X-Linked 102 and syndromic 13; Obesity; Ocular albinism, type 1; Oculocutaneous albinism type 1B, type 3, and type 4; Oculodentodigital dysplasia; Odontohypophosphatasia; Odontotrichomelic syndrome; Oguchi disease; Oligodontia-colorectal cancer syndrome; Opitz G/BBB syndrome; Optic atrophy 9; Oral-facial-digital syndrome; Ornithine aminotransferase deficiency; Orofacial cleft 11 and 7, Cleft lip/palate-ectodermal dysplasia syndrome; Orstavik Lindemann Solberg syndrome; Osteoarthritis with mild chondrodysplasia; Osteochondritis dissecans; Osteogenesis imperfecta type 12, type 5, type 7, type 8, type I, type III, with normal sclerae, dominant form, recessive perinatal lethal; Osteopathia striata with cranial sclerosis; Osteopetrosis autosomal dominant type 1 and 2, recessive 4, recessive 1, recessive 6; Osteoporosis with pseudoglioma; Oto-palato-digital syndrome, types I and II; Ovarian dysgenesis 1; Ovarioleukodystrophy; Pachyonychia congenita 4 and type 2; Paget disease of bone, familial; Pallister-Hall syndrome; Palmoplantar keratoderma, nonepidermolytic, focal or diffuse; Pancreatic agenesis and congenital heart disease; Papillon-Lef\xc3\xa8vre syndrome; Paragangliomas 3; Paramyotonia congenita of von Eulenburg; Parathyroid carcinoma; Parkinson disease 14, 15, 19 (juvenile-onset), 2, 20 (early-onset), 6, (autosomal recessive early-onset, and 9; Partial albinism; Partial hypoxanthine-guanine phosphoribosyltransferase deficiency; Patterned dystrophy of retinal pigment epithelium; PC-K6a; Pelizaeus-Merzbacher disease; Pendred syndrome; Peripheral demyelinating neuropathy, central dysmyelination; Hirschsprung disease; Permanent neonatal diabetes mellitus; Diabetes mellitus, permanent neonatal, with neurologic features; Neonatal insulin-dependent diabetes mellitus; Maturity-onset diabetes of the young, type 2; Peroxisome biogenesis disorder 14B, 2A, 4A, 5B, 6A, 7A, and 7B; Perrault syndrome 4; Perry syndrome; Persistent hyperinsulinemic hypoglycemia of infancy; familial hyperinsulinism; Phenotypes; Phenylketonuria; Pheochromocytoma; Hereditary Paraganglioma-Pheochromocytoma Syndromes; Paragangliomas 1; Carcinoid tumor of intestine; Cowden syndrome 3; Phosphoglycerate dehydrogenase deficiency; Phosphoglycerate kinase 1 deficiency; Photosensitive trichothiodystrophy; Phytanic acid storage disease; Pick disease; Pierson syndrome; Pigmentary retinal dystrophy; Pigmented nodular adrenocortical disease, primary, 1; Pilomatrixoma; Pitt-Hopkins syndrome; Pituitary dependent hypercortisolism; Pituitary hormone deficiency, combined 1, 2, 3, and 4; Plasminogen activator inhibitor type 1 deficiency; Plasminogen deficiency, type I; Platelet-type bleeding disorder 15 and 8; Poikiloderma, hereditary fibrosing, with tendon contractures, myopathy, and pulmonary fibrosis; Polycystic kidney disease 2, adult type, and infantile type; Polycystic lipomembranous osteodysplasia with sclerosing leukoencephalopathy; Polyglucosan body myopathy 1 with or without immunodeficiency; Polymicrogyria, asymmetric, bilateral frontoparietal; Polyneuropathy, hearing loss, ataxia, retinitis pigmentosa, and cataract; Pontocerebellar hypoplasia type 4; Popliteal pterygium syndrome; Porencephaly 2; Porokeratosis 8, disseminated superficial actinic type; Porphobilinogen synthase deficiency; Porphyria cutanea tarda; Posterior column ataxia with retinitis pigmentosa; Posterior polar cataract type 2; Prader-Willi-like syndrome; Premature ovarian failure 4, 5, 7, and 9; Primary autosomal recessive microcephaly 10, 2, 3, and 5; Primary ciliary dyskinesia 24; Primary dilated cardiomyopathy; Left ventricular noncompaction 6; 4, Left ventricular noncompaction 10; Paroxysmal atrial fibrillation; Primary hyperoxaluria, type I, type, and type III; Primary hypertrophic osteoarthropathy, autosomal recessive 2; Primary hypomagnesemia; Primary open angle glaucoma juvenile onset 1; Primary pulmonary hypertension; Primrose syndrome; Progressive familial heart block type 1B; Progressive familial intrahepatic cholestasis 2 and 3; Progressive intrahepatic cholestasis; Progressive myoclonus epilepsy with ataxia; Progressive pseudorheumatoid dysplasia; Progressive sclerosing poliodystrophy; Prolidase deficiency; Proline dehydrogenase deficiency; Schizophrenia 4; Properdin deficiency, X-linked; Propionic academia; Proprotein convertase 1/3 deficiency; Prostate cancer, hereditary, 2; Protan defect; Proteinuria; Finnish congenital nephrotic syndrome; Proteus syndrome; Breast adenocarcinoma; Pseudoachondroplastic spondyloepiphyseal dysplasia syndrome; Pseudohypoaldosteronism type 1 autosomal dominant and recessive and type 2; Pseudohypoparathyroidism type 1A, Pseudopseudohypoparathyroidism; Pseudoneonatal adrenoleukodystrophy; Pseudoprimary hyperaldosteronism; Pseudoxanthoma elasticum; Generalized arterial calcification of infancy 2; Pseudoxanthoma elasticum-like disorder with multiple coagulation factor deficiency; Psoriasis susceptibility 2; PTEN hamartoma tumor syndrome; Pulmonary arterial hypertension related to hereditary hemorrhagic telangiectasia; Pulmonary Fibrosis And/Or Bone Marrow Failure, Telomere-Related, 1 and 3; Pulmonary hypertension, primary, 1, with hereditary hemorrhagic telangiectasia; Purine-nucleoside phosphorylase deficiency; Pyruvate carboxylase deficiency; Pyruvate dehydrogenase E1-alpha deficiency; Pyruvate kinase deficiency of red cells; Raine syndrome; Rasopathy; Recessive dystrophic epidermolysis bullosa; Nail disorder, nonsyndromic congenital, 8; Reifenstein syndrome; Renal adysplasia; Renal carnitine transport defect; Renal coloboma syndrome; Renal dysplasia; Renal dysplasia, retinal pigmentary dystrophy, cerebellar ataxia and skeletal dysplasia; Renal tubular acidosis, distal, autosomal recessive, with late-onset sensorineural hearing loss, or with hemolytic anemia; Renal tubular acidosis, proximal, with ocular abnormalities and mental retardation; Retinal cone dystrophy 3B; Retinitis pigmentosa; Retinitis pigmentosa 10, 11, 12, 14, 15, 17, and 19; Retinitis pigmentosa 2, 20, 25, 35, 36, 38, 39, 4, 40, 43, 45, 48, 66, 7, 70, 72; Retinoblastoma; Rett disorder; Rhabdoid tumor predisposition syndrome 2; Rhegmatogenous retinal detachment, autosomal dominant; Rhizomelic chondrodysplasia punctata type 2 and type 3; Roberts-SC phocomelia syndrome; Robinow Sorauf syndrome; Robinow syndrome, autosomal recessive, autosomal recessive, with brachy-syn-polydactyly; Rothmund-Thomson syndrome; Rapadilino syndrome; RRM2B-related mitochondrial disease; Rubinstein-Taybi syndrome; Salla disease; Sandhoff disease, adult and infantil types; Sarcoidosis, early-onset; Blau syndrome; Schindler disease, type 1; Schizencephaly; Schizophrenia 15; Schneckenbecken dysplasia; Schwannomatosis 2; Schwartz Jampel syndrome type 1; Sclerocornea, autosomal recessive; Sclerosteosis; Secondary hypothyroidism; Segawa syndrome, autosomal recessive; Senior-Loken syndrome 4 and 5; Sensory ataxic neuropathy, dysarthria, and ophthalmoparesis; Sepiapterin reductase deficiency; SeSAME syndrome; Severe combined immunodeficiency due to ADA deficiency, with microcephaly, growth retardation, and sensitivity to ionizing radiation, atypical, autosomal recessive, T cell-negative, B cell-positive, NK cell-negative of NK-positive; Severe congenital neutropenia; Severe congenital neutropenia 3, autosomal recessive or dominant; Severe congenital neutropenia and 6, autosomal recessive; Severe myoclonic epilepsy in infancy; Generalized epilepsy with febrile seizures plus, types 1 and 2; Severe X-linked myotubular myopathy; Short QT syndrome 3; Short stature with nonspecific skeletal abnormalities; Short stature, auditory canal atresia, mandibular hypoplasia, skeletal abnormalities; Short stature, onychodysplasia, facial dysmorphism, and hypotrichosis; Primordial dwarfism; Short-rib thoracic dysplasia 11 or 3 with or without polydactyly; Sialidosis type I and II; Silver spastic paraplegia syndrome; Slowed nerve conduction velocity, autosomal dominant; Smith-Lemli-Opitz syndrome; Snyder Robinson syndrome; Somatotroph adenoma; Prolactinoma; familial, Pituitary adenoma predisposition; Sotos syndrome 1 or 2; Spastic ataxia 5, autosomal recessive, Charlevoix-Saguenay type, 1, 10, or 11, autosomal recessive; Amyotrophic lateral sclerosis type 5; Spastic paraplegia 15, 2, 3, 35, 39, 4, autosomal dominant, 55, autosomal recessive, and 5A; Bile acid synthesis defect, congenital, 3; Spermatogenic failure 11, 3, and 8; Spherocytosis types 4 and 5; Spheroid body myopathy; Spinal muscular atrophy, lower extremity predominant 2, autosomal dominant; Spinal muscular atrophy, type II; Spinocerebellar ataxia 14, 21, 35, 40,and 6; Spinocerebellar ataxia autosomal recessive 1 and 16; Splenic hypoplasia; Spondylocarpotarsal synostosis syndrome; Spondylocheirodysplasia, Ehlers-Danlos syndrome-like, with immune dysregulation, Aggrecan type, with congenital joint dislocations, short limb-hand type, Sedaghatian type, with cone-rod dystrophy, and Kozlowski type; Parastremmatic dwarfism; Stargardt disease 1; Cone-rod dystrophy 3; Stickler syndrome type 1; Kniest dysplasia; Stickler syndrome, types 1(nonsyndromic ocular) and 4; Sting-associated vasculopathy, infantile-onset; Stormorken syndrome; Sturge-Weber syndrome, Capillary malformations, congenital, 1; Succinyl-CoA acetoacetate transferase deficiency; Sucrase-isomaltase deficiency; Sudden infant death syndrome; Sulfite oxidase deficiency, isolated; Supravalvar aortic stenosis; Surfactant metabolism dysfunction, pulmonary, 2 and 3; Symphalangism, proximal. 1b; Syndactyly Cenani Lenz type; Syndactyly type 3; Syndromic X-linked mental retardation 16; Talipes equinovarus; Tangier disease; TARP syndrome; Tay-Sachs disease, B1 variant, Gm2-gangliosidosis (adult), Gm2-gangliosidosis (adult-onset); Temtamy syndrome; Tenorio Syndrome; Terminal osseous dysplasia; Testosterone 17-beta-dehydrogenase deficiency; Tetraamelia, autosomal recessive; Tetralogy of Fallot; Hypoplastic left heart syndrome 2; Truncus arteriosus; Malformation of the heart and great vessels; Ventricular septal defect 1; Thiel-Behnke corneal dystrophy; Thoracic aortic aneurysms and aortic dissections; Marfanoid habitus; Three M syndrome 2; Thrombocytopenia, platelet dysfunction, hemolysis, and imbalanced globin synthesis; Thrombocytopenia, X-linked; Thrombophilia, hereditary, due to protein C deficiency, autosomal dominant and recessive; Thyroid agenesis; Thyroid cancer, follicular; Thyroid hormone metabolism, abnormal; Thyroid hormone resistance, generalized, autosomal dominant; Thyrotoxic periodic paralysis and Thyrotoxic periodic paralysis 2; Thyrotropin-releasing hormone resistance, generalized; Timothy syndrome; TNF receptor-associated periodic fever syndrome (TRAPS); Tooth agenesis, selective, 3 and 4; Torsades de pointes; Townes-Brocks-branchiootorenal-like syndrome; Transient bullous dermolysis of the newborn; Treacher collins syndrome 1; Trichomegaly with mental retardation, dwarfism and pigmentary degeneration of retina; Trichorhinophalangeal dysplasia type I; Trichorhinophalangeal syndrome type 3; Trimethylaminuria; Tuberous sclerosis syndrome; Lymphangiomyomatosis; Tuberous sclerosis 1 and 2; Tyrosinase-negative oculocutaneous albinism; Tyrosinase-positive oculocutaneous albinism; Tyrosinemia type I; UDPglucose-4-epimerase deficiency; Ullrich congenital muscular dystrophy; Ulna and fibula absence of with severe limb deficiency; Upshaw-Schulman syndrome; Urocanate hydratase deficiency; Usher syndrome, types 1, 1B, 1D, 1G, 2A, 2C, and 2D; Retinitis pigmentosa 39; UV-sensitive syndrome; Van der Woude syndrome; Van Maldergem syndrome 2; Hennekam lymphangiectasia-lymphedema syndrome 2; Variegate porphyria; Ventriculomegaly with cystic kidney disease; Verheij syndrome; Very long chain acyl-CoA dehydrogenase deficiency; Vesicoureteral reflux 8; Visceral heterotaxy 5, autosomal; Visceral myopathy; Vitamin D-dependent rickets, types land 2; Vitelliform dystrophy; von Willebrand disease type 2M and type 3; Waardenburg syndrome type 1, 4C, and 2E (with neurologic involvement); Klein-Waardenberg syndrome; Walker-Warburg congenital muscular dystrophy; Warburg micro syndrome 2 and 4; Warts, hypogammaglobulinemia, infections, and myelokathexis; Weaver syndrome; Weill-Marchesani syndrome 1 and 3; Weill-Marchesani-like syndrome; Weissenbacher-Zweymuller syndrome; Werdnig-Hoffmann disease; Charcot-Marie-Tooth disease; Werner syndrome; WFS1-Related Disorders; Wiedemann-Steiner syndrome; Wilson disease; Wolfram-like syndrome, autosomal dominant; Worth disease; Van Buchem disease type 2; Xeroderma pigmentosum, complementation group b, group D, group E, and group G; X-linked agammaglobulinemia; X-linked hereditary motor and sensory neuropathy; X-linked ichthyosis with steryl-sulfatase deficiency; X-linked periventricular heterotopia; Oto-palato-digital syndrome, type I; X-linked severe combined immunodeficiency; Zimmermann-Laband syndrome and Zimmermann-Laband syndrome 2; and Zonular pulverulent cataract 3.

### Subject in Need Thereof

A subject in need thereof” refers to an individual who has a disease, a sign and/or symptom of a disease, or a predisposition toward a disease, with the purpose to cure, heal, alleviate, relieve, alter, remedy, ameliorate, improve, or affect the disease, the symptom of the disease, or the predisposition toward the disease. In some embodiments, the subject is a mammal. In some embodiments, the subject is a non-human primate. In some embodiments, the subject is human. In some embodiments, the mammal is a rodent. In some embodiments, the rodent is a mouse. In some embodiments, the rodent is a rat. In some embodiments, the mammal is a companion animal. A “companion animal” refers to pets and other domestic animals. Non-limiting examples of companion animals include dogs and cats; livestock, such as horses, cattle, pigs, sheep, goats, and chickens; and other animals, such as mice, rats, guinea pigs, and hamsters.

### Therapeutically Effective Amount

In some embodiments, a therapeutically effective amount of any one of nucleic acids designed for autoregulating Cas13d expression and described herein is administered to a subject in need thereof. “A therapeutically effective amount” as used herein refers to the amount of each therapeutic agent (e.g., a nucleic acid encoding a Cas13d capable of autoregulation, or a plasmid, vector, viral vector or AAV encoding said nucleic acid) described in the present disclosure required to confer therapeutic effect on the subject, either alone or in combination with one or more other therapeutic agents. Effective amounts vary, as recognized by those skilled in the art, depending on the particular condition being treated, the severity of the condition, the individual subject parameters including age, physical condition, size, gender, and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a subject may insist upon a lower dose or tolerable dose for medical reasons, psychological reasons or for virtually any other reasons. Empirical considerations, such as the half-life, generally will contribute to the determination of the dosage. For example, therapeutic agents that are compatible with the human immune system, such as polypeptides comprising regions from humanized antibodies or fully human antibodies, may be used to prolong half-life of the polypeptide and to prevent the polypeptide being attacked by the host's immune system.

### Delivery

The various aspect described in this disclosure can also involve delivery the autoregulatory Cas13d nucleic acid molecules to cells and/or subjects. Any suitable means for delivery is contemplated and would be well-known to the skilled person. In some aspects, the invention provides methods comprising delivering one or more polynucleotides, or one or more vectors as described herein, one or more transcripts thereof, and/or one or proteins transcribed therefrom, to a host cell. In some aspects, the invention further provides cells produced by such methods, and organisms (such as animals, plants, or fungi) comprising or produced from such cells. In some embodiments, a nucleic acid comprising an autoregulated Cas13d as described herein in combination with (and optionally complexed with) a guide sequence is delivered to a cell.

In some embodiments, the method of delivery provided comprises nucleofection, microinjection, biolistics, virosomes, liposomes, immunoliposomes, polycation or lipid:nucleic acid conjugates, naked DNA, artificial virions, and agent-enhanced uptake of DNA.

Exemplary methods of delivery of nucleic acids include lipofection, nucleofection, electoporation, stable genome integration (e.g., piggybac), microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™, Lipofectin™ and SF Cell Line 4D-Nucleofector X Kit™ (Lonza)). Cationic and neutral lipids that are suitable for efficient receptor-recognition lipofection of polynucleotides include those of Feigner, WO 91/17424; WO 91/16024. Delivery may be to cells (e.g., in vitro or ex vivo administration) or target tissues (e.g., in vivo administration). Delivery may be achieved through the use of RNP complexes.

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

Additional methods for the delivery of nucleic acids to cells are known to those skilled in the art. See, for example, US 2003/0087817, incorporated herein by reference.

Other aspects of the present disclosure provide methods of delivering the Cas13d autoregulatory constructs into a cell. For example, in some embodiments, a cell is contacted with a construct, plasmid or vector described herein (e.g., constructs comprising Cas13d autoregulatory constructs or AAV particles containing nucleic acid vectors comprising such nucleotide sequences). In some embodiments, the contacting results in the delivery of such nucleotide sequences into a cell.

It should be appreciated that any rAAV particle, nucleic acid molecule or composition provided herein may be introduced into the cell in any suitable way, either stably or transiently. In some embodiments, the disclosed proteins may be transfected into the cell. In some embodiments, the cell may be transduced or transfected with a nucleic acid molecule. For example, a cell may be transduced (e.g., with a virus encoding a Cas13d autoregulatory construct), or transfected (e.g., with a plasmid encoding a Cas13d autoregulatory construct) with a nucleic acid molecule, or an rAAV particle containing a viral genome encoding one or more nucleic acid molecules. Such transduction may be a stable or transient transduction. In some embodiments, cells expressing a split protein or containing a split protein may be transduced or transfected with one or more guide RNA sequences. In some embodiments, a plasmid expressing a Cas13d autoregulatory construct may be introduced into cells through electroporation, transient (e.g., lipofection) and stable genome integration (e.g., piggybac) and viral transduction or other methods known to those of skill in the art.

In certain embodiments, the compositions provided herein comprise a lipid and/or polymer. In certain embodiments, the lipid and/or polymer is cationic. The preparation of such lipid particles is well known. See, e.g., U.S. Pat. Nos. 4,880,635; 4,906,477; 4,911,928; 4,917,951; 4,920,016; 4,921,757; and 9,737,604, each of which is incorporated herein by reference.

The compositions of this disclosure may be administered or packaged as a unit dose, for example. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent, i.e., a carrier or vehicle.

Treatment of a disease or disorder includes delaying the development or progression of the disease, or reducing disease severity. Treating the disease does not necessarily require curative results.

As used therein, “delaying” the development of a disease means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset.

As used herein “onset” or “occurrence” of a disease includes initial onset and/or recurrence. Conventional methods, known to those of ordinary skill in the art of medicine, can be used to administer the isolated polypeptide or pharmaceutical composition to the subject, depending upon the type of disease to be treated or the site of the disease.

### Vectors for Delivery

In some aspects, a vector is used to deliver the autoregulatory Cas13d nucleic acid molecules described herein to cells and/or subjects. The term “vector,” as used herein, refers to a nucleic acid that can be modified to encode a gene of interest and that is able to enter into a host cell, mutate and replicate within the host cell, and then transfer a replicated form of the vector into another host cell. Exemplary suitable vectors include viral vectors, such as retroviral vectors or bacteriophages and filamentous phage, and conjugative plasmids. Adeno-associated vectors are also suitable for delivery of a construct of interest (e.g., a nucleic acid encoding the Cas13d fusion protein). An “adeno-associated virus” or “AAV” is a virus which infects humans and some other primate species. The wild-type AAV genome is a single-stranded deoxyribonucleic acid (ssDNA), either positive- or negative-sensed. The genome comprises two inverted terminal repeats (ITRs), one at each end of the DNA strand, and two open reading frames (ORFs): rep and cap between the ITRs. The rep ORF comprises four overlapping genes encoding Rep proteins required for the AAV life cycle. The cap ORF comprises overlapping genes encoding capsid proteins: VP1, VP2 and VP3, which interact together to form the viral capsid. VP1, VP2 and VP3 are translated from one mRNA transcript, which can be spliced in two different manners: either a longer or shorter intron can be excised resulting in the formation of two isoforms of mRNAs: a ˜2.3 kb- and a ˜2.6 kb-long mRNA isoform. The capsid forms a supramolecular assembly of approximately 60 individual capsid protein subunits into a non-enveloped, T-1 icosahedral lattice capable of protecting the AAV genome. The mature capsid is composed of VP1, VP2, and VP3 (molecular masses of approximately 87, 73, and 62 kDa respectively) in a ratio of about 1:1:10. In some embodiments, the vector after infection of a first cell is not able to transfer to another host cell. In some embodiments, the vector does not replicate after entering the host cell.

In some embodiments an rAAV vector comprises any one of the constructs disclosed herein. In some embodiments, the rAAV vector of the present disclosure comprises one or more regulatory elements to control the expression of the heterologous nucleic acid region (e.g., promoters, transcriptional terminators, and/or other regulatory elements). In some embodiments, the first and/or second nucleotide sequence is operably linked to one or more (e.g., 1, 2, 3, 4, 5, or more) transcriptional terminators. Non-limiting examples of transcriptional terminators that may be used in accordance with the present disclosure include transcription terminators of the bovine growth hormone gene (bGH), human growth hormone gene (hGH), SV40, CW3, 4, or combinations thereof. In some embodiments, the transcriptional terminator used in the present disclosure is a bGH transcriptional terminator. In some embodiments, the rAAV vector further comprises a Woodchuck Hepatitis Virus Posttranscriptional Regulatory Element (WPRE). In certain embodiments, the WPRE is a truncated WPRE sequence, such as “W3.” In some embodiments, the WPRE is inserted 5′ of the transcriptional terminator. Such sequences, when transcribed, create a tertiary structure which enhances expression, in particular, from viral vectors.

In some embodiments, the vectors used herein may encode the Cas13d fusion proteins or Cas13d encoded on the same transcript as the Cas13d processing sequence (e.g., pre-guide RNA). The vectors may be capable of driving expression of one or more coding sequences in a cell. In some embodiments, the cell may be a prokaryotic cell, such as, e.g., a bacterial cell. In some embodiments, the cell may be a eukaryotic cell, such as, e.g., a yeast, plant, insect, or mammalian cell. In some embodiments, the eukaryotic cell may be a mammalian cell. In some embodiments, the eukaryotic cell may be a rodent cell. In some embodiments, the eukaryotic cell may be a human cell. Suitable promoters to drive expression in different types of cells are known in the art. In some embodiments, the promoter may be wild-type. In other embodiments, the promoter may be modified for more efficient or efficacious expression. In yet other embodiments, the promoter may be truncated yet retain its function. For example, the promoter may have a normal size or a reduced size that is suitable for proper packaging of the vector into a virus.

In some embodiments, the promoters that may be used in the vectors disclosed herein may be constitutive, inducible, or tissue-specific. In some embodiments, the promoters may be a constitutive promoters. Non-limiting exemplary constitutive promoters include cytomegalovirus immediate early promoter (CMV), simian virus (SV40) promoter, adenovirus major late (MLP) promoter, Rous sarcoma virus (RSV) promoter, mouse mammary tumor virus (MMTV) promoter, phosphoglycerate kinase (PGK) promoter, elongation factor-alpha (EF1a) promoter, ubiquitin promoters, actin promoters, tubulin promoters, immunoglobulin promoters, a functional fragment thereof, or a combination of any of the foregoing. In some embodiments, the promoter may be a CMV promoter. In some embodiments, the promoter may be a truncated CMV promoter. In other embodiments, the promoter may be an EF1a promoter. In some embodiments, the promoter may be an inducible promoter. Non-limiting exemplary inducible promoters include those inducible by heat shock, light, chemicals, peptides, metals, steroids, antibiotics, or alcohol. In some embodiments, the inducible promoter may be one that has a low basal (non-induced) expression level, such as, e.g., the Tet-On® promoter (Clontech). In some embodiments, the promoter may be a tissue-specific promoter. In some embodiments, the tissue-specific promoter is exclusively or predominantly expressed in liver tissue. Non-limiting exemplary tissue-specific promoters include B29 promoter, CD14 promoter, CD43 promoter, CD45 promoter, CD68 promoter, desmin promoter, elastase-1 promoter, endoglin promoter, fibronectin promoter, Flt-1 promoter, GFAP promoter, GPIIb promoter, ICAM-2 promoter, INF-β promoter, Mb promoter, Nphsl promoter, OG-2 promoter, SP-B promoter, SYN1 promoter, and WASP promoter.

In some embodiments, the vectors disclosed herein (e.g., vectors comprising Cas13d capable of autoregulation) may comprise inducible promoters to start expression only after it is delivered to a target cell. Non-limiting exemplary inducible promoters include those inducible by heat shock, light, chemicals, peptides, metals, steroids, antibiotics, or alcohol. In some embodiments, the inducible promoter may be one that has a low basal (non-induced) expression level, such as, e.g., the Tet-On® promoter (Clontech).

In additional embodiments, the vectors disclosed herein (e.g., vectors comprising Cas13d capable of autoregulation) may comprise tissue-specific promoters to start expression only after it is delivered into a specific tissue. Non-limiting exemplary tissue-specific promoters include B29 promoter, CD14 promoter, CD43 promoter, CD45 promoter, CD68 promoter, desmin promoter, elastase-1 promoter, endoglin promoter, fibronectin promoter, Flt-1 promoter, GFAP promoter, GPIIb promoter, ICAM-2 promoter, INF-β promoter, Mb promoter, Nphsl promoter, OG-2 promoter, SP-B promoter, SYN1 promoter, and WASP promoter.

In some embodiments, the nucleotide sequence encoding cas13d or Cas13d fusion protein may be operably linked to at least one transcriptional or translational control sequence. In some embodiments, the nucleotide sequence encoding the guide RNA may be operably linked to at least one promoter. In some embodiments, the promoter may be recognized by RNA polymerase III (Pol III). Non-limiting examples of Pol III promoters include U6, HI and tRNA promoters. In some embodiments, the nucleotide sequence encoding the guide RNA may be operably linked to a mouse or human U6 promoter. In other embodiments, the nucleotide sequence encoding the guide RNA may be operably linked to a mouse or human HI promoter. In some embodiments, the nucleotide sequence encoding the guide RNA may be operably linked to a mouse or human tRNA promoter. In embodiments with more than one guide RNA, the promoters used to drive expression may be the same or different. In some embodiments, the nucleotide encoding the crRNA of the guide RNA and the nucleotide encoding the tracr RNA of the guide RNA may be provided on the same vector. In some embodiments, the nucleotide encoding the crRNA and the nucleotide encoding the tracr RNA may be driven by the same promoter. In some embodiments, the crRNA and tracr RNA may be transcribed into a single transcript. For example, the crRNA and tracr RNA may be processed from the single transcript to form a double-molecule guide RNA. Alternatively, the crRNA and tracr RNA may be transcribed into a single-molecule guide RNA.

The Cas13d autoregulation system may comprise one vector or two vectors or more. In some embodiments, the vector system may comprise one single vector, which encodes both the Cas13d and the guide RNA. In other embodiments, the vector system may comprise two vectors, wherein one vector encodes the Cas13d fusion protein and the other encodes the gRNA.

In some embodiments, the composition comprising the rAAV particle (in any form contemplated herein) further comprises a pharmaceutically acceptable carrier. In some embodiments, the composition is formulated in appropriate pharmaceutical vehicles for administration to human or animal subjects.

In some embodiments, the AAV comprising Cas13d autoregulation system is an AAV6 AAV. In some embodiments, an AAV comprises a nucleic acid sequence having at least 80%, 85%, 90%, 95%, or 99% or more sequence identity with SEQ ID NO: 178. In some embodiments, an AAV comprises a nucleic acid sequence of SEQ ID NO: 178. In some embodiments, an AAV consists of a nucleic acid sequence of SEQ ID NO: 178. In some embodiments, an AAV comprises a nucleic acid sequence having at least 80%, 85%, 90%, 95%, or 99% or more sequence identity with SEQ ID NO: 179. In some embodiments, an AAV comprises a nucleic acid sequence of SEQ ID NO: 179. In some embodiments, an AAV consists of a nucleic acid sequence of SEQ ID NO: 179.

Additional suitable vectors will be apparent to those of skill in the art based on the instant disclosure.

### Type of Cells

In some aspects, the Cas13d nucleic acid molecules described herein are delivered to and/or contained, expressed, or otherwise present within cells and/or cell lines under in vitro, in vivo, and/or ex vivo conditions.

Cells that may contain any of the compositions, constructs, plasmid or vectors described herein include prokaryotic cells and eukaryotic cells. The methods described herein are used to deliver a construct, plasmid or vector of the disclosure into a eukaryotic cell (e.g., a mammalian cell, such as a human cell). In some embodiments, the cell is in vitro (e.g., cultured cell. In some embodiments, the cell is in vivo (e.g., in a subject such as a human subject). In some embodiments, the cell is ex vivo (e.g., isolated from a subject and may be administered back to the same or a different subject).

Mammalian cells of the present disclosure include human cells, primate cells (e.g., vero cells), rat cells (e.g., GH3 cells, OC23 cells) or mouse cells (e.g., MC3T3 cells). There are a variety of human cell lines, including, without limitation, human embryonic kidney (HEK) cells, HeLa cells, cancer cells from the National Cancer Institute's 60 cancer cell lines (NCI60), DU145 (prostate cancer) cells, Lncap (prostate cancer) cells, MCF-7 (breast cancer) cells, MDA-MB-438 (breast cancer) cells, PC3 (prostate cancer) cells, T47D (breast cancer) cells, THP-1 (acute myeloid leukemia) cells, U87 (glioblastoma) cells, SHSY5Y human neuroblastoma cells (cloned from a myeloma) and Saos-2 (bone cancer) cells. In some embodiments, rAAV vectors are delivered into human embryonic kidney (HEK) cells (e.g., HEK 293 or HEK 293T cells). In some embodiments, rAAV vectors are delivered into stem cells (e.g., human stem cells) such as, for example, pluripotent stem cells (e.g., human pluripotent stem cells including human induced pluripotent stem cells (hiPSCs)). A stem cell refers to a cell with the ability to divide for indefinite periods in culture and to give rise to specialized cells. A pluripotent stem cell refers to a type of stem cell that is capable of differentiating into all tissues of an organism, but not alone capable of sustaining full organismal development. A human induced pluripotent stem cell refers to a somatic (e.g., mature or adult) cell that has been reprogrammed to an embryonic stem cell-like state by being forced to express genes and factors important for maintaining the defining properties of embryonic stem cells (see, e.g., Takahashi and Yamanaka, Cell 126 (4): 663-76, 2006, incorporated by reference herein). Human induced pluripotent stem cell cells express stem cell markers and are capable of generating cells characteristic of all three germ layers (ectoderm, endoderm, mesoderm).

Additional non-limiting examples of cell lines that may be used in accordance with the present disclosure include 293-T, 293-T, 3T3, 4T1, 721, 9L, A-549, A172, A20, A253, A2780, A2780ADR, A2780cis, A431, ALC, B16, B35, BCP-1, BEAS-2B, bEnd.3, BHK-21, BR 293, BxPC3, C2C12, C3H-10T1/2, C6, C6/36, Cal-27, CGR8, CHO, CML T1, CMT, COR-L23, COR-L23/5010, COR-L23/CPR, COR-L23/R23, COS-7, COV-434, CT26, D17, DH82, DU145, DuCaP, E14Tg2a, EL4, EM2, EM3, EMT6/AR1, EMT6/AR10.0, FM3, H1299, H69, HB54, HB55, HCA2, Hepa1c1c7, High Five cells, HL-60, HMEC, HT-29, HUVEC, J558L cells, Jurkat, JY cells, K562 cells, KCL22, KG1, Ku812, KYO1, LNCap, Ma-Mel 1, 2, 3 . . . 48, MC-38, MCF-10A, MCF-7, MDA-MB-231, MDA-MB-435, MDA-MB-468, MDCK II, MG63, MONO-MAC 6, MOR/0.2R, MRC5, MTD-1A, MyEnd, NALM-1, NCI-H69/CPR, NCI-H69/LX10, NCI-H69/LX20, NCI-H69/LX4, NIH-3T3, NW-145, OPCN/OPCT Peer, PNT-1A/PNT 2, PTK2, Raji, RBL cells, RenCa, RIN-5F, RMA/RMAS, S2, Saos-2 cells, Sf21, Sf9, SiHa, SKBR3, SKOV-3, T-47D, T2, T84, THP1, U373, U87, U937, VCaP, WM39, WT-49, X63, YAC-1 and YAR cells.

Some aspects of this disclosure provide cells comprising any of the nucleic acid molecules disclosed herein. In some embodiments, a host cell is transiently or non-transiently transfected with one or more vectors described herein. In some embodiments, a cell is transfected as it naturally occurs in a subject. In some embodiments, a cell that is transfected is taken from a subject. In some embodiments, the cell is derived from cells taken from a subject, such as a cell line. A wide variety of cell lines for tissue culture are known in the art. Examples of cell lines include, but are not limited to, C8161, CCRF-CEM, MOLT, mIMCD-3, NHDF, HeLa-S3, Huh1, Huh4, Huh7, HUVEC, HASMC, HEKn, HEKa, MiaPaCell, Panc1, PC-3, TF1, CTLL-2, CIR. Rat6, CV1, RPTE, A10, T24, J82, A375, ARH-77, Calu1, SW480, SW620, SKOV3, SK-UT, CaCo2, P388D1, SEM-K2, WEHI-231, HB56, T1B55, Jurkat, J45.01, LRMB, Bcl-1, BC-3, IC21, DLD2, Raw264.7, NRK, NRK-52E, MRC5, MEF, Hep G2, HeLa B, HeLa T4, COS. COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A 172, A20, A253, A431, A-549, ALC, B16, B35, BCP-1 cells, BEAS-2B, bEnd.3, BHK-21, BR 293. BxPC3. C3H-10T1/2, C6/36, Cal-27, CHO, CHO-7, CHO-IR, CHO-K1, CHO-K2, CHO-T, CHO Dhfr −/−, COR-L23, COR-L23/CPR, COR-L23/5010, COR-L23/R23, COS-7, COV-434, CML T1, CMT, CT26, D17, DH82, DU145. DuCaP, EL4, EM2, EM3, EMT6/AR1, EMT6/AR10.0, FM3, H1299, H69, HB54, HB55, HCA2, HEK-293, HeLa, Hepa1c1c7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK 11, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, YAR, and transgenic varieties thereof.

Cell lines are available from a variety of sources known to those with skill in the art (see, e.g., the American Type Culture Collection (ATCC) (Manassas, Va.)). In some embodiments, a cell transfected with one or more vectors described herein is used to establish a new cell line comprising one or more vector-derived sequences. In some embodiments, a cell transiently transfected with the components of a CRISPR system as described herein (such as by transient transfection of one or more vectors, or transfection with RNA), and modified through the activity of a CRISPR complex, is used to establish a new cell line comprising cells containing the modification but lacking any other exogenous sequence. In some embodiments, cells transiently or non-transiently transfected with one or more vectors described herein, or cell lines derived from such cells are used in assessing one or more test compounds.

### Pharmaceutical Composition

In some aspects, pharmaceutical compositions comprise the Cas13d nucleic acid molecules described herein. In some embodiments, the pharmaceutical composition further comprises a pharmaceutically acceptable carrier. “Pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. A “pharmaceutically acceptable carrier” may be a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation and not injurious to the tissue of the patient (e.g., physiologically compatible, sterile, physiologic pH, etc.). The term “carrier” denotes an organic or inorganic ingredient, natural or synthetic, with which the active ingredient is combined to facilitate the application. The components of the pharmaceutical compositions also are capable of being co-mingled with the molecules of the present disclosure, and with each other, in a manner such that there is no interaction which would substantially impair the desired pharmaceutical efficacy. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and tale; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation.

The pharmaceutical compositions may conveniently be presented in unit dosage form and may be prepared by any of the methods well-known in the art of pharmacy. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

The formulation of the pharmaceutical composition may dependent upon the route of administration. Injectable preparations suitable for parenteral administration or intratumoral, peritumoral, intralesional or perilesional administration include, for example, sterile injectable aqueous or oleaginous suspensions and may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3 propanediol or 1,3 butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil may be employed including synthetic mono- or di-glycerides. In addition, fatty acids such as oleic acid find use in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

Compositions suitable for oral administration may be presented as discrete units, such as capsules, tablets, lozenges, each containing a predetermined amount of the anti-inflammatory agent. Other compositions include suspensions in aqueous liquids or non-aqueous liquids such as a syrup, elixir or an emulsion.

In some embodiments, the pharmaceutical compositions used for therapeutic administration must be sterile. Sterility is readily accomplished by filtration through sterile filtration membranes (e.g., 0.2 micron membranes). Alternatively, preservatives can be used to prevent the growth or action of microorganisms. Various preservatives are well known and include, for example, phenol and ascorbic acid. The pharmaceutical composition ordinarily will be stored in lyophilized form or as an aqueous solution if it is highly stable to thermal and oxidative denaturation. The pH of the preparations typically will be about from 6 to 8, although higher or lower pH values can also be appropriate in certain instances.

In another aspect, provided herein are pharmaceutical compositions comprising the constructs described herein and a pharmaceutically acceptable carrier.

In another aspect, provided herein are pharmaceutical compositions comprising the constructs described herein and optionally a pharmaceutically acceptable carrier.

### Kits

The nucleic acids, vectors, constructions, plasmid, compositions and cells of the present disclosure may be assembled into kits. In some embodiments, the kit comprises nucleic acid vectors for the expression of any of the constructs, plasmids or vectors describe herein. In other embodiments, the kit further comprises appropriate guide nucleotide sequences or nucleic acid vectors for the expression of such guide nucleotide sequences, to target the Cas13d protein to the desired target transcript.

The kit described herein may include one or more containers housing components for performing the methods described herein and optionally instructions for use. Any of the kit described herein may further comprise components needed for performing the assay methods. Each component of the kits, where applicable, may be provided in liquid form (e.g., in solution) or in solid form, (e.g., a dry powder). In certain cases, some of the components may be reconstitutable or otherwise processible (e.g., to an active form), for example, by the addition of a suitable solvent or other species (for example, water), which may or may not be provided with the kit.

In some embodiments, the kits may optionally include instructions and/or promotion for use of the components provided. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, USB drive, digital media, etc.), Internet, and/or web-based communications, etc. The written instructions may be in a form prescribed by a governmental agency regulating the manufacture, use, or sale of pharmaceuticals or biological products, which can also reflect approval by the agency of manufacture, use or sale for animal administration. As used herein, “promoted” includes all methods of doing business including methods of education, hospital and other clinical instruction, scientific inquiry, drug discovery or development, academic research, pharmaceutical industry activity including pharmaceutical sales, and any advertising or other promotional activity including written, oral and electronic communication of any form, associated with the disclosure. Additionally, the kits may include other components depending on the specific application, as described herein.

The kits may contain any one or more of the components described herein in one or more containers. The components may be prepared sterilely, packaged in a syringe and shipped refrigerated. Alternatively, it may be housed in a vial or other container for storage. A second container may have other components prepared sterilely. Alternatively, the kits may include the active agents premixed and shipped in a vial, tube, or other container.

The kits may have a variety of forms, such as a blister pouch, a shrink wrapped pouch, a vacuum sealable pouch, a sealable thermoformed tray, or a similar pouch or tray form, with the accessories loosely packed within the pouch, one or more tubes, containers, a box or a bag. The kits may be sterilized after the accessories are added, thereby allowing the individual accessories in the container to be otherwise unwrapped. The kits can be sterilized using any appropriate sterilization techniques, such as radiation sterilization, heat sterilization, or other sterilization methods known in the art. The kits may also include other components, depending on the specific application, for example, containers, cell media, salts, buffers, reagents, syringes, needles, a fabric, such as gauze, for applying or removing a disinfecting agent, disposable gloves, a support for the agents prior to administration, etc.

Other aspects of this disclosure provide kits comprising one or more nucleic acid constructs encoding the various components of the Cas13d autoregulation system described herein, e.g., the comprising a nucleotide sequence encoding the Cas13d autoregulation systems capable of knocking down expression of a target sequence and reducing collateral damage (compared to a kit without Cas13d autoregulation). In some embodiments, the nucleotide sequence comprises a heterologous promoter that drives expression of the Cas13d autoregulation system components.

Some aspects of this disclosure provide kits comprising a nucleic acid construct, comprising (a) a nucleotide sequence encoding a Cas13d protein fused to ZnF domain and KRAB domain and (b) a heterologous promoter that drives expression of the sequence of (a).

Some aspects of this disclosure provide kits comprising a nucleic acid construct, comprising (a) a nucleotide sequence encoding on the same transcript a Cas13d protein and at least one guide RNA (b) a heterologous promoter that drives expression of the sequence of (a).

TABLE 2
SEQUENCES

| SEQ ID | | |
|---|---|---|
| NO: | Description | Sequence |
| 1 | AdmCas13d | MNNKRKTKAKAAGLKSVFFDQKQAVLTTFAKGNNSQIE |
| | domain | KKVVNSEVKDLRQPPAFDLELKEKTFYISGKNNINTSREN |
| | | PLASASLPLSKRQRIRAERIKRAREENRPYHNVKRVGEDD |
| | | LRAKADLEKHYFGKEYSDNLKIQIIYNILDINKIISPYINDI |
| | | VYSMNNLARNDEYIDGKIDVIGSLSSTTDYSSFMSPNKDL |
| | | EKEKKFSFHRENYKKFVEASKPYMRYYGKVFIRDVKKSK |
| | | LSTGKGEKIEVMYRSDEEIFTIFQILSYVRQSIMHNDIGNK |
| | | SSILAIEKYPARFVGFLSDLLKTKTNDVNRMFIDNNSQTN |
| | | FWVLFSIFGLQDHTSGADKICRNFYDFVIKADSKNLGFSL |
| | | KKIRELMLDLPNANMLRDHQFDTVRSKFYTLLDFIIYQHY |
| | | LEEKSRIDNMVEKLRMTLKEEEKEVLYAAEAKIVWNAIG |
| | | AKVINKLVPMMNGDALKEIKRKNRDRKLPQSVIATVQV |
| | | NSDANVFSGLIYFLTLFLDGKEINEMVSNLITKFENIDSLL |
| | | HVDREIYKSDEKDLDLEIEKLALFFKGVVRPNAKTDTGA |
| | | GEISKSFSIFQSAERIIEELKFIKNVTRMDNEIFPSEGVFLDA |
| | | ANVLGVRGDDFDFSNEFVGDDLHSDANKKIINKINGTKE |
| | | DRNLRNFIINNVVKSRRFQYIARHMNTHYVKQLANNETL |
| | | NRFVLNKMGDAKIINRYYESISGNTPNIEVRSQIDYLVKR |
| | | LRSFSFEDLNDVKQKVRPGTNESIEKEKKKALVGLCLTIQ |
| | | YLVYKNLVNINARYTTAFYCLERDSKLKGFGVDVWRDF |
| | | ESYTALTNHFIKEGYLPVRKAEILRANLKHLDCEDGFKYY |
| | | RNQVTHLNAIRVAYKYINEIKSVHSYFALYHYIMQRHLY |
| | | DSLQAKAKDSSGFVIDALKKSFEHKIYSKDLLHVLHSPFG |
| | | YNTARYKNLSIEALFDKNESRPEVNPLSTNDYPYDVPDY |
| | | A |
| 2 | EsCas13d | MGKKIHARDLREQRKTDRTEKFADQNKKREAERAVPKK |
| | domain | DAAVSVKSVSSVSSKKDNVTKSMAKAAGVKSVFAVGNT |
| | | VYMTSFGRGNDAVLEQKIVDTSHEPLNIDDPAYQLNVVT |
| | | MNGYSVTGHRGETVSAVTDNPLRRFNGRKKDEPEQSVPT |
| | | DMLCLKPTLEKKFFGKEFDDNIHIQLIYNILDIEKILAVYST |
| | | NAIYALNNMSADENIENSDFFMKRTTDETFDDFEKKKES |
| | | TNSREKADFDAFEKFIGNYRLAYFADAFYVNKKNPKGKA |
| | | KNVLREDKELYSVLTLIGKLRHWCVHSEEGRAEFWLYKL |
| | | DELKDDFKNVLDVVYNRPVEEINNRFIENNKVNIQILGSV |
| | | YKNTDIAELVRSYYEFLITKKYKNMGFSIKKLRESMLEGK |
| | | GYADKEYDSVRNKLYQMTDFILYTGYINEDSDRADDLV |
| | | NTLRSSLKEDDKTTVYCKEADYLWKKYRESIREVADALD |
| | | GDNIKKLSKSNIEIQEDKLRKCFISYADSVSEFTKLIYLLTR |
| | | FLSGKEINDLVTTLINKEDNIRSFLEIMDELGLDRTFTAEY |
| | | SFFEGSTKYLAELVELNSFVKSCSFDINAKRTMYRDALDI |
| | | LGIESDKTEEDIEKMIDNILQIDANGDKKLKKNNGLRNFIA |
| | | SNVIDSNRFKYLVRYGNPKKIRETAKCKPAVRFVLNEIPD |
| | | AQIERYYEACCPKNTALCSANKRREKLADMIAEIKFENFS |
| | | DAGNYQKANVTSRTSEAEIKRKNQAIRLYLTVMYIMLK |
| | | NLVNVNARYVIAFHCVERDTKLYAESGLEVGNIEKNKTN |
| | | LTMAVMGVKLENGIIKTEFDKSFAENAANRYLRNARWY |
| | | KLILDNLKKSERAVVNEFRNTVCHLNAIRNININIKEIKEV |
| | | ENYFALYHYLIQKHLENRFADKKVERDTGDFISKLEEHK |
| | | TYCKDFVKAYCTPFGYNLVRYKNLTIDGLEDKNYPGKD |
| | | DSDEQKYPYDVPDYA |
| 3 | P1E0Cas13d | MEREVKKPPKKSLAKAAGLKSTFVISPQEKELAMTAFGR |
| | domain | GNDALLQKRIVDGVVRDVAGEKQQFQVQRQDESRFRLQ |
| | | NSRLADRTVTADDPLHRAETPRRQPLGAGMDQLRRKAIL |
| | | EQKYFGRTFDDNIHIQLIYNILDIHKMLAVPANHIVHTLNL |
| | | LGGYGETDFVGMLPAGLPYDKLRVVKKKNGDTVDIKAD |
| | | IAAYAKRPQLAYLGAAFYDVTPGKSKRDAARGRVKREQ |
| | | DVYAILSLMSLLRQFCAHDSVRIWGQNTTAALYHLQALP |
| | | QDMKDLLDDGWRRALGGVNDHELDTNKVNLLTLFEYY |
| | | GAETKQARVALTQDFYRFVVLKEQKNMGFSLRRLREELL |
| | | KLPDAAYLTGQEYDSVRQKLYMLLDFLLCRLYAQERAD |
| | | RCEELVSALRCALSDEEKDTVYQAEAAALWQALGDTLR |
| | | RKLLPLLKGKKLQDKDKKKSDELGLSRDVLDGVLFRPAQ |
| | | QGSRANADYFCRLMHLSTWFMDGKEINTLLTTLISKLENI |
| | | DSLRSVLESMGLAYSFVPAYAMEDHSRYIAGQLRVVNNI |
| | | ARMRKPAIGAKREMYRAAVVLLGVDSPEAAAAITDDLL |
| | | QIDPETGKVRPRSDSARDTGLRNFIANNVVESRRFTYLLR |
| | | YMTPEQARVLAQNEKLIAFVLSTVPDTQLERYCRTCGRE |
| | | DITGRPAQIRYLTAQIMGVRYESFTDVEQRGRGDNPKKE |
| | | RYKALIGLYLTVLYLAVKNMVNCNARYVIAFYCRDR.DT |
| | | ALYQKEVCWYDLEEDKKSGKQRQVEDYTALTRYFVSQG |
| | | YLNRHACGYLRSNMNGISNSLLTAYRNAVDHLNAIPPLG |
| | | SLCRDIGRVDSYFALYHYAVQQYLNGRYYRKTPREQELF |
| | | AAMAQHRTWCSDLVKALNTPFGYNLARYKNLSIDGLFD |
| | | REGDHVVREDGEKPAEYPYDVPDYA |
| 4 | RaCas13d | MAKKSKGMSLREKRELEKQKRIQKAAVNSVNDTPEKTE |
| | domain | EANVVSVNVRTSAENKHSKKSAAKALGLKSGLVIGDELY |
| | | LTSFGRGNEAKLEKKISGDTVEKLGIGAFEVAERDESTLT |
| | | LESGRIKDKTARPKDPRHITVDTQGKFKEDMLGIRSVLEK |
| | | KIFGKTFDDNIHVQLAYNILDVEKIMAQYVSDIVYMLHN |
| | | TDKTERNDNLMGYMSIRNTYKTFCDTSNLPDDTKQKVE |
| | | NQKREFDKIIKSGRLGYFGEAFMVNSGNSTKLRPEKEIYH |
| | | IFALMASLRQSYFHGYVKDTDYQGTTWAYTLEDKLKGP |
| | | SHEFRETIDKIFDEGFSKISKDFGKMNKVNLQILEQMIGEL |
| | | YGSIERQNLTCDYYDFIQLKKHKYLGFSIKRLRETMLETT |
| | | PAECYKAECYNSERQKLYKLIDFLIYDLYYNRKPARIEEI |
| | | VDKLRESVNDEEKESIYSVEAKYVYESLSKVLDKSLKNS |
| | | VSGETIKDLQKRYDDETANRIWDISQHSISGNVNCFCKLI |
| | | YIMTLMLDGKEINDLLTTLVNKFDNIASFIDVMDELGLEH |
| | | SFTDNYKMFADSKAICLDLQFINSFARMSKIDDEKSKRQL |
| | | FRDALVILDIGNKDETWINNYLDSDIFKLDKEGNKLKGAR |
| | | HDFRNFIANNVIKSSRFKYLVKYSSADGMIKLKTNEKLIG |
| | | FVLDKLPETQIDRYYESCGLDNAVVDKKVRIEKLSGLIRD |
| | | MKFDDFSGVKTSNKAGDNDKQDKAKYQAIISLYLMVLY |
| | | QIVKNMIYVNSRYVIAFHCLERDFGMYGKDFGKYYQGC |
| | | RKLTDHFIEEKYMKEGKLGCNKKVGRYLKNNISCCTDGL |
| | | INTYRNQVDHFAVVRKIGNYAAYIKSIGSWFELYHYVIQR |
| | | IVFDEYRFALNNTESNYKNSIIKHHTYCKDMVKALNTPFG |
| | | YDLPRYKNLSIGDLFDRNNYLNKTKESIDANSSIDSQYPY |
| | | DVPDYA |
| 5 | RffCas13d | MKKKMSLREKREAEKQAKKAAYSAASKNTDSKPAEKK |
| | domain | AETPKPAEIISDNSRNKTAVKAAGLKSTIISGDKLYMTSFG |
| | | KGNAAVIEQKIDINDYSFSAMKDTPSLEVDKAESKEISFSS |
| | | HHPFVKNDKLTTYNPLYGGKDNPEKPVGRDMLGLKDKL |
| | | EERYFGCTFNDNLHIQIIYNILDIEKILAVHSANITTALDHM |
| | | VDEDDEKYLNSDYIGYMNTINTYDVEMDPSKNSSLSPKD |
| | | RKNIDNSRAKFEKLLSTKRLGYFGFDYDANGKDKKKNEE |
| | | IKKRLYHLTAFAGQLRQWSFHSAGNYPRTWLYKLDSLD |
| | | KEYLDTLDHYFDKRENDINDDFVTKNATNLYILKEVFPE |
| | | ANFKDIADLYYDFIVIKSHKNMGFSIKKLREKMLECDGA |
| | | DRIKEQDMDSVRSKLYKLIDFCIFKYYHEFPELSEKNVDIL |
| | | RAAVSDTKKDNLYSDEAARLWSIFKEKFLGFCDKIVVWV |
| | | TGEHEKDITSVIDKDAYRNRSNVSYFSKLMYAMCFELDG |
| | | KEINDLLTTLINKFDNIANQIKTAKELGINTAFVKNYDFEN |
| | | HSEKYVDELNIVKNIARMKKPSSNAKKAMYHDALTILGI |
| | | PEDMDEKALDEELDLILEKKTDPVTGKPLKGKNPLRNFIA |
| | | NNVIENSRFIYLIKFCNPENVRKIVNNTKVTEFVLKRIPDA |
| | | QIERYYKSCTDSEMNPPTEKKITELAGKLKDMNFGNFRN |
| | | VRQSAKENMEKERFKAVIGLYLTVVYRVVKNLVDVNSR |
| | | YIMAFHSLERDSQLYNVSVDNDYLALTDTLVKEGDNSRS |
| | | RYLAGNKRLRDCVKQDIDNAKKWFVSDKYNSITKYRNN |
| | | VAHLTAVRNCAEFIGDITKIDSYFALYHYLIQRQLAKGLD |
| | | HERSGFDRNYPQYAPLFKWHTYVKDVVKALNAPFGYNI |
| | | PRFKNLSIDALFDRNEIKKNDGEKKSDDYPYDVPDYA |
| 6 | RfxCas13d | MIEKKKSFAKGMGVKSTLVSGSKVYMTTFAEGSDARLE |
| | domain | KIVEGDSIRSVNEGEAFSAEMADKNAGYKIGNAKFSHPK |
| | | GYAVVANNPLYTGPVQQDMLGLKETLEKRYFGESADGN |
| | | DNICIQVIHNILDIEKILAEYITNAAYAVNNISGLDKDIIGF |
| | | GKFSTVYTYDEFKDPEHHRAAFNNNDKLINAIKAQYDEF |
| | | DNFLDNPRLGYFGQAFFSKEGRNYIINYGNECYDILALLS |
| | | GLRHWVVHNNEEESRISRTWLYNLDKNLDNEYISTLNYL |
| | | YDRITNELTNSFSKNSAANVNYIAETLGINPAEFAEQYFRF |
| | | SIMKEQKNLGFNITKLREVMLDRKDMSEIRKNHKVFDSIR |
| | | TKVYTMMDFVIYRYYIEEDAKVAAANKSLPDNEKSLSEK |
| | | DIFVINLRGSENDDQKDALYYDEANRIWRKLENIMHNIKE |
| | | FRGNKTREYKKKDAPRLPRILPAGRDVSAFSKLMYALTM |
| | | FLDGKEINDLLTTLINKEDNIQSFLKVMPLIGVNAKFVEEY |
| | | AFFKDSAKIADELRLIKSFARMGEPIADARRAMYIDAIRIL |
| | | GTNLSYDELKALADTFSLDENGNKLKKGKHGMRNFIINN |
| | | VISNKRFHYLIRYGDPAHLHEIAKNEAVVKFVLGRIADIQ |
| | | KKQGQNGKNQIDRYYETCIGKDKGKSVSEKVDALTKIIT |
| | | GMNYDQFDKKRSVIEDTGRENAEREKFKKIISLYLTVIYH |
| | | ILKNIVNINARYVIGFHCVERDAQLYKEKGYDINLKKLEE |
| | | KGFSSVTKLCAGIDETAPDKRKDVEKEMAERAKESIDSLE |
| | | SANPKLYANYIKYSDEKKAEEFTRQINREKAKTALNAYL |
| | | RNTKWNVIIREDLLRIDNKTCTLFRNKAVHLEVARYVHA |
| | | YINDIAEVNSYFQLYHYIMQRIIMNERYEKSSGKVSEYED |
| | | AVNDEKKYNDRLLKLLCVPFGYCIPRFKNLSIEALFDRNE |
| | | AAKFDKEKKKVSGNS |
| 7 | UrCas13d | MAKKNKMKPRELREAQKKARQLKAAEINNNAAPAIAA |
| | domain | MPAAEVIAPVAEKKKSSVKAAGMKSILVSENKMYITSFG |
| | | KGNSAVLEYEVDNNDYNKTQLSSKDNSNIELGDVNEVNI |
| | | TFSSKHGFGSGVEINTSNPTHRSGESSPVRGDMLGLKSEL |
| | | EKRFFGKTFDDNIHIQLIYNILDIEKILAVYVTNIVYALNN |
| | | MLGIKDSESYDDFMGYLSARNTYEVFTHPDKSNLSDKVK |
| | | GNIKKSLSKENDLLKTKRLGYFGLEEPKTKDTRASEAYK |
| | | KRVYHMLAIVGQIRQCVFHDKSGAKRFDLYSFINNIDPEY |
| | | RDTLDYLVEERLKSINKDFIEGNKVNISLLIDMMKGYEAD |
| | | DIIRLYYDFIVLKSQKNLGFSIKKLREKMLEEYGFRFKDK |
| | | QYDSVRSKMYKLMDFLLFCNYYRNDVAAGEALVRKLRF |
| | | SMTDDEKEGIYADEAAKLWGKERNDFENIADHMNGDVI |
| | | KELGKADMDFDEKILDSEKKNASDLLYFSKMIYMLTYFL |
| | | DGKEINDLLTTLISKFDNIKEFLKIMKSSAVDVECELTAGY |
| | | KLFNDSQRITNELFIVKNIASMRKPAASAKLTMERDALTIL |
| | | GIDDNITDDRISEILKLKEKGKGIHGLRNFITNNVIESSRFV |
| | | YLIKYANAQKIREVAKNEKVVMFVLGGIPDTQIERYYKS |
| | | CVEFPDMNSSLEAKRSELARMIKNISFDDFKNVKQQAKG |
| | | RENVAKERAKAVIGLYLTVMYLLVKNLVNVNARYVIAI |
| | | HCLERDFGLYKEIIPELASKNLKNDYRILSQTLCELCDDR |
| | | NESSNLFLKKNKRLRKCVEVDINNADSSMTRKYRNCIAH |
| | | LTVVRELKEYIGDIRTVDSYFSIYHYVMQRCITKRGDDTK |
| | | QEEKIKYEDDLLKNHGYTKDFVKALNSPFGYNIPRFKNLS |
| | | IEQLFDRNEYLTEK |
| 8 | HLTR3 ZnF | AQAALEPGEKPYACPECGKSFSRSDDLVRHQRTHTGEKP |
| | domain | YKCPECGKSFSRSDHLTTHQRTHTGEKPYKCPECGKSFSD |
| | | CRDLARHQRTHTGEKPYACPECGKSFSRSDVLVRHQRTH |
| | | TGEKPYKCPECGKSFSDPGHLVRHQRTHTGEKPYKCPEC |
| | | GKSFSQSSHLVRHQRTHTGKKTSGQAGQY |
| 9 | HLTR3 binding | GGAGGCGTGGCCTGGGCG |
| | motif | |
| 10 | HLTR1 ZnF | AQAALEPGEKPYACPECGKSFSTSGHLVRHQRTHTGEKP |
| | domain | YKCPECGKSFSRSDVLVRHQRTHTGEKPYKCPECGKSFS |
| | | QSGDLRRHQRTHTGEKPYACPECGKSFSQSSNLASHQRT |
| | | HTGEKPYKCPECGKSFSTSGELVRHQRTHTGEKPYKCPE |
| | | CGKSFSRSDNLVRHQRTHTGKKTSGQAGQY |
| 11 | HLTR1 binding | GAGGCTTAAGCAGTGGGT |
| | motif | |
| 12 | HLTR6 ZnF | AQAALEPGEKPYACPECGKSFSSPADLTRHQRTHTGEKP |
| | domain | YKCPECGKSFSDPGALVRHQRTHTGEKPYKCPECGKSFS |
| | | QLAHLRAHQRTHTGEKPYACPECGKSFSDKKDLTRHQRT |
| | | HTGEKPYKCPECGKSFSTSGSLVRHQRTHTGEKPYKCPEC |
| | | GKSFSQRHSLTEHQRTHTGKKTSGQAGQY |
| 13 | HLTR6 binding | CTAGTTACCAGAGTCACA |
| | motif | |
| 14 | mTYR ZnF | TTCCAGATCTCTGATGGCCATTTTCCTCGAGCCTGTGCC |
| | domain | TCCTCTAAGAACTTGTTGGCAAAAGAATGCTGCCCACC |
| | | ATGGATGGGTGATGGGAGTCCCTGCGGCCAGCTTTCAG |
| | | GCAGAGGTTCCTGCCAGGATATCCTTCTGTCCAGTGCA |
| | | CCATCTGGACCTCAGTTCCCCTTCAAA |
| | | GGGGTGGATGACCGTGAGTCCTGGCCCTCTGTGTTTTA |
| | | TAATAGGACCTGCCAGTGCTCAGGCAACTTCATGGGTT |
| | | TCAACTGCGGAAACTGTAAGTTTGGATTTGGGGGCCCA |
| | | AATTGTACAGAGAAGCGAGTCTTGATTAGAAGAAACA |
| | | TTTTTGATTTGAGTGTCTCCGAAAAGAAT |
| | | AAGTTCTTTTCTTACCTCACTTTAGCAAAACATACTATC |
| | | AGCTCAGTCTATGTCATCCCCACAGGCACCTATGGCCA |
| | | AATGAACAATGGGTCAACACCCATGTTTAATGATATCA |
| | | ACATCTACGACCTCTTTGTATGGAT |
| | | GCATTACTATGTGTCAAGGGACACACTGCTTGGGGGCT |
| | | CT |
| 15 | mTYR binding | TTCCCCTTCNNNNNNGTGGATGAC |
| | motif | |
| 16 | mCFTR ZnF | ATTGGAATAATTGGACGCAAGAAAGGGATAAGTAATT |
| | domain | TGATCAAACAATTTAGCTGTTGTTTTTATTTGTAGACAT |
| | | CACTCCTGATGTTGATTTTGGGAGAACTGGAAGCTTCA |
| | | GAGGGAATTATTAAGCACAGTGGAAGAGTTTCATTCTG |
| | | CTCTCAATTTTCTTGGATTATGCCGGGT |
| | | ACTATCAAAGAAAATATCATCTTTGGTGTTTCCTATGA |
| | | TGAGTACAGATATAAGAGTGTTGTCAAAGCTTGCCAAC |
| | | TACAGCAGGTAAGCATATTTATGAAAAATGCTGATTGT |
| | | GTTAGCTACTTGTGTCAGTGTTGTGA |
| | | TAAAATTGCTTGACTACTCACCTTGAAAAGGGTTTTAT |
| | | TT |
| | | TAAATTCTTTTCAGGGATGATACCGTCCATCTTGGCAA |
| | | AGGAGGGGCAGGAATGGGAAGATGGCGAGACATGTTA |
| | | TATCCATAGTCAGGA |
| | | AGCAGACAGCCAGCAGGAAGTGGGGCTTCA |
| 17 | mCFTR binding | TCACTCCTGNNNNNNNNNTTGGGAGAA |
| | motif | |
| 18 | hDMPK ZnF | GCCCAGGAGCCGCCCGCGCTCCCTGAACCCTAGAACTG |
| | domain | TCTTCGACTCCGGGGCCCCGTTGGAAGACTGAGTGCCC |
| | | GGGGCACGGCACAGAAGCCGCGCCCACCGCCTGCCAG |
| | | TTCACAACCGCTCCGAGCGTGGGTCTCCGCCCAGCTCC |
| | | AGTCCTGTGTACCGGGCCCGCCCCCTAGCGGCCGGGGA |
| | | GGGAGGGGCCGGGTCCGCGGCCGGCGAACGGGGCTCG |
| | | AAGGGTCCTTGTAGCCGGGAATGCTGCTGCTGCTGCTG |
| | | CTGCTGCTGCTGCTGCTGGGGGGATCACAGACCATTTC |
| | | TTTCTTTCGGCCAGGCTGAGGCCCTGACGTGGATGGGC |
| | | AAACTGCAGGCCTGGGAAGGCAGCAAGCCGGGCCGTC |
| | | CGTGTTCCATCCTCCACGCACCCCCACCTATCGTTGGTT |
| | | CGCAAAGTGCAAAGCTTTCTTGTGCATGACGCCCTGCT |
| | | CTGGGGAGCGTCTGGCGCGATCTCTG |
| 19 | mDMPK | GCCCGCCCCNNNNNNGCCGGGGAG |
| | binding motif | |
| 20 | hCCRS ZnF | SRGAGAGAGAGAGSRFQCRICMRNFSDRSNLSRHIRTHT |
| | domain | GEKPFACDICGRKFAISSNLNSHTKIHTGSQKPFQCRICMR |
| | | NFSRSDNLARHIRTHTGEKPFACDICGRKFATSGNLTRHT |
| | | KIHLRGSQLSIVDAPEQREGASQVSVS |
| 21 | hCCR5 binding | gtcatcctcatc |
| | motif | |
| 22 | SID | MNIQMLLEAADYLERREREAEHGYASMLP |
| | transcriptional | |
| | repressor | |
| | domain | |
| 23 | SID4X | ASPKKKRKVEASGSGMNIQMLLEAADYLERREREAEHG |
| | transcriptional | YASMLPGSGMNIQMLLEAADYLERREREAEHGYASMLP |
| | repressor | GSGMNIQMLLEAADYLERREREAEHGYASMLPGSGMNI |
| | domain | QMLLEAADYLERREREAEHGYASMLPSRSR |
| 24 | KRAB-A | VTFEDVAVLFTRDEWKKLDLSQRSLYREVMLENYSNLAS |
| | domain | MA |
| 25 | Linker-1 | GGGGS |
| 26 | Linker-2 | GGGGSGGGGS |
| 27 | Linker-3 | GGGGSGGGGSGGGGS |
| 28 | Linker-4 | GGGGGG |
| 29 | Linker-5 | EAAAKEAAAKEAAAK |
| 30 | Linker-6 | EAAAKEAAAK |
| 31 | Linker-7 | GGGGGGGG |
| 32 | Linker-8 | EAAAK |
| 33 | Linker-9 | AEAAAKEAAAKEAAAKEAAAKALEAEAAAKEAAAKEA |
| | | AAKEAAAKA |
| 34 | Linker-10 | PAPAP |
| 35 | Linker-11 | AEAAAKEAAAKA |
| 36 | NLS-1 | KRTADGSEFEPKKKRKV |
| 37 | NUC-NLS-2 | KRPAATKKAGQAKKKK |
| 38 | NLS-3 | KKTELQTTNAENKTKKL |
| 39 | NLS-4 | KRGINDRNFWRGENGRKTR |
| 40 | NLS-5 | RKSGKIAAIVVKRPRK |
| 41 | SV40-NLS-6 | PKKKRKV |
| 42 | EGL-13-NLS-7 | MSRRRKANPTKLSENAKKLAKEVEN |
| 43 | NLS-8 | MDSLLMNRRKFLYQFKNVRWAKGRRETYLC |
| 44 | TU-NLS-9 | KLKIKRPVK |
| 45 | c-Myc-NLS-10 | PAAKRVKLD |
| 46 | COX4-MLS-1 | MLSLRQSIRFFKPATRTLCSSRYLL |
| 47 | TOM7-MLS-2 | MVKLSKEAKQRLQQLFKGSQFAIRWGFIPLVIYLGFKRG |
| | | ADPGMPEPTVLSLLWG |
| 48 | ActA-MLS-3 | KLIAKSAEDEKAKEEPGNHTTLILAMLAIGVFSLGAFIKII |
| | | QLRKNN |
| 49 | HA-PT-1 | YPYDVPDYA |
| 50 | FLAG-PT-2 | DYKDDDDK |
| 51 | His-PT-3 | HHHHHH |
| 52 | Myc-PT-4 | EQKLISEEDL |
| 53 | V5-PT-5 | GKPIPNPLLGLDST |
| 54 | AviTag-PT-6 | GLNDIFEAQKIEWHE |
| 55 | The nucleic | atgggccccaagaagaagagaaaggtggaggccagcatcgaaaaaaaaaagtccttcg |
| | acid sequence | ccaagggcatgggcgtgaagtccacactcgtgtccggctccaaagtgtacatgacaacctt |
| | for the Cas13d | cgccgaaggcagcgacgccaggctggaaaagatcgtggagggcgacagcatcaggag |
| | fusion protein | cgtgaatgagggcgaggccttcagcgctgaaatggccgataaaaacgccggctataagat |
| | of Example 1. | cggcaacgccaaattcagccatcctaagggctacgccgtggtggctaacaaccctctgtat |
| | | acaggacccgtccagcaggatatgctcggcctgaaggaaactcctggaaaagaggtacttc |
| | | ggcgagagcgctgatggcaatgacaatatttgtatccaggtgatccataacatcctggacat |
| | | tgaaaaaatcctcgccgaatacattaccaacgccgcctacgccgtcaacaatatctccggc |
| | | ctggataaggacattattggattcggcaagttctccacagtgtatacctacgacgaattcaaa |
| | | gaccccgagcaccatagggccgctttcaacaataacgataagctcatcaacgccatcaag |
| | | gcccagtatgacgagttcgacaacttcctcgataaccccagactcggctatttcggccagg |
| | | cctttttcagcaaggagggcagaaattacatcatcaattacggcaacgaatgctatgacattc |
| | | tggccctcctgagcggactgaggcactgggtggtccataacaacgaagaagagtccagg |
| | | atctccaggacctggctctacaacctcgataagaacctcgacaacgaatacatctccaccct |
| | | caactacctctacgacaggatcaccaatgagctgaccaactccttctccaagaactccgcc |
| | | gccaacgtgaactatattgccgaaactctgggaatcaaccctgccgaattcgccgaacaat |
| | | atttcagattcagcattatgaaagagcagaaaaacctcggattcaatatcaccaagctcagg |
| | | gaagtgatgctggacaggaaggatatgtccgagatcaggaaaaatcataaggtgttcgact |
| | | ccatcaggaccaaggtctacaccatgatggactttgtgatttataggtattacatcgaagagg |
| | | atgccaaggtggctgccgccaataagtccctccccgataatgagaagtccctgagcgaga |
| | | aggatatctttgtgattaacctgaggggctccttcaacgacgaccagaaggatgccctctac |
| | | tacgatgaagctaatagaatttggagaaagctcgaaaatatcatgcacaacatcaaggaatt |
| | | taggggaaacaagacaagagagtataagaagaaggacgcccctagactgcccagaatc |
| | | ctgcccgctggccgtgatgtttccgccttcagcaaactcatgtatgccctgaccatgttcctg |
| | | gatggcaaggagatcaacgacctcctgaccaccctgattaataaattcgataacatccaga |
| | | gcttcctgaaggtgatgcctctcatcggagtcaacgctaagttcgtggaggaatacgcctttt |
| | | tcaaagactccgccaagatcgccgatgagctgaggctgatcaagtccttcgctagaatggg |
| | | agaacctattgccgatgccaggagggccatgtatatcgacgccatccgtattttaggaacca |
| | | acctgtcctatgatgagctcaaggccctcgccgacaccttttccctggacgagaacggaaa |
| | | caagctcaagaaaggcaagcacggcatgagaaatttcattattaataacgtgatcagcaata |
| | | aaaggttccactacctgatcagatacggtgatcctgcccacctccatgagatcgccaaaaa |
| | | cgaggccgtggtgaagttcgtgctcggcaggatcgctgacatccagaaaaaacagggcc |
| | | agaacggcaagaaccagatcgacaggtactacgaaacttgtatcggaaaggataagggc |
| | | aagagcgtgagcgaaaaggtggacgctctcacaaagatcatcaccggaatgaactacga |
| | | ccaattcgacaagaaaaggagcgtcattgaggacaccggcagggaaaacgccgagagg |
| | | gagaagtttaaaaagatcatcagcctgtacctcaccgtgattaccacatcctcaagaatatt |
| | | gtcaatatcaacgccaggtacgtcatcggattccattgcgtcgagcgtgatgctcaactgta |
| | | caaggagaaaggctacgacatcaatctcaagaaactggaagagaagggattcagctccgt |
| | | caccaagctctgcgctggcattgatgaaactgcccccgataagagaaaggacgtggaaaa |
| | | ggagatggctgaaagagccaaggagagcattgacagcctcgagagcgccaaccccaag |
| | | ctgtatgccaattacatcaaatacagcgacgagaagaaagccgaggagttcaccaggcag |
| | | attaacagggagaaggccaaaaccgccctgaacgcctacctgaggaacaccaagtggaa |
| | | tgtgatcatcagggaggacctcctgagaattgacaacaagacatgtaccctgttcagaaac |
| | | aaggccgtccacctggaagtggccaggtatgtccacgcctatatcaacgacattgccgag |
| | | gtcaattcctacttccaactgtaccattacatcatgcagagaattatcatgaatgagaggtac |
| | | gagaaaagcagcggaaaggtgtccgagtacttcgacgctgtgaatgacgagaagaagtac |
| | | aacgataggctcctgaaactgctgtgtgtgcctttcggctactgtatccccaggtttaagaac |
| | | ctgagcatcgaggccctgttcgataggaacgaggccgccaagttcgacaaggagaaaaa |
| | | gaaggtgtccggcaattccggatccggacctaagaaaaagaggaaggtggcggccgctt |
| | | acccatacgatgttccagattacgctggaggaggtggaagcggaggaggaggaagcgg |
| | | aggaggaggtagctctaggggagctggcgctggagcgggtgcaggggctggctctaga |
| | | ttccagtgccggatctgcatgcggaacttcagcgaccggtccaacctgagcaggcacatc |
| | | agaacccacaccggagaaaagcccttcgcctgcgacatttgcggccggaagttcgccatc |
| | | agcagcaacctgaacagccacaccaagatccacactggcagccagaaacctttccagtg |
| | | cagaatttgtatgagaaactttagcagaagcgacaacctggccagacacatccggacacat |
| | | actggtgaaaaaccttttgcctgtgatatctgtggcagaaagtttgccacctccggcaatctg |
| | | acccggcacacaaagattcacctgcggggcagccagctatcgattgtcgacgctcctgaa |
| | | caacgtgaaggtgcttctcaagtttctgtttctgttacttttgaagatgttgctgttctttt |
| | | tactcgtgatgaatggaaaaaacttgatctttctcaacgttctctttatcgtgaagttatgc |
| | | ttgaaaattattctaatcttgcttctatggctggatcccccaagaaaaagcgcaaggtataa |
| 56 | The amino acid | MGPKKKRKVEASIEKKKSFAKGMGVKSTLVSGSKVYMT |
| | sequence for the | TFAEGSDARLEKIVEGDSIRSVNEGEAFSAEMADKNAGY |
| | Cas13d fusion | KIGNAKFSHPKGYAVVANNPLYTGPVQQDMLGLKETLE |
| | protein of | KRYFGESADGNDNICIQVIHNILDIEKILAEYITNAAYAVN |
| | Example 1. | NISGLDKDIIGFGKFSTVYTYDEFKDPEHHRAAFNNNDKL |
| | | INAIKAQYDEFDNFLDNPRLGYFGQAFFSKEGRNYIINYG |
| | | NECYDILALLSGLRHWVVHNNEEESRISRTWLYNLDKNL |
| | | DNEYISTLNYLYDRITNELTNSFSKNSAANVNYIAETLGIN |
| | | PAEFAEQYFRFSIMKEQKNLGFNITKLREVMLDRKDMSEI |
| | | RKNHKVFDSIRTKVYTMMDFVIYRYYIEEDAKVAAANKS |
| | | LPDNEKSLSEKDIFVINLRGSENDDQKDALYYDEANRIWR |
| | | KLENIMHNIKEFRGNKTREYKKKDAPRLPRILPAGRDVSA |
| | | FSKLMYALTMFLDGKEINDLLTTLINKEDNIQSFLKVMPLI |
| | | GVNAKFVEEYAFFKDSAKIADELRLIKSFARMGEPIADAR |
| | | RAMYIDAIRILGTNLSYDELKALADTFSLDENGNKLKKG |
| | | KHGMRNFIINNVISNKRFHYLIRYGDPAHLHEIAKNEAVV |
| | | KFVLGRIADIQKKQGQNGKNQIDRYYETCIGKDKGKSVS |
| | | EKVDALTKIITGMNYDQFDKKRSVIEDTGRENAEREKFK |
| | | KIISLYLTVIYHILKNIVNINARYVIGFHCVERDAQLYKEK |
| | | GYDINLKKLEEKGFSSVTKLCAGIDETAPDKRKDVEKEM |
| | | AERAKESIDSLESANPKLYANYIKYSDEKKAEEFTRQINR |
| | | EKAKTALNAYLRNTKWNVIIREDLLRIDNKTCTLFRNKA |
| | | VHLEVARYVHAYINDIAEVNSYFQLYHYIMQRIIMNERY |
| | | EKSSGKVSEYFDAVNDEKKYNDRLLKLLCVPFGYCIPRF |
| | | KNLSIEALFDRNEAAKFDKEKKKVSGNSGSGPKKKRKVA |
| | | AAYPYDVPDYAGGGGSGGGGSGGGGSSRGAGAGAGAG |
| | | AGSRFQCRICMRNFSDRSNLSRHIRTHTGEKPFACDICGR |
| | | KFAISSNLNSHTKIHTGSQKPFQCRICMRNFSRSDNLARHI |
| | | RTHTGEKPFACDICGRKFATSGNLTRHTKIHLRGSQLSIV |
| | | DAPEQREGASQVSVSVTFEDVAVLFTRDEWKKLDLSQRS |
| | | LYREVMLENYSNLASMAGSPKKKRKV |
| 57 | Cas13d domain + | ctcctgggcaacgtgctggttattgtgctgtctcatcattttggcaaagaattcggtaccat |
| | CUG-1 guide | tgtacacggccgcataatcgaaattaatacgactcactatagggaagtaaacccctaccaact |
| | RNA nucleic | ggtcggggtttgaaacgcagcagcagcagcagcagcagcaagtaaacccctaccaact |
| | acid sequence | ggtcggggtttgaaacccgctgagcaataactagcataacccggtaccgcgggcccggg |
| | (guide RNA is | cgccaccatgggccccaagaagaagagaaaggtggaggccagcatcgaaaaaaaaaa |
| | bolded, and | gtccttcgccaagggcatgggcgtgaagtccacactcgtgtccggctccaaagtgtacatg |
| | Cas 13d is | acaaccttcgccgaaggcagcgacgccaggctggaaaagatcgtggagggcgacagca |
| | underlined) | tcaggagcgtgaatgagggcgaggccttcagcgctgaaatggccgataaaaacgccggc |
| | | tataagatcggcaacgccaaattcagccatcctaagggctacgccgtggtggctaacaacc |
| | | ctctgtatacaggacccgtccagcaggatatgctcggcctgaaggaaactctggaaaaga |
| | | ggtacttcggcgagagcgctgatggcaatgacaatatttgtatccaggtgatccataacatc |
| | | ctggacattgaaaaaatcctcgccgaatacattaccaacgccgcctacgccgtcaacaatat |
| | | ctccggcctggataaggacattattggattcggcaagttctccacagtgtatacctacgacg |
| | | aattcaaagaccccgagcaccatagggccgctttcaacaataacgataagctcatcaacgc |
| | | catcaaggcccagtatgacgagttcgacaacttcctcgataaccccagactcggctatttcg |
| | | gccaggcctttttcagcaaggagggcagaaattacatcatcaattacggcaacgaatgctat |
| | | gacattctggccctcctgagcggactgaggcactgggtggtccataacaacgaagaagag |
| | | tccaggatctccaggacctggctctacaacctcgataagaacctcgacaacgaatacatct |
| | | ccaccctcaactacctctacgacaggatcaccaatgagctgaccaactccttctccaagaa |
| | | ctccgccgccaacgtgaactatattgccgaaactctgggaatcaaccctgccgaattcgcc |
| | | gaacaatatttcagattcagcattatgaaagagcagaaaaacctcggattcaatatcaccaa |
| | | gctcagggaagtgatcctcgacaggaaggatatgtccgagatcaggaaaaatcataagct |
| | | gttcgactccatcaggaccaagctctacaccatgatggactttctcatttataggtattacat |
| | | cgaagaggatcccaaggtggctcccgccaataagtccctccccgataatgagaagtccctg |
| | | agcgagaaggatatctttctgattaacctgaggggctccttcaacgacgaccagaaggatg |
| | | ccctctactacgatgaagctaatagaatttggagaaagctcgaaaatatcatgcacaacatc |
| | | aaggaatttaggggaaacaagacaagagagtataagaagaaggacgcccctagactgcc |
| | | cagaatcctgcccgctggccgtgatgtttccgccttcagcaaactcatgtatgccctgaccat |
| | | gttcctggatggcaaggagatcaacgacctcctgaccaccctgattaataaattcgataaca |
| | | tccagagcttcctgaaggtgatgcctctcatcggagtcaacgctaagttcgtggaggaatac |
| | | gcctttttcaaagactccgccaagatcgccgatgagctgaggctgatcaagtccttcgctag |
| | | aatgggagaacctattgccgatgccaggagggccatgtatatcgacgccatccgtattttag |
| | | gaaccaacctctcctatgatgagctcaaggccctcgccgacaccttttccctggacgagaa |
| | | cggaaacaagctcaagaaaggcaagcacggcatgagaaatttcattattaataacgtgatc |
| | | agcaataaaaggttccactacctgatcagatacggtgatcctgcccacctccatgagatcgc |
| | | caaaaacgagcccgtggtgaagttcgtgctcggcaggatcgctgacatccagaaaaaac |
| | | agggccagaacggcaagaaccaatcgacaggtactacgaaacttgtatcggaaaggat |
| | | aagggcaagagcgtgagcgaaaaggtggacgctctcacaaagatcatcaccggaatgaa |
| | | ctacgaccaattcgacaagaaaaggagcctcattgaggacaccggcagggaaaacgcc |
| | | agagggagaagtttaaaaagatcatcagcctgtacctcaccgtgatctaccacatcctcaa |
| | | gaatattgtcaatatcaacgccaggtacgtcatcggattccattgcgtcgagcgtgatgctca |
| | | actgtacaaggagaaagcctacgacatcaatctcaagaaactggaagagaagggattcag |
| | | ctccgtcaccaagctctgcgctggcattgatgaaactgcccccgataagagaaaggacgt |
| | | ggaaaaggagatggctgaaagagccaaggagagcattgacagcctcgagagcgccaac |
| | | cccaagctgtatgccaattacatcaaatacagcgacgagaagaaagccgaggagttcacc |
| | | aggcagattaacagggagaaggccaaaaccgccctgaacgcctacctgaggaacacca |
| | | agtggaatctgatcatcagggaggacctcctgagaattgacaacaagacatgtaccctgtt |
| | | cagaaacaaggccgtccacctggaagtggccaggtatgtccacgcctatatcaacgacatt |
| | | gccgaggtcaattcctacttccaactgtaccattacatcatgcagagaattatcatgaatgag |
| | | aggtacgagaaaagcagccgaaagctctccgagtacttcgacgctgtgaatgacgagaa |
| | | gaagtacaacgataggctcctgaaactgctgtgtgtgcctttcggctactgtatccccagctt |
| | | taagaacctgagcatcgaggccctcttcgataggaacgaggccgccaagttcgacaagg |
| | | agaaaaagaaggtgtccggcaattccggatccggacctaagaaaaagaggaaggtggc |
| | | ggccgcttacccatacgatgttccagattacgcttaa |
| 58 | Transcript- | 5′-CTGCTGCTGCTGCTGCTGCTGC-3′ (which corresponds |
| | target (Gene | to a gRNA spacer sequence of 3′- |
| | sequence) | GACGACGACGACGACGACGACG-5′ (SEQ ID NO: 182)) |
| | CUG-1 | |
| 59 | Transcript- | 5′-GCTGCTGCTGCTGCTGCTGCTG-3′ (which corresponds |
| | target (Gene | to a gRNA spacer sequence of 3′- |
| | sequence) | CGACGACGACGACGACGACGAC-5′ (SEQ ID NO: 183) |
| | CUG-2 | |
| 60 | Transcript- | 5′-TGCTGCTGCTGCTGCTGCTGCT-3′ (which corresponds |
| | target (Gene | to a gRNA spacer sequence of 3′- |
| | sequence) | ACGACGACGACGACGACGACGA-5′ (SEQ ID NO: 184)) |
| | CUG-3 | |
| 61 | Transcript- | 5′-CAGCAGCAGCAGCAGCAGCAGC |
| | target (Gene | |
| | sequence) | |
| | CAG-1 | |
| 62 | Transcript- | 5′-GCAGCAGCAGCAGCAGCAGCAG |
| | target (Gene | |
| | sequence) | |
| | CAG-2 | |
| 63 | Transcript- | 5′-GGGGCCGGGGCCGGGGCCGGGG |
| | target (Gene | |
| | sequence) | |
| | G4C2-1 | |
| 64 | Transcript- | 5′-GGCCGGGGCCGGGGCCGGGGCC |
| | target (Gene | |
| | sequence) | |
| | G4C2-2 | |
| 65 | Transcript- | 5′-TGCCTGCCTGCCTGCCTGCCTG |
| | target (Gene | |
| | sequence) | |
| | CCUG-1 | |
| 66 | Transcript- | 5′-CCTGCCTGCCTGCCTGCCTGCC |
| | target (Gene | |
| | sequence) | |
| | CCUG-2 | |
| 67 | Transcript- | 5′-CTATTCTATTCTATTCTATTCT |
| | target (Gene | |
| | sequence) | |
| | AUUCU-1 | |
| 68 | Transcript- | 5′-ATTCTATTCTATTCTATTCTAT |
| | target (Gene | |
| | sequence) | |
| | AUUCU-2 | |
| 69 | Transcript- | 5′-GCCAGTTCACAACCGCTCCGAG |
| | target (Gene | |
| | sequence) | |
| | DMPK-1 | |
| 70 | Transcript- | 5′-GGCTCGAAGGGTCCTTGTAGCC |
| | target (Gene | |
| | sequence) | |
| | DMPK-2 | |
| 71 | Transcript- | 5′-ACTCGCTGACAGGCTACAGGAC |
| | target (Gene | |
| | sequence) | |
| | DMPK-3 | |
| 72 | Transcript- | 5′-TCCTCGGTATTTATTGTCTGTC |
| | target (Gene | |
| | sequence) | |
| | DMPK-4 | |
| 73 | Transcript- | 5′-CCTAGAACTGTCTTCGACTCCG |
| | target (Gene | |
| | sequence) | |
| | DMPK-5 | |
| 74 | Transcript- | 5′-CCTATCGTTGGTTCGCAAAGTG |
| | target (Gene | |
| | sequence) | |
| | DMPK-6 | |
| 75 | Transcript- | 5′-AGTTGCAGGAGCGGATGGAGTT |
| | target (Gene | |
| | sequence) | |
| | DMPK-7 | |
| 77 | Transcript- | 5′-GCGTGTATAGACACCTGGAGGA |
| | target (Gene | |
| | sequence) | |
| | DMPK-8 | |
| 78 | AdmCas13d | atgaacaacaaacgcaaaaccaaagcgaaagcggcgggcctgaaaagcgtgttttttgat |
| | domain (nucleic | cagaaacaggcggtgctgaccacctttgcgaaaggcaacaacagccagattgaaaaaaa |
| | acid sequence) | agtggtgaacagcgaagtgaaagatctgcgccagccgccggcgtttgatctggaactgaa |
| | | agaaaaaaccttttatattagcggcaaaaacaacattaacaccagccgcgaaaaccgctg |
| | | gcgagcgcgagcctgccgctgagcaaacgccagcgcattcgcgcggaacgcattaaac |
| | | gcgcgcgcgaagaaaaccgcccgtatcataacgtgaaacgcgtgggcgaagatgatctg |
| | | cgcgcgaaagcggatctggaaaaacattattttggcaaagaatatagcgataacctgaaaa |
| | | ttcagattatttataacattctggatattaacaaaattattagcccgtatattaacgatatt |
| | | gtgtatagcatgaacaacctggcgcgcaacgatgaatatattgatggcaaaattgatgtgat |
| | | tggcagcctgagcagcaccaccgattatagcagctttatgagcccgaacaaagatctggaaa |
| | | aagaaaaaaaatttagctttcatcgcgaaaactataaaaaatttgtggaagcgagcaaaccg |
| | | tatatgcgctattatggcaaagtgtttattcgcgatgtgaaaaaaagcaaactgagcaccgg |
| | | caaaggcgaaaaaattgaagtgatgtatcgcagcgatgaagaaatttttaccatttttcaga |
| | | ttctgagctatgtgcgccagagcattatgcataacgatattggcaacaaaagcagcattctg |
| | | gcgattgaaaaatatccggcgcgctttgtgggctttctgagcgatctgctgaaaaccaaaac |
| | | caacgatgtgaaccgcatgtttattgataacaacagccagaccaacttttgggtgctgttta |
| | | gcatttttggcctgcaggatcataccagcggcgcggataaaatttgccgcaacttttatgat |
| | | tttgtgattaaagcggatagcaaaaacctgggctttagcctgaaaaaaattcgcgaactgat |
| | | gctggatctgccgaacgcgaacatgctgcgcgatcatcagtttgataccgtgcgcagcaaat |
| | | tttataccctgctggattttattatttatcagcattatctggaagaaaaaagccgcattgat |
| | | aacatggtggaaaaactgcgcatgaccctgaaagaagaagaaaaagaagtgctgtatgcggc |
| | | ggaagcgaaaattgtgtggaacgcgattggcgcgaaagtgattaacaaactggtgccgatga |
| | | tgaacggcgatgcgctgaaagaaattaaacgcaaaaaccgcgatcgcaaactgccgcagagc |
| | | gtgattgcgaccgtgcaggtgaacagcgatgcgaacgtgtttagcggcctgatttattttct |
| | | gaccctgtttctggatggcaaagaaattaacgaaatggtgagcaacctgattaccaaatttg |
| | | aaaacattgatagcctgctgcatgtggatcgcgaaatttataaaagcgatgaaaaagatctg |
| | | gatctggaaattgaaaaactggcgctgttttttaaaggcgtggtgcgcccgaacgcgaaaac |
| | | cgataccggcgcgggcgaaattagcaaaagctttagcatttttcagagcgcggaacgcatta |
| | | ttgaagaactgaaatttattaaaaacgtgacccgcatggataacgaaatttttccgagcgaa |
| | | ggcgtgtttctggatgcggcgaacgtgctgggcgtgcgcggcgatgattttgattttagcaa |
| | | cgaatttgtgggcgatgatctgcatagcgatgcgaacaaaaaaattattaacaaaattaacg |
| | | gcaccaaagaagatcgcaacctgcgcaactttattattaacaacgtggtgaaaagccgcgct |
| | | ttcagtatattgcgcgccatatgaacacccattatgtgaaacagctggcgaacaacgaaacc |
| | | ctgaaccgctttgtgctgaacaaaatgggcgatgcgaaaattattaaccgctattatgaaag |
| | | cattagcggcaacaccccgaacattgaagtgcgcagccagattgattatctggtgaaacgcc |
| | | tgcgcagctttagctttgaagatctgaacgatgtgaaacagaaagtgcgcccgggcaccaac |
| | | gaaagcattgaaaaagaaaaaaaaaaagcgctgggggcctgtgcctgaccattcagtat |
| | | ctggtgtataaaaacctggtgaacattaacgcgcgctataccaccgcgttttattgcctggaa |
| | | cgcgatagcaaactgaaaggctttggcgtggatgtgtggcgcgattttgaaagctataccg |
| | | cgctgaccaaccattttattaaagaaggctatctgccggtgcgcaaagcggaaattctgcgc |
| | | gcgaacctgaaacatctggattgcgaagatggctttaaatattatcgcaaccaggtgaccca |
| | | tctgaacgcgattcgcgtggcgtataaatatattaacgaaattaaaagcgtgcatagctatttt |
| | | gcgctgtatcattatattatgcagcgccatctgtatgatagcctgcaggcgaaagcgaaaga |
| | | tagcagcggctttgtgattgatgcgctgaaaaaaagctttgaacataaaatttatagcaaaga |
| | | tctgctgcatgtgctgcatagcccgtttggctataacaccgcgcgctataaaaacctgagca |
| | | ttgaagcgctgtttgataaaaacgaaagccgcccggaagtgaacccgctgagcaccaacg |
| | | attatccgtatgatgtgccggattatgcg |
| 79 | EsCas13d | atgggcaaaaaaattcatgcgcgcgatctgcgcgaacagcgcaaaaccgatcgcaccga |
| | domain (nucleic | aaaatttgcggatcagaacaaaaaacgcgaagcggaacgcgcggtgccgaaaaaagat |
| | acid sequence) | gcggcggtgagcgtgaaaagcgtgagcagcgtgagcagcaaaaaagataacgtgacca |
| | | aaagcatggcgaaagcggcgggcgtgaaaagcgtgtttgcggtgggcaacaccgtgtat |
| | | atgaccagctttggccgcggcaacgatgcggtgctggaacagaaaattgtggataccagc |
| | | catgaaccgctgaacattgatgatccggcgtatcagctgaacgtggtgaccatgaacggct |
| | | atagcgtgaccggccatcgcggcgaaaccgtgagcgcggtgaccgataacccgctgcg |
| | | ccgctttaacggccgcaaaaaagatgaaccggaacagagcgtgccgaccgatatgtgt |
| | | gcctgaaaccgaccctggaaaaaaaattttttggcaaagaatttgatgataacattcatatt |
| | | cagctgatttataacattctggatattgaaaaaattctggcggtgtatagcaccaacgcgat |
| | | ttatgcgctgaacaacatgagcgcggatgaaaacattgaaaacagcgatttttttatgaaac |
| | | gcaccaccgatgaaacctttgatgattttgaaaaaaaaaaagaaagcaccaacagccgcgaa |
| | | aaagcggattttgatgcgtttgaaaaatttattggcaactatcgcctggcgtattttgcgga |
| | | tgcgttttatgtgaacaaaaaaaacccgaaaggcaaagcgaaaaacgtgctgcgcgaagata |
| | | aagaactgtatagcgtgctgaccctgattggcaaactgcgccattggtgcgtgcatagcgaa |
| | | gaaggccgcgcggaattttggctgtataaactggatgaactgaaagatgattttaaaaacgt |
| | | gctggatgtggtgtataaccgcccggtggaagaaattaacaaccgctttattgaaaacaaca |
| | | aagtgaacattcagattctgggcagcgtgtataaaaacaccgatattgcggaactggtgcg |
| | | cagctattatgaatttctgattaccaaaaaatataaaaacatgggctttagcattaaaaaact |
| | | gcgcgaaagcatgctggaaggcaaaggctatgcggataaagaatatgatagcgtgcgcaa |
| | | caaactgtatcagatgaccgattttattctgtataccggctatattaacgaagataggatcgc |
| | | gcggatgatctggtgaacaccctgcgcagcagcctgaaagaagatgataaaaccaccgt |
| | | gtattgcaaagaagcggattatctgtggaaaaaatatcgcgaaagcattcgcgaagtggcg |
| | | gatgcgctggatggcgataacattaaaaaactgagcaaaagcaacattgaaattcaggaa |
| | | gataaactgcgcaaatgctttattagctatgcggatagcgtgagcgaatttaccaaactgatt |
| | | tatctgctgacccgctttctgagcggcaaagaaattaacgatctggtgaccaccctgattaac |
| | | aaatttgataacattcgcagctttctggaaattatggatgaactgggcctggatcgcaccttt |
| | | accgcggaatatagcttttttgaaggcagcaccaaatatctggcggaactggtggaactgaa |
| | | cagctttgtgaaaagctgcagctttgatattaacgcgaaacgcaccatgtatcgcgatgcgc |
| | | tggatattctgggcattgaaagcgataaaaccgaagaagatattgaaaaaatgattgataac |
| | | attctgcagattgatgcgaacggcgataaaaaactgaaaaaaaacaacggcctgcgcaac |
| | | tttattgcgagcaacgtgattgatagcaaccgctttaaatatctggtgcgctatggcaacccg |
| | | aaaaaaattcgcgaaaccgcgaaatgcaaaccggcggtgcgctttgtgctgaacgaaatt |
| | | ccggatgcgcagattgaacgctattatgaagcgtgctgcccgaaaaacaccgcgctgtgc |
| | | agcgcgaacaaacgccgcgaaaaactggcggatatgattgcggaaattaaatttgaaaac |
| | | tttagcgatgcgggcaactatcagaaagcgaacgtgaccagccgcaccagcgaagcgga |
| | | aattaaacgcaaaaaccaggcgattattcgcctgtatctgaccgtgatgtatattatgctgaa |
| | | aaacctggtgaacgtgaacgcgcgctatgtgattgcgtttcattgcgtggaacgcgatacca |
| | | aactgtatgcggaaagcggcctggaagtgggcaacattgaaaaaaacaaaaccaacctg |
| | | accatggcggtgatgggcgtgaaactggaaaacggcattattaaaaccgaatttgataaaa |
| | | gctttgcggaaaacgcggcgaaccgctatctgcgcaacgcgcgctggtataaactgattct |
| | | ggataacctgaaaaaaagcgaacgcgcggtggtgaacgaatttcgcaacaccgtgtgcc |
| | | atctgaacgcgattcgcaacattaacattaacattaaagaaattaaagaagtggaaaactatt |
| | | ttgcgctgtatcattatctgattcagaaacatctggaaaaccgctttgcggataaaaaagtgg |
| | | aacgcgataccggcgattttattagcaaactggaagaacataaaacctattgcaaagattttg |
| | | tgaaagcgtattgcaccccgtttggctataacctggtgcgctataaaaacctgaccattgatg |
| | | gcctgtttgataaaaactatccgggcaaagatgatagcgatgaacagaaatatccgtatgat |
| | | gtgccggattatgcg |
| 80 | P1e0Cas13d | atggaacgcgaagtgaaaaaaccgccgaaaaaaagcctggcgaaagcggcgggcctg |
| | domain (nucleic | aaaagcacctttgtgattagcccgcaggaaaaagaactggcgatgaccgcgtttggccgc |
| | acid sequence) | ggcaacgatgcgctgctgcagaaacgcattgtggatggcgtggtgcgcgatgtggggg |
| | | cgaaaaacagcagtttcaggtgcagcgccaggatgaaagccgctttcgcctgcagaaca |
| | | gccgcctggcggatcgcaccgtgaccgcggatgatccgctgcatcgcgcggaaacccc |
| | | gcgccgccagccgctgggcgcgggcatggatcagctgcgccgcaaagcgattctggaa |
| | | cagaaatattttggccgcacctttgatgataacattcatattcagctgatttataacattct |
| | | ggatattcataaaatgctggcggtgccggcgaaccatattgtgcataccctgaacctgctgg |
| | | gcggctatggcgaaaccgattttgtgggcatgctgccgggggcctgccgtatgataaactgc |
| | | gcgtggtgaaaaaaaaaaacggcgataccgtggatattaaagcggatattgcggcgtatgc |
| | | gaaacgcccgcagctggcgtatctgggcgcggcgttttatgatgtgaccccgggcaaaag |
| | | caaacgcgatgcggcgcgcggccgcgtgaaacgcgaacaggatgtgtatgcgattctga |
| | | gcctgatgagcctgctgcgccagttttgcgcgcatgatagcgtgcgcatttggggccagaa |
| | | caccaccgcggcgctgtatcatctgcaggcgctgccgcaggatatgaaagatctgctgga |
| | | tgatggctggcgccgcgcgctgggggcgtgaacgatcattttctggataccaacaaagt |
| | | gaacctgctgaccctgtttgaatattatggcgcggaaaccaaacaggcgcgcgtggcgct |
| | | gacccaggatttttatcgctttgtggtgctgaaagaacagaaaaacatgggctttagcctgc |
| | | gccgcctgcgcgaagaactgctgaaactgccggatgcggcgtatctgaccggccaggaa |
| | | tatgatagcgtgcgccagaaactgtatatgctgctggattttctgctgtgccgcctgtatgcg |
| | | caggaacgcgcggatcgctgcgaagaactggtgagcgcgctgcgctgcgcgctgagcg |
| | | atgaagaaaaagataccgtgtatcaggcggaagcggcggcgctgtggcaggcgctggg |
| | | cgataccctgcgccgcaaactgctgccgctgctgaaaggcaaaaaactgcaggataaag |
| | | ataaaaaaaaaagcgatgaactgggcctgagccgcgatgtgctggatggcgtgctgtttcg |
| | | cccggcgcagcagggcagccgcgcgaacgcggattatttttgccgcctgatgcatctgag |
| | | cacctggtttatggatggcaaagaaattaacaccctgctgaccaccctgattagcaaactgg |
| | | aaaacattgatagcctgcgcagcgtgctggaaagcatgggcctggcgtatagctttgtgcc |
| | | ggcgtatgcgatgtttgatcatagccgctatattgcgggccagctgcgcgtggtgaacaac |
| | | attgcgcgcatgcgcaaaccggcgattggcgcgaaacgcgaaatgtatcgcgcggcggt |
| | | ggtgctgctgggcgtggatagcccggaagcggcggcggcgattaccgatgatctgctgc |
| | | agattgatccggaaaccggcaaagtgcgcccgcgcagcgatagcgcgcgcgataccgg |
| | | cctgcgcaactttattgcgaacaacgtggtggaaagccgccgctttacctatctgctgcgct |
| | | atatgaccccggaacaggcgcgcgtgctggcgcagaacgaaaaactgattgcgtttgtgc |
| | | tgagcaccgtgccggatacccagctggaagctattgccgcacctgcggccgcgaagat |
| | | attaccggccgcccggcgcagattcgctatctgaccgcgcagattatgggcgtgcgctatg |
| | | aaagctttaccgatgtggaacagcgcggccgcggcgataacccgaaaaaagaacgctat |
| | | aaagcgctgattggcctgtatctgaccgtgctgtatctggcggtgaaaaacatggtgaactg |
| | | caacgcgcgctatgtgattgcgttttattgccgcgatcgcgataccgcgctgtatcagaaag |
| | | aagtgtgctggtatgatctggaagaagataaaaaaagcggcaaacagcgccaggtggaa |
| | | gattataccgcgctgacccgctattttgtgagccagggctatctgaaccgccatgcgtgcgg |
| | | ctatctgcgcagcaacatgaacggcattagcaacagcctgctgaccgcgtatcgcaacgc |
| | | ggtggatcatctgaacgcgattccgccgctgggcagcctgtgccgcgatattggccgcgt |
| | | ggatagctattttgcgctgtatcattatgcggtgcagcagtatctgaacggccgctattatcg |
| | | caaaaccccgcgcgaacaggaactgtttgcggcgatggcgcagcatcgcacctggtgcag |
| | | cgatctggtgaaagcgctgaacaccccgtttggctataacctggcgcgctataaaaacctg |
| | | agcattgatggcctgtttgatcgcgaaggcgatcatgtggtgcgcgaagatggcgaaaaa |
| | | ccggcggaatatccgtatgatgtgccggattatgcg |
| 81 | RaCas13d | atggcgaaaaaaagcaaaggcatgagcctgcgcgaaaaacgcgaactggaaaaacaga |
| | domain (nucleic | aacgcattcagaaagcggcggtgaacagcgtgaacgataccccggaaaaaaccgaaga |
| | acid sequence) | agcgaacgtggtgagcgtgaacgtgcgcaccagcgcggaaaacaaacatagcaaaaaa |
| | | agcgcggcgaaagcgctgggcctgaaaagcggcctggtgattggcgatgaactgtatct |
| | | gaccagctttggccgcggcaacgaagcgaaactggaaaaaaaaattagcggcgataccg |
| | | tggaaaaactgggcattggcgcgtttgaagtggcggaacgcgatgaaagcaccctgacc |
| | | ctggaaagcggccgcattaaagataaaaccgcgcgcccgaaagatccgcgccatattac |
| | | cgtggatacccagggcaaatttaaagaagatatgctgggcattcgcagcgtgctggaaaa |
| | | aaaaatttttggcaaaacctttgatgataacattcatgtgcagctggcgtataacattctgga |
| | | tgtggaaaaaattatggcgcagtatgtgagcgatattgtgtatatgctgcataacaccgataa |
| | | aaccgaacgcaacgataacctgatgggctatatgagcattcgcaacacctataaaaccttttg |
| | | cgataccagcaacctgccggatgataccaaacagaaagtggaaaaccagaaacgcgaat |
| | | ttgataaaattattaaaagcggccgcctgggctattttggcgaagcgtttatggtgaacagcg |
| | | gcaacagcaccaaactgcgcccggaaaaagaaatttatcatatttttgcgctgatggcgag |
| | | cctgcgccagagctattttcatggctatgtgaaagataccgattatcagggcaccacctggg |
| | | cgtataccctggaagataaactgaaaggcccgagccatgaatttcgcgaaaccattgataa |
| | | aatttttgatgaaggctttagcaaaattagcaaagattttggcaaaatgaacaaagtgaacct |
| | | gcagattctggaacagatgattggcgaactgtatggcagcattgaacgccagaacctgacc |
| | | tgcgattattatgattttattcagctgaaaaaacataaatatctgggctttagcattaaacgc |
| | | ctgcgcgaaaccatgctggaaaccaccccggcggaatgctataaagcggaatgctataaca |
| | | gcgaacgccagaaactgtataaactgattgattttctgatttatgatctgtattataaccgca |
| | | aaccggcgcgcattgaagaaattgtggataaactgcgcgaaagcgtgaacgatgaagaaaa |
| | | agaaagcatttatagcgtggaagcgaaatatgtgtatgaaagcctgagcaaagtgctggat |
| | | aaaagcctgaaaaacagcgtgagcggcgaaaccattaaagatctgcagaaacgctatgat |
| | | gatgaaaccgcgaaccgcatttgggatattagccagcatagcattagcggcaacgtgaact |
| | | gcttttgcaaactgatttatattatgaccctgatgctggatggcaaagaaattaacgatctgc |
| | | tgaccaccctggtgaacaaatttgataacattgcgagctttattgatgtgatggatgaactgg |
| | | gcctggaacatagctttaccgataactataaaatgtttgcggatagcaaagcgatttgcctgg |
| | | atctgcagtttattaacagctttgcgcgcatgagcaaaattgatgatgaaaaaagcaaacgcc |
| | | agctgtttcgcgatgcgctggtgattctggatattggcaacaaagatgaaacctggattaac |
| | | aactatctggataggatatttttaaactggataaagaaggcaacaaactgaaaggcgcgc |
| | | gccatgattttcgcaactttattgcgaacaacgtgattaaaagcagccgctttaaatatctgg |
| | | tgaaatatagcagcgcggatggcatgattaaactgaaaaccaacgaaaaactgattggcttt |
| | | gtgctggataaactgccggaaacccagattgatcgctattatgaaagctgcggcctggata |
| | | acgcggtggtggataaaaaagtgcgcattgaaaaactgagcggcctgattcgcgatatga |
| | | aatttgatgattttagcggcgtgaaaaccagcaacaaagcggggataacgataaacagg |
| | | ataaagcgaaatatcaggcgattattagcctgtatctgatggtgctgtatcagattgtgaaaa |
| | | acatgatttatgtgaacagccgctatgtgattgcgtttcattgcctggaacgcgattttggca |
| | | tgtatggcaaagattttggcaaatattatcagggctgccgcaaactgaccgatcattttattg |
| | | aagaaaaatatatgaaagaaggcaaactgggctgcaacaaaaaagtgggcgctatctgaaa |
| | | aacaacattagctgctgcaccgatggcctgattaacacctatcgcaaccaggtggatcatttt |
| | | gcggtggtgcgcaaaattggcaactatgcggcgtatattaaaagcattggcagctggtttga |
| | | actgtatcattatgtgattcagcgcattgtgtttgatgaatatcgctttgcgctgaacaacac |
| | | cgaaagcaactataaaaacagcattattaaacatcatacctattgcaaagatatggtgaaagc |
| | | gctgaacaccccgtttggctatgatctgccgcgctataaaaacctgagcattggcgatctgtt |
| | | tgatcgcaacaactatctgaacaaaaccaaagaaagcattgatgcgaacagcagcattgata |
| | | gccagtatccgtatgatgtgccggattatgcg |
| 82 | RffCas13d | atgaaaaaaaaaatgagcctgcgcgaaaaacgcgaagcggaaaaacaggcgaaaaaa |
| | domain (nucleic | gcggcgtatagcgcggcgagcaaaaacaccgatagcaaaccggcggaaaaaaaagcg |
| | acid sequence) | gaaaccccgaaaccggcggaaattattagcgataacagccgcaacaaaaccgcggtgaa |
| | | agcggcgggcctgaaaagcaccattattagcggcgataaactgtatatgaccagctttggc |
| | | aaaggcaacgcggcggtgattgaacagaaaattgatattaacgattatagctttagcgcgat |
| | | gaaagataccccgagcctggaagtggataaagcggaaagcaaagaaattagctttagca |
| | | gccatcatccgtttgtgaaaaacgataaactgaccacctataacccgctgtatggcggcaaa |
| | | gataacccggaaaaaccggtgggccgcgatatgctgggcctgaaagataaactggaaga |
| | | acgctattttggctgcacctttaacgataacctgcatattcagattatttataacattctgg |
| | | atattgaaaaaattctggcggtgcatagcgcgaacattaccaccgcgctggatcatatggtg |
| | | gatgaagatgatgaaaaatatctgaacagcgattatattggctatatgaacaccattaacac |
| | | ctatgatgtgtttatggatccgagcaaaaacagcagcctgagcccgaaagatcgcaaaaaca |
| | | ttgataacagccgcgcgaaatttgaaaaactgctgagcaccaaacgcctgggctattttggc |
| | | tttgattatgatgcgaacggcaaagataaaaaaaaaaacgaagaaattaaaaaacgcctgta |
| | | tcatctgaccgcgtttgcgggccagctgcgccagtggagctttcatagcgcgggcaactatcc |
| | | gcgcacctggctgtataaactggatagcctggataaagaatatctggataccctggatcatta |
| | | ttttgataaacgctttaacgatattaacgatgattttgtgaccaaaaacgcgaccaacctgta |
| | | tattctgaaagaagtgtttccggaagcgaactttaaagatattgcggatctgtattatgattt |
| | | tattgtgattaaaagccataaaaacatgggctttagcattaaaaaactgcgcgaaaaaatgct |
| | | ggaatgcgatggcgcggatcgcattaaagaacaggatatggatagcgtgcgcagcaaactgta |
| | | taaactgattgatttttgcatttttaaatattatcatgaatttccggaactgagcgaaaaaaa |
| | | cgtggatattctgcgcgcggcggtgagcgataccaaaaaagataacctgtatagcgatgaagc |
| | | ggcgcgcctgtggagcatttttaaagaaaaatttctgggcttttgcgataaaattgtggtgtg |
| | | ggtgaccggcgaacatgaaaaagatattaccagcgtgattgataaagatgcgtatcgcaacc |
| | | gcagcaacgtgagctattttagcaaactgatgtatgcgatgtgcttttttctggatggcaaag |
| | | aaattaacgatctgctgaccaccctgattaacaaatttgataacattgcgaaccagattaaaa |
| | | ccgcgaaagaactgggcattaacaccgcgtttgtgaaaaactatgatttttttaaccatagcg |
| | | aaaaatatgtggatgaactgaacattgtgaaaaacattgcgcgcatgaaaaaaccgagcagc |
| | | aacgcgaaaaaagcgatgtatcatgatgcgctgaccattctgggcattccggaagatatgg |
| | | atgaaaaagcgctggatgaagaactggatctgattctggaaaaaaaaaccgatccggtga |
| | | ccggcaaaccgctgaaaggcaaaaacccgctgcgcaactttattgcgaacaacgtgattg |
| | | aaaacagccgctttatttatctgattaaattttgcaacccggaaaacgtgcgcaaaattgtga |
| | | acaacaccaaagtgaccgaatttgtgctgaaacgcattccggatgcgcagattgaacgctatt |
| | | ataaaagctgcaccgatagcgaaatgaacccgccgaccgaaaaaaaaattaccgaactg |
| | | gcgggcaaactgaaagatatgaactttggcaactttcgcaacgtgcgccagagcgcgaaa |
| | | gaaaacatggaaaaagaacgctttaaagcggtgattggcctgtatctgaccgtggtgtatcg |
| | | cgtggtgaaaaacctggtggatgtgaacagccgctatattatggcgtttcatagcctggaac |
| | | gcgatagccagctgtataacgtgagcgtggataacgattatctggcgctgaccgataccct |
| | | ggtgaaagaaggcgataacagccgcagccgctatctggcgggcaacaaacgcctgcgc |
| | | gattgcgtgaaacaggatattgataacgcgaaaaaatggtttgtgagcgataaatataacag |
| | | cattaccaaatatcgcaacaacgtggcgcatctgaccgcggtgcgcaactgcgcggaattt |
| | | attggcgatattaccaaaattgatagctattttgcgctgtatcattatctgattcagcgccag |
| | | ctggcgaaaggcctggatcatgaacgcagcggctttgatcgcaactatccgcagtatgcgcc |
| | | gctgtttaaatggcatacctatgtgaaagatgtggtgaaagcgctgaacgcgccgtttggct |
| | | ataacattccgcgctttaaaaacctgagcattgatgcgctgtttgatcgcaacgaaattaaaa |
| | | aaaacgatggcgaaaaaaaaagcgatgattatccgtatgatgtgccggattatgcg |
| 83 | RfxCas13d | atgatcgaaaaaaaaaagtccttcgccaagggcatgggcgtgaagtccacactcgtgtcc |
| | Domain | ggctccaaagtgtacatgacaaccttcgccgaaggcagcgacgccaggctggaaaagat |
| | (nucleic acid | cgtggagggcgacagcatcaggagcgtgaatgagggcgaggccttcagcgctgaaatg |
| | sequence) | gccgataaaaacgccggctataagatcggcaacgccaaattcagccatcctaagggctac |
| | | gccgtggtggctaacaaccctctgtatacaggacccgtccagcaggatatgctcggcctga |
| | | aggaaactctggaaaagaggtacttcggcgagagcgctgatggcaatgacaatatttgtat |
| | | ccaggtgatccataacatcctggacattgaaaaaatcctcgccgaatacattaccaacgcc |
| | | gcctacgccgtcaacaatatctccggcctggataaggacattattggattcggcaagttctc |
| | | cacagtgtatacctacgacgaattcaaagaccccgagcaccatagggccgctttcaacaat |
| | | aacgataagctcatcaacgccatcaaggcccagtatgacgagttcgacaacttcctcgata |
| | | accccagactcggctatttcggccaggcctttttcagcaaggagggcagaaattacatcatc |
| | | aattacggcaacgaatgctatgacattctggccctcctgagcggactgaggcactgggtgg |
| | | tccataacaacgaagaagagtccaggatctccaggacctggctctacaacctcgataaga |
| | | acctcgacaacgaatacatctccaccctcaactacctctacgacaggatcaccaatgagct |
| | | gaccaactccttctccaagaactccgccgccaacgtgaactatattgccgaaactctggga |
| | | atcaaccctgccgaattcgccgaacaatatttcagattcagcattatgaaagagcagaaaaa |
| | | cctcggattcaatatcaccaagctcagggaagtgatgctggacaggaaggatatgtccga |
| | | gatcaggaaaaatcataaggtgttcgactccatcaggaccaaggtctacaccatgatggac |
| | | tttgtgatttataggtattacatcgaagaggatgccaaggtggctgccgccaataagtccctc |
| | | cccgataatgagaagtccctgagcgagaaggatatctttgtgattaacctgaggggctcctt |
| | | caacgacgaccagaaggatgccctctactacgatgaagctaatagaatttggagaaagctc |
| | | gaaaatatcatgcacaacatcaaggaatttaggggaaacaagacaagagagtataagaag |
| | | aaggacgcccctagactgcccagaatcctgcccgctggccgtgatgtttccgccttcagca |
| | | aactcatgtatgccctgaccatgttcctggatggcaaggagatcaacgacctcctgaccac |
| | | cctgattaataaattcgataacatccagagcttcctgaaggtgatgcctctcatcggagtcaa |
| | | cgctaagttcgtggaggaatacgcctttttcaaagactccgccaagatcgccgatgagctg |
| | | aggctgatcaagtccttcgctagaatgggagaacctattgccgatgccaggagggccatgt |
| | | atatcgacgccatccgtattttaggaaccaacctgtcctatgatgagctcaaggccctcgcc |
| | | gacaccttttccctggacgagaacggaaacaagctcaagaaaggcaagcacggcatgag |
| | | aaatttcattattaataacgtgatcagcaataaaaggttccactacctgatcagatacggtg |
| | | atcctgcccacctccatgagatcgccaaaaacgaggccgtggtgaagttcgtgctcggcagg |
| | | atcgctgacatccagaaaaaacagggccagaacggcaagaaccagatcgacaggtacta |
| | | cgaaacttgtatcggaaaggataagggcaagagcgtgagcgaaaaggtggacgctctca |
| | | caaagatcatcaccggaatgaactacgaccaattcgacaagaaaaggagcgtcattgagg |
| | | acaccggcagggaaaacgccgagagggagaagtttaaaaagatcatcagcctgtacct |
| | | accgtgatctaccacatcctcaagaatattgtcaatatcaacgccaggtacgtcatcggattc |
| | | cattgcgtcgagcgtgatgctcaactgtacaaggagaaaggctacgacatcaatctcaaga |
| | | aactggaagagaagggattcagctccgtcaccaagctctgcgctggcattgatgaaactgc |
| | | ccccgataagagaaaggacgtggaaaaggagatggctgaaagagccaaggagagcatt |
| | | gacagcctcgagagcgccaaccccaagctgtatgccaattacatcaaatacagcgacgag |
| | | aagaaagccgaggagttcaccaggcagattaacagggagaaggccaaaaccgccctga |
| | | acgcctacctgaggaacaccaagtggaatgtgatcatcagggaggacctcctgagaattg |
| | | acaacaagacatgtaccctgttcagaaacaaggccgtccacctggaagtggccaggtatg |
| | | tccacgcctatatcaacgacattgccgaggtcaattcctacttccaactgtaccattacatc |
| | | atgcagagaattatcatgaatgagaggtacgagaaaagcagcggaaaggtgtccgagtactt |
| | | cgacgctgtgaatgacgagaagaagtacaacgataggctcctgaaactgctgtgtgtgcct |
| | | ttcggctactgtatccccaggtttaagaacctgagcatcgaggccctgttcgataggaacga |
| | | ggccgccaagttcgacaaggagaaaaagaaggtgtccggcaattcctaa |
| 84 | UrCas13d | atggcaaaaaaaaataaaatgaaaccgcgtgaactgcgtgaagcacagaaaaaagcacg |
| | domain (nucleic | tcagctgaaagcagcagaaattaataataatgcagcaccggcaattgcagcaatgccggc |
| | acid sequence) | agcagaagttattgcaccggttgcagaaaaaaaaaaaagcagcgttaaagcagcaggtat |
| | | gaaaagcattctggttagcgaaaataaaatgtatattaccagctttggtaaaggtaatagcgc |
| | | agttctggaatatgaagttgataataatgattataataaaacccagctgagcagcaaagataa |
| | | tagcaatattgaactgggtgatgttaatgaagttaatattacctttagcagcaaacatggttt |
| | | tggtagcggtgttgaaattaataccagcaatccgacccatcgtagcggtgaaagcagcccggt |
| | | tcgtggtgatatgctgggtctgaaaagcgaactggaaaaacgtttttttggtaaaacctttga |
| | | tgataatattcatattcagctgatttataatattctggatattgaaaaaattctggcagttta |
| | | tgttaccaatattgtttatgcactgaataatatgctgggtattaaagatagcgaaagctatga |
| | | tgattttatgggttatctgagcgcacgtaatacctatgaagtttttacccatccggataaaag |
| | | caatctgagcgataaagttaaaggtaatattaaaaaaagcctgagcaaatttaatgatctgct |
| | | gaaaaccaaacgtctgggttattttggtctggaagaaccgaaaaccaaagatacccgtgcaag |
| | | cgaagcatataaaaaacgtgtttatcacatgctggcaattgttggtcagattcgtcagtgtg |
| | | tttttcatgataaaagcggtgcaaaacgttttgatctgtatagctttattaataatattgatc |
| | | cggaatatcgtgataccctggattatctggttgaagaacgtctgaaaagcattaataaagat |
| | | tttattgaaggtaataaagttaatattagcctgctgattgatatgatgaaaggttatgaagc |
| | | agatgatattattcgtctgtattatgattttattgttctgaaaagccagaaaaatctgggtt |
| | | ttagcattaaaaaactgcgtgaaaaaatgctggaagaatatggttttcgttttaaagataaa |
| | | cagtatgatagcgttcgtagcaaaatgtataaactgatggattttctgctgttttgtaatta |
| | | ttatcgtaatgatgttgcagcaggtgaagcactggttcgtaaactgcgttttagcatgaccg |
| | | atgatgaaaaagaaggtatttatgcagatgaagcagcaaaactgtggggtaaatttcgtaat |
| | | gattttgaaaatattgcagatcacatgaatggtgatgttattaaagaactgggtaaagcaga |
| | | tatggattttgatgaaaaaattctggatagcgaaaaaaaaaatgcaagcgatctgctgtatt |
| | | ttagcaaaatgatttatatgctgacctattttctggatggtaaagaaattaatgatctgctg |
| | | accaccctgattagcaaatttgataatattaaagaatttctgaaaattatgaaaagcagcgc |
| | | agttgatgttgaatgtgaactgaccgcaggttataaactgtttaatgatagccagcgtatta |
| | | ccaatgaactgtttattgttaaaaatattgcaagcatgcgtaaaccggcagcaagcgcaaaa |
| | | ctgaccatgtttcgtgatgcactgaccattctgggtattgatgataatattaccgatgatcg |
| | | tattagcgaaattctgaaactgaaagaaaaaggtaaaggtattcatggtctgcgtaatttta |
| | | ttaccaataatgttattgaaagcagccgttttgtttatctgattaaatatgcaaatgcacag |
| | | aaaattcgtgaagttgcaaaaaatgaaaaagttgttatgtttgttctgggtggtattccgga |
| | | tacccagattgaacgttattataaaagctgtgttgaatttccggatatgaatagcagcctgg |
| | | aagcaaaacgtagcgaactggcacgtatgattaaaaatattagctttgatgattttaaaaat |
| | | gttaaacagcaggcaaaaggtcgtgaaaatgttgcaaaagaacgtgcaaaagcagttattgg |
| | | tctgtatctgaccgttatgtatctgctggttaaaaatctggttaatgttaatgcacgttatg |
| | | ttattgcaattcattgtctggaacgtgattttggtctgtataaagaaattattccggaactg |
| | | gcaagcaaaaatctgaaaaatgattatcgtattctgagccagaccctgtgtgaactgtgtga |
| | | tgatcgtaatgaaagcagcaatctgtttctgaaaaaaaataaacgtctgcgtaaatgtg |
| | | ttgaagttgatattaataatgcagatagcagcatgacccgtaaatatcgtaattgtattgca |
| | | catctgaccgttgttcgtgaactgaaagaatatattggtgatattcgtaccgttgatagcta |
| | | attttgcatttatcattatgttatgcagcgttgtattaccaaacgtggtgatgataccaaac |
| | | aggaagaaaaaattaaatatgaagatgatctgctgaaaaatcatggttataccaaagatttt |
| | | gttaaagcactgaatagcccgtttggttataatattccgcgttttaaaaatctgagcattga |
| | | acagctgtttgatcgtaatgaatatctgaccgaaaaataa |
| 85 | SV40 bipartite | KRTADGSEFESPKKKRKV |
| | NLS | |
| 178 | AAV_CMV- | gcgcccaatacgcaaaccgcctctccccgcgcgttggccgattcattaatgcagctggcac |
| | GENO_CUGI | gacaggtttcccgactggaaagcgggcagtgagcgcaacgcaattaatgtgagttagctc |
| | | actcattaggcaccccaggctttacactttatgcttccggctcgtatgttgtgtggaattgt |
| | | gagcggataacaatttcacacaggaaacagctatgaccatgattacgccaagcttgcatgcc |
| | | ctgcaggcagctgcgcgctcgctcgctcactgaggccgcccgggcaaagcccgggcgtcg |
| | | ggcgacctttggtcgcccggcctcagtgagcgagcgagcgcgcagagagggagtg |
| | | gccaactccatcactaggggttcctgcggccgcagccgtgttttagagctagaaatagcgt |
| | | catcctcatcggtacccgttacataacttacggtaaatggcccgcctggctgaccgcccaac |
| | | gacccccgcccattgacgtcaataatgacgtatgttcccatagtaacgccaatagggacttt |
| | | ccattgacgtcaatgggtggagtatttacggtaaactgcccacttggcagtacatcaagtgta |
| | | tcatatgccaagtacgccccctattgacgtcaatgacggtaaatggcccgcctggcattatg |
| | | cccagtacatgaccttatgggactttcctacttggcagtacatctacgtattagtcatcgcta |
| | | ttaccatggtgatgcggttttggcagtacatcaatgggcgtggatagcggtttgactcacggg |
| | | gatttccaagtctccaccccattgacgtcaatgggagtttgttttggcaccaaaatcaacggg |
| | | actttccaaaatgtcgtaacaactccgccccattgacgcaaatgggggtaggcgtgtacg |
| | | gtgggaggtctatataagcagagctctctggctaactaccggtcgaaattaatacgactcac |
| | | tatagggCAAGTAAACCCCTACCAACTGGTCGGGGTTTGA |
| | | AACGCAGCAGCAGCAGCAGCAGCAGCAAGTAAACCCC |
| | | TACCAACTGGTCGGGGTTTGAAACccgctgagcaataactagcata |
| | | acccggtaccgcgggcccgggcgccaccatgGGCCCCAAGAAgAAGAG |
| | | aAAGGTGGAGGCCAGCATCGAAAAAAAAAAGTCCTTC |
| | | GCCAAGGGCATGGGCGTGAAGTCCACACTCGTGTCCG |
| | | GCTCCAAAGTGTACATGACAACCTTCGCCGAAGGCAG |
| | | CGACGCCAGGCTGGAAAAGATCGTGGAGGGCGACAGC |
| | | ATCAGGAGCGTGAATGAGGGCGAGGCCTTCAGCGCTG |
| | | AAATGGCCGATAAAAACGCCGGCTATAAGATCGGCAA |
| | | CGCCAAATTCAGCCATCCTAAGGGCTACGCCGTGGTGG |
| | | CTAACAACCCTCTGTATACAGGACCCGTCCAGCAGGAT |
| | | ATGCTCGGCCTGAAGGAAACTCTGGAAAAGAGGTACT |
| | | TCGGCGAGAGCGCTGATGGCAATGACAATATTTGTATC |
| | | CAGGTGATCCATAACATCCTGGACATTGAAAAAATCCT |
| | | CGCCGAATACATTACCAACGCCGCCTACGCCGTCAACA |
| | | ATATCTCCGGCCTGGATAAGGACATTATTGGATTCGGC |
| | | AAGTTCTCCACAGTGTATACCTACGACGAATTCAAAGA |
| | | CCCCGAGCACCATAGGGCCGCTTTCAACAATAACGATA |
| | | AGCTCATCAACGCCATCAAGGCCCAGTATGACGAGTTC |
| | | GACAACTTCCTCGATAACCCCAGACTCGGCTATTTCGG |
| | | CCAGGCCTTTTTCAGCAAGGAGGGCAGAAATTACATCA |
| | | TCAATTACGGCAACGAATGCTATGACATTCTGGCCCTC |
| | | CTGAGCGGACTGAGGCACTGGGTGGTCCATAACAACG |
| | | AAGAAGAGTCCAGGATCTCCAGGACCTGGCTCTACAA |
| | | CCTCGATAAGAACCTCGACAACGAATACATCTCCACCC |
| | | TCAACTACCTCTACGACAGGATCACCAATGAGCTGACC |
| | | AACTCCTTCTCCAAGAACTCCGCCGCCAACGTGAACTA |
| | | TATTGCCGAAACTCTGGGAATCAACCCTGCCGAATTCG |
| | | CCGAACAATATTTCAGATTCAGCATTATGAAAGAGCAG |
| | | AAAAACCTCGGATTCAATATCACCAAGCTCAGGGAAG |
| | | TGATGCTGGACAGGAAGGATATGTCCGAGATCAGGAA |
| | | AAATCATAAGGTGTTCGACTCCATCAGGACCAAGGTCT |
| | | ACACCATGATGGACTTTGTGATTTATAGGTATTACATC |
| | | GAAGAGGATGCCAAGGTGGCTGCCGCCAATAAGTCCC |
| | | TCCCCGATAATGAGAAGTCCCTGAGCGAGAAGGATAT |
| | | CTTTGTGATTAACCTGAGGGGCTCCTTCAACGACGACC |
| | | AGAAGGATGCCCTCTACTACGATGAAGCTAATAGAATT |
| | | TGGAGAAAGCTCGAAAATATCATGCACAACATCAAGG |
| | | AATTTAGGGGAAACAAGACAAGAGAGTATAAGAAGAA |
| | | GGACGCCCCTAGACTGCCCAGAATCCTGCCCGCTGGCC |
| | | GTGATGTTTCCGCCTTCAGCAAACTCATGTATGCCCTG |
| | | ACCATGTTCCTGGATGGCAAGGAGATCAACGACCTCCT |
| | | GACCACCCTGATTAATAAATTCGATAACATCCAGAGCT |
| | | TCCTGAAGGTGATGCCTCTCATCGGAGTCAACGCTAAG |
| | | TTCGTGGAGGAATACGCCTTTTTCAAAGACTCCGCCAA |
| | | GATCGCCGATGAGCTGAGGCTGATCAAGTCCTTCGCTA |
| | | GAATGGGAGAACCTATTGCCGATGCCAGGAGGGCCAT |
| | | GTATATCGACGCCATCCGTATTTTAGGAACCAACCTGT |
| | | CCTATGATGAGCTCAAGGCCCTCGCCGACACCTTTTCC |
| | | CTGGACGAGAACGGAAACAAGCTCAAGAAAGGCAAGC |
| | | ACGGCATGAGAAATTTCATTATTAATAACGTGATCAGC |
| | | AATAAAAGGTTCCACTACCTGATCAGATACGGTGATCC |
| | | TGCCCACCTCCATGAGATCGCCAAAAACGAGGCCGTG |
| | | GTGAAGTTCGTGCTCGGCAGGATCGCTGACATCCAGAA |
| | | AAAACAGGGCCAGAACGGCAAGAACCAGATCGACAG |
| | | GTACTACGAAACTTGTATCGGAAAGGATAAGGGCAAG |
| | | AGCGTGAGCGAAAAGGTGGACGCTCTCACAAAGATCA |
| | | TCACCGGAATGAACTACGACCAATTCGACAAGAAAAG |
| | | GAGCGTCATTGAGGACACCGGCAGGGAAAACGCCGAG |
| | | AGGGAGAAGTTTAAAAAGATCATCAGCCTGTACCTCA |
| | | CCGTGATCTACCACATCCTCAAGAATATTGTCAATATC |
| | | AACGCCAGGTACGTCATCGGATTCCATTGCGTCGAGCG |
| | | TGATGCTCAACTGTACAAGGAGAAAGGCTACGACATC |
| | | AATCTCAAGAAACTGGAAGAGAAGGGATTCAGCTCCG |
| | | TCACCAAGCTCTGCGCTGGCATTGATGAAACTGCCCCC |
| | | GATAAGAGAAAGGACGTGGAAAAGGAGATGGCTGAA |
| | | AGAGCCAAGGAGAGCATTGACAGCCTCGAGAGCGCCA |
| | | ACCCCAAGCTGTATGCCAATTACATCAAATACAGCGAC |
| | | GAGAAGAAAGCCGAGGAGTTCACCAGGCAGATTAACA |
| | | GGGAGAAGGCCAAAACCGCCCTGAACGCCTACCTGAG |
| | | GAACACCAAGTGGAATGTGATCATCAGGGAGGACCTC |
| | | CTGAGAATTGACAACAAGACATGTACCCTGTTCAGAA |
| | | ACAAGGCCGTCCACCTGGAAGTGGCCAGGTATGTCCA |
| | | CGCCTATATCAACGACATTGCCGAGGTCAATTCCTACT |
| | | TCCAACTGTACCATTACATCATGCAGAGAATTATCATG |
| | | AATGAGAGGTACGAGAAAAGCAGCGGAAAGGTGTCCG |
| | | AGTACTTCGACGCTGTGAATGACGAGAAGAAGTACAA |
| | | CGATAGGCTCCTGAAACTGCTGTGTGTGCCTTTCGGCT |
| | | ACTGTATCCCCAGGTTTAAGAACCTGAGCATCGAGGCC |
| | | CTGTTCGATAGGAACGAGGCCGCCAAGTTCGACAAGG |
| | | AGAAAAAGAAGGTGTCCGGCAATTCCGGATCCggacctaa |
| | | gaaaaagaggaaggtggcggccgctTACCCATACGATGTTCCAGAT |
| | | TACGCTTAActtaagatacattgatgagtttggacaaaccacaactagaatgcagtg |
| | | aaaaaaatgctttatttgtgaaatttgtgatgctattgctttatttgtaaccattataagct |
| | | gcaataaacaagttctgattttgtagttaacacgtgcgaccgagcggccgcaggaaccccta |
| | | gtgatggagttggccactccctctctgcgcgctcgctcgctcactgaggccgggcgaccaaa |
| | | ggtcgcccgacgcccgggctttgcccgggcggcctcagtgagcgagcgagcgcgcagctg |
| | | cctgcaggggcgcctgatgcggtattttctccttacgcatctgtgcggtatttcacaccgcat |
| | | atggtgcactctcagtacaatctgctctgatgccgcatagttaagccagccccgacacccg |
| | | ccaacacccgctgacgcgccctgacgggcttgtctgctcccggcatccgcttacagacaa |
| | | gctgtgaccgtctccgggagctgcatgtgtcagaggttttcaccgtcatcaccgaaacgcg |
| | | cgagacgaaagggcctcgtgatacgcctatttttataggttaatgtcatgataataatggttt |
| | | cttagacgtcaggtggcacttttcggggaaatgtgcgcggaacccctatttgtttatttttct |
| | | aaatacattcaaatatgtatccgctcatgagacaataaccctgataaatgcttcaataatatt |
| | | gaaaaaggaagagtatgagtattcaacatttccgtgtcgcccttattcccttttttgcggca |
| | | ttttgccttcctgtttttgctcacccagaaacgctggtgaaagtaaaagatgctgaagatca |
| | | gttgggtgcacgagtgggttacatcgaactggatctcaacagcggtaagatccttgagagtt |
| | | ttcgccccgaagaacgttttccaatgatgagcacttttaaagttctgctatgtggcgcggta |
| | | ttatcccgtattgacgccgggcaagagcaactcggtcgccgcatacactattctcagaatga |
| | | cttggttgagtactcaccagtcacagaaaagcatcttacggatggcatgacagtaagagaat |
| | | tatgcagtgctgccataaccatgagtgataacactgcggccaacttacttctgacaacgatc |
| | | ggaggaccgaaggagctaaccgcttttttgcacaacatgggggatcatgtaactcgccttga |
| | | tcgttgggaaccggagctgaatgaagccataccaaacgacgagcgtgacaccacgatgcctg |
| | | tagcaatggcaacaacgttgcgcaaactattaactggcgaactacttactctagcttcccgg |
| | | caacaattaatagactggatggaggggataaagttgcaggaccacttctgcgctcggccctt |
| | | ccggctggctggtttattgctgataaatctggagccggtgagcgtgggtctcgcggtatcat |
| | | tgcagcactggggccagatggtaagccctcccgtatcgtagttatctacacgacggggagtc |
| | | aggcaactatggatgaacgaaatagacagatcgctgagataggtgcctcactgattaagcat |
| | | tggtaactgtcagaccaagtttactcatatatactttagattgatttaaaacttcattttta |
| | | atttaaaaggatctaggtgaagatcctttttgataatctcatgaccaaaatcccttaacgtg |
| | | agttttcgttccactgagcgtcagaccccgtagaaaagatcaaaggatcttcttgagatcct |
| | | ttttttctgcgcgtaatctgctgcttgcaaacaaaaaaaccaccgctaccagcggtggtttg |
| | | tttgccggatcaagagctaccaactctttttccgaaggtaactggcttcagcagagcgcaga |
| | | taccaaatactgttcttctagtgtagccgtagttaggccaccacttcaagaactctgtagca |
| | | ccgcctacatacctcgctctgctaatcctgttaccagtggctgctgccagtggcgataagtc |
| | | gtgtcttaccgggttggactcaagacgatagttaccggataaggcgcagcggtcgggctgaa |
| | | cggggggttcgtgcacacagcccagcttggagcgaacgacctacaccgaactgagataccta |
| | | cagcgtgagctatgagaaagcgccacgcttcccgaagggagaaaggcggacaggtatccggt |
| | | aagcggcagggtcggaacaggagagcgcacgagggagcttccagggggaaacgcctggtatc |
| | | tttatagtcctgtcgggtttcgccacctctgacttgagcgtcgatttttgtgatgctcgtca |
| | | ggggggcggagcctatggaaaaacgccagcaacgcggcctttttacggttcctggccttttg |
| | | ctggccttttgctcacatgttctttcctgcgttatcccctgattctgtggataaccgtatta |
| | | ccgcctttgagtgagctgataccgctcgccgcagccgaacgaccgagcgcagcgagtcagtg |
| | | agcgaggaagcggaaga |
| 179 | AAV_CMV_ | gcgcccaatacgcaaaccgcctctccccgcgcgttggccgattcattaatgcagctggcac |
| | GENO_NT (non- | gacaggtttcccgactggaaaggggcagtgagcgcaacgcaattaatgtgagttagctc |
| | targeting) | actcattaggcaccccaggctttacactttatgcttccggctcgtatgttgtgtggaattgt |
| | | gagcggataacaatttcacacaggaaacagctatgaccatgattacgccaagcttgcatgcc |
| | | ctgcaggcagctgcgcgctcgctcgctcactgaggccgcccgggcaaagcccgggcgtcg |
| | | ggcgacctttggtcgcccggcctcagtgagcgagcgagcgcgcagagagggagtg |
| | | gccaactccatcactaggggttcctgcggccgcagccgtgttttagagctagaaatagcgt |
| | | catcctcatcggtacccgttacataacttacggtaaatggcccgcctggctgaccgcccaac |
| | | gacccccgcccattgacgtcaataatgacgtatgttcccatagtaacgccaatagggacttt |
| | | ccattgacgtcaatgggtggagtatttacggtaaactgcccacttggcagtacatcaagtgta |
| | | tcatatgccaagtacgccccctattgacgtcaatgacggtaaatggcccgcctggcattatg |
| | | cccagtacatgaccttatgggactttcctacttggcagtacatctacgtattagtcatcgcta |
| | | ttaccatggtgatgcggttttggcagtacatcaatgggcgtggatagcggtttgactcacggg |
| | | gatttccaagtctccaccccattgacgtcaatgggagtttgttttggcaccaaaatcaacggg |
| | | actttccaaaatgtcgtaacaactccgccccattgacgcaaatgggggtaggcgtgtacg |
| | | gtgggaggtctatataagcagagctctctggctaactaccggtcgaaattaatacgactcac |
| | | tatagggCAAGTAAACCCCTACCAACTGGTCGGGGTTTGA |
| | | AACCGAGGGCGACTTAACCTTAGGTCAAGTAAACCCCT |
| | | ACCAACTGGTCGGGGTTTGAAACccgctgagcaataactagcataac |
| | | ccggtaccgcgggcccgggcgccaccatgGGCCCCAAGAAgAAGAGa |
| | | AAGGTGGAGGCCAGCATCGAAAAAAAAAAGTCCTTCG |
| | | CCAAGGGCATGGGCGTGAAGTCCACACTCGTGTCCGG |
| | | CTCCAAAGTGTACATGACAACCTTCGCCGAAGGCAGC |
| | | GACGCCAGGCTGGAAAAGATCGTGGAGGGCGACAGCA |
| | | TCAGGAGCGTGAATGAGGGCGAGGCCTTCAGCGCTGA |
| | | AATGGCCGATAAAAACGCCGGCTATAAGATCGGCAAC |
| | | GCCAAATTCAGCCATCCTAAGGGCTACGCCGTGGTGGC |
| | | TAACAACCCTCTGTATACAGGACCCGTCCAGCAGGATA |
| | | TGCTCGGCCTGAAGGAAACTCTGGAAAAGAGGTACTT |
| | | CGGCGAGAGCGCTGATGGCAATGACAATATTTGTATCC |
| | | AGGTGATCCATAACATCCTGGACATTGAAAAAATCCTC |
| | | GCCGAATACATTACCAACGCCGCCTACGCCGTCAACAA |
| | | TATCTCCGGCCTGGATAAGGACATTATTGGATTCGGCA |
| | | AGTTCTCCACAGTGTATACCTACGACGAATTCAAAGAC |
| | | C |
| | | CCGAGCACCATAGGGCCGCTTTCAACAATAACGATAA |
| | | GCTCATCAACGCCATCAAGGCCCAGTATGACGAGTTCG |
| | | ACAACTTCCTCGATAACCCCAGACTCGGCTATTTCGGC |
| | | CAGGCCTTTTTCAGCAAGGAGGGCAGAAATTACATCAT |
| | | CAATTACGGCAACGAATGCTATGACATTCTGGCCCTCC |
| | | TGAGCGGACTGAGGCACTGGGTGGTCCATAACAACGA |
| | | AGAAGAGTCCAGGATCTCCAGGACCTGGCTCTACAAC |
| | | CTCGATAAGAACCTCGACAACGAATACATCTCCACCCT |
| | | CAACTACCTCTACGACAGGATCACCAATGAGCTGACCA |
| | | ACTCCTTCTCCAAGAACTCCGCCGCCAACGTGAACTAT |
| | | ATTGCCGAAACTCTGGGAATCAACCCTGCCGAATTCGC |
| | | CGAACAATATTTCAGATTCAGCATTATGAAAGAGCAG |
| | | AAAAACCTCGGATTCAATATCACCAAGCTCAGGGAAG |
| | | TGATGCTGGACAGGAAGGATATGTCCGAGATCAGGAA |
| | | AAATCATAAGGTGTTCGACTCCATCAGGACCAAGGTCT |
| | | ACACCATGATGGACTTTGTGATTTATAGGTATTACATC |
| | | GAAGAGGATGCCAAGGTGGCTGCCGCCAATAAGTCCC |
| | | TCCCCGATAATGAGAAGTCCCTGAGCGAGAAGGATAT |
| | | CTTTGTGATTAACCTGAGGGGCTCCTTCAACGACGACC |
| | | AGAAGG |
| | | ATGCCCTCTACTACGATGAAGCTAATAGAATTTGGAGA |
| | | AAGCTCGAAAATATCATGCACAACATCAAGGAATTTA |
| | | GGGGAAACAAGACAAGAGAGTATAAGAAGAAGGACG |
| | | CCCCTAGACTGCCCAGAATCCTGCCCGCTGGCCGTGAT |
| | | GTTTCCGCCTTCAGCAAACTCATGTATGCCCTGACCAT |
| | | GTTCCTGGATGGCAAGGAGATCAACGACCTCCTGACCA |
| | | CCCTGATTAATAAATTCGATAACATCCAGAGCTTCCTG |
| | | AAGGTGATGCCTCTCATCGGAGTCAACGCTAAGTTCGT |
| | | GGAGGAATACGCCTTTTTCAAAGACTCCGCCAAGATCG |
| | | CCGATGAGCTGAGGCTGATCAAGTCCTTCGCTAGAATG |
| | | GGAGAACCTATTGCCGATGCCAGGAGGGCCATGTATA |
| | | TCGACGCCATCCGTATTTTAGGAACCAACCTGTCCTAT |
| | | GATGAGCTCAAGGCCCTCGCCGACACCTTTTCCCTGGA |
| | | CGAGAACGGAAACAAGCTCAAGAAAGGCAAGCACGG |
| | | CATGAGAAATTTCATTATTAATAACGTGATCAGCAATA |
| | | AAAGGTTCCACTACCTGATCAGATACGGTGATCCTGCC |
| | | CACCTCCATGAGATCGCCAAAAACGAGGCCGTGGTGA |
| | | AGTTCGTGCTCGGCAGGATCGCTGACATCCAGAAAAA |
| | | ACAGGGCCAGAACGGCAAGAACCAGATCGACAGGTAC |
| | | TACGAAACTTGTATCGGAAAGGATAAGGGCAAGAGCG |
| | | TGAGCGAAAAGGTGGACGCTCTCACAAAGATCATCAC |
| | | CGGAATGAACTACGACCAATTCGACAAGAAAAGGAGC |
| | | GTCATTGAGGACACCGGCAGGGAAAACGCCGAGAGGG |
| | | AGAAGTTTAAAAAGATCATCAGCCTGTACCTCACCGTG |
| | | ATCTACCACATCCTCAAGAATATTGTCAATATCAACGC |
| | | CAGGTACGTCATCGGATTCCATTGCGTCGAGCGTGATG |
| | | CTCAACTGTACAAGGAGAAAGGCTACGACATCAATCT |
| | | CAAGAAACTGGAAGAGAAGGGATTCAGCTCCGTCACC |
| | | AAGCTCTGCGCTGGCATTGATGAAACTGCCCCCGATAA |
| | | GAGAAAGGACGTGGAAAAGGAGATGGCTGAAAGAGC |
| | | CAAGGAGAGCATTGACAGCCTCGAGAGCGCCAACCCC |
| | | AAGCTGTATGCCAATTACATCAAATACAGCGACGAGA |
| | | AGAAAGCCGAGGAGTTCACCAGGCAGATTAACAGGGA |
| | | GAAGGCCAAAACCGCCCTGAACGCCTACCTGAGGAAC |
| | | ACCAAGTGGAATGTGATCATCAGGGAGGACCTCCTGA |
| | | GAATTGACAACA |
| | | AGACATGTACCCTGTTCAGAAACAAGGCCGTCCACCTG |
| | | GAAGTGGCCAGGTATGTCCACGCCTATATCAACGACAT |
| | | TGCCGAGGTCAATTCCTACTTCCAACTGTACCATTACA |
| | | TCATGCAGAGAATTATCATGAATGAGAGGTACGAGAA |
| | | AAGCAGCGGAAAGGTGTCCGAGTACTTCGACGCTGTG |
| | | AATGACGAGAAGAAGTACAACGATAGGCTCCTGAAAC |
| | | TGCTGTGTGTGCCTTTCGGCTACTGTATCCCCAGGTTTA |
| | | AGAACCTGAGCATCGAGGCCCTGTTCGATAGGAACGA |
| | | GGCCGCCAAGTTCGACAAGGAGAAAAAGAAGGTGTCC |
| | | GGCAATTCCGGATCCggacctaagaaaaagaggaaggtggcggccgctT |
| | | ACCCATACGATGTTCCAGATTACGCTTAActtaagatacattgat |
| | | gagtttggacaaaccacaactagaatgcagtgaaaaaaatgctttatttgtgaaatttgtgat |
| | | gctattgctttatttgtaaccattataagctgcaataaacaagttctgattttgtagttaaca |
| | | cgtgcgaccgagcggccgcaggaacccctagtgatggagttggccactccctctctgcgcgct |
| | | cgctcgctcactgaggccgggcgaccaaaggtcgcccgacgcccgggctttgcccgggcg |
| | | gcctcagtgagcgagcgagcgcgcagctgcctgcaggggcgcctgatgcggtattttctc |
| | | cttacgcatctgtgcggtatttcacaccgcatatggtgcactctcagtacaatctgctctgat |
| | | gccgcatagttaagccagccccgacacccgccaacacccgctgacgcgccctgacgggct |
| | | tgtctgctcccggcatccgcttacagacaagctgtgaccgtctccgggagctgcatgtgtca |
| | | gaggttttcaccgtcatcaccgaaacgcgcgagacgaaagggcctcgtgatacgcctattt |
| | | ttataggttaatgtcatgataataatggtttcttagacgtcaggtggcacttttcggggaaat |
| | | gtgcgcggaacccctatttgtttatttttctaaatacattcaaatatgtatccgctcatgaga |
| | | caataaccctgataaatgcttcaataatattgaaaaaggaagagtatgagtattcaacatttc |
| | | cgtgtcgcccttattcccttttttgcggcattttgccttcctgtttttgctcacccagaaacg |
| | | ctggtgaaagtaaaagatgctgaagatcagttgggtgcacgagtgggttacatgaactggatc |
| | | tcaacagcggtaagatccttgagagttttcgccccgaagaacgttttccaatgatgagcactt |
| | | ttaaagttctgctatgtggcgcggtattatcccgtattgacgccgggcaagagcaactcggt |
| | | cgccgcatacactattctcagaatgacttggttgagtactcaccagtcacagaaaagcatct |
| | | tacggatggcatgacagtaagagaattatgcagtgctgccataaccatgagtgataacactg |
| | | cggccaacttacttctgacaacgatcggaggaccgaaggagctaaccgcttttttgcacaac |
| | | atgggggatcatgtaactcgccttgatcgttgggaaccggagctgaatgaagccataccaaa |
| | | cgacgagcgtgacaccacgatgcctgtagcaatggcaacaacgttgcgcaaactattaactg |
| | | gcgaactacttactctagcttcccggcaacaattaatagactggatggaggggataaagttgc |
| | | aggaccacttctgcgctcggcccttccggctggctggtttattgctgataaatctggagccg |
| | | gtgagcgtgggtctcgcggtatcattgcagcactggggccagatggtaagccctcc |
| | | cgtatcgtagttatctacacgacggggagtcaggcaactatggatgaacgaaatagacaga |
| | | tcgctgagataggtgcctcactgattaagcattggtaactgtcagaccaagtttactcatata |
| | | tactttagattgatttaaaacttcatttttaatttaaaaggatctaggtgaagatccttttt |
| | | gataatctcatgaccaaaatcccttaacgtgagttttcgttccactgagcgtcagaccccgt |
| | | agaaaagatcaaaggatcttcttgagatcctttttttctgcgcgtaatctgctgcttgcaaac |
| | | aaaaaaaccaccgctaccagcggtggtttgtttgccggatcaagagctaccaactctttttcc |
| | | gaaggtaactggcttcagcagagcgcagataccaaatactgttcttctagtgtagccgtagtt |
| | | aggccaccacttcaagaactctgtagcaccgcctacatacctcgctctgctaatcctgttacc |
| | | agtggctgctgccagtggcgataagtcgtgtcttaccgggttggactcaagacgatagttacc |
| | | ggataaggcgcagcggtcgggctgaacggggggttcgtgcacacagcccagcttggagcgaac |
| | | gacctacaccgaactgagatacctacagcgtgagctatgagaaagcgccacgcttcccgaa |
| | | gggagaaaggcggacaggtatccggtaagcggcagggtcggaacaggagagcgcac |
| | | gagggagcttccagggggaaacgcctggtatctttatagtcctgtcgggtttcgccacctct |
| | | gacttgagcgtcgatttttgtgatgctcgtcaggggggcggagcctatggaaaaacgccag |
| | | caacgcggcctttttacggttcctggccttttgctggccttttgctcacatgttctttcctgc |
| | | gttatcccctgattctgtggataaccgtattaccgcctttgagtgagctgataccgctcgcgc |
| | | agccgaacgaccgagcgcagcgagtca |
| | | gtgagcgaggaagcggaaga |
| 186 | EsCas13d DR | 5′- |
| | | GAACUACACCCGUGCAAAAAUGCAGGGGUCUAAAAC- |
| | | 3′ |
| 187 | RffCas13d DR | 5′- |
| | | GAACUAUAGUAGUGUGAAUUUACACUACUCUAAAAC- |
| | | 3′ |
| 188 | UrCas13d DR | 5′- |
| | | CUACUACACUGGUGCAAAUUUGCACUAGUCUAAAAC- |
| | | 3′ |
| 189 | RaaCas13d DR | 5′- |
| | | CUACUAUACUAGUGUGAUUUUACACUAGUCUAAAAC- |
| | | 3′ |
| 190 | P1E0Cas13d | 5′- |
| | DR | GCACUACACCCCCCUGAAACAUGAGGGGUCUAAAAC- |
| | | 3′ |
| 191 | AdmCas13d | 5′- |
| | DR | GACCAACACCUCUGCAAAACUGCAGGGGUCUAAAAC- |
| | | 3′ |
| 192 | RfxCas13d DR | 5′- |
| | | CAAGUAAACCCCUACCAACUGGUCGGGGUUUGAAAC- |
| | | 3′ |

Sequences for use in the present disclosure any sequences having at least 70%, or 75%, or 80%, or 85%, or 90%, or 95%, or 99% or up to 100% sequence identity with any of the sequences of Table 2.

### EXAMPLES

### Example 1: Cas13d Collateral Damage and Autoregulation

Cas13 is a family of RNA-targeting CRISPR effector endonucleases with demonstrated potency, specificity, and modularity that set it apart as a therapeutic candidate for RNA-mediated disease, including repeat expansion diseases like myotonic dystrophy type 1 (DM1). CRISPR-Cas13d from Ruminococcus flavefaciens (RfxCas13d) is a small ortholog capable of efficient packaging into AAV gene delivery vectors and effective transcript knockdown in eukaryotes.¹ A danger associated Cas13 is collateral damage, which is off-target degradation of transcripts. Collateral damage, if present in vivo, poses a significant safety risk to any therapeutic use in humans. Early studies of Cas13d did not observe collateral damage in eukaryotic cells suggesting Cas13d collateral damage will not pose a safety risk in the clinic^{1,7}. Surprisingly, in this work, through alternative experimental designs, it is demonstrated that collateral damage is indeed prevalent in human cell lines when expressing Cas13d and a targeting guide RNA (gRNA). This surprising result necessitates strategies to decrease Cas13d collateral damage.

To mitigate Cas13d collateral damage, multiple negative autoregulatory approaches have been developed to minimize and tightly control expression of Cas13 in the cell, which are shown herein to reduce collateral damage while maintaining on-target potency at repetitive targets. These novel applications of negative autoregulation are disclosed herein to improve the safety of therapeutics based on Cas13d. These improvements may be essential to enable safe and effective use of Cas13d in therapeutic applications.

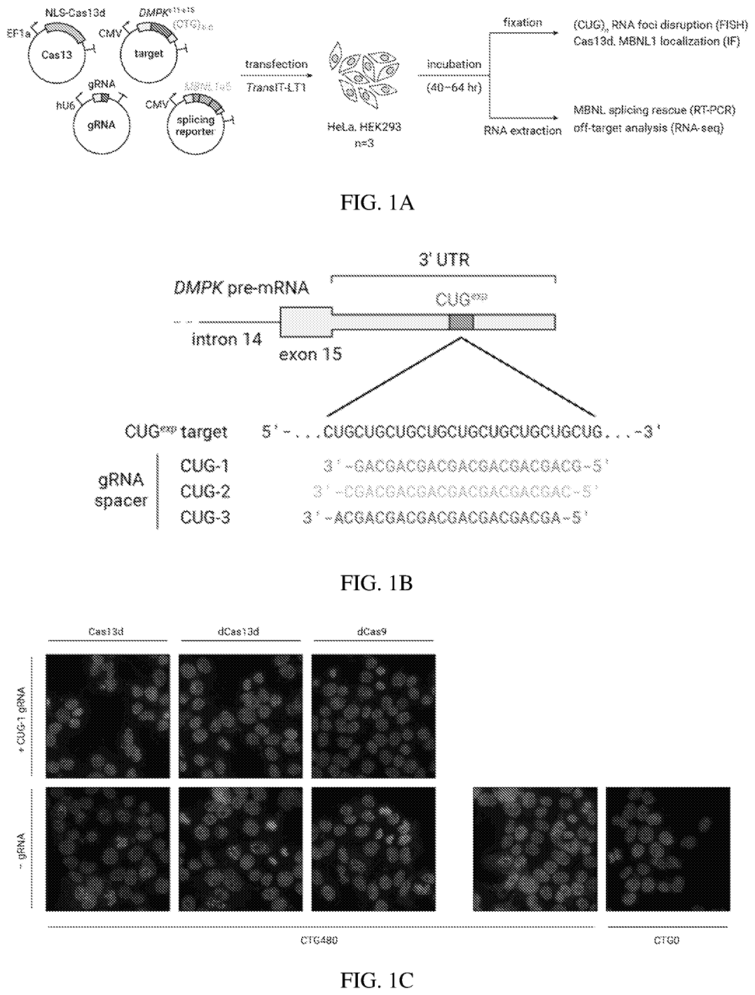

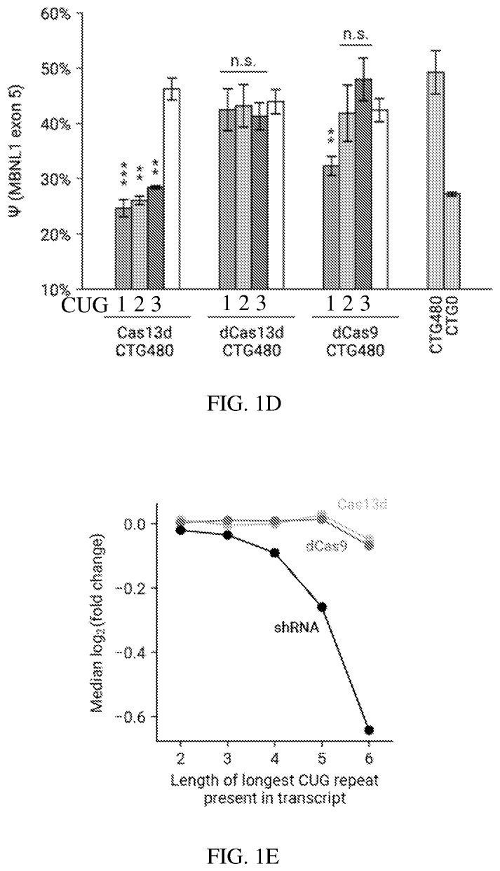

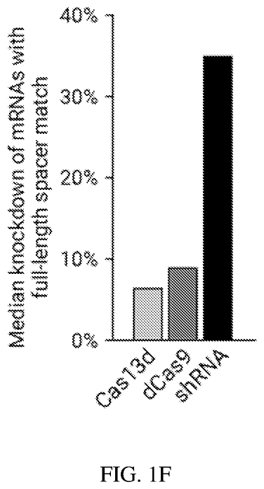

CRISPR-Cas13d efficiently destroys pathogenic CUGexp RNA and restores MBNL1-mediated alternative splicing. The most common adult-onset muscular dystrophy, myotonic dystrophy is a repeat expansion disease that presents a multisystemic progressive syndrome including myotonia, muscle atrophy, hypersomnolence, cataracts, and cardiac arrhythmia. Myotonic dystrophy type 1 (DM1) is caused by a CTG repeat expansion in the 3′ UTR of DMPK, and the resulting toxic CUG^{exp} RNA aggregates into nuclear inclusions and sequesters MBNL, an important family of RNA-binding proteins that regulates alternative splicing across the transcriptome. It was hypothesized that Cas13d targeted to the repetitive CUG^{exp} sequence would yield highly effective knockdown and phenotypic rescue in cell models of DM1 (FIGS. 1A-1F).

Three CUG^{exp}-targeting guide RNAs (gRNAs) were tested for Cas13d spanning the three possible registers of the repetitive CUG sequence in the DMPK mRNA. In HeLa and HEK293 cells, plasmids containing each gRNA were transfected, either Cas13d or a catalytically inactive variant (dCas13d), a DMPK minigene containing 480 CTG repeats, and a minigene used to assay MBNL1-mediated splicing (FIGS. 1A and 1B). For a positive control, plasmids containing catalytically inactive Cas9 (dCas9), and equivalent gRNAs were also tested, which were previously shown to successfully block transcription elongation at the CTG repeat only when the gRNA aligns with an acceptable NAG PAM site.⁴ By fluorescence in situ hybridization (FISH), stark reduction in the number and intensity of RNA foci characteristic of myotonic dystrophy was observed when treated with Cas13d and CUG^{exp}-targeting gRNA (FIG. 1C). Additionally, measurement of minigene splicing by RT-PCR showed restoration of MBNL1 alternative splicing with active Cas13d and any of the three gRNAs (FIG. 1D). It was observed that binding the pathogenic RNA with nuclease-inactive dCas13d is not sufficient to rescue aberrant splicing. These results suggest that targeted cleavage of pathogenic repetitive RNA sequences by Cas13d may prove a viable therapeutic option for myotonic dystrophy and other repeat expansion diseases.

The targeting specificity of Cas13d was assessed using RNA-seq and differential expression analysis, and this specificity was compared to other repeat-targeting approaches including dCas9 and short hairpin RNA (shRNA). It was observed that repeat-targeting shRNA had poor specificity, disrupting many RNAs across the transcriptome containing short CUG tracts (FIG. 1E). In contrast, it was found that the CRISPR approaches were far more specific for long CUG repeats (FIG. 1F), which provides these technologies with robust selectivity for expanded alleles.

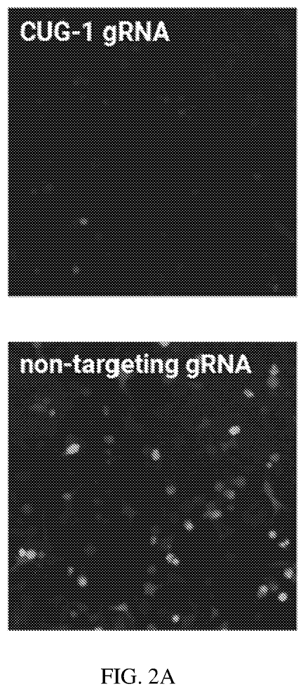

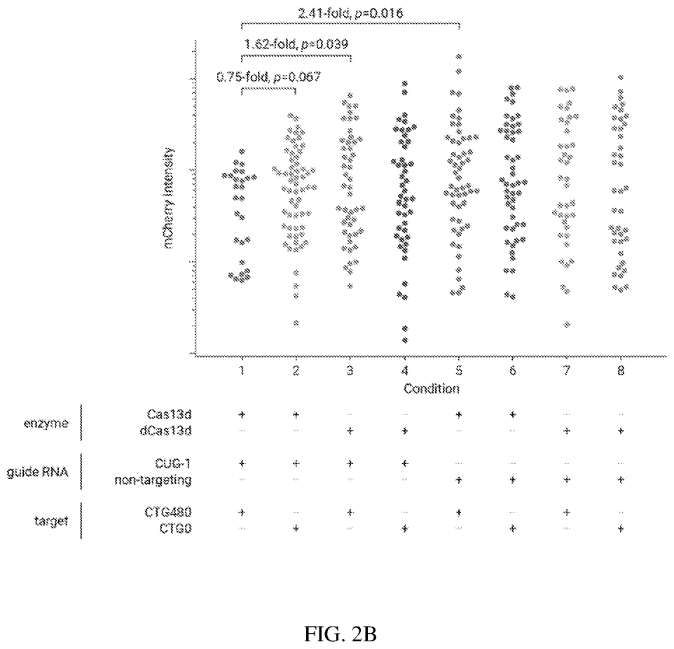

Cas13d exhibits collateral damage in human cells. When Cas13d was expressed in HeLa cells with a repeat-targeting gRNA and CUG^{exp} target RNA, it was observed that expression of Cas13 was sharply reduced when compared to a non-targeting gRNA (FIG. 2A). Additionally, a stark increase in cell death was noticed in the repeat-targeting condition compared to the non-targeting control (data not shown). It was suspected that this may be the result of non-specific collateral damage after Cas13d activation, which at the time of this study had only been observed in vitro.

To quantify collateral damage in human cells, a constitutively expressed mCherry reporter plasmid was co-transfected with the Cas13d targeting machinery. It was hypothesized that collateral damage, if observed, would reduce the expression of the otherwise orthogonal mCherry reporter. Indeed, a 61% reduction in mCherry expression was observed by fluorescence microscopy when all components of the targeting system were present, and no statistically significant reduction in mCherry was observed when any one of the components (Cas13d, gRNA, and CUG^{exp} target) was removed (FIG. 2B).

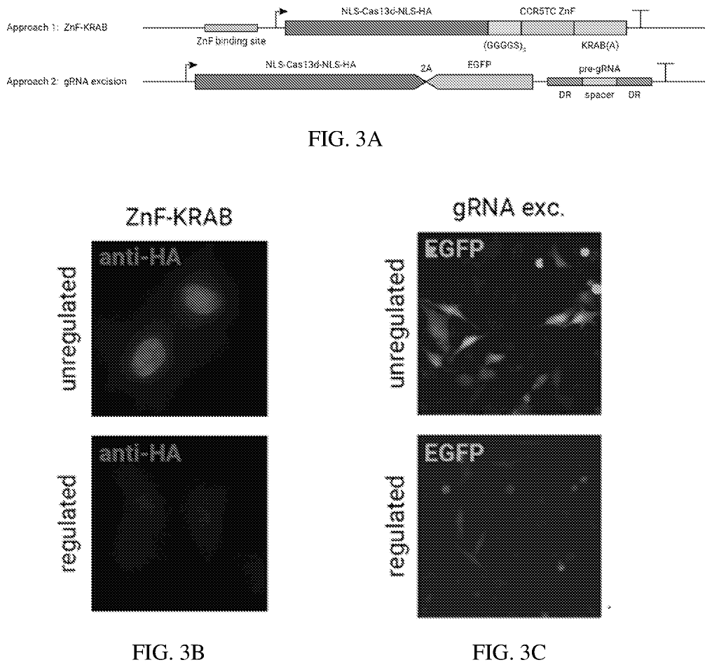

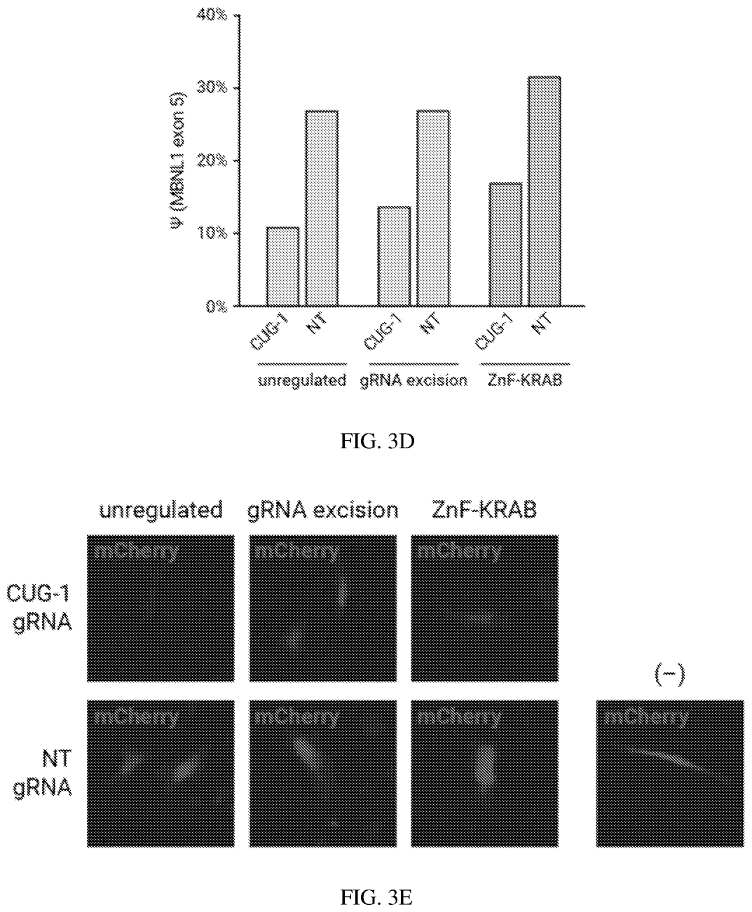

Negative autoregulation reduces collateral damage and maintains on-target activity. It was hypothesized that minimization of Cas13d expression would reduce the number of cleavage-activated complexes in the cell, abating collateral damage. Thus, to mitigate collateral activity in the context of Cas13d therapeutics, multiple approaches were developed to apply negative autoregulation to synthetically control and minimize expression (FIG. 3A).

Approach 1 utilizes a fusion of Cas13d to a synthetic zinc finger and a Krüppel associated box (KRAB) domain to add transcriptional repression functionality to the Cas13d protein. When coupled with its own zinc finger binding site upstream of the Cas13d promoter, this fusion results in stark reduction of Cas13d expression (FIG. 3B).

In Approach 2, rather than utilizing its own promoter, the pre-gRNA is inserted directly into the Cas13d mRNA. This novel approach takes advantage of the pre-gRNA processing activity of Cas13d: when Cas13d is translated and binds the pre-gRNA present within its transcript, the pre-gRNA is excised, producing unprotected termini that are degraded by RNA exonucleases. This yields effective autoregulatory knockdown of Cas13d (FIG. 3C).

In a HeLa cell line expressing 480 CTG repeats, these autoregulatory designs were transfected with the MBNL splicing minigene. Similar rescue of MBNL alternative splicing activity was observed with both Approach 1 and 2 when compared to unregulated Cas13d (FIG. 3D), indicating that on-target activity is maintained even with reduced Cas13d expression. When co-transfected with the mCherry reporter, it was found that both approaches partially rescued mCherry expression (FIG. 3E), indicating a reduction of collateral damage with Cas13d autoregulation. It is believed that negative autoregulation thus provides a convenient and useful solution to mitigate collateral damage in Cas13d therapies.

### REFERENCES

1. Konermann, S. et al. Transcriptome Engineering with RNA-Targeting Type VI-D CRISPR Effectors. Cell 173, 665-676.e14 (2018).
2. Abudayyeh, O. O. et al. C2c2 is a single-component programmable RNA-guided RNA-targeting CRISPR effector. Science 353, aaf5573 (2016).
3. Abudayyeh, O. O. et al. RNA targeting with CRISPR-Cas13. Nature 550, 280-284 (2017).
4. Pinto, B. S. et al. Impeding Transcription of Expanded Microsatellite Repeats by Deactivated Cas9. Mol. Cell 68, 479-490.e5 (2017).
5. Wang et al., Advanced Science Communications, 2019. The CRISPR-Cas13a Gene-Editing System Induces Collateral Cleavage of RNA in Glioma Cells.
6. Lin, Ping, et al. “CRISPR-Cas13 inhibitors block RNA editing in bacteria and mammalian cells.” Molecular Cell (2020).
7. Mahas et al., Genome Biology, 2019. CRISPR-Cas13d mediates robust RNA virus interference in plants.

### Example 2: Negative Autoregulation Mitigates Collateral RNase Activity of Repeat-Targeting CRISPR-Cas13d in Mammalian Cells

Introduction

CRISPR-Cas13 is a recently discovered family of RNA-guided RNA endonucleases capable of sequence-specific binding and potent RNA cleavage [2016 Abudayyeh 27256883]. In class 2 type VI CRISPR systems, Cas13 effectors confer bacterial immunity to phage through recognition of a target RNA sequence complementary to the spacer of the crRNA, or guide RNA (gRNA) [2019 O'Connell 29940185]. Upon target binding, two higher eukaryotes and prokaryotes nucleotide (HEPN) domains undergo conformational change, forming a dual-R-X4-H catalytic site distal to the RNA binding cleft that exhibits potent ribonuclease (RNase) activity [2018 Zhang 30241607, 2019 Bo Zhang Nat Comms]. The programmable nature of its RNA targeting has enabled broad application of Cas13, as well as nuclease-deactivated dCas13 variants and fusion proteins, in eukaryotic cells to reduce expression of endogenous transcripts [2017 Abudayyeh 28976959, 2020 Kushawah 32768421, 2021 Li 33288960], introduce directed base edits [2017 Cox 29070703, 2021 Kannan 34462587, 2021 Xu 33941935] and modifications [2020 Wilson], visualize RNAs in live cells [2019 Yang 31757757], modulate alternative splicing [2018 Konermann 29551272, 2020 Du 32532987], and capture RNA-protein interactions [2020 Han 32839320]. In particular, Cas13d is a family of small orthologs that is especially suited for mammalian applications, with efficient AAV packaging for viral delivery [2018 Konermann 29551272], well-studied determinants of gRNA activity [2020 Wessels 32518401], and no protospacer flanking sequence (PFS) constraints [2018 Yan 29551514]. As a result, Cas13d is a promising candidate for a novel class of RNA-targeting therapeutics that avoids the potential risks of permanent genome editing or DNA binding.

A peculiar feature observed in biochemical and bacterial contexts is that, upon sequence-specific binding of the Cas13:gRNA binary complex to the target RNA, Cas13 unleashes non-specific RNase activity capable of cleaving bystander RNAs in trans [2016 Abudayyeh 27256883, 2016 East-Selesky 27669025]. This phenomenon, often referred to as collateral activity, is a robust feature of all known Cas13 orthologs when expressed in bacteria [2019 O'Connell 29940185] and has been leveraged for rapid detection of nucleic acids at attomolar sensitivity [2017 Gootenberg 28408723]. However, the extent of collateral activity of Cas13 in mammalian cells remains disputed. Many groups have observed no evidence of Cas13 collateral activity in eukaryotic cells using a variety of experimental strategies [2017 Abudayyeh 28976959, 2018 Konermann 29551272, 2020 Kushawah 32768421, 2020 Huynh 33203452], and Cas13 has been used effectively in other studies without mention of any trans cleavage effects [2017 Cox 29070703, 2020 Zhou 32272060, 2020 Wessels 32518401, 2021 Li 33288960, 2020 He 32185621]. Yet, there is a small but growing body of evidence that collateral activity in mammalian cells substantially depletes cellular RNAs [2019 Wang 31637166 (Cas13a), 2021 Xu 33941935, 2021 Wang 33391497, 2021 Ozcan 34489594] and that Cas13 is toxic in eukaryotes [2020 Buchman 32584145]. The ambiguity surrounding collateral activity calls into question the experimental utility of Cas13 in the laboratory and presents substantial risk to safety and efficacy of Cas13-based therapeutics, if Cas13 does have collateral activity in mammalian cells and collateral activity cannot be minimized.

In particular, the risks of collateral activity may be magnified in development of Cas13 therapies for repeat expansion diseases, in which it can be advantageous to target the repeated sequence directly [2009 Hu 19412185, 2009 Mulders 19667189, 2012 Lee 22371589]. One example of this class of diseases is myotonic dystrophy type 1 (DM1), a progressive genetic disorder with multisystemic symptoms including myotonia, muscle wasting, and hypersomnolence [2006 Ranum 16776586]. DM1 is caused by a CTG repeat expansion in the 3′ UTR of DMPK [1992 Brook 1310900, 1992 Mahadevan 1546325, 1992 Fu 1546326], which exerts toxicity primarily through RNA gain-of-function mechanisms including sequestration of muscleblind-like (MBNL) RNA-binding proteins [2000 Miller 10970838], upregulation of CELF1 [2007 Kuyumcu-Martinez 17936705], and repeat-associated non-AUG (RAN) translation [2011 Zu 21173221]. In an ensemble of intra- and intermolecular RNA and protein interactions [2017 Jain 28562589, 2012 Krzyzosiak 21908410, 2011 Querido 21511730], MBNL proteins and DMPK mRNAs containing expanded CUG repeats cluster into nuclear foci [1995 Taneja 7896884, 2000 Miller 10970838], preventing MBNL from regulating alternative splicing and shifting isoform ratios transcriptome-wide [2019 Wang 30561649, 2021 Otero]. Although alleles of >50 CTG motifs are associated with diagnosis of DM1, expansion lengths vary widely across the affected population and correlate strongly with severity and age of onset [2018 Paulson 29325606]. In addition, somatic instability yields wide variation in repeat length within a patient's own cells and across tissues [2012 Morales 22595968], resulting in expansions frequently reaching up to thousands of repeat units in skeletal muscle [1994 Thornton 8285579] and brain [2021 Otero]. These effects, coupled with differential expression of DMPK across tissues, create a unique profile of repeat load and toxicity for each cell within each individual. Therapeutic strategies that directly target the repeat RNA sequence may provide a simple mechanism to address this complexity by titrating probability of target cleavage, and thus therapeutic potency, with repeat length at the cellular level. In support of this, this study showed that binding of the CTG expansion by deactivated Streptococcus pyogenes Cas9 (dCas9) inhibits transcription in a repeat-length-dependent manner [2017 Pinto]. With potent on-target activity, lack of protospacer flanking site (PFS) constraints, and efficient AAV packaging [2018 Konermann 29551272, 2018 Yan 29551514], Cas13d programmed with repeat-targeting gRNAs is a strong candidate for developing a therapeutic platform for DM1 and other repeat expansion diseases with RNA or protein gain-of-function mechanisms. Yet, if present, collateral activity of Cas13d may be exacerbated by the many repeated and overlapping protospacers on each target RNA.

Here, the extent of collateral activity of Cas13d was investigated in mammalian cells in the context of a CUG-targeting therapy for DM1. In cell culture experiments, it was found that Cas13d was effective at depleting toxic CUGn RNA and ameliorating MBNL sequestration. However, in both human and mouse cell lines, results showed that Cas13d collateral activity dramatically depletes orthogonal reporter transcripts when targeting CUGn RNA as well as other repetitive and non-repetitive transgenic sequences. Results also showed evidence of collateral activity when targeting mRNAs from highly-expressed endogenous genes at unique protospacers. To combat collateral activity for repeat-targeting Cas13d therapies, a gRNA excision for negative-autoregulatory optimization (GENO) was introduced: a strategy utilizing crRNA processing to minimize and stabilize Cas13d expression that can be easily implemented within AAV packaging constraints. Results showed that GENO substantially reduces collateral activity of Cas13d in human cells and ex vivo mouse myofibers while retaining on-target efficacy for CUGn target RNA. These observations and developments help shed light on the enzymatic properties of Cas13d in mammalian cells and suggest a new class of treatments powered by Cas13d for a broad range of human genetic diseases.

Results

Cas13d Efficiently Reduces Accumulation of Toxic CUGn RNA in Nuclear Foci and Rescues MBNL-Dependent Alternative Splicing

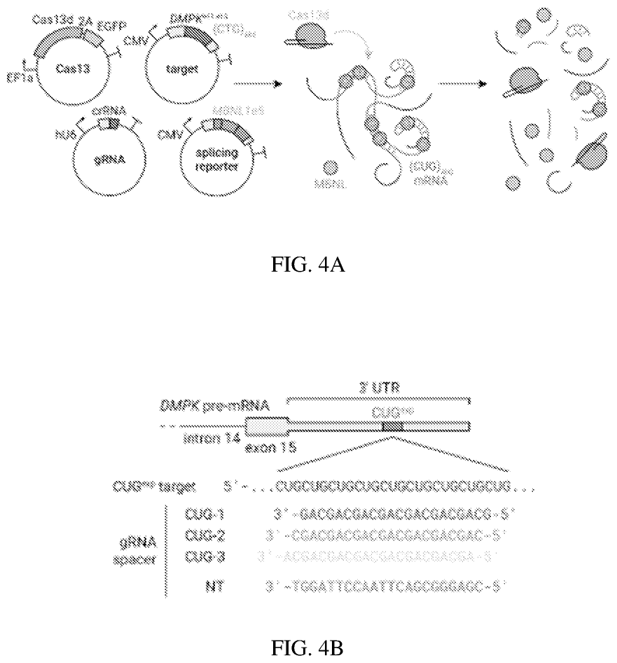

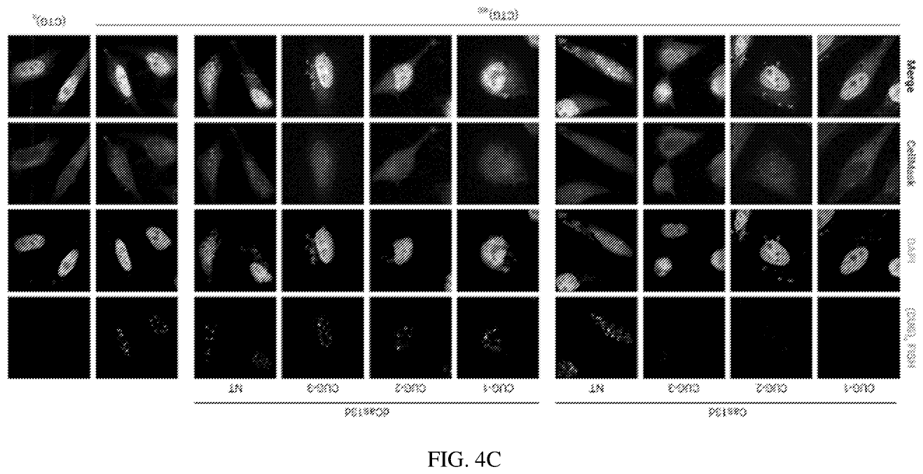

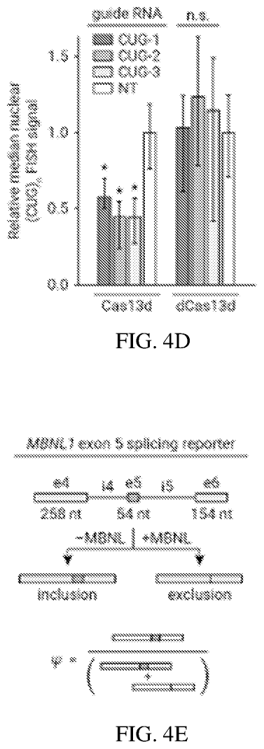

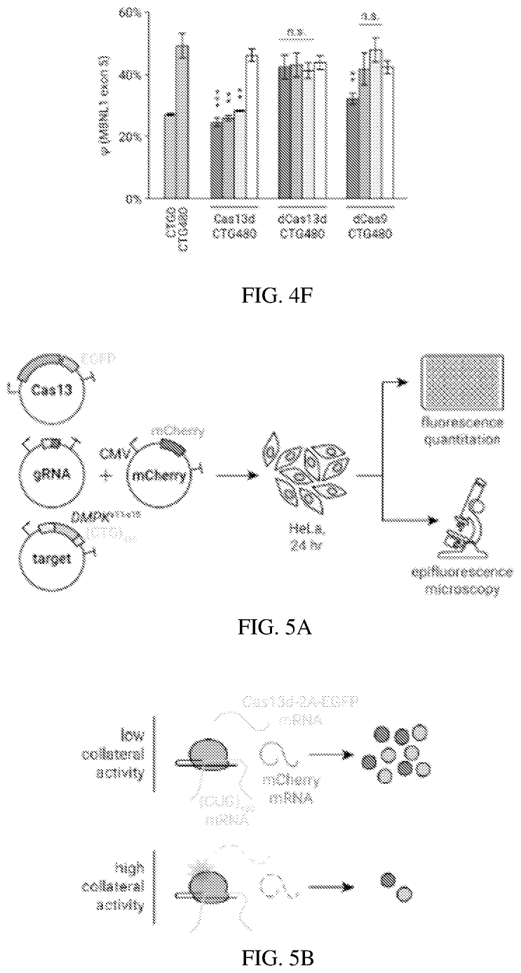

The potential of a CRISPR-Cas13d therapeutic platform for DM1 was evaluated by targeting CUG repeat RNA in a HeLa cell model and assessing the ability of Cas13d to disrupt nuclear RNA foci and rescue MBNL-mediated alternative splicing (FIG. 4A). HeLa cells were transfected simultaneously with plasmids expressing HA-tagged Ruminococcus flavefaciens Cas13d (RfxCas13d) with two SV40 nuclear localization signals (NLS) and an unfused EGFP marker, a target RNA consisting of exons 11 to 15 of DMPK containing 480 CUG repeats (CUG480), and a CUG-targeting or non-targeting guide RNA (gRNA) (FIG. 4A). As Cas13d exhibits no observable protospacer flanking sequence (PFS) requirements [2018 Konermann, 2018 Yan], it was hypothesized that gRNAs targeting all three registers of the CUG repeat would initiate RNA cleavage (FIG. 4B, see Supplemental Table 1).

The on-target efficacy of Cas13d was investigated by performing fluorescence in situ hybridization (FISH) for the CUG480 RNA to measure the disruption of nuclear CUG RNA foci (FIG. 4C), and the median FISH signal was quantified in transfected nuclei across multiple widefield epifluorescence images per condition using an automated analysis pipeline (FIG. 4D). As expected, a stark reduction in the intensity of nuclear foci and median nuclear FISH signal was observed when Cas13d was paired with any of the three repeat-targeting gRNAs compared to a non-targeting gRNA (p<0.05, overlap of 95% confidence intervals (CI)). Significant reduction of nuclear FISH signal was not observed by nuclease-deactivated Cas13d (dCas13d) [2018 Konermann] when expressed with any CUG-targeting gRNA (p>0.05, CI overlap), indicating that target-activated RNA cleavage, rather than binding alone, is required to reduce accumulation of nuclear CUG RNA in this setting.

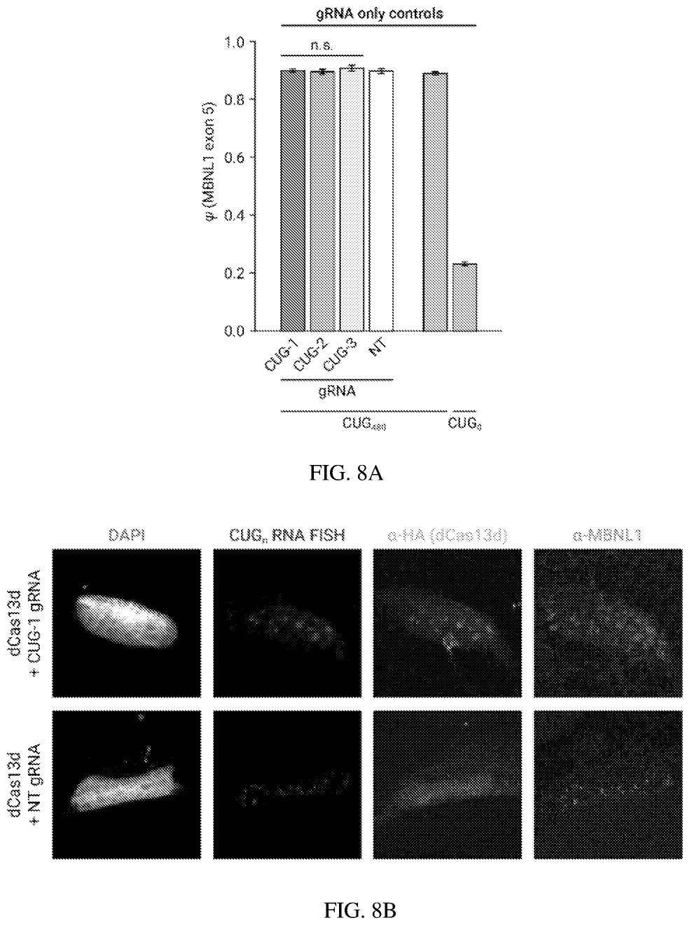

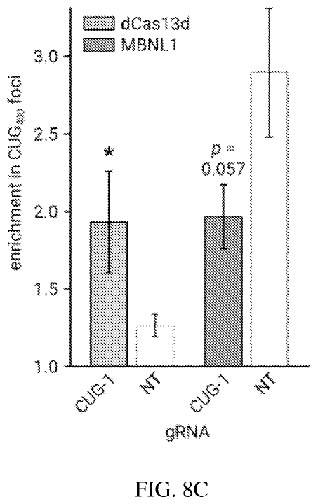

To evaluate the downstream impact of knockdown of CUG repeat RNA, MBNL-dependent alternative splicing was measured using a minigene consisting of MBNL1 exons 4 through 6 with intervening introns (FIG. 4E). Inclusion of MBNL1 exon 5 is suppressed by high concentration of available MBNL proteins in the nucleus but instead is promoted by sequestration of MBNL on CUGn RNA [2011 Gates 21832083]. This splicing minigene was transfected along with the Cas13d, CUG480 target, and gRNA plasmids into HeLa cells and measured the ratio of inclusion of MBNL1 exon 5 (W) by RT-PCR and capillary electrophoresis. Targeting the CUG480 transcript with Cas13d and any CUG-targeting gRNA was found to significantly reduced MBNL1 exon 5 inclusion compared to a non-targeting gRNA (FIG. 4F, p<0.01, two-tailed Student's t test), suggesting that disruption of CUG480 RNA by Cas13d robustly reestablishes normal MBNL-dependent splicing in this model. As expected, similar reduction of exon inclusion was not observed when gRNAs were transfected in the absence of Cas13d (FIG. 8A), indicating that the splicing rescue observed upon Cas13d targeting is not a result of activation of endogenous RNA interference machinery.

Similar changes to splicing with nuclease-inactive dCas13d (FIG. 4F, p>0.05, two-tailed Student's t test) were not observed. To assess if dCas13d is able to successfully bind the CUG480 RNA, FISH was performed simultaneously with immunofluorescence (IF) using antibodies against MBNL1 and HA-tagged dCas13d (FIG. 8B). colocalization of dCas13d with CUG480 RNA foci observed in the nucleus increased when paired with CUG-targeting gRNA rather than non-targeting gRNA (1.93 vs. 1.27 enrichment ratio, p<0.05, two-sided Mann-Whitney U test, FIG. 8C), indicating that dCas13d successfully binds expanded CUG RNA in a sequence-specific manner. Intriguingly, enrichment of MBNL1 in foci appeared only partially reduced by CUG-targeting dCas13d (1.97 vs. 2.90 enrichment ratio, p=0.057, two-sided Mann-Whitney U test, FIG. 8C). These results suggest that although dCas13d may compete with MBNL1 at binding sites within CUG RNA foci, partial displacement is insufficient to restore MBNL1-mediated splicing in this model system. This may result from transient overexpression of the target plasmid, which could produce sufficient amounts of CUG480 RNA to support binding of dCas13d without complete displacement of MBNL1. However, these experiments may also indicate that the affinity of dCas13d to CUGn RNA is inadequate to sufficiently exclude MBNL1 from RNA foci. Further experimentation in model systems with near-endogenous levels of repeat expression are needed to discern between these conclusions.

Nuclease-deactivated Streptococcus pyogenes Cas9 (dCas9) paired with a CUG-targeting gRNA was previous shown to efficiently block transcription of CUGn RNA [2017 Pinto]. The dCas9 gene was cloned into an expression context matching that of Cas13d, and this dCas9 plasmid was transfected into HeLa cells along with CUG480 target, the MBNL1 exon 5 minigene, and CTG-targeting or non-targeting gRNA with spacers matching the Cas13d gRNAs. Confirming the original findings, dCas9 was observed to rescue splicing of MBNL1 exon 5 only when paired with the CUG-1 gRNA (FIG. 4F, p<0.01, two-tailed Student's t test). The spacer sequence of this gRNA aligns to the register of the CTG480 repeat with a CAG protospacer adjacent motif (PAM), which is acceptable for binding of dSpCas9 [2013 Jiang 23360965, 2013 Hsu 23873081]. gRNAs targeting registers with AGC or GCA PAMs (CUG-2 and CUG-3, respectively) did not rescue MBNL alternative splicing (FIG. 4F, p>0.05, two-tailed Student's t test). In contrast, all three registers supported rescue of splicing by Cas13d, suggesting that the lack of PFS requirements for binding and activation of Cas13d expands the available sequence targeting space for CRISPR therapies.

CUG-Targeted Cas13d Prevents Expression of EGFP

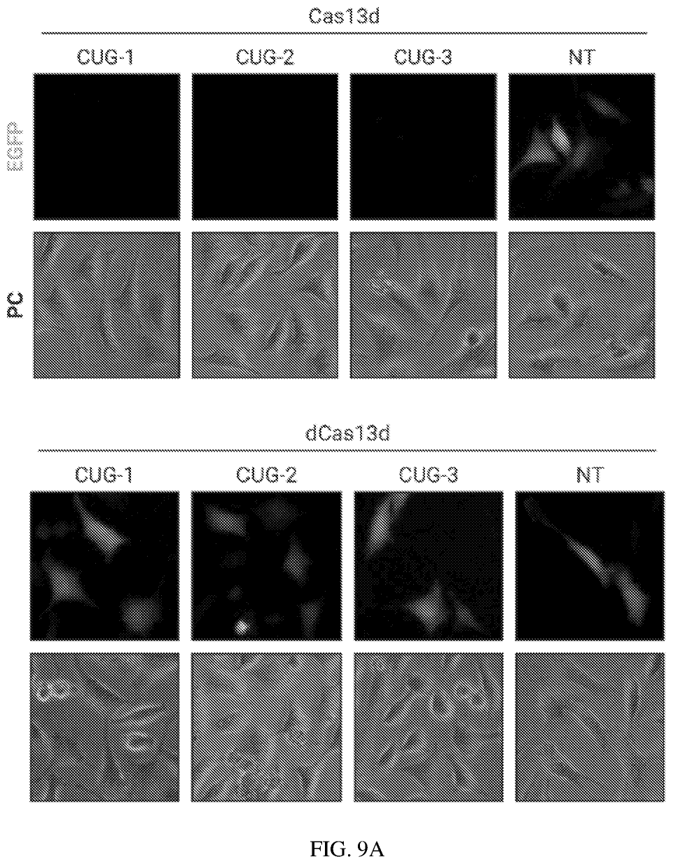

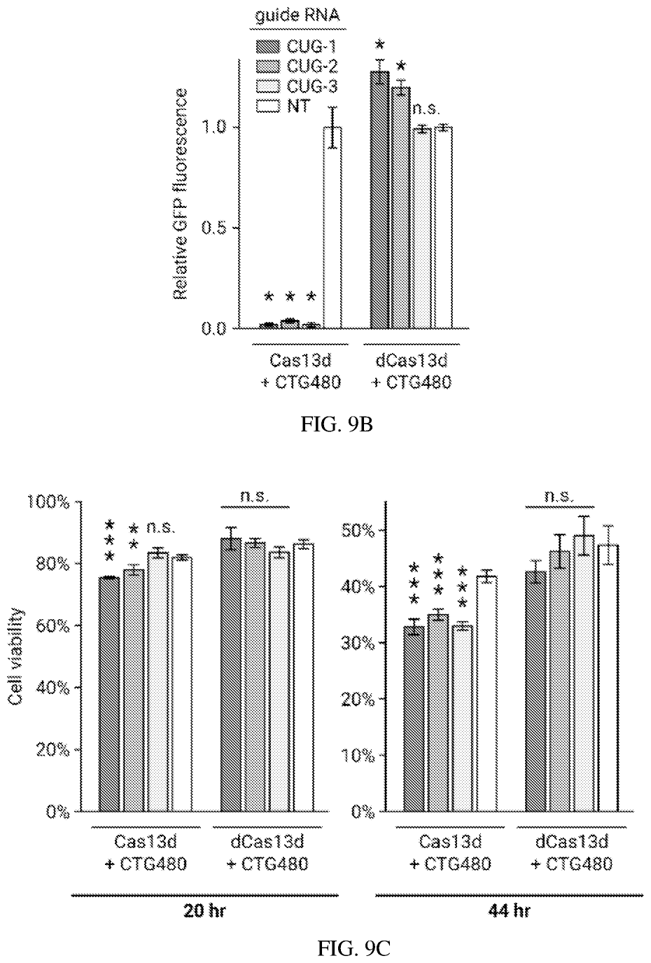

Although Cas13d was effective at reducing CUGn RNA accumulation and restoring MBNL-mediated splicing, the expression of the EGFP marker could not be visualized after transfection of Cas13d, CUG-targeting gRNA, and CUG480 target plasmids, but EGFP expression was apparent when either dCas13d or non-targeting gRNA were substituted (FIG. 9A). To quantitate this phenomenon, target plasmid was transfected into HeLa cells along with either Cas13d or dCas13d and with either CUG-targeting or non-targeting gRNA, and EGFP fluorescence was measured 20 hr after transfection. EGFP expression was found to be reduced by >96% upon transfection with Cas13d and any CUG-targeting gRNA compared to the non-targeting control (FIG. 9B, p<0.05, two-tailed Student's t test). The same effect was not observed for dCas13d (FIG. 9B), indicating that loss of EGFP fluorescence results from Cas13d RNase activity.

To ensure that this reduction of EGFP fluorescence does not merely reflect differences in cell survival, a resazurin cell viability assay was performed 20 hr and 44 hr after transfection. After 20 hr, only a slight reduction in cell viability was observed when Cas13d was transfected with CUG-1 or CUG-2 gRNA compared to non-targeting gRNA (FIG. 9C, 8.0% reduction for CUG-1 and 4.8% for CUG-2, p<0.01, two-tailed Student's t test), indicating that the observed loss of EGFP signal after 20 hr is not explained by death of transfected cells. However, a larger reduction in cell viability was observed 44 hr after transfection for all CUG-targeting gRNAs (FIG. 9C, >16% reduction for all gRNAs, p<0.001, two-tailed Student's t test). These results suggest that persistent targeting of CUGn RNA by Cas13d is indeed cytotoxic, yet measurements taken 20 hr after transfection are mostly unaffected by cell mortality.

CUG-Targeted Cas13d Upregulates Stress Response and Apoptosis Signaling Pathways

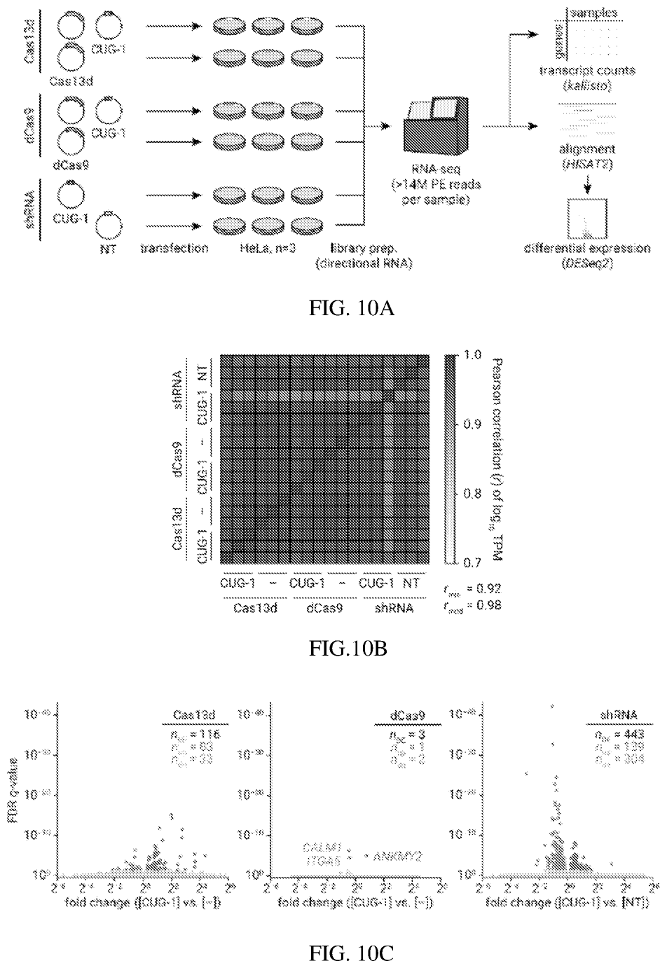

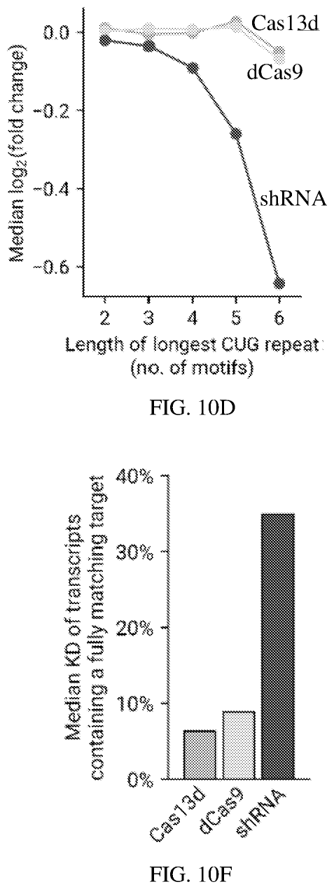

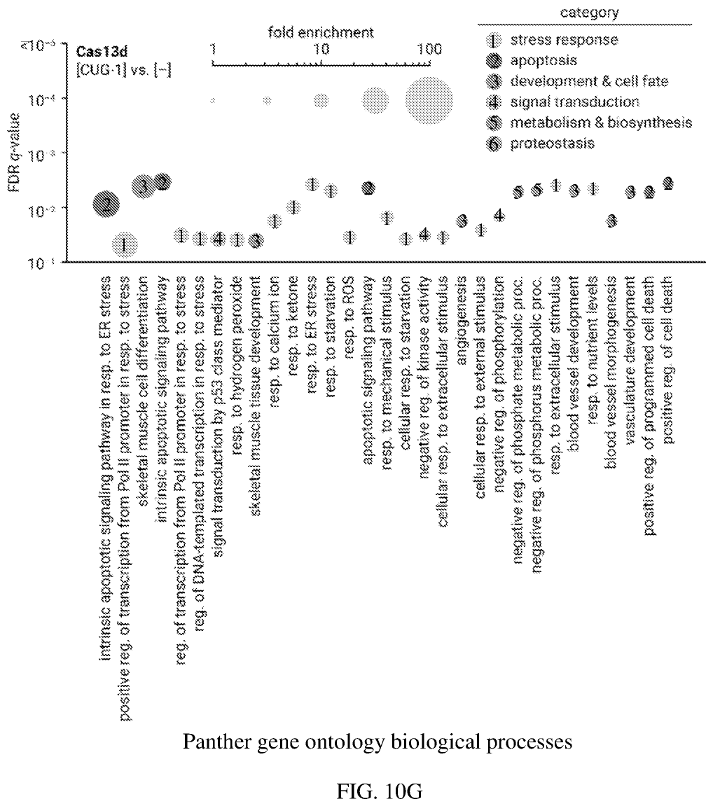

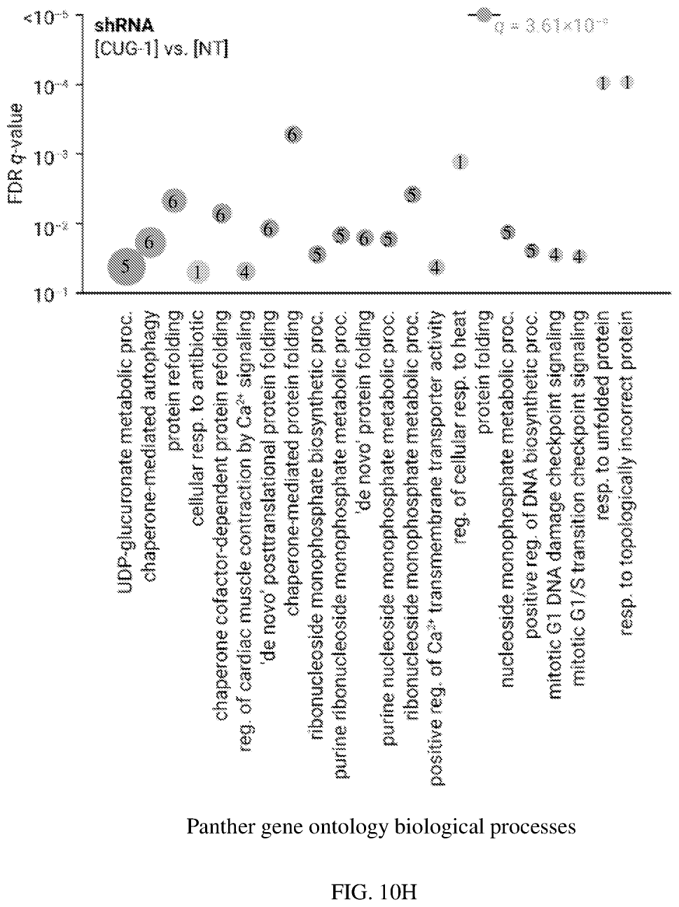

To investigate this further, RNA sequencing (RNA-seq) was performed to profile biological pathways disrupted by CUG-targeting Cas13d and to compare these transcriptomic changes to the effects of other repeat-targeted technologies (FIG. 10). HeLa cells were transfected with Cas13d and with or without CUG-1 gRNA and sequenced RNA collected 68 hr after transfection (FIG. 10A). For comparison, dSpCas9 was also tested with and without CUG-1 gRNA as well as sequence-matched CUG-targeting and non-targeting short hairpin RNAs (shRNAs), which utilize the endogenous RNA interference machinery for knockdown [2002 Paddison 11959843] and are frequently used as comparators for Cas13 off-targeting [2017 Abudayyeh 28976959]. Transcripts from 57 genes in the human reference genome (hg19) contain CUGn repeats that extend beyond the length of the Cas13d spacer (Supplemental Table 2) and therefore are likely off-targets of CUG-targeting approaches. The CUGn target plasmid was omitted in this experiment to enrich for these off-target events. Consistently strong correlations of transcripts per million (TPM) estimates were observed between RNA-seq libraries across all conditions (FIG. 10B, minimum Pearson r of log(TPM)=0.92, median=0.98), suggesting low variance introduced during library preparation.

CUG-targeting Cas13d disrupted fewer off-target genes than shRNA (116 vs. 443 genes differentially expressed (DE) between targeting and non-targeting conditions, false discovery rate (FDR) q<0.05) but many more genes than dCas9 (3 DE genes, FDR q<0.05) (FIG. 10C). 69% of genes disrupted by CUG-targeting shRNA were downregulated (304 vs. 139 genes), consistent with the notion that RISC-mediated RNA cleavage is the dominant mechanism underlying differential expression. In contrast, most of the genes perturbed by Cas13d were upregulated (83 vs. 33 genes), suggesting that a mechanism other than cis RNA cleavage is responsible for most DE genes.

Of the genes disrupted by dCas9, two were downregulated (CALM1 and ITGA5, 30% reduction) and one was upregulated (ANKMY2, 70% increase). The 5′ UTR of CALM1 contains a perfectly matching protospacer with a CGG PAM, and ITGA5 exon 1 contains a protospacer with a single mismatch and CGG PAM. Both prospective targets fall <150 bp from the transcription start site (TSS), suggesting steric inhibition of transcription elongation. A perfect protospacer with a TGG PAM is located 385 bp upstream of the TSS of ANKMY2 and falls within an H3K27ac-enriched active enhancer, suggesting that dCas9 may interact with cis or trans factors within this enhancer to increase transcription. In all three cases, prospective dCas9 target sites found within, or near off-target gene loci strongly suggest that differential expression is driven by sequence-specific interactions.

To determine if the differences in off-target profiles could reflect differences in the strength of knockdown of RNAs containing CUG repeats, we binned transcripts by the length of their longest CUG repeat in the hg19 reference genome and calculated the median fold change between targeting and non-targeting conditions. A strong relationship between off-target knockdown by shRNA and CUG repeat length for genes with repeats shorter than the shRNA targeting sequence was found (FIG. 10D), and an average of 35% knockdown of transcripts was observed with CUG repeats >22 nt (Supplemental FIG. 10E). In contrast, both Cas13d and dCas9 exhibited substantially reduced knockdown of off-target RNAs containing short CUG repeats, with very similar profiles between the two CRISPR effectors (FIG. 10D). In fact, the median knockdown of transcripts with CUG repeats >22 nt was slightly weaker for Cas13d than dCas9 (6.4% vs. 8.9%, FIG. 10E). This suggests that knockdown of endogenous genes containing CUG repeats does not fully explain the extensive off-target profile of CUG-targeted Cas13d.

Gene ontology (GO) analysis was performed using PANTHER [2019 Mi 30804569] to investigate the biological pathways enriched in these differentially expressed genes. The majority of biological processes perturbed by Cas13d were found to be involved in either stress response (48%) or apoptosis (16%) signaling pathways (FIG. 10F, enrichment >5, FDR q<0.05). In contrast, reduced enrichment of stress response pathways (18% of enriched processes) and lack of enrichment of apoptosis signaling was observed when cells were treated with CUG-targeting shRNAs (FIG. 10G-H). These results are consistent with the notion that Cas13d activation by CUG repeats leads to toxicity and cell death beyond what is expected from cis cleavage of off-target RNAs alone.

CUG-Targeted Cas13d Reduces Expression of Orthogonal mCherry Reporter in Mammalian Cells

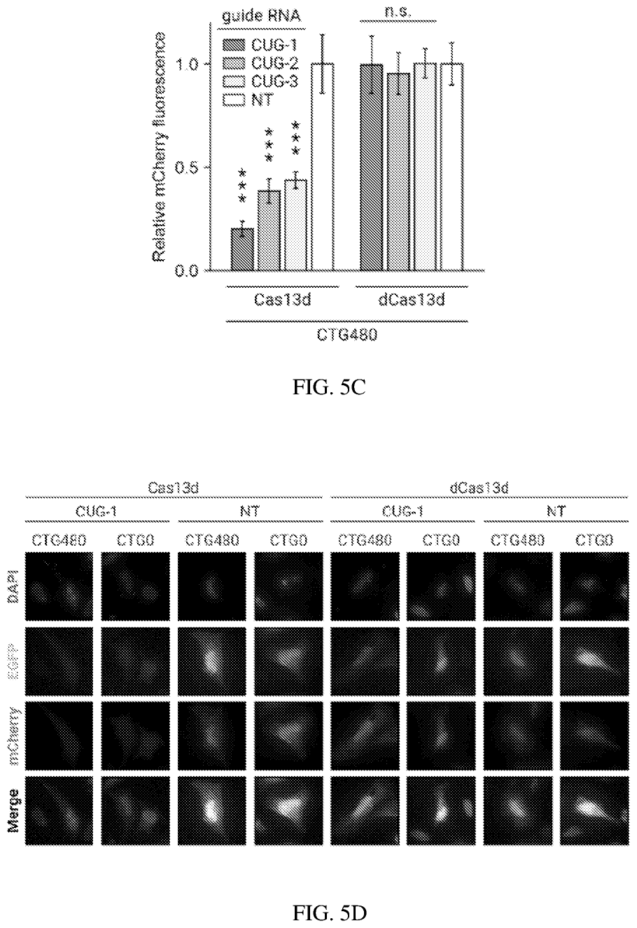

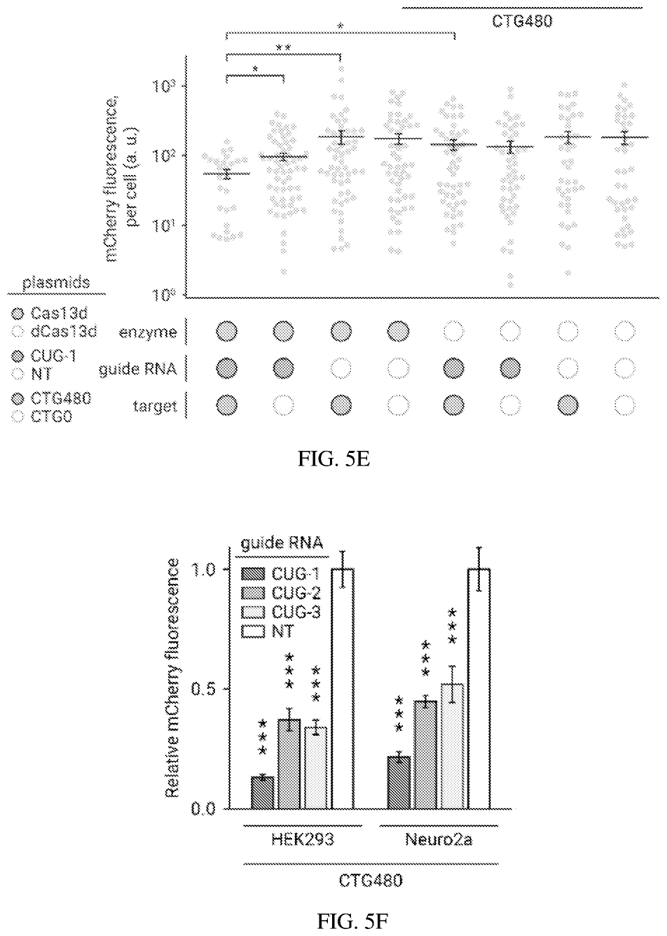

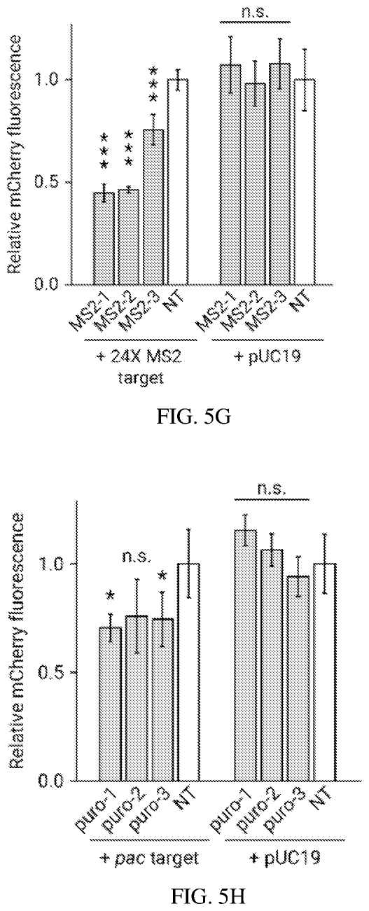

It was hypothesized that inhibition of EGFP expression and induction of stress response genes may result from mass depletion of cellular RNAs by Cas13d collateral RNase activity upon activation by CUGn RNA. To assay for collateral activity, a plasmid constitutively expressing mCherry as an orthogonal reporter gene was transfected into HeLa cells along with Cas13d, gRNA, and CUG480 target plasmid, and mCherry fluorescence was measured in response to Cas13d targeting of CUG RNA (FIG. 5A). It was reasoned that if Cas13d collateral activity is extensive, a general reduction of cellular transcripts, including transgenic mCherry mRNA, should be observed, which would reduce subsequent translation and fluorescence (FIG. 5B). mCherry fluorescence 20 hr after transfection was measured to minimize the impact of cell viability on bulk fluorescence measurements.

Strikingly, a >56% reduction in mCherry fluorescence in cells transfected with Cas13d and any CUG-targeting gRNA relative to non-targeting gRNA was observed (FIG. 5C, p<0.001, two-tailed Student's t test). Reduction in mCherry fluorescence with dCas13d and any gRNA was not observed, indicating that this effect results from Cas13d RNase activity. To assay fluorescence at the single-cell level, widefield fluorescence microscopy was performed and mCherry intensity for each cell was quantified (FIG. 5D). On average, a >61% decrease in mCherry fluorescence was observed when cells were transfected with Cas13d, CUG-1 gRNA, and CUG480 target compared to conditions when either dCas13d or NT gRNA were substituted and a 43% decrease compared to a condition where the target was substituted for CUG0 (FIG. 5E, p<0.05, one-sided Mann-Whitney U test). A smaller reduction in mCherry intensity (44%) was also observed between CUG-targeting and non-targeting conditions in the absence of CUG480 target (p<0.05, one-sided Mann-Whitney U test), likely due to activation of Cas13d collateral activity by endogenous transcripts. Overall, these results provide strong evidence of abundant Cas13d collateral activity in HeLa cells when targeting CUG repeat RNA.

Other researchers have observed that the extent of collateral activity of Cas13a is dependent on cell type, finding that collateral activity was apparent in human glioma cells but not detectable in the non-cancerous HEK293 line [2019 Wang 31637166]. To determine if collateral activity of Cas13d is specific to HeLa cells, the same experiment was performed in HEK293 and the mouse neuroblastoma Neuro2a line. Similar levels of collateral activity across all cell lines were observed (FIG. 5F, p<0.001, two-tailed Student's t test), suggesting that collateral activity of Cas13d in mammalian cells is a general phenomenon.

Cas13d Reduces mCherry Expression in a Target-Dependent Manner

To determine whether collateral RNase activity is specific to targeting CUG repeats, three gRNAs complementary to the MS2 hairpins from a plasmid expressing 24 MS2 repeats in tandem were designed (Supplemental Table 1). These gRNAs were transfected into HeLa cells along with Cas13d and either the 24×MS2 target plasmid or a control plasmid without MS2 hairpins (pUC19). A significant reduction of mCherry fluorescence was observed with all three MS2-targeting gRNAs when the 24×MS2 target was present (FIG. 5G, average 44% reduction, p<0.001, two-tailed Student's t test), but not with the control target (p>0.05, two-tailed Student's t test). This experiment illustrates that collateral activity is not specific to CUG repeat targets and is only detected when suitable protospacers are present in the transcriptome.

Due to their repetitive nature, it is plausible that repeat expansion RNAs would greatly increase collateral activity by binding many Cas13d molecules simultaneously. Experiments were used determine whether collateral activity is also detectable at unique protospacer sequences. Three gRNAs that target unique sequences within the puromycin acetyltransferase (pac) gene were designed (Supplemental Table 1), and these gRNAs were transfected into HeLa along with Cas13d and either a pac-expressing target plasmid or a control plasmid (pUC19). A significant decrease in mCherry signal was detected for two of the three gRNAs only when co-transfected with the pac target plasmid (FIG. 5H, average 28% reduction, p<0.05, two-tailed Student's t test) and not with the control target (p>0.05, two-tailed Student's t test). These data suggest that although collateral activity may be weaker at unique protospacers than repetitive targets, it may still have a deleterious impact on cellular RNAs and should be thoroughly evaluated prior to use of Cas13d for RNA knockdown in mammalian cells.

Rescue of MBNL Splicing by DMPK-Targeted Cas13d Correlates with Inhibition of mCherry

The above observations support the notion that collateral activity is a general property of the Cas13d enzyme, the rate of which is likely a function of many variables, including biochemical and biological context, concentration of target protospacers, and affinity between the Cas13d:gRNA binary complex and the target RNA. As cis RNase activity of the target RNA by Cas13d after binding has been shown to be largely independent of flanking sequences [2018 Yan] (apart from a preference for uracil bases [2018 Konermann]), it was reasoned that both on-target and collateral RNase activities may be driven primarily by the apparent intracellular KD of the Cas13d:gRNA:target ternary complex and thus may be positively correlated for a given target RNA. To test this hypothesis, eight gRNAs that target unique protospacers within the 3′ UTR of DMPK (all of which are present in the CUG480 target RNA, see Supplemental Table 1) were designed, and co-transfected into HeLa cells with Cas13d, CUG480 target, the MBNL1 exon 5 splicing minigene, and mCherry plasmids. To probe on-target activity, the ratio of inclusion of MBNL1 exon 5 was measured, and bulk mCherry fluorescence was measured to assay collateral activity. A positive correlation between MBNL splicing rescue and mCherry fluorescence (FIG. 5I, Pearson r=0.81) was observed, suggesting that on-target and collateral activity of Cas13d are strongly linked.

Collateral RNase Activity of Cas13d is Observed when Targeting Endogenous Genes

Previous observations of collateral activity were made while targeting overexpressed transgenes with Cas13d in a transient transfection format. It was reasoned that the extent of collateral activity, as a biochemical process driven by the Cas13d ternary complex, would depend stoichiometrically on both Cas13d protein and target RNA concentrations. Experiments were designed to determine whether collateral activity may still be detectable and/or toxic when targeting endogenous genes with genomically encoded Cas13d. gRNAs against six endogenous genes across a wide range of expression levels in HeLa cells (LDHA, CD63, CD81, LGMN, SYBU, EPOR; see Supplemental Table 3) were designed. In a comprehensive high-throughput CRISPR screening study [2015 Hart 26627737], none of these genes were classified as core fitness genes, and all had low confidence scores for essentiality in HeLa cells (Bayes factor <−10), making them strong candidates for assaying the detrimental effects of collateral activity.

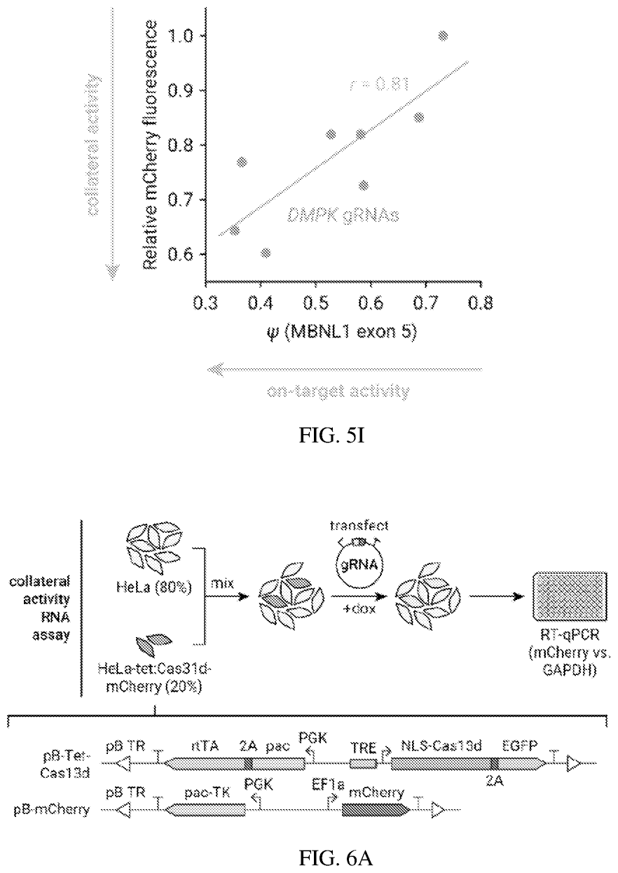

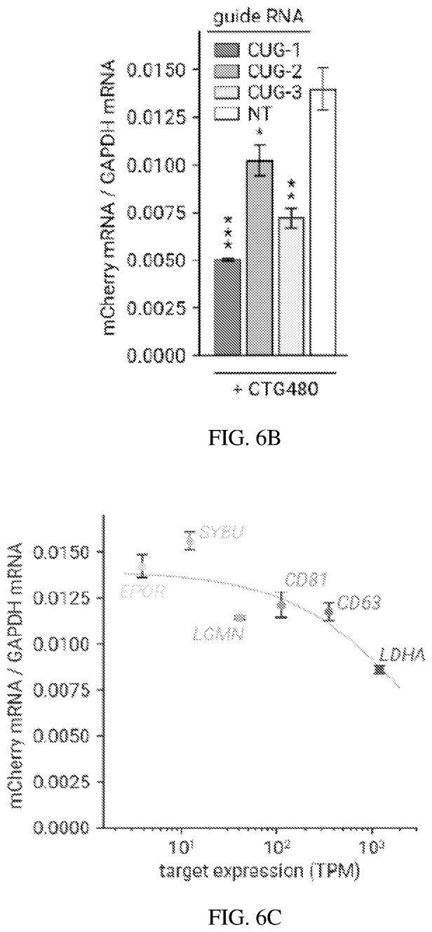

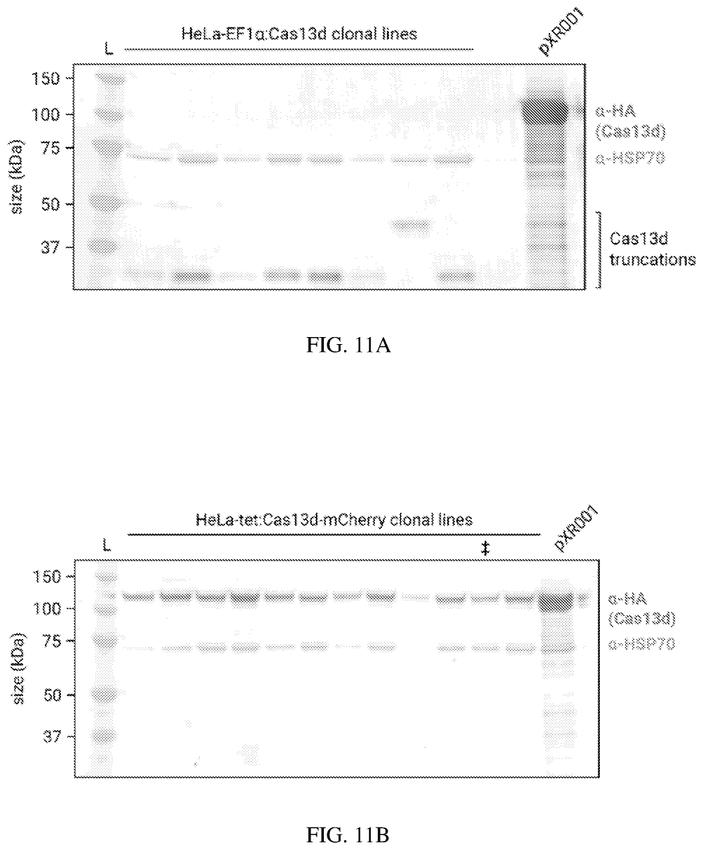

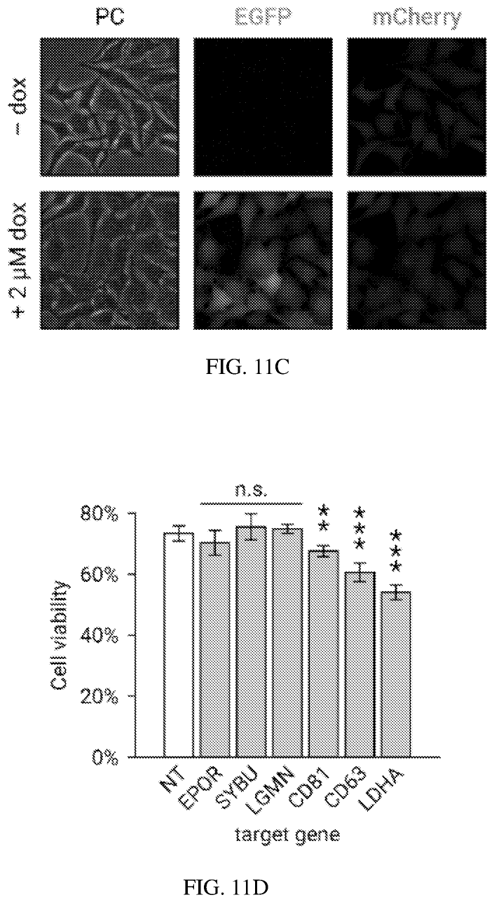

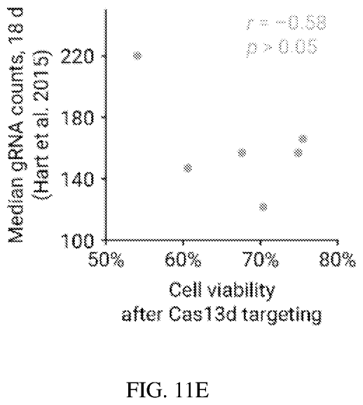

In an effort to generate a cell line constitutively expressing Cas13d, HeLa cells were treated with lentivirus packaged with the Cas13d-2A-EGFP gene under an EF1a promoter and selected GFP-positive single cells by flow cytometry, yet no clonal lines were identified without substantial truncations of Cas13d after expansion (FIG. 11A). Instead, a HeLa cell line with chemically inducible Cas13d was generated by integrating a piggyBac transposon [Cadinanos 2007 17576687] expressing Cas13d-2A-EGFP under a tetracycline-inducible promoter (FIG. 6A). As a reporter for collateral activity, a second cassette constitutively expressing mCherry was also integrated, and a clonal line (HeLa-tet:Cas13d-mCherry) was expanded and validated the expression of full-length Cas13d by Western blot and EGFP and mCherry by fluorescence microscopy after incubation with 2 μM doxycycline for 44 hr (FIG. 114B, 4C).

To measure collateral RNase activity at the RNA level, RT-qPCR experiment was performed to compare abundance of mCherry reporter and control gene (GAPDH) transcripts (FIG. 6A). As collateral activity was expected to degrade RNAs nonspecifically, HeLa-tet:Cas13d-mCherry cells were mixed with nontransgenic HeLa cells at a 1:4 ratio. This co-culture was transfected with gRNA-encoding plasmids, and Cas13d expression was induced with 2 μM doxycycline for 44 hr prior to RNA extraction. As the nontransgenic HeLa cells did not express Cas13d or mCherry, they provided a stable baseline level of GAPDH transcripts without influencing mCherry transcript abundance, enabling measurement of collateral activity by RT-qPCR. To validate the method, CUG-targeting and non-targeting gRNAs were transfected along with CUG480 target, and reduction of mCherry RNA abundance was observed with all CUG-targeting gRNAs comparable to the fluorescence assays (FIG. 6B, average 46% reduction vs. NT gRNA, p<0.05, one-tailed Student's t test of ΔCq).

Using this approach, a statistically significant reduction of mCherry RNA was observed when targeting two of the six endogenous genes in the panel (LDHA and LGMN; FIG. 6C, p<0.05, one-tailed Student's t test of ΔCq). An overall negative correlation between target expression level and mCherry RNA abundance (Pearson r=−0.87 between [mCherry] and log(target TPM)) was noticed. These data suggest that although collateral activity may be strong when targeting highly expressed genes (eg. 34% depletion for LDHA), its effects may be difficult to detect for weakly expressed targets. From this analysis, collateral activity of Cas13d is arguably most problematic when targeting highly expressed and/or repetitive RNAs, it is important to screen for its effects with any target to ensure that nonspecific RNA depletion is not confounding experiments or exerting toxicity during a treatment.

To investigate if collateral activity activated by endogenous genes is sufficient to produce toxicity, a resazurin cell viability assay was performed on HeLa-tet:Cas13d-mCherry cells transfected with gRNA plasmids and induced with 2 μM doxycycline for 44 hr. Statistically significant reductions in cell viability were observed for the three most highly expressed targets (LDHA, CD63, CD81; FIG. 11D, p<0.05, two-tailed Student's t test) and a clear trend of increasing cell mortality with target expression level. Results showed that cell viability did not correlate with depletion of gRNAs targeting these genes in a high-throughput CRISPR screen in HeLa cells [2015 Hart 26627737] (FIG. 11E, p>0.05, beta distribution c.d.f.), providing further evidence that the observed toxicity was caused by collateral activity of Cas13d rather than on-target knockdown effects. It is important to note that cell viability may explain part of the observed reduction of mCherry RNA abundance (FIG. 6C), yet both measurements likely represent different manifestations of the same collateral activity phenomenon. Overall, these experiments suggest that while collateral activity of Cas13d is strongest at repetitive and/or abundant targets, its effects at moderately or weakly expressed endogenous targets should not be ignored.

Negative Autoregulation by gRNA Excision Reduces Expression of Cas13d

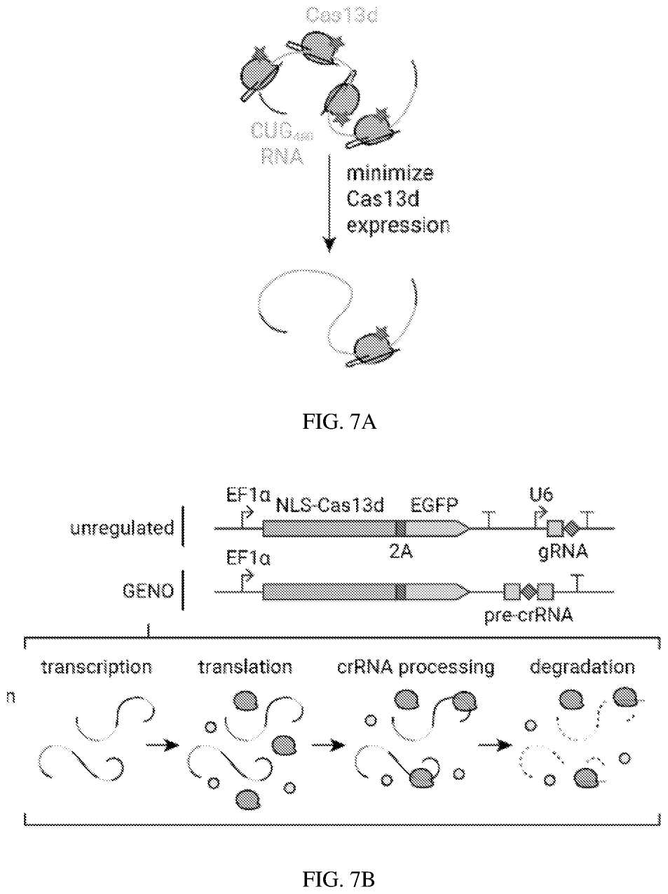

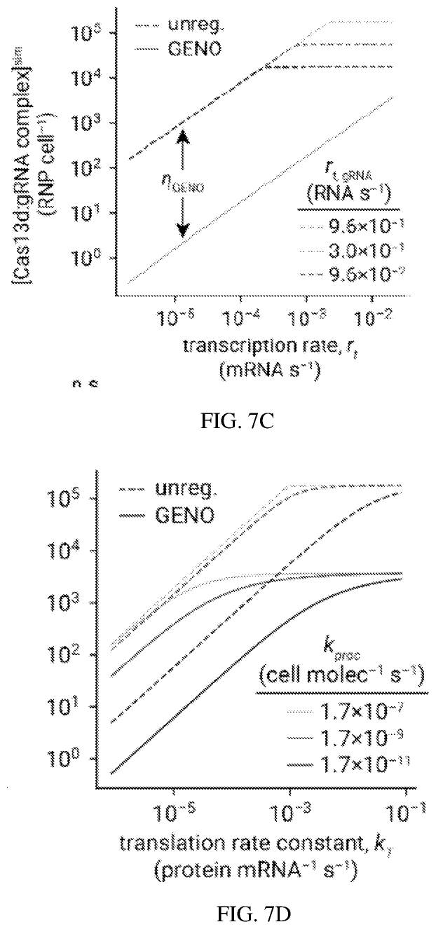

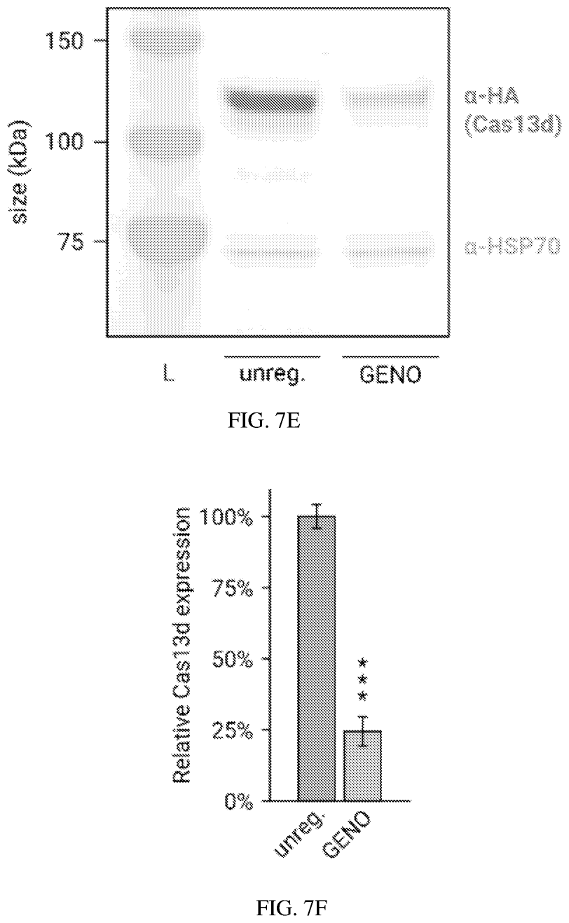

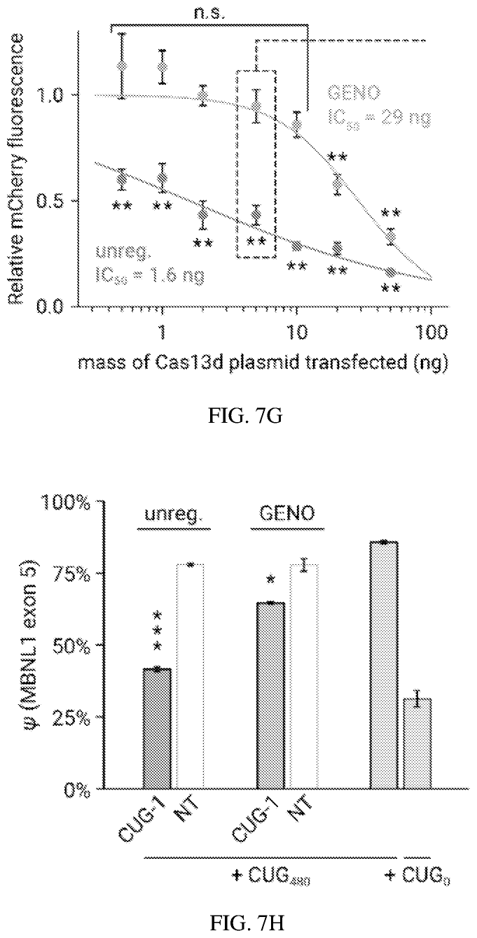

We reasoned that the presence of many overlapping protospacers on the CUG repeat RNA may exacerbate collateral activity by binding many copies of Cas13d:gRNA complex simultaneously (FIG. 7A). Therefore, it was hypothesized that reduction of Cas13d expression may reduce collateral activity while maintaining significant on-target cleavage of the CUGn RNA. A negative autoregulation strategy, termed gRNA excision for negative-autoregulatory optimization (GENO), was developed to sharply reduce and control Cas13d expression by leveraging its crRNA processing activity for self-knockdown (FIG. 7B). In GENO, a pre-crRNA containing the spacer flanked by two direct repeats is placed within a UTR of the Cas13d mRNA. After transcription and translation, Cas13d excises and processes the gRNA to form the binary complex, cleaving its mRNA in the process and leading to degradation and prevention of further translation.

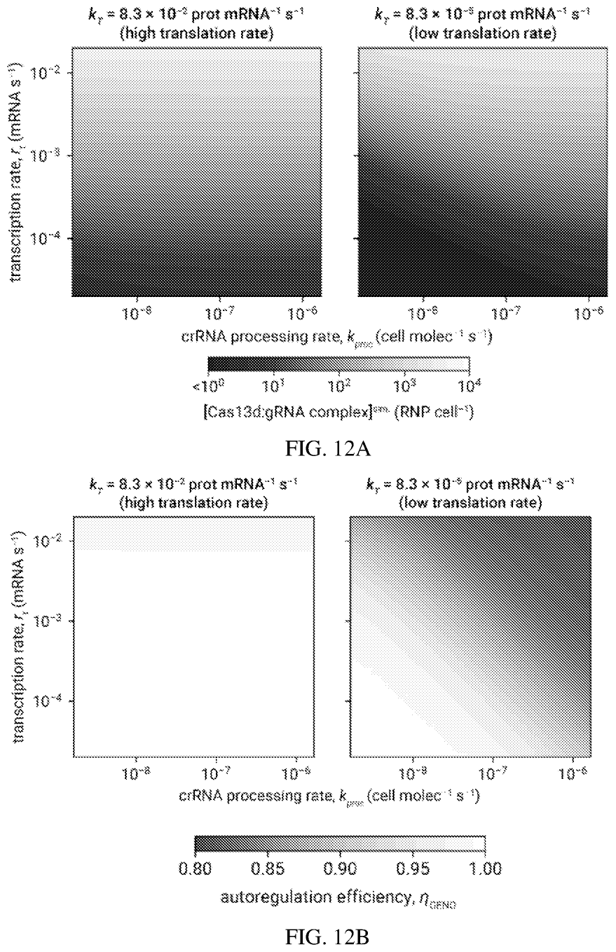

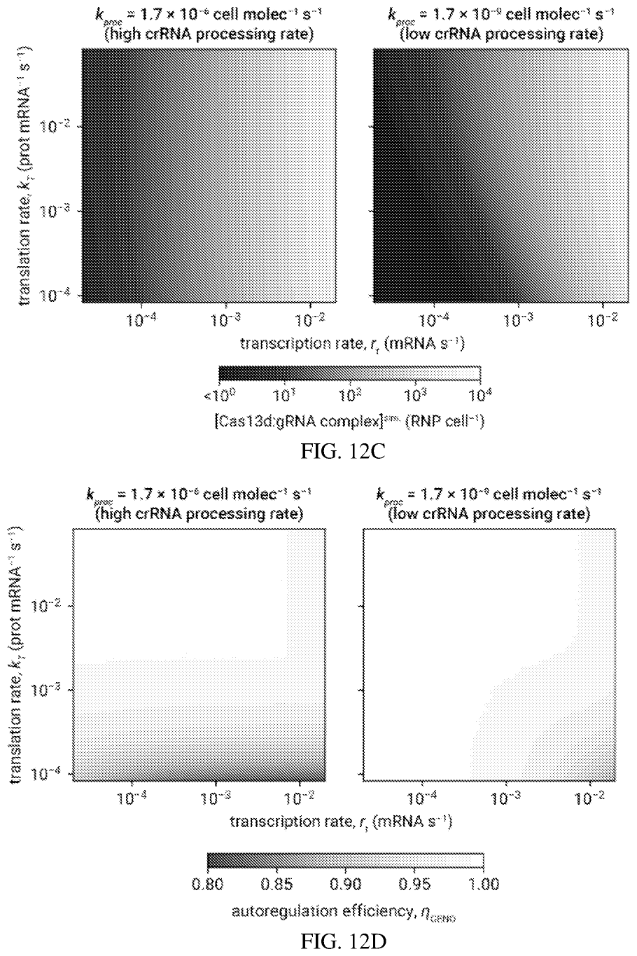

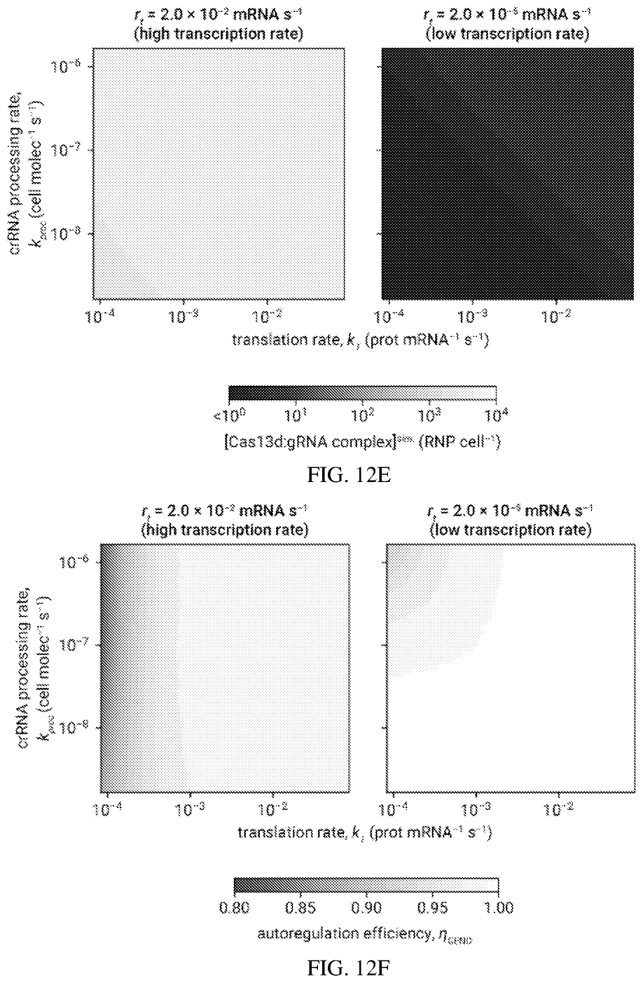

A differential equation model was constructed to describe the dynamics of GENO, and suggested that GENO strictly reduces Cas13d mRNA expression and binary complex production compared to an unregulated reference design for all possible transcription, translation, and crRNA processing rates (see Supplemental Note). To estimate how efficiently Cas13d:gRNA binary complex concentration was reduced by GENO, simulations of the dynamical model across wide ranges of plausible biochemical parameters were performed (FIG. 12). From this analysis, it was predicted that GENO robustly reduces binary complex concentration at equilibrium across a wide range of transcription rates of the Cas13d promoter (FIG. 7C), yet this reduction begins to break down at very high expression levels, at which the concentration in the unregulated design plateaus due to limited gRNA availability. Autoregulation efficiency (ηGENO, defined as the difference of unity and the ratio of equilibrium binary complex concentration in GENO vs. unregulated designs, see Supplemental Note) was predicted to increase with Cas13d translation rate (FIG. 7D). Interestingly, a more complex relationship between autoregulation efficiency and crRNA processing rate was found, exhibiting a positive correlation at high translation rates and a negative correlation at low translation rates (FIG. 7D). Overall, these insights from analytical solutions and dynamical simulations suggest that GENO is a simple and robust approach to regulate Cas13d expression in mammalian cells.

To experimentally determine if GENO reduces Cas13d expression, plasmids encoding GENO-regulated and unregulated Cas13d were transfected into HeLa cells and measured Cas13d protein expression by Western blot (FIG. 7E). In this context, GENO was found to reduce Cas13d protein concentration by 76% (FIG. 7F; p<0.001, two-tailed Student's t test), validating its utility as a strategy for regulating Cas13d expression.

Negative Autoregulation Reduces Collateral Activity while Maintaining Partial On-Target Rescue

To confirm that regulation of Cas13d expression with GENO reduces the extent of collateral activity upon targeting CUG repeat RNA, plasmids encoding GENO-regulated or unregulated Cas13d and gRNA were transfected into HeLa cells along with CUG480 target and mCherry plasmids, and relative mCherry fluorescence between CUG-targeting and non-targeting conditions was measured. Cas13d plasmids were transfected across a wide range of concentrations (0.5 to 50 ng per transfection) to estimate the window of therapeutic benefit provided by GENO. Strikingly, with unregulated Cas13d expression, a strong reduction in mCherry fluorescence was observed at all plasmid concentrations tested (FIG. 7G; p<0.01, two-tailed Student's t test), underscoring the need for a solution to collateral activity for repeat-targeting Cas13d therapies. Results showed that GENO substantially reduced collateral activity to the point where it was statistically undetectable in the assay for all but the two highest plasmid concentrations (p>0.05, two-tailed Student's t test). By fitting logistic functions to these data, it was determined that GENO increases the IC50 of collateral activity by 18 times (1.6 ng to 29 ng), producing a wide window in which collateral activity is improved.

To evaluate if on-target activity at CUGn RNA is maintained upon GENO regulation, a Cas13d plasmid concentration (5 ng per transfection) at which collateral activity was not detected with GENO was chosen, and we measured MBNL alternative splicing activity using the MBNL1 exon 5 minigene assay. It was found that GENO-regulated Cas13d partially rescued MBNL-mediated splicing of this event (FIG. 7H; 36% of rescue observed without regulation, p<0.05, two-tailed Student's t test). Overall, substantial on-target activity of GENO-regulated Cas13d with minimal levels of collateral activity was observed, validating the foundational hypothesis regarding the utility of autoregulation to control Cas13d expression. Further experimentation and optimization will be needed to maximize desired on-target knockdown while maintaining collateral effects below a tolerable threshold.

Methods

Plasmids and molecular cloning. Plasmids encoding NLS-RfxCas13d-NLS-HA-T2A-EGFP (pXR001) and NLS-dRfxCas13d-NLS-HA-T2A-EGFP (pXR002) under the EF1α promoter were purchased from Addgene (#109049 and #109050, respectively), as was a plasmid encoding a gRNA cloning cassette driven by the U6 promoter (pXR003, #109053) [Konermann CasRx]. All plasmids in this study were propagated in NEB Stable chemically competent E. coli (New England Biolabs (NEB), #C3040) at 30° C. and purified using the Zyppy Plasmid Miniprep kit (Zymo Research, #D4036) or ZymoPURE II Plasmid Midiprep kit (Zymo Research, #D4200). Spacer sequences were cloned into pXR003 by BbsI digestion and Gibson assembly (NEB, #E2611) with synthesized DNA duplexes containing the spacer sequence flanked by 19 bp homology arms (Integrated DNA Technologies (IDT)). Plasmids encoding nuclease-inactive Streptococcus pyogenes Cas9 (dCas9), and gRNAs were utilized from a previous study [Pinto 2017 29056323]. To match the expression context of Cas13d, dCas9 was cloned into the pXR001 vector by removing Cas13d with BsiWI and NheI and inserting a PCR-amplified dCas9 amplicon by Gibson assembly. (CUG)n-targeting and non-targeting short hairpin RNAs (shRNAs) matching the corresponding Cas13d spacer sequences were cloned into the pLKO.1 vector (Addgene, #10878) by AgeI and EcoRI digestion and ligation of 5′-phosphorylated DNA duplexes using T4 DNA ligase (NEB, #M0202). Target plasmids expressing 0 and 480 CTG repeats in the context of DMPK exons 11-15 (DMPKS and DT480, respectively) were gifted by Tom Cooper (Baylor College of Medicine).

To investigate collateral activity, a plasmid expressing mCherry under the CMV promoter (pmCherry) was cloned by removing EGFP from pEGFP-CI (Clontech) with AgeI and BglII and assembling the vector with a synthesized mCherry gene fragment (IDT) using In-Fusion cloning (Takara Bio, #102518). pB-Tet-Cas13d was cloned using Gibson assembly by inserting a fragment PCR-amplified from pXR001 containing NLS-RfxCas13d-NLS-HA-T2A-EGFP into a piggyBac transposon vector [Cadinanos 2007 17576687] expressing the insert in a Tet-On cassette and constitutively expressing puromycin acetyltransferase (pac) and reverse tetracycline-controlled transactivator (rtTA) [1995 Gossen 7792603]. pB-mCherry was cloned using Gibson assembly by inserting a PCR-amplified mCherry gene from a synthetic gene fragment (IDT) into a piggyBac transposon vector expressing the insert constitutively under an EF1α promoter and constitutively expressing a pac-thymidine kinase (TK) fusion protein.

To implement the gRNA excision negative feedback design, a synthetic DNA fragment containing CUG-1 or NT pre-crRNA (22 nt spacer flanked by two 30 nt direct repeats (DR)) was assembled by annealing two complementary oligonucleotides (IDT Ultramer) and inserted into the transcribed region of pXR001 at the KpnI site by Gibson assembly. Plasmids for recombinant AAV preparation were generated by cloning NLS-RfxCas13d-NLS-HA and gRNA (either separately driven by U6 for the unregulated design or within the 3′ UTR of the Cas13d gene as pre-crRNA for negative autoregulation) into a vector containing an MHCK7 expression cassette flanked by AAV2 inverted terminal repeats (ITRs).

Cell culture and transfection. HEK293, Neuro2a, HeLa, and HeLa-derived cell lines were maintained in 10% FBS growth medium (Dulbecco's modified eagle medium (DMEM)+10% fetal bovine serum (FBS)+1% penicillin/streptomycin) in a humidity-controlled C02 incubator at 37° C. For transient transfection experiments, unless otherwise stated, cells were passaged to 12-well tissue culture plates at a density of 1.5×105 cells/well (3.9×104 cells/cm2) and transfected with 500 ng plasmid DNA using 2 uL of TransIT-X2 transfection reagent (Mirus Bio, #MIR6005) and 100 uL Opti-MEM I reduced serum medium (ThermoFisher Scientific, #31985088) according to the manufacturer's protocol. When transfecting multiple plasmids, plasmids were mixed at equimolar concentrations unless otherwise noted. Cells were incubated with transfection reagent for 20 hr, after which the culture medium was aspirated and replaced with 10% FBS growth medium. Cells were then incubated for an additional 24 hr for 2-day experiments or 48 hr for 3-day experiments, followed by fixation or RNA isolation for further analysis.

Fluorescence in situ hybridization. Nuclear foci formed by (CUG)n RNA were visualized by FISH using a previously described protocol [Pinto 2017 29056323]. Briefly, cells in 4-well coated glass chamber slides (ThermoFisher Scientific, #154917) were washed with phosphate-buffered saline (PBS) and fixed in 4% paraformaldehyde in PBS at room temperature (RT) for 10 min, washed 3× with PBS, and permeabilized with ice-cold 70% ethanol in water overnight at −20° C. Cells were washed for 30 min at 30° C. in wash buffer (25% formamide, 2× saline sodium citrate (SSC) in water). (CAG)10 FISH probe labeled with Alexa Fluor 594 (Biosearch Technologies, #SS151541-01) was diluted to a working concentration of 380 ng/mL in hybridization buffer (100 mg/mL dextran sulfate, 1 mg/mL yeast tRNA, 2 mM ribonucleoside vanadyl complex. 200 ug/mL bovine serum albumin (BSA), 25% formamide, 2×SSC in water) and incubated with cells overnight at 30° C. The following day, the probe solution was aspirated and replaced with wash buffer for 30 min at 30° C. For nuclear and whole-cell staining, 5 ng/uL DAPI and 1× CellMask Green Plasma Membrane Stain (ThermoFisher Scientific, #C37608) in PBS was added to the cells for 5 min at RT, followed by three washes with PBS of 5 min each. Slides were mounted with glass #1.5 coverslips in Fluoroshield antifade mounting medium (Sigma-Aldrich, #F6182), sealed with clear nail polish, and stored at −20° C. until imaging. Widefield epifluorescence and confocal Airyscan imaging was performed on a Zeiss LSM 880 microscope with a Plan-Apochromat 40×/1.3 Oil DIC M27 objective lens. 10 or more images were collected for each condition. For confocal images, Airyscan processing was performed in Zeiss ZEN software (version 2.1 SP3 FP3 black 14.0.20.201).

Image processing and FISH quantitation were performed in Python 3. 3D epifluorescence images in CZI format were separated into FISH, DAPI, and CellMask channels, and each channel was collapsed to 2D by maximum intensity projection along the z-dimension. Nuclei were segmented from the DAPI channel using Cellpose 0.0.2.0 [2020 Stringer Cellpose]. Transfected cells were identified as those with a mean FISH intensity in the nucleus >50% higher than the background intensity, calculated as the median FISH intensity in the region outside the nuclear mask. 8 or more transfection-positive nuclei were detected for each condition. The median nuclear FISH signal for each condition was calculated, and the 95% confidence interval was estimated by bootstrapping.

MBNL1 exon 5 splicing assay. To measure MBNL-mediated alternative splicing activity, an MBNL-regulated splicing reporter minigene spanning MBNL1 exons 4-6 cloned into the RG6 vector (RG6-MBNL1e5) [Orengo . . . Cooper] was used. RG6-MBNL1e5 was cotransfected into HeLa cells with plasmids encoding Cas13d, (CUG)n-targeting or non-targeting gRNA, and (CUG)480 target. n=3 transfections per condition. 44 hr after transfection, RNA was extracted from cells using 300 uL TRIzol (Zymo Research, #R2050) and purified using the Direct-Zol RNA Miniprep kit (Zymo Research, #R2051) according to the manufacturer's protocol. 100 ng RNA was reverse-transcribed into cDNA using the iScript Reverse Transcription Supermix (Bio-Rad, #1708841). Minigene isoforms were amplified by Taq PCR from 2 uL cDNA using forward and reverse primers complementary to the vector sequence (RG6_F, RG6_R; 54° C. annealing; 28 cycles). Exon 5 inclusion ratios were quantitated from 4 uL of PCR reaction by capillary electrophoresis (Fragment Analyzer, Advanced Analytical 1.1.0.11).

EGFP fluorescence assays. To visualize loss of EGFP expression upon Cas13d targeting. HeLa cells were transfected with plasmids encoding Cas13d and EGFP, (CUG)n-targeting or non-targeting gRNA, and (CUG)480 target. n=3 transfections per condition. 20 hr after transfection, cells were imaged on an EVOS FL digital inverted fluorescence microscope (Life Technologies, #AMF4300PM) at 10× magnification in phase-contrast and GFP-fluorescence channels. Representative images are shown in FIG. 9A.

EGFP quantitation was performed using a plate reader assay. HeLa cells were transfected with identical plasmids as above and plated in a 96-well clear tissue culture plate (Celltreat, #229195). n=5 transfections per condition, 2×104 cells per well. Untransfected cells were plated as a negative control. After 20 hr, growth media was aspirated from the cells and replaced with 100 uL PBS. EGFP fluorescence in each well was measured on a Bio-Tek Cytation 3 plate reader (470 nm excitation, 510 nm emission, 146 gain). Baseline intensity was determined by measuring EGFP fluorescence of untransfected cells, and the mean baseline intensity was subtracted from experimental fluorescence measurements.

Cell viability assay. The PrestoBlue resazurin assay (Invitrogen, #A13261) was used to quantify cell viability in response to (CUG)n-targeting Cas13d. HeLa cells were transfected in a 96-well format with plasmids encoding Cas13d, (CUG)n-targeting or non-targeting gRNA, and (CUG)480 target. n=5 transfections per condition, 2×104 cells per well, with a total volume of 100 uL growth medium per well. 5 wells each of untransfected cells and media alone were also plated. 20 hr after transfection, 11 uL PrestoBlue reagent was added to each well and mixed gently by repeated pipetting. Cells were incubated with PrestoBlue reagent at 37° C. for 30 min before measuring fluorescence on a Bio-Tek Cytation 3 plate reader (550 nm excitation, 590 nm emission, 60 gain). Baseline intensity was calculated as the mean fluorescence of cell-free media and was subtracted from all measurements. Cell viability was calculated by normalizing experimental measurements by the mean fluorescence intensity of untransfected cells.

Cas13d specificity transcriptomic analysis. RNA-seq was performed to screen for transcriptomic off-targets of repeat-targeting approaches (Cas13d, dCas9, and shRNA). (CUG)n-targeted systems and non-targeting controls (no gRNA or non-targeting shRNA) were transfected in triplicate into HeLa cells. (CUG)n target plasmid was omitted to enrich for off-target events. After 68 hr incubation, RNA was extracted with 300 uL TRIzol and purified using the Direct-zol RNA Miniprep Kit. Ribosomal RNA was depleted from 300 ng total RNA using the NEBNext rRNA Depletion Kit (NEB, #E6310L), and libraries were generated for next-generation sequencing using the NEBNext Ultra II Directional RNA Library Prep Kit for Illumina (NEB, #E7760L) according to the manufacturer protocol. Libraries were multiplexed with Illumina i5 and i7 barcoding primers, pooled at 4 nM, and sequenced in a 2×76 bp paired-end format on an Illumina NextSeq 500. >14 million paired end reads were sequenced per library and exported to FASTQ format.

Transcript expression was quantified by pseudoalignment to the hg19 human reference genome using kallisto 0.43.0 [2016 Bray Pachter]. Reads were aligned to hg19 using HISAT2 2.0.0-beta [2019 Kim Salzburg]. For each targeting technology (Cas13d, dCas9, shRNA), differential gene expression analysis was performed using DESeq2 1.30.1 [2014 Love 25516281] to compare (CUG)n-targeting and non-targeting conditions, with gene counts calculated by htseq-count 0.11.2 [2015 Anders 25260700] provided as input. Off-targets were defined as differentially expressed genes with a false discovery rate (FDR) q<0.05. For Cas13d and shRNA libraries, gene ontology (GO) analysis was performed using PANTHER [2019 Mi 30804569] (annotation version 2021-05-01) to identify biological processes associated with each set of off-targets. GO biological processes with FDR q<0.05 were considered significant and were assigned to categories according to their descriptions.

For all annotated human transcripts, longest (CUG)n repeat tract length (in C, U, or G registers) was determined from the NCBI RefSeq reference mRNA sequence. Transcripts were grouped by maximum (CUG)n length, and median log 2 fold-change of TPM between (CUG)n-targeting and non-targeting conditions was calculated for each transcript group and targeting technology.

Collateral activity mCherry fluorescence assays. A plate reader assay was developed to quantitate bulk mCherry fluorescence in transfected cells. HeLa, HEK293, or Neuro2a cells were transfected in a 96-well format with pXR001, targeting or non-targeting gRNA in pXR003, target or control plasmid, and pmCherry. n=5 transfections per condition, 2×104 cells per well, with a total volume of 100 uL growth medium per well. 5 wells of untransfected cells were also plated. After 20 hr incubation, transfection media was aspirated and replaced with 100 uL PBS. mCherry fluorescence was measured on a Bio-Tek Cytation 3 plate reader (587 nm excitation, 627 nm emission, 202 gain). Baseline intensity was defined as the mean mCherry fluorescence of untransfected cells and was subtracted from experimental measurements.

For single-cell measurement of mCherry expression, HeLa cells were transfected in 4-well coated glass chamber slides with plasmids encoding Cas13d and EGFP, CUG-1 or NT gRNA, (CUG)480 target or (CUG)0 control RNA, and mCherry. After 20 hr incubation, cells were washed with phosphate-buffered saline (PBS) and fixed in 4% paraformaldehyde in PBS at RT for 10 min. Cells were washed 3× with PBS, and nuclei were stained with 5 ng/uL DAPI in PBS for 5 min at RT, followed by an additional three PBS washes. Slides were mounted with glass #1.5 coverslips in Fluoroshield antifade mounting medium, sealed with clear nail polish, and stored at −20° C. until imaging. Widefield epifluorescence imaging was performed on a Zeiss LSM 880 microscope with a Plan-Apochromat 40×/1.3 Oil DIC M27 objective lens. 5 or more images were collected for each condition at unbiased non-overlapping fields of view and were processed in Fiji v2.0.0-rc-69/1.52p [2012 Schindelin]. In each image, all EGFP-positive cells were manually segmented, and total mCherry fluorescence intensity was measured for each cell. >26 cells were measured for each condition. Distributions of mCherry expression were compared between conditions using the two-sided Mann-Whitney U test.

Collateral activity RNA assay. To enable precise measurement of Cas13d collateral RNase activity when targeting transgenic or endogenous RNAs, a HeLa cell line was developed containing genomically integrated cassettes expressing mCherry constitutively and Cas13d and EGFP under a tetracycline-inducible promoter (HeLa-tet:Cas13d-mCherry). First, HeLa cells were transfected in a 12-well format (1.5×105 cells) with 200 ng pB-Tet-Cas13d and 800 ng of plasmid encoding codon-optimized piggyBac transposase (mPB) [Cadinanos 2007 17576687]. After 2 days incubation, transfection media was replaced with 10% FBS growth media containing 2 pg/mL puromycin (AG Scientific, #P-1033-SOL) to select for transposon integration. Cells were passaged to a 10-cm dish upon reaching confluency, and puromycin selection was maintained for 2 weeks. 1.5×105 cells were then passaged to a 12-well dish and transfected with 200 ng pB-mCherry and 800 ng mPB plasmid to integrate a constitutively expressing mCherry gene. Once confluent, cells were again passaged to a 10-cm dish. Two weeks after integration. Cas13d and EGFP expression was induced by incubation with 2 μM doxycycline (Sigma, #D3447-500MG) for 2 days, and EGFP- and mCherry-positive single cells were sorted into 96-well plates using a BD FACSAria II cell sorter at the University of Florida Interdisciplinary Center for Biotechnology Research (ICBR). Twelve EGFP- and mCherry-positive clones were manually identified using an EVOS FL microscope and expanded. The clone exhibiting the strongest expression of both EGFP and mCherry upon addition of doxycycline was chosen for the collateral activity RNA assay.

To perform the assay, HeLa-tet:Cas13d-mCherry and wild-type HeLa cells were mixed at a 1:4 ratio in growth media containing 2 μM doxycycline and plated in a 24-well plate format at a density of 7.5×104 cells/well. Cells were transfected with 125 ng gRNA plasmid and either 125 ng (CUG)480 plasmid (for (CUG)n-targeting validation experiment) or 125 ng pUC19 as inert carrier DNA (for endogenous gene targeting experiment). n=3 transfections per condition. After 44 hr, RNA was extracted with 300 uL TRIzol and purified using the Direct-zol RNA Miniprep Kit. cDNA was reverse-transcribed from 50 ng RNA using the iScript Reverse Transcription Supermix and diluted 10-fold in water. mCherry and control (GAPDH) expression levels were measured separately by qPCR from 4 uL of diluted cDNA using Taq DNA Polymerase (NEB, #M0270L), dsGreen DNA detection dye (Lumiprobe, #11010), and 200 nM each of forward and reverse primers (mCherry_F/mCherry_R and GAPDH_F/GAPDH_R, respectively). qPCR reactions were performed in duplicate on a C1000 Touch thermocycler (Bio-Rad) using a two-step cycling protocol: initial denaturation at 95° C. for 3 min, followed by 40 cycles of denaturation at 95° C. for 15 s and annealing and extension at 60° C. for 45 s. dsGreen fluorescence was measured after the extension step of each cycle. Cq values were calculated using Bio-Rad CFX Manager 3.1 software, and the difference between mCherry and GAPDH Cq (ΔCq) was calculated and averaged across the two qPCR replicates. The ratio of mCherry to GAPDH mRNA expression (defined as 2-ΔCq) was plotted for each condition. ΔCq values in targeting and non-targeting conditions were compared using the one-tailed Student's t test (independent samples, equal variance).

Modeling of negative autoregulation by gRNA excision. An ordinary differential equation (ODE) model was constructed that describes the dynamics of GENO autoregulation of Cas13d expression:

R≐r_t−k_proc RA-γ_R R (1)

A≐k_T R−k_proc RA-γ_A A (2)

B≐k_proc RA−γ_B B (3)

where R is the concentration of Cas13d mRNA (also containing the pre-crRNA), A is the concentration of Cas13d apoprotein, B is the concentration of Cas13d:gRNA binary complex, rt is the rate of RNA polymerase II transcription of the Cas13d mRNA, kT is the translation rate constant of Cas13d, kproc is the rate constant of crRNA processing, and γi is the degradation rate constant of species i. Transcription is modeled by zero-order kinetics, translation and degradation by first-order kinetics, and crRNA processing by second-order kinetics. Further details of the dynamical model, a reference model for unregulated Cas13d, and analytical proofs are presented in Supplemental Note.

To calculate equilibrium binary complex concentration across ranges of biochemical parameters, simulations were conducted by ODE integration using Python 3. RNA and protein degradation rates were estimated from median half-lives determined from the literature (10 hr [2003 Yang 12902380] and 36 hr [2011 Cambridge 22050367], respectively), and the degradation rates of Cas13d apoprotein and binary complex were estimated to be equivalent. In the unregulated reference model, the half-life of free gRNA was estimated as 2 hr. For each vector of parameters rt, kT, and kproc, the dynamical model was integrated over 480 hr with 5000 timesteps to reach steady state. Autoregulation efficiency ηGENO was calculated for each parameter vector as the difference of unity and the ratio of equilibrium binary complex concentrations between GENO and unregulated conditions (see Supplemental Note).

Western blot. Cas13d protein expression in unregulated and autoregulated conditions was measured by Western blot. HeLa cells were transfected in 12-well format with 500 ng Cas13d plasmid (pXR001 or pGENO-NT, +100 ng NT gRNA plasmid for unregulated Cas13d). n=3 transfections per condition. After 44 hr incubation, cells were washed with 1 mL PBS, and protein was extracted for 15 min on ice in 150 uL radioimmunoprecipitation assay (RIPA) buffer (Thermo Scientific, #89901) containing SIGMAFAST Protease Inhibitor Cocktail (Sigma-Aldrich, #S8830) and 1 mM phenylmethylsulfonyl fluoride (Sigma-Aldrich, #10837091001). Extractions were transferred to microcentrifuge tubes and centrifuged at 12,000 RCF for 15 min at 4° C. Total protein content in supernatant was measured using the Pierce BCA Protein Assay (Thermo Scientific, #23225). For each sample, 10 μg protein was mixed with 5.4 uL 4× NuPage LDS Sample Buffer (Invitrogen, #NP(008), 0.6 uL 100 mM dithiothreitol (NEB, #B1034A), and water to a total volume of 24 uL. Protein was denatured at 95° C. for 5 min prior to loading on a NuPage 4-12% Bis-Tris polyacrylamide gel (NP0336), with 3 uL Precision Plus Protein All Blue prestained standards (Bio-Rad, #1610373) loaded as a ladder. Gel electrophoresis was performed at 120 V for 45 min in MOPS running buffer (10.46 mg/mL 3-(N-morpholino)propanesulfonic acid (MOPS), 6.06 mg/mL Tris base, 1 mg/mL SDS, 0.3 mg/mL EDTA in water). Protein was transferred to a methanol-activated polyvinylidene fluoride (PVDF) membrane (Bio-Rad, #1620264) using the iBlot 2 dry transfer system (ThermoFisher Scientific, #IB21001).

The membrane was incubated in 5 mL SEA BLOCK Blocking Buffer (Thermo Scientific, #37527) for 30 min at RT in a dark container on a rocking mixer. Blocking buffer was removed and replaced with primary antibody solution (rabbit anti-HA (C29F4, 1:1000, Cell Signaling Technology) and mouse anti-HSP70 (5A5, 1:500. Invitrogen) in 5 mL SEA BLOCK+0.2% Tween 20 (Fisher Bioreagents, #BP152-1)), and the membrane was placed in a 4° C. room on a rocking mixer overnight. The next day, the primary antibody solution was removed, and the membrane was washed 3× in PBS-T (PBS+0.1% Tween 20). After washing, the secondary antibody solution was added (IRDye 800CW donkey anti-rabbit and 680RD donkey anti-mouse (1:5000 each, LI-COR) in 5 mL SEA BLOCK+0.2% Tween 20) and incubated for 1 hr at RT on a rocking mixer. The secondary antibodies were removed, and the plate was washed 3× in PBS-T. The blot was imaged on a LI-COR Odyssey CLx scanner at 700 and 800 nm excitation wavelengths. Image processing was performed in Fiji, and blot images were inverted for publication.

Mathematical Models

The following model is proposed to describe the kinetics and equilibria of a Cas13d negative autoregulation strategy mediated by gRNA excision (GENO):

R=transcription−processing−degradation

A=translation−processing−degradation

B=processing−degradation

This description relies on the following assumptions:
1. A deterministic model describes the mean dynamics of the underlying stochastic process with reasonable accuracy.
2. gRNA processing is performed by apoprotein only, upon Cas13d:gRNA binary complex formation.
3. Nascent Cas13d translation products quickly diffuse away from the domain of their mRNAs.
4. Cas13d:gRNA binary complex formation is irreversible.
5. Nuclear/cytoplasmic compartmentalization affects crRNA processing negligibly.
   These biochemical dynamics and assumptions produce the differential equation model (GENO) in FIGS. 13A-13B where:
R: concentration of Cas13d mRNA
A: concentration of Cas13d apoprotein
B: concentration of Cas13d:gRNA binary complex
rt: rate of Pol II transcription of Cas13d mRNA
kT: rate of translation of Cas13d protein
kp: rate of crRNA processing
γi: rate of degradation of species i
   A reference model (REF) is also presented for comparison, in which autoregulation is absent and gRNA processing and Cas13d expression are independent (FIGS. 13D-13G), where:
G: concentration of unbound gRNA
rG: rate of Pol III transcription of gRNA

### Equilibrium Analysis

### 2.1 GENO Model

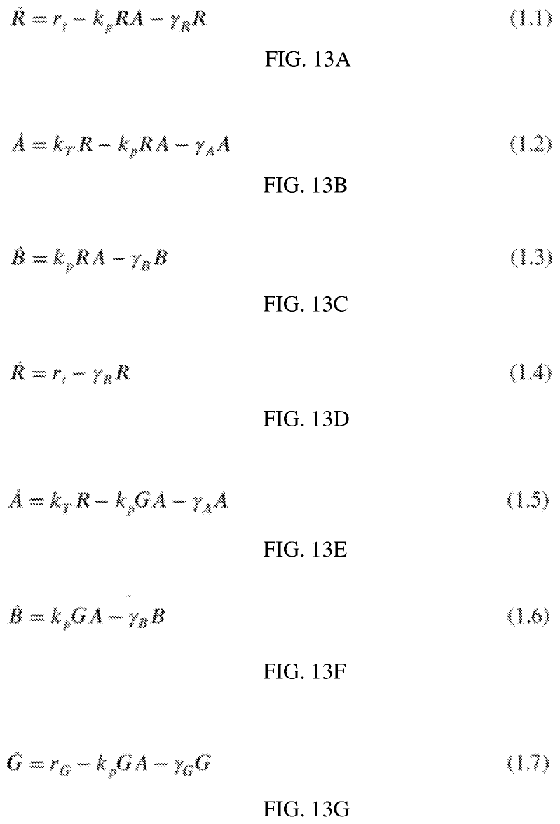

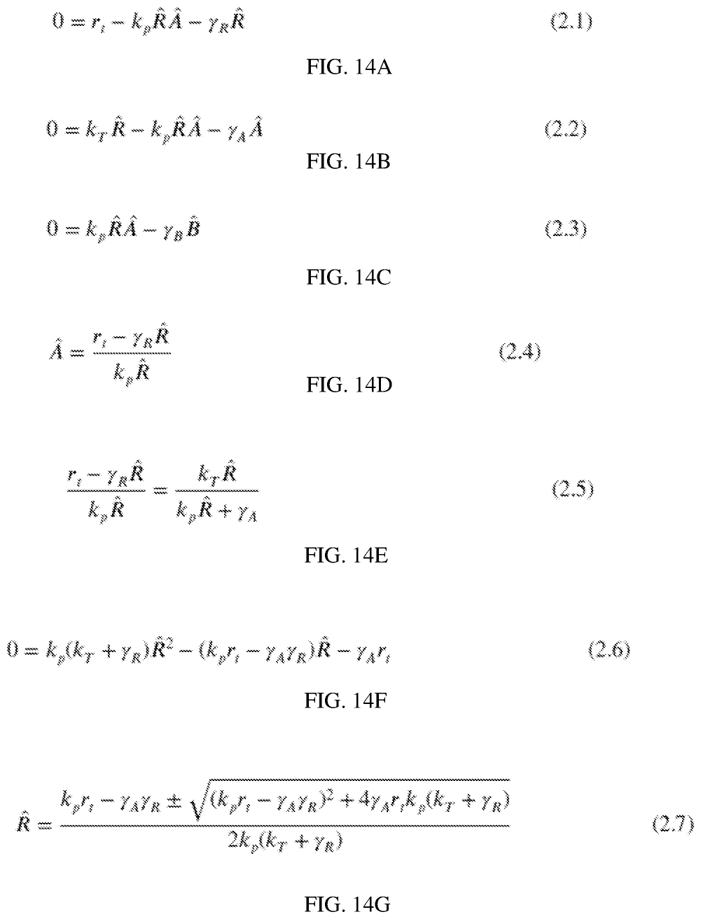

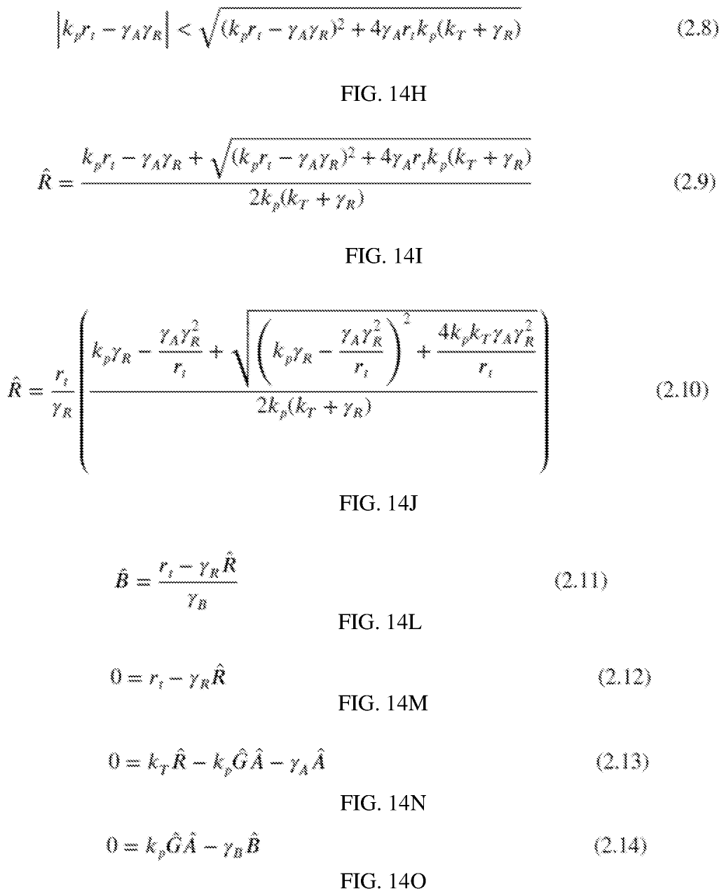

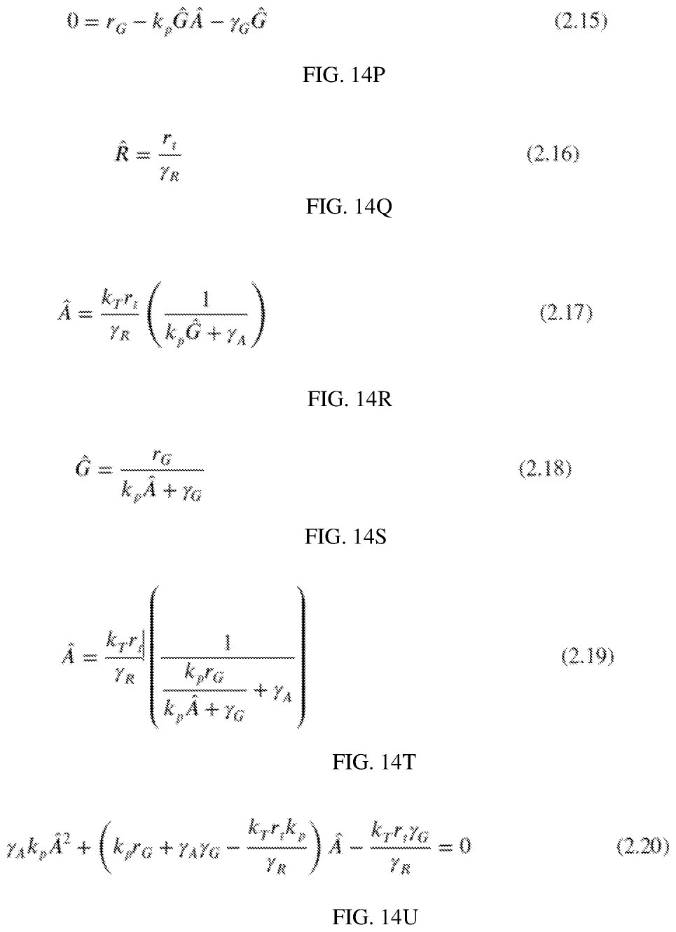

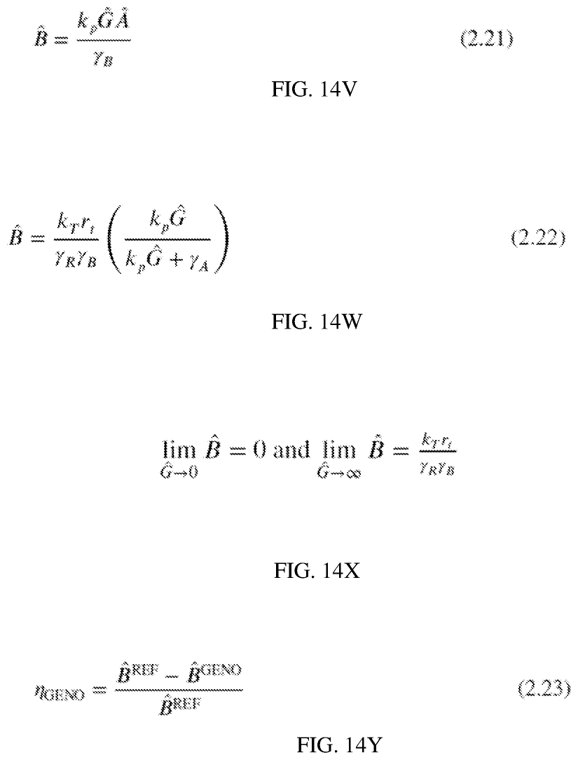

At equilibrium, the GENO model reduces as shown in FIGS. 14A-14C. From 2.1 (FIG. 4A) FIG. 14D. By substitution of 2.4 (FIG. 14D) into 2.2 (FIG. 14B), 3.5 (FIG. 14E), which reduces to the quadratic equation 2.6 (FIG. 14F). The solutions of this quadratic equation are 2.7 (FIG. 14G). By inspection 2.8 (FIG. 14H). Therefore, regardless of the sign of the term |kₚrₜ−γ_{A}γ_{R}|, the quadratic equation 2.6 has a single positive solution given by 2.9 (FIG. 14I). Equivalently, 2.10 (FIG. 14J). From 2.3 (FIG. 14C) and 2.4 (FIG. 14D), 2.11 (FIG. 14L). Thus, the system described by the GENO model has a single equilibrium point provided at \hat{R}^{GENO}, Â^{GENO}, \hat{B}^{GENO}, Ĝ^{GENO} the solutions provided in 2.10 (FIG. 14), 2.4 (FIG. 14B), and 2.11 (FIG. 14L).

### 2.2 REF Model

At equilibrium, the REF model reduces to FIG. 14M-14P. From 2.12 (FIG. 14M) FIG. 14Q. From (2.13) and (2.16) FIG. 14R. From (2.15) FIG. 14S. Substituting 2.18 into 2.17 yields FIG. 14T, which simplifies to the quadratic equation 2.20 (FIG. 14U). From 2.14, 2.21 (FIG. 14V). Substituting (2.17) into (2.21) yields 2.22 (FIG. 14W). Thus, the system described by the REF model has a single equilibrium point (\hat{R}^{REF}, Â^{REF}, \hat{B}^{REF}, Ĝ^{REF}) with the solution fully constrained by (2.16), (2.20), (2.22), and (2.18), respectively. Importantly, in the REF model, the concentration of binary complex CB takes the form of a Hill function where (FIG. 14X). The autoregulation efficiency n_{GENO} is defined as FIG. 14Y.

### 3 Proofs

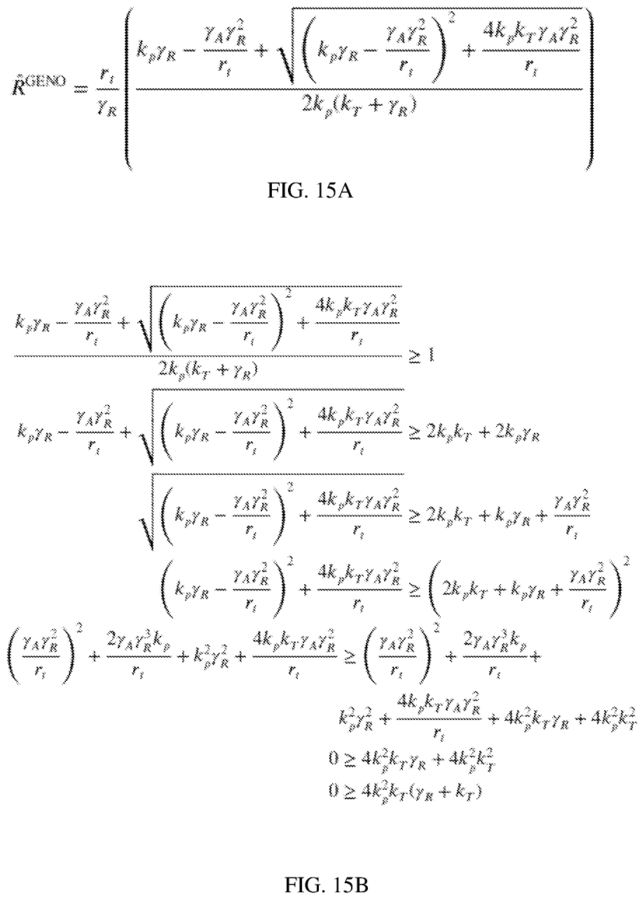

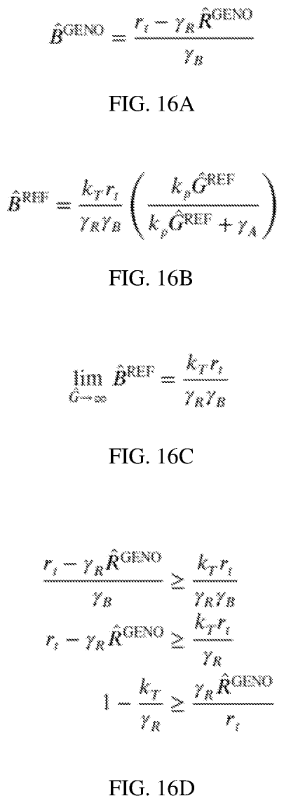

Theorem 1. At equilibrium, negative autoregulation by gRNA excision reduces the expression of the Cas13d mRNA compared to the reference model.
Proof. Proof by contradiction (FIGS. 15A-15C). The equilibrium mRNA concentration in the GENO model is provided in 2.10 (FIG. 14J) is in FIG. 15A. In the REF model, the equilibrium concentration is provided in 2.16 (FIG. 14Q). Assume \hat{R}^{GENO}≥\hat{R}^{REF}. Equivalently FIG. 15B. This inequality cannot be satisfied, as all biological parameters in the right-hand term are strictly positive. Therefore, \hat{R}^{GENO}≥\hat{R}^{REF}.
Theorem 2. Assume that gRNA is highly expressed in the reference model and is present in excess (Ĝ»γ_{A}/kₚ), and that Cas13d protein translation is faster than mRNA degradation (k_{T}>γₖ) At equilibrium, negative autoregulation by gRNA excision reduces the concentration of active Cas13d:gRNA binary complex compared to the reference model.
Proof. Proof by contradiction (FIGS. 16A-16D). The equilibrium binary complex concentration in the GENO model is provided in (2.11) (FIG. 14W, FIG. 16A). In the REF model, the equilibrium concentration is provided in (2.22) (FIG. 14W, FIG. 16B): If gRNA is expressed in excess in the REF model, the binary complex equilibrium concentration approaches its maximum value (FIG. 16C). Assume

$${\hat{R}}^{GENO} \geq {lim}_{G \to ∞} {\hat{B}}^{REF} .$$

### Equivalently FIG. 16D.

The right-hand term of this inequality is strictly positive. However, as k_{T}>γ_{R} the left-hand term is strictly negative. This inequality cannot be satisfied. Thus,

$${\hat{B}}^{GENO} \geq {lim}_{G \to ∞} {\hat{B}}^{REF}$$

under the following conditions:
1. gRNA expression in the reference system is high and in excess.
2. On average, more than one Cas13d protein molecule is translated from each Cas13d mRNA
in the reference system.

### Example 3: Negative Autoregulation Reduces AAV-Delivered Cas13d Expression in Human DM1 Myoblasts

As a proof-of-concept for applying negative autoregulation to an AAV-Cas13d therapy, it was investigated if GENO efficiently regulates expression when delivering Cas13d by AAV and if GENO-regulated Cas13d reduces CUGₙ RNA accumulation in patient-derived DM1 myoblasts. Recombinant AAV6 vectors carrying viral genomes encoding unregulated or GENO-regulated Cas13d driven by a CMV promoter with either CUG1 or NT gRNAs were synthesized. Despite multiple attempts, the unregulated CUG-targeting Cas13d design could not be packaged, possibly as a result of toxicity of overexpressed CUG-targeting Cas13d during virus production. As a result, the ability to compare the targeting and collateral activities of unregulated and GENO-regulated Cas13d in DM1 myoblasts was limited.

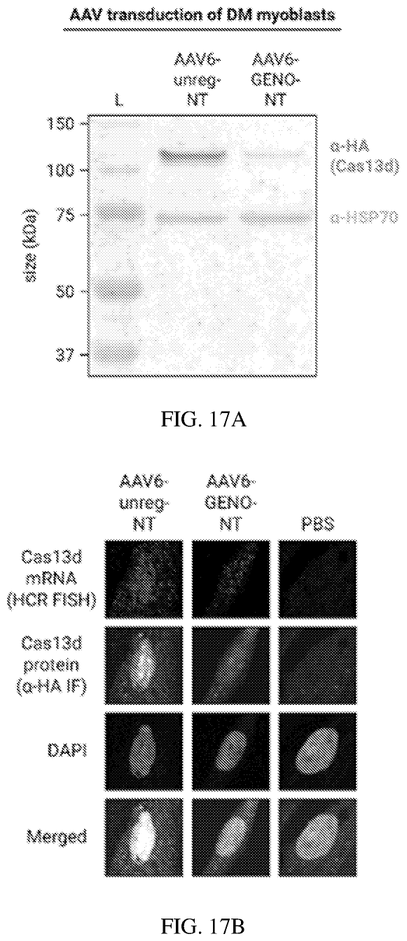

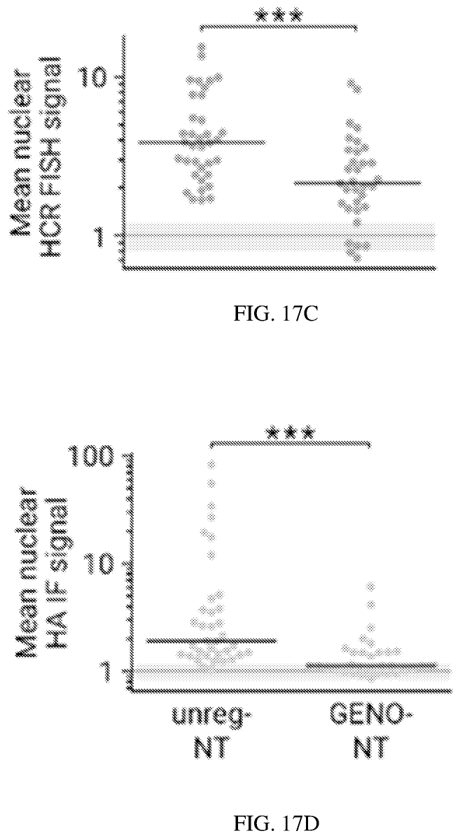

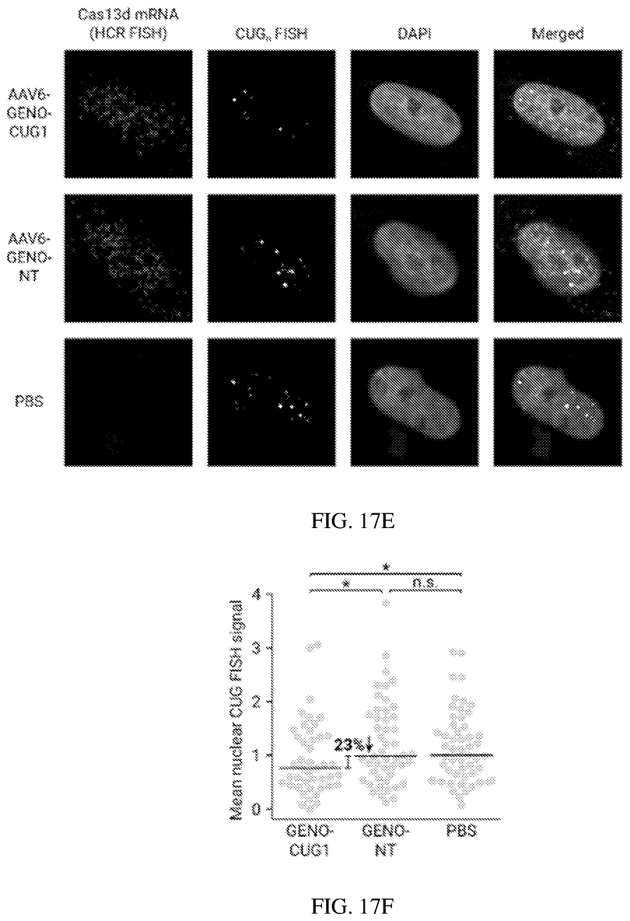

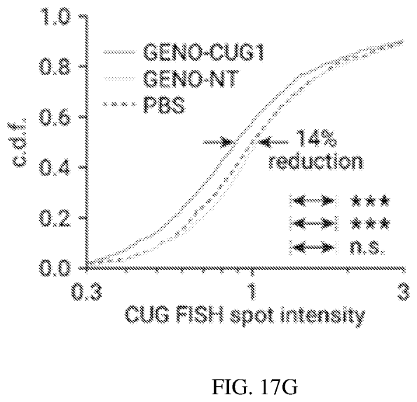

Undifferentiated DM1 myoblasts were treated with AAV for 6 days, and Western blot was performed to measure Cas13d protein expression (FIG. 17A). We found that GENO reduced Cas13d protein production by 87% (p<0.05, two-tailed Student's t test, n=3). To probe RNA and protein expression at the single-cell level, hybridization chain reaction FISH (HCR FISH) was performed [2018 Choi 29945988] to image single molecules of Cas13d mRNA and IF to measure Cas13d protein (FIG. 17B). and confocal microscopy images were quantified using automated image analysis techniques (see Methods). In images taken at 40× magnification, a mean of 144 diffraction-limited HCR FISH spots per nucleus in cells treated with AAV containing unregulated Cas13d and NT gRNA and 1.3 spots per nucleus in PBS-treated cells were detected, highlighting the sensitivity and specificity of this approach for measuring Cas13d RNA expression. Additionally, all nuclei in AAV treatment conditions (n=104) contained >5 HCR FISH spots, indicating nearly 100% efficiency of AAV transduction.

It observed that GENO reduced Cas13d mRNA and protein expression when treating DM1 myoblasts with non-targeting Cas13d AAV (FIG. 17B). By quantifying mean HCR FISH intensity in all imaged nuclei, it was found that GENO reduced median Cas13d mRNA expression by 60% after baseline subtraction of the median in the PBS-treated control (FIG. 17C, n>33 nuclei per condition, p<0.001, two-sided Mann-Whitney U test). Similarly, by performing the same analysis on α-HA IF, an 83% reduction in median Cas13d protein expression with GENO was found (FIG. 17D, n>33 nuclei per condition, p<0.001, two-sided Mann-Whitney U test). These results illustrate that reduction of Cas13d expression by negative autoregulation is efficient across cell types and transfection methods and is directly applicable to an AAV therapy.

Autoregulated Cas13d reduces CUGₙ RNA accumulation in human DM1 myoblasts

To evaluate if GENO-regulated Cas13d can reduce nuclear CUGₙ RNA foci in patient-derived cells, DM1 myoblasts were transduced with AAV for 6 days, and FISH was simultaneously performed with a fluorescent CAG₁₀ probe to detect CUGₙ RNAs from the expanded DMPK allele and HCR FISH for Cas13d mRNA to mark transduced nuclei (FIG. 17E). Confocal images were collected at 40× magnification to visualize discrete diffraction-limited spots in both channels, and FISH spots and quantified fluorescence intensities were detected using automated image analysis (see Methods).

By quantifying mean CUG FISH intensity across many nuclei, a modest 23% reduction in median CUGₙ RNA expression was found when treating myoblasts with AAV containing GENO-regulated Cas13d and CUG-1 gRNA compared with NT gRNA and PBS-treated controls (FIG. 17F, n>43 nuclei, p<0.05, one-sided Mann-Whitney U test). A difference in nuclear FISH signal between non-targeting Cas13d and PBS-treated conditions was not observed (p>0.05, n>43 nuclei, two-sided Mann-Whitney U test). Interestingly, a similar reduction in Cas13d HCR FISH intensity between targeting and non-targeting conditions was not observed, which suggests that the extent of collateral activity of GENO-regulated Cas13d in DM1 myoblasts may be low enough to preserve RNA homeostasis.

A significant difference in the number of CUGₙ RNA foci per nucleus between any conditions (p>0.05, two-sided Mann-Whitney U test) was not observed. To investigate if the difference in mean nuclear FISH intensity reflects a reduction of RNA accumulation caused by Cas13d targeting activity, the intensities of all individual diffraction-limited FISH spots detected in nuclei from each condition were measured (FIG. 170). The median intensity of CUGₙ RNA foci was reduced by 14% in the targeting condition compared to the non-targeting control (n>745 spots per condition, p<0.001, two-sided Mann-Whitney U test). A difference in foci intensity between non-targeting Cas13d and PBS-treated conditions was not observed (p>0.05, two-sided Mann-Whitney U test). These results suggest that GENO-regulated Cas13d reduces accumulation of expanded CUGₙ RNAs in DM1 patient-derived cells by cleaving and dispersing multimeric CUGₙ RNA structures and initiating RNA decay.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of.” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example. “at least one of A and B” (or, equivalently, “at least one of A or B,” or, equivalently “at least one of A and/or B”) can refer, in one embodiment, to at least one, optionally including more than one, A, with no B present (and optionally including elements other than B): in another embodiment, to at least one, optionally including more than one, B, with no A present (and optionally including elements other than A); in yet another embodiment, to at least one, optionally including more than one, A, and at least one, optionally including more than one, B (and optionally including other elements): etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B”, the disclosure also contemplates the alternative embodiments “a composition consisting of A and B” and “a composition consisting essentially of A and B”.

We claim:

1. A nucleic acid molecule comprising a zinc finger binding site, and a nucleotide sequence encoding a Cas13d fusion protein, wherein the Cas13d fusion protein comprises a Cas13d domain, a zinc finger (ZnF) domain, and a transcriptional repressor domain, and wherein the Cas13d fusion protein is capable of binding to the zinc finger binding site, thereby repressing transcription of the nucleotide sequence encoding the Cas13d fusion protein, optionally wherein the zinc finger binding site is 5′ to the Cas13d fusion protein.

2. The nucleic acid molecule of claim 1, wherein the zinc finger binding site comprises a nucleotide sequence of any one of SEQ ID NOs: 9, 11, 13, 15, 17, 19, or 21.

3. The nucleic acid molecule of claim 1 further comprising a promoter.

4. The nucleic acid molecule of claim 1, wherein the promoter is an inducible promoter

5. The nucleic acid molecule of claim 1, wherein the promoter is a constitutive promoter.

6. The nucleic acid molecule of claim 1, wherein the promoter is a synthetic promoter.

7. The nucleic acid molecule of claim 1, wherein the promoter is a eukaryotic promoter.

8. The nucleic acid molecule of claim 6, wherein the eukaryotic promoter is a CMV promoter, a EF1a promoter, a CAG promoter, a PGK promoter, a TRE promoter, a U6 promoter, or a UAS promoter.

9. The nucleic acid molecule of claim 1, wherein the promoter is a bacterial promoter.

10. The nucleic acid molecule of claim 9, wherein the bacterial promoter is a T7 promoter, a T71ac promoter, a Sp6 promoter, a lac promoter, an araBad promoter, a trp promoter, a Ptac promoter, a P1 promoter, a T3 promoter, or a Ptet promoter.

11. The nucleic acid molecule of any one of claims 1-9, wherein the Cas13d domain comprises an amino acid sequence of any one of SEQ ID NOs: 1-7, or an amino acid sequence having at least 90% sequence identity with any of SEQ ID NOs: 1-7.

12. The nucleic acid molecule of any one of claims 1-10, wherein the zinc finger (ZnF) domain comprises an amino acid sequence of any one of SEQ ID NOs: 8, 10, 12, 14, 16, 18, or 20, or an amino acid sequence having at least 90% sequence identity with any of SEQ ID NOs: 8, 10, 12, 14, 16, 18, or 20.

13. The nucleic acid molecule of any one of claims 1-12, wherein the transcriptional repressor domain comprises an amino acid sequence of any one of SEQ ID NOs: 22-24, or an amino acid sequence having at least 90% sequence identity with any of SEQ ID NOs: 22-24.

14. The nucleic acid molecule of any one of claims 1-13, wherein the transcriptional repressor domain is a KRAB domain comprising an amino acid sequence of SEQ ID NOs: 24, or an amino acid sequence having at least 90% sequence identity with SEQ ID NOs: 24.

15. The nucleic acid molecule of any one of claims 1-14, wherein the Cas13d fusion protein comprises one or more linkers.

16. The nucleic acid molecule of claim 15, wherein the one or more linkers have an amino acid sequence selected from the group consisting of any of SEQ ID NOs: 25-35, or an amino acid sequence having at least 90% sequence identity with any one of SEQ ID NOs: 25-35.

17. The nucleic acid molecule of claim 15 or 16, wherein the Cas13d fusion protein comprises a linker joining the Cas13d domain and the zinc finger (ZnF) domain.

18. The nucleic acid molecule of any one of claims 15-17, wherein the Cas13d fusion protein comprises a linker joining the zinc finger (ZnF) domain and the transcriptional repressor domain.

19. The nucleic acid molecule of any one of claims 1-18, further comprising a first nuclear localization signal (NLS) sequence.

20. The nucleic acid molecule of claim 19, further comprising a second nuclear localization signal (NLS) sequence.

21. The nucleic acid molecule of claim 19 or 20, wherein the first NLS is fused to the N-terminus of the Cas13d domain.

22. The nucleic acid molecule of claim 20 or 21, wherein the second NLS is fused to the C-terminus of the Cas13d domain.

23. The nucleic acid molecule of any one of claims 1-22, further comprising a peptide tag.

24. The nucleic acid molecule of claim 23, wherein the peptide tag is selecting from the group consisting of a His-tag (SEQ ID NO: 51), HA-tag (SEQ ID NO: 49), Flag-tag (SEQ ID NO: 50), a Myc tag (SEQ ID NO: 52), a V5 tag (SEQ ID NO: 53), or an AviTag-PT-6 (SEQ ID NO: 54).

25. The nucleic acid molecule of claim 1, comprising SEQ ID NO: 55.

26. A nucleic acid plasmid comprising the nucleic acid molecule of any of claims 1-25.

27. A viral vector comprising the nucleic acid molecule of any of claims 1-25.

28. The viral vector of claim 27, wherein the viral vector is an adenovirus vector, an adeno-associated virus vector, or a lentivirus vector.

29. The viral vector of claim 27, wherein the adeno-associated virus vector is an AAV1, AAV2, AAV3, AAV4, AAV5, AAV6, AAV7, AAV8, AAV9 . . . or chimera thereof (define chimera as have a genome from at least two different serotypes)

30. The nucleic acid plasmid of claim 25 or the viral vector of any of claims 27-29, further comprising a nucleotide sequence encoding one or more guide RNAs which are capable of targeting the Cas13d domain to a transcript target.

31. The plasmid or vector of claim 30, wherein the transcript target comprises CUG repeat expansions (CUGexp).

32. The plasmid or vector of claim 31, wherein the transcript target encodes a myotonic dystrophy protein kinase comprising CUGexp.

33. The plasmid or vector of claim 30, wherein one or more of the one or more guide RNAs comprise a nucleotide sequence complementary to any one of SEQ ID NO:58-77.

34. A composition comprising one or more of the nucleic acid molecules of any one of claims 1-25, the nucleic acid plasmid of any one of claim 26 or 30-33, or the viral vector of any one of claims 29-33, and a pharmaceutically acceptable excipient.

35. A composition comprising one or more nucleic acid molecule of any one of claims 1-25, the nucleic acid plasmid of any one of claim 26 or 30-33, or the viral vector of any one of claims 27-33, and optionally a nucleic acid molecule encoding one or more guide RNAs which are capable of targeting the Cas13d domain to a transcript target, and a pharmaceutically acceptable excipient.

36. A cell comprising any one of the nucleic acid molecules of any of claims 1-24, the nucleic acid plasmid of any one of claim 26 or 30-33, the viral vector of any one of claims 27-33, and the compositions of claim 34 or 35.

37. A kit comprising one or more of the nucleic acid molecules of any one of claims 1-24, the nucleic acid plasmid of any one of claim 26 or 30-33, the viral vector of any one of claims 27-33, and the compositions of claim 34 or 35.

38. A method of transcript target knockdown using an autoregulated Cas13d domain, comprising expression of any one of the nucleic acid molecules of any one of claims 1-24, the nucleic acid plasmid of any one of claim 26 or 30-33, the viral vector of any one of claims 27-33, and the compositions of claim 34 or 35.

39. A method of transcript target knockdown using an autoregulated Cas13d domain, comprising expression of a nucleic acid molecule comprising in the 5′ to 3′ direction a zinc finger binding site, a promoter, and a nucleotide sequence encoding a Cas13d fusion protein, wherein the Cas13d fusion protein comprises a Cas13d domain, a zinc finger (ZnF) domain, and a transcriptional repressor domain, and wherein the Cas13d fusion protein binds to the zinc finger binding site and represses transcription of the nucleotide sequence encoding the Cas13d fusion protein.

40. The method of claim 38 or 39, wherein autoregulation of the Cas13d domain reduces collateral damage.

41. The method of any one of claims 38-40, wherein the transcript target is knocked down by a greater amount than can be achieved using RNA interference or CRISPR (Cas9) interference.

42. The method of any one of claims 38-41, wherein the means for autoregulation of the Cas13d domain comprises binding of the Cas13d fusion protein ZnF domain to the ZnF sequence motif, thereby causing the transcriptional repressor domain to inhibit transcription of the sequence encoding the Cas13d fusion protein.

43. A nucleic acid molecule comprising a nucleotide sequence encoding a Cas13d domain and a Cas13d processing sequence on a single Cas13d autoregulatory transcript.

44. The nucleic acid molecule of claim 1, wherein the Cas13d processing sequence comprises a direct repeat.

45. The nucleic acid molecule of claim 1, wherein the Cas13d binding region comprises a pre-guide RNA.

46. The nucleic acid molecule of claim 43, wherein the Cas13d domain and the Cas13d processing sequence are at least 20 base pairs apart.

47. The nucleic acid molecule of claim 43 or 44, wherein the Cas13d processing sequence is 5′ of the Cas13d domain.

48. The nucleic acid molecule of claim 43 or 44, wherein the Cas13d processing sequence is 3′ of the Cas13d domain.

49. The nucleic acid molecule of claim 43, wherein the Cas13d processing sequence is inserted into an intron of the Cas13d domain.

50. The nucleic acid molecule of claim 43, wherein the Cas13d processing sequence is inserted into the Cas13d domain.

51. The nucleic acid molecule of claim 47, wherein the Cas13d processing sequence is inserted into the Cas13d domain at position 143 of SEQ ID NO 57.

52. The nucleic acid molecule of claim 43, wherein the Cas13d processing sequence of the nucleic acid encoding the Cas13d autoregulatory transcript Cas13d autoregulation transcript is located within the Cas13d domain 5′ untranslated region or the Cas13d domain 3′ untranslated region.

53. The nucleic acid molecule of any one of claims 43-49, further comprising a promoter.

54. The nucleic acid molecule of claim 53, wherein the promoter is an inducible promoter.

55. The nucleic acid molecule of claim 53, wherein the promoter is a constitutive promoter.

56. The nucleic acid molecule of claim 53, wherein the promoter is a synthetic promoter.

57. The nucleic acid molecule of claim 53, wherein the promoter is a eukaryotic promoter.

58. The nucleic acid molecule of claim 52, wherein the eukaryotic promoter is a CMV promoter, a EF1α promoter, a CAG promoter, a PGK promoter, a TRE promoter, a U6 promoter, or a UAS promoter.

59. The nucleic acid molecule of claim 49, wherein the promoter is a bacterial promoter.

60. The nucleic acid molecule of claim 54, wherein the bacterial promoter is a T7 promoter, a T71ac promoter, a Sp6 promoter, a lac promoter, an araBad promoter, a trp promoter, a Ptac promoter, a P1 promoter, a T3 promoter, or a Ptet promoter.

61. The nucleic acid molecule of any one of claims 42-55, wherein the Cas13d domain comprises an amino acid sequence of any one of SEQ ID NOs: 1-7, or an amino acid sequence having at least 90% sequence identity with any of SEQ ID NOs: 1-7.

62. The nucleic acid molecule of any one of claims 42-56, further comprising a first nuclear localization signal (NLS) sequence.

63. The nucleic acid molecule of claim 57 further comprising a second nuclear localization signal (NLS) sequence.

64. The nucleic acid molecule of claim 57 or 58, wherein the first NLS is fused to the n-terminus of the cas13d domain.

65. The nucleic acid molecule of claim 58 or 59, wherein the second NLS is fused to the c-terminus of the cas13d domain.

66. The nucleic acid molecule of any one of claim 42-60, further comprising a peptide tag.

67. The nucleic acid molecule of claim 61, wherein the peptide tag is selecting from the group consisting of is selecting from the group consisting of a His-tag (SEQ ID NO: 51), HA-tag (SEQ ID NO: 49), Flag-tag (SEQ ID NO: 50), a Myc tag (SEQ ID NO: 52), a V5 tag (SEQ ID NO: 53), or an AviTag-PT-6 (SEQ ID NO: 54).

68. The nucleic acid molecule of claim 42, comprising SEQ ID NO: 2.

69. The nucleic acid molecule of any one of claims 44-63, further comprising a nucleotide sequence encoding one or more additional pre-guide RNAs, wherein the pre-guide RNAs encoded in the nucleic acid molecule are capable of targeting the Cas13d domain to a transcript target.

70. A nucleic acid plasmid comprising the nucleic acid molecule of any one of claims 42-64.

71. A viral vector comprising the nucleic acid molecule of any one of claims 42-64.

72. The viral vector of claim 71, wherein the viral vector is an adenovirus vector, an adeno-associated virus vector, or a lentivirus vector.

73. The viral vector of claim 72, wherein the adeno-associated virus vector is an AAV1, AAV2, AAV3, AAV4, AAV5, AAV6, AAV7, AAV8, AAV9 . . . or chimera thereof (define chimera as have a genome from at least two different serotypes)

74. The nucleic acid molecule of claim 64, the plasmid of claim 70, or the vector of any one of claims 71-73, wherein the transcript target comprises CUG repeat expansions (CUGexp).

75. The nucleic acid molecule of claim 64, the plasmid of claim 66, or the vector of any one of claims 67-69, wherein the transcript target encodes myotonic dystrophy protein kinase and comprises CUG repeat expansions (CUGexp).

76. The nucleic acid molecule of claim 64, the plasmid of claim 66, or the vector of any one of claims 67-69, wherein at least one of the one or more guide RNAs comprise a nucleotide sequence capable of targeting of any one of SEQ ID NO: 59-78.

77. The nucleic acid molecule of claim 65, the plasmid of claim 66, or the vector of any one of claims 67-69, wherein all the guide RNAs are pre-guide RNAs, each pre-guide RNA comprises a spacer flanked by direct repeat regions.

78. A composition comprising one or more of the nucleic acid molecules of any one of claims 43-65, the nucleic acid plasmid of any one of claim 66 or 64-67, or the viral vector of any one of claims 61-67, and a pharmaceutically acceptable excipient.

79. A composition comprising one or more of the nucleic acid molecules of any one of claims 42-59 or 65-68, the nucleic acid plasmid of any one of claim 61 or 70-73, or the viral vector of any one of claims 67-73, and optionally a nucleic acid molecule encoding one or more guide RNAs which are capable of targeting the Cas13d domain to a transcript target, and a pharmaceutically acceptable excipient.

80. A cell comprising any one of the nucleic acid molecules of any one of claims 42-59 or 65-68, the nucleic acid plasmid of any one of claim 61 or 70-73, or the viral vector of any one of claims 67-73, and the compositions of any one of claim 74 or 75.

81. A kit comprising one or more of the nucleic acid molecules of any of claims 42-59 or 65-68, the nucleic acid plasmid of any one of claim 61 or 70-73, or the viral vector of any one of claims 67-73, and the compositions of any one of claim 74 or 75.

82. A method of target transcript knockdown using an autoregulated Cas13d domain, comprising expression of any one of the nucleic acid molecule of any one of claims 42-59 or 65-68, the nucleic acid plasmid of any one of claim 61 or 70-73, or the viral vector of any one of claims 67-73, and the compositions of any one of claim 74 or 75.

83. A method of target transcript knockdown using an autoregulated Cas13d domain, comprising expression of a nucleic acid molecule comprising in the 5′ to 3′ direction a nucleotide sequence encoding a Cas13d domain and Cas13d processing sequence on a single Cas13d autoregulatory transcriptCas13d autoregulation transcript.

84. The method of claim 78 or 79, wherein autoregulation of the Cas13d domain reduces collateral damage.

85. The method of any one of claims 78-80, wherein the target transcript is knocked down by a greater amount than can be achieved using RNA interference or CRISPR (Cas9) interference.

86. The method of any one of claims 78-81, wherein the means for autoregulation of Cas13d expression comprises RNA exonuclease degradation of the Cas13d autoregulatory transcriptCas13d autoregulation transcript following Cas13d protein-dependent cleavage of the sequence encoding the pre-guide RNA.

87. A method of administering a therapeutically effective amount of the compositions of any one of claims 34, 35, 74, or 75 to a subject in need thereof.

88. The method of claim 83, wherein the subject has a disease associated with transcriptional dysregulation.

89. The method of claim 83, wherein the disease is associated with mRNA aggregation.

90. The method of any one of claims 84-85, wherein the disease is Spinocerebellar ataxia type 12, Fragile X-associated tremor/ataxia syndrome, Neuronal intranuclear inclusion disease, C9ORF72 amyotrophic lateral sclerosis/frontotemporal dementia, Benign adult familial myoclonic epilepsy (familial adult myoclonic epilepsy 1), Cerebellar ataxia, neuropathy, vestibular areflexia syndrome, Myotonic dystrophy type 2, Fuchs endothelial corneal dystrophy, Spinocerebellar ataxia type 10, Spinocerebellar ataxia type 31, Spinocerebellar ataxia type 36 (Asidan, Costa da Morte ataxia), Spinocerebellar ataxia type 37, Dentatorubral-pallidoluysian atrophy (Haw River syndrome, Naito-Oyanagi disease), Huntington's disease, Spinal-bulbar muscular atrophy, Spinocerebellar ataxia type 1, Spinocerebellar ataxia type 2, Spinocerebellar ataxia type 3 (Machado-Joseph disease), Spinocerebellar ataxia type 6, Spinocerebellar ataxia type 7, Spinocerebellar ataxia type 8, Spinocerebellar ataxia type 17, Myotonic dystrophy type 1, Huntington's disease-like 2, Blepharophimosis syndrome, Cleidocranial dysplasia, Congenital central hypoventilation syndrome, Hand-foot-genital syndrome, Holoprosencephaly, Oculopharyngeal muscular dystrophy, Synpolydactyly syndrome, X-linked mental retardation and abnormal genitalia, X-linked mental retardation, X-linked mental retardation and growth hormone deficit, Pseudoachondroplasia and multiple epiphyseal dysplasia, Familial adult myoclonic epilepsy 2, Familial adult myoclonic epilepsy 3, Familial adult myoclonic epilepsy 4, Familial adult myoclonic epilepsy 6, Familial adult myoclonic epilepsy 7, Oculopharyngeal myopathy with leukoencephalopathy, Oculopharyngodistal myopathy 1, or Oculopharyngodistal myopathy 2.

91. The method of any one of claims 84-85, wherein the disease is muscular dystrophy.

92. The viral vector of any one of claims 27-29 or 67-69 comprising a nucleic acid sequence of any one of SEQ ID NOs: 178-179.


SEQUENCE LISTING (186 sequences)

SEQ ID NO: 1 - protein, 955 aa, Unknown
  Anaerobic digester metagenome 15706
MNNKRKTKAKAAGLKSVFFDQKQAVLTTFAKGNNSQIEKKVVNSEVKDLRQPPAFDLELKEKTFYISGKNNINTSRENPLASASLPLSKRQRIRAERIKRAREENRPYHNVKRVGEDDLRAKADLEKHYFGKEYSDNLKIQIIYNILDINKIISPYINDIVYSMNNLARNDEYIDGKIDVIGSLSSTTDYSSFMSPNKDLEKEKKFSFHRENYKKFVEASKPYMRYYGKVFIRDVKKSKLSTGKGEKIEVMYRSDEEIFTIFQILSYVRQSIMHNDIGNKSSILAIEKYPARFVGFLSDLLKTKTNDVNRMFIDNNSQTNFWVLFSIFGLQDHTSGADKICRNFYDFVIKADSKNLGFSLKKIRELMLDLPNANMLRDHQFDTVRSKFYTLLDFIIYQHYLEEKSRIDNMVEKLRMTLKEEEKEVLYAAEAKIVWNAIGAKVINKLVPMMNGDALKEIKRKNRDRKLPQSVIATVQVNSDANVFSGLIYFLTLFLDGKEINEMVSNLITKFENIDSLLHVDREIYKSDEKDLDLEIEKLALFFKGVVRPNAKTDTGAGEISKSFSIFQSAERIIEELKFIKNVTRMDNEIFPSEGVFLDAANVLGVRGDDFDFSNEFVGDDLHSDANKKIINKINGTKEDRNLRNFIINNVVKSRRFQYIARHMNTHYVKQLANNETLNRFVLNKMGDAKIINRYYESISGNTPNIEVRSQIDYLVKRLRSFSFEDLNDVKQKVRPGTNESIEKEKKKALVGLCLTIQYLVYKNLVNINARYTTAFYCLERDSKLKGFGVDVWRDFESYTALTNHFIKEGYLPVRKAEILRANLKHLDCEDGFKYYRNQVTHLNAIRVAYKYINEIKSVHSYFALYHYIMQRHLYDSLQAKAKDSSGFVIDALKKSFEHKIYSKDLLHVLHSPFGYNTARYKNLSIEALFDKNESRPEVNPLSTNDYPYDVPDYA

SEQ ID NO: 2 - protein, 963 aa, Eubacterium siraeum
MGKKIHARDLREQRKTDRTEKFADQNKKREAERAVPKKDAAVSVKSVSSVSSKKDNVTKSMAKAAGVKSVFAVGNTVYMTSFGRGNDAVLEQKIVDTSHEPLNIDDPAYQLNVVTMNGYSVTGHRGETVSAVTDNPLRRFNGRKKDEPEQSVPTDMLCLKPTLEKKFFGKEFDDNIHIQLIYNILDIEKILAVYSTNAIYALNNMSADENIENSDFFMKRTTDETFDDFEKKKESTNSREKADFDAFEKFIGNYRLAYFADAFYVNKKNPKGKAKNVLREDKELYSVLTLIGKLRHWCVHSEEGRAEFWLYKLDELKDDFKNVLDVVYNRPVEEINNRFIENNKVNIQILGSVYKNTDIAELVRSYYEFLITKKYKNMGFSIKKLRESMLEGKGYADKEYDSVRNKLYQMTDFILYTGYINEDSDRADDLVNTLRSSLKEDDKTTVYCKEADYLWKKYRESIREVADALDGDNIKKLSKSNIEIQEDKLRKCFISYADSVSEFTKLIYLLTRFLSGKEINDLVTTLINKFDNIRSFLEIMDELGLDRTFTAEYSFFEGSTKYLAELVELNSFVKSCSFDINAKRTMYRDALDILGIESDKTEEDIEKMIDNILQIDANGDKKLKKNNGLRNFIASNVIDSNRFKYLVRYGNPKKIRETAKCKPAVRFVLNEIPDAQIERYYEACCPKNTALCSANKRREKLADMIAEIKFENFSDAGNYQKANVTSRTSEAEIKRKNQAIIRLYLTVMYIMLKNLVNVNARYVIAFHCVERDTKLYAESGLEVGNIEKNKTNLTMAVMGVKLENGIIKTEFDKSFAENAANRYLRNARWYKLILDNLKKSERAVVNEFRNTVCHLNAIRNININIKEIKEVENYFALYHYLIQKHLENRFADKKVERDTGDFISKLEEHKTYCKDFVKAYCTPFGYNLVRYKNLTIDGLFDKNYPGKDDSDEQKYPYDVPDYA

SEQ ID NO: 3 - protein, 915 aa, Unknown
  Gut metagenome assembly P1E0-k21
MEREVKKPPKKSLAKAAGLKSTFVISPQEKELAMTAFGRGNDALLQKRIVDGVVRDVAGEKQQFQVQRQDESRFRLQNSRLADRTVTADDPLHRAETPRRQPLGAGMDQLRRKAILEQKYFGRTFDDNIHIQLIYNILDIHKMLAVPANHIVHTLNLLGGYGETDFVGMLPAGLPYDKLRVVKKKNGDTVDIKADIAAYAKRPQLAYLGAAFYDVTPGKSKRDAARGRVKREQDVYAILSLMSLLRQFCAHDSVRIWGQNTTAALYHLQALPQDMKDLLDDGWRRALGGVNDHFLDTNKVNLLTLFEYYGAETKQARVALTQDFYRFVVLKEQKNMGFSLRRLREELLKLPDAAYLTGQEYDSVRQKLYMLLDFLLCRLYAQERADRCEELVSALRCALSDEEKDTVYQAEAAALWQALGDTLRRKLLPLLKGKKLQDKDKKKSDELGLSRDVLDGVLFRPAQQGSRANADYFCRLMHLSTWFMDGKEINTLLTTLISKLENIDSLRSVLESMGLAYSFVPAYAMFDHSRYIAGQLRVVNNIARMRKPAIGAKREMYRAAVVLLGVDSPEAAAAITDDLLQIDPETGKVRPRSDSARDTGLRNFIANNVVESRRFTYLLRYMTPEQARVLAQNEKLIAFVLSTVPDTQLERYCRTCGREDITGRPAQIRYLTAQIMGVRYESFTDVEQRGRGDNPKKERYKALIGLYLTVLYLAVKNMVNCNARYVIAFYCRDRDTALYQKEVCWYDLEEDKKSGKQRQVEDYTALTRYFVSQGYLNRHACGYLRSNMNGISNSLLTAYRNAVDHLNAIPPLGSLCRDIGRVDSYFALYHYAVQQYLNGRYYRKTPREQELFAAMAQHRTWCSDLVKALNTPFGYNLARYKNLSIDGLFDREGDHVVREDGEKPAEYPYDVPDYA

SEQ ID NO: 4 - protein, 953 aa, Ruminococcus albus
MAKKSKGMSLREKRELEKQKRIQKAAVNSVNDTPEKTEEANVVSVNVRTSAENKHSKKSAAKALGLKSGLVIGDELYLTSFGRGNEAKLEKKISGDTVEKLGIGAFEVAERDESTLTLESGRIKDKTARPKDPRHITVDTQGKFKEDMLGIRSVLEKKIFGKTFDDNIHVQLAYNILDVEKIMAQYVSDIVYMLHNTDKTERNDNLMGYMSIRNTYKTFCDTSNLPDDTKQKVENQKREFDKIIKSGRLGYFGEAFMVNSGNSTKLRPEKEIYHIFALMASLRQSYFHGYVKDTDYQGTTWAYTLEDKLKGPSHEFRETIDKIFDEGFSKISKDFGKMNKVNLQILEQMIGELYGSIERQNLTCDYYDFIQLKKHKYLGFSIKRLRETMLETTPAECYKAECYNSERQKLYKLIDFLIYDLYYNRKPARIEEIVDKLRESVNDEEKESIYSVEAKYVYESLSKVLDKSLKNSVSGETIKDLQKRYDDETANRIWDISQHSISGNVNCFCKLIYIMTLMLDGKEINDLLTTLVNKFDNIASFIDVMDELGLEHSFTDNYKMFADSKAICLDLQFINSFARMSKIDDEKSKRQLFRDALVILDIGNKDETWINNYLDSDIFKLDKEGNKLKGARHDFRNFIANNVIKSSRFKYLVKYSSADGMIKLKTNEKLIGFVLDKLPETQIDRYYESCGLDNAVVDKKVRIEKLSGLIRDMKFDDFSGVKTSNKAGDNDKQDKAKYQAIISLYLMVLYQIVKNMIYVNSRYVIAFHCLERDFGMYGKDFGKYYQGCRKLTDHFIEEKYMKEGKLGCNKKVGRYLKNNISCCTDGLINTYRNQVDHFAVVRKIGNYAAYIKSIGSWFELYHYVIQRIVFDEYRFALNNTESNYKNSIIKHHTYCKDMVKALNTPFGYDLPRYKNLSIGDLFDRNNYLNKTKESIDANSSIDSQYPYDVPDYA

SEQ ID NO: 5 - protein, 942 aa, Ruminococcus flavefaciens
MKKKMSLREKREAEKQAKKAAYSAASKNTDSKPAEKKAETPKPAEIISDNSRNKTAVKAAGLKSTIISGDKLYMTSFGKGNAAVIEQKIDINDYSFSAMKDTPSLEVDKAESKEISFSSHHPFVKNDKLTTYNPLYGGKDNPEKPVGRDMLGLKDKLEERYFGCTFNDNLHIQIIYNILDIEKILAVHSANITTALDHMVDEDDEKYLNSDYIGYMNTINTYDVFMDPSKNSSLSPKDRKNIDNSRAKFEKLLSTKRLGYFGFDYDANGKDKKKNEEIKKRLYHLTAFAGQLRQWSFHSAGNYPRTWLYKLDSLDKEYLDTLDHYFDKRFNDINDDFVTKNATNLYILKEVFPEANFKDIADLYYDFIVIKSHKNMGFSIKKLREKMLECDGADRIKEQDMDSVRSKLYKLIDFCIFKYYHEFPELSEKNVDILRAAVSDTKKDNLYSDEAARLWSIFKEKFLGFCDKIVVWVTGEHEKDITSVIDKDAYRNRSNVSYFSKLMYAMCFFLDGKEINDLLTTLINKFDNIANQIKTAKELGINTAFVKNYDFFNHSEKYVDELNIVKNIARMKKPSSNAKKAMYHDALTILGIPEDMDEKALDEELDLILEKKTDPVTGKPLKGKNPLRNFIANNVIENSRFIYLIKFCNPENVRKIVNNTKVTEFVLKRIPDAQIERYYKSCTDSEMNPPTEKKITELAGKLKDMNFGNFRNVRQSAKENMEKERFKAVIGLYLTVVYRVVKNLVDVNSRYIMAFHSLERDSQLYNVSVDNDYLALTDTLVKEGDNSRSRYLAGNKRLRDCVKQDIDNAKKWFVSDKYNSITKYRNNVAHLTAVRNCAEFIGDITKIDSYFALYHYLIQRQLAKGLDHERSGFDRNYPQYAPLFKWHTYVKDVVKALNAPFGYNIPRFKNLSIDALFDRNEIKKNDGEKKSDDYPYDVPDYA

SEQ ID NO: 6 - protein, 967 aa, Ruminococcus flavefaciens
MIEKKKSFAKGMGVKSTLVSGSKVYMTTFAEGSDARLEKIVEGDSIRSVNEGEAFSAEMADKNAGYKIGNAKFSHPKGYAVVANNPLYTGPVQQDMLGLKETLEKRYFGESADGNDNICIQVIHNILDIEKILAEYITNAAYAVNNISGLDKDIIGFGKFSTVYTYDEFKDPEHHRAAFNNNDKLINAIKAQYDEFDNFLDNPRLGYFGQAFFSKEGRNYIINYGNECYDILALLSGLRHWVVHNNEEESRISRTWLYNLDKNLDNEYISTLNYLYDRITNELTNSFSKNSAANVNYIAETLGINPAEFAEQYFRFSIMKEQKNLGFNITKLREVMLDRKDMSEIRKNHKVFDSIRTKVYTMMDFVIYRYYIEEDAKVAAANKSLPDNEKSLSEKDIFVINLRGSFNDDQKDALYYDEANRIWRKLENIMHNIKEFRGNKTREYKKKDAPRLPRILPAGRDVSAFSKLMYALTMFLDGKEINDLLTTLINKFDNIQSFLKVMPLIGVNAKFVEEYAFFKDSAKIADELRLIKSFARMGEPIADARRAMYIDAIRILGTNLSYDELKALADTFSLDENGNKLKKGKHGMRNFIINNVISNKRFHYLIRYGDPAHLHEIAKNEAVVKFVLGRIADIQKKQGQNGKNQIDRYYETCIGKDKGKSVSEKVDALTKIITGMNYDQFDKKRSVIEDTGRENAEREKFKKIISLYLTVIYHILKNIVNINARYVIGFHCVERDAQLYKEKGYDINLKKLEEKGFSSVTKLCAGIDETAPDKRKDVEKEMAERAKESIDSLESANPKLYANYIKYSDEKKAEEFTRQINREKAKTALNAYLRNTKWNVIIREDLLRIDNKTCTLFRNKAVHLEVARYVHAYINDIAEVNSYFQLYHYIMQRIIMNERYEKSSGKVSEYFDAVNDEKKYNDRLLKLLCVPFGYCIPRFKNLSIEALFDRNEAAKFDKEKKKVSGNS

SEQ ID NO: 7 - protein, 922 aa, Unknown
  Uncultured Ruminococcus species
MAKKNKMKPRELREAQKKARQLKAAEINNNAAPAIAAMPAAEVIAPVAEKKKSSVKAAGMKSILVSENKMYITSFGKGNSAVLEYEVDNNDYNKTQLSSKDNSNIELGDVNEVNITFSSKHGFGSGVEINTSNPTHRSGESSPVRGDMLGLKSELEKRFFGKTFDDNIHIQLIYNILDIEKILAVYVTNIVYALNNMLGIKDSESYDDFMGYLSARNTYEVFTHPDKSNLSDKVKGNIKKSLSKFNDLLKTKRLGYFGLEEPKTKDTRASEAYKKRVYHMLAIVGQIRQCVFHDKSGAKRFDLYSFINNIDPEYRDTLDYLVEERLKSINKDFIEGNKVNISLLIDMMKGYEADDIIRLYYDFIVLKSQKNLGFSIKKLREKMLEEYGFRFKDKQYDSVRSKMYKLMDFLLFCNYYRNDVAAGEALVRKLRFSMTDDEKEGIYADEAAKLWGKFRNDFENIADHMNGDVIKELGKADMDFDEKILDSEKKNASDLLYFSKMIYMLTYFLDGKEINDLLTTLISKFDNIKEFLKIMKSSAVDVECELTAGYKLFNDSQRITNELFIVKNIASMRKPAASAKLTMFRDALTILGIDDNITDDRISEILKLKEKGKGIHGLRNFITNNVIESSRFVYLIKYANAQKIREVAKNEKVVMFVLGGIPDTQIERYYKSCVEFPDMNSSLEAKRSELARMIKNISFDDFKNVKQQAKGRENVAKERAKAVIGLYLTVMYLLVKNLVNVNARYVIAIHCLERDFGLYKEIIPELASKNLKNDYRILSQTLCELCDDRNESSNLFLKKNKRLRKCVEVDINNADSSMTRKYRNCIAHLTVVRELKEYIGDIRTVDSYFSIYHYVMQRCITKRGDDTKQEEKIKYEDDLLKNHGYTKDFVKALNSPFGYNIPRFKNLSIEQLFDRNEYLTEK

SEQ ID NO: 8 - protein, 186 aa, Artificial Sequence
  Synthetic
AQAALEPGEKPYACPECGKSFSRSDDLVRHQRTHTGEKPYKCPECGKSFSRSDHLTTHQRTHTGEKPYKCPECGKSFSDCRDLARHQRTHTGEKPYACPECGKSFSRSDVLVRHQRTHTGEKPYKCPECGKSFSDPGHLVRHQRTHTGEKPYKCPECGKSFSQSSHLVRHQRTHTGKKTSGQAGQY

SEQ ID NO: 9 - DNA, 18 bp, Artificial Sequence
  Synthetic
ggaggcgtggcctgggcg

SEQ ID NO: 10 - protein, 186 aa, Artificial Sequence
  Synthetic
AQAALEPGEKPYACPECGKSFSTSGHLVRHQRTHTGEKPYKCPECGKSFSRSDVLVRHQRTHTGEKPYKCPECGKSFSQSGDLRRHQRTHTGEKPYACPECGKSFSQSSNLASHQRTHTGEKPYKCPECGKSFSTSGELVRHQRTHTGEKPYKCPECGKSFSRSDNLVRHQRTHTGKKTSGQAGQY

SEQ ID NO: 11 - DNA, 18 bp, Artificial Sequence
  Synthetic
gaggcttaagcagtgggt

SEQ ID NO: 12 - protein, 186 aa, Artificial Sequence
  Synthetic
AQAALEPGEKPYACPECGKSFSSPADLTRHQRTHTGEKPYKCPECGKSFSDPGALVRHQRTHTGEKPYKCPECGKSFSQLAHLRAHQRTHTGEKPYACPECGKSFSDKKDLTRHQRTHTGEKPYKCPECGKSFSTSGSLVRHQRTHTGEKPYKCPECGKSFSQRHSLTEHQRTHTGKKTSGQAGQY

SEQ ID NO: 13 - DNA, 18 bp, Artificial Sequence
  Synthetic
ctagttaccagagtcaca

SEQ ID NO: 14 - DNA, 540 bp, Mus musculus
ttccagatctctgatggccattttcctcgagcctgtgcctcctctaagaacttgttggcaaaagaatgctgcccaccatggatgggtgatgggagtccctgcggccagctttcaggcagaggttcctgccaggatatccttctgtccagtgcaccatctggacctcagttccccttcaaaggggtggatgaccgtgagtcctggccctctgtgttttataataggacctgccagtgctcaggcaacttcatgggtttcaactgcggaaactgtaagtttggatttgggggcccaaattgtacagagaagcgagtcttgattagaagaaacatttttgatttgagtgtctccgaaaagaataagttcttttcttacctcactttagcaaaacatactatcagctcagtctatgtcatccccacaggcacctatggccaaatgaacaatgggtcaacacccatgtttaatgatatcaacatctacgacctctttgtatggatgcattactatgtgtcaagggacacactgcttgggggctct

SEQ ID NO: 15 - DNA, 24 bp, Artificial Sequence
  Synthetic
  misc_feature (10)..(15): n is a, c, g, or t
ttccccttcnnnnnngtggatgac

SEQ ID NO: 16 - DNA, 480 bp, Mus musculus
attggaataattggacgcaagaaagggataagtaatttgatcaaacaatttagctgttgtttttatttgtagacatcactcctgatgttgattttgggagaactggaagcttcagagggaattattaagcacagtggaagagtttcattctgctctcaattttcttggattatgccgggtactatcaaagaaaatatcatctttggtgtttcctatgatgagtacagatataagagtgttgtcaaagcttgccaactacagcaggtaagcatatttatgaaaaatgctgattgtgttagctacttgtgtcagtgttgtgataaaattgcttgactactcaccttgaaaagggttttattttaaattcttttcagggatgataccgtccatcttggcaaaggaggggcaggaatgggaagatggcgagacatgttatatccatagtcaggaagcagacagccagcaggaagtggggcttca

SEQ ID NO: 17 - DNA, 27 bp, Artificial Sequence
  Synthetic
  misc_feature (10)..(18): n is a, c, g, or t
tcactcctgnnnnnnnnnttgggagaa

SEQ ID NO: 18 - DNA, 480 bp, Homo sapiens
gcccaggagccgcccgcgctccctgaaccctagaactgtcttcgactccggggccccgttggaagactgagtgcccggggcacggcacagaagccgcgcccaccgcctgccagttcacaaccgctccgagcgtgggtctccgcccagctccagtcctgtgtaccgggcccgccccctagcggccggggagggaggggccgggtccgcggccggcgaacggggctcgaagggtccttgtagccgggaatgctgctgctgctgctgctgctgctgctgctgctggggggatcacagaccatttctttctttcggccaggctgaggccctgacgtggatgggcaaactgcaggcctgggaaggcagcaagccgggccgtccgtgttccatcctccacgcacccccacctatcgttggttcgcaaagtgcaaagctttcttgtgcatgacgccctgctctggggagcgtctggcgcgatctctg

SEQ ID NO: 19 - DNA, 24 bp, Artificial Sequence
  Synthetic
  misc_feature (10)..(15): n is a, c, g, or t
gcccgccccnnnnnngccggggag

SEQ ID NO: 20 - protein, 147 aa, Artificial Sequence
  Synthetic
SRGAGAGAGAGAGSRFQCRICMRNFSDRSNLSRHIRTHTGEKPFACDICGRKFAISSNLNSHTKIHTGSQKPFQCRICMRNFSRSDNLARHIRTHTGEKPFACDICGRKFATSGNLTRHTKIHLRGSQLSIVDAPEQREGASQVSVS

SEQ ID NO: 21 - DNA, 12 bp, Artificial Sequence
  Synthetic
gtcatcctcatc

SEQ ID NO: 22 - protein, 29 aa, Artificial Sequence
  Synthetic
MNIQMLLEAADYLERREREAEHGYASMLP

SEQ ID NO: 23 - protein, 144 aa, Artificial Sequence
  Synthetic
ASPKKKRKVEASGSGMNIQMLLEAADYLERREREAEHGYASMLPGSGMNIQMLLEAADYLERREREAEHGYASMLPGSGMNIQMLLEAADYLERREREAEHGYASMLPGSGMNIQMLLEAADYLERREREAEHGYASMLPSRSR

SEQ ID NO: 24 - protein, 41 aa, Artificial Sequence
  Synthetic
VTFEDVAVLFTRDEWKKLDLSQRSLYREVMLENYSNLASMA

SEQ ID NO: 25 - protein, 5 aa, Artificial Sequence
  Synthetic
GGGGS

SEQ ID NO: 26 - protein, 10 aa, Artificial Sequence
  Synthetic
GGGGSGGGGS

SEQ ID NO: 27 - protein, 15 aa, Artificial Sequence
  Synthetic
GGGGSGGGGSGGGGS

SEQ ID NO: 28 - protein, 6 aa, Artificial Sequence
  Synthetic
GGGGGG

SEQ ID NO: 29 - protein, 15 aa, Artificial Sequence
  Synthetic
EAAAKEAAAKEAAAK

SEQ ID NO: 30 - protein, 10 aa, Artificial Sequence
  Synthetic
EAAAKEAAAK

SEQ ID NO: 31 - protein, 8 aa, Artificial Sequence
  Synthetic
GGGGGGGG

SEQ ID NO: 32 - protein, 5 aa, Artificial Sequence
  Synthetic
EAAAK

SEQ ID NO: 33 - protein, 46 aa, Artificial Sequence
  Synthetic
AEAAAKEAAAKEAAAKEAAAKALEAEAAAKEAAAKEAAAKEAAAKA

SEQ ID NO: 34 - protein, 5 aa, Artificial Sequence
  Synthetic
PAPAP

SEQ ID NO: 35 - protein, 12 aa, Artificial Sequence
  Synthetic
AEAAAKEAAAKA

SEQ ID NO: 36 - protein, 17 aa, Artificial Sequence
  Synthetic
KRTADGSEFEPKKKRKV

SEQ ID NO: 37 - protein, 16 aa, Artificial Sequence
  Synthetic
KRPAATKKAGQAKKKK

SEQ ID NO: 38 - protein, 17 aa, Artificial Sequence
  Synthetic
KKTELQTTNAENKTKKL

SEQ ID NO: 39 - protein, 19 aa, Artificial Sequence
  Synthetic
KRGINDRNFWRGENGRKTR

SEQ ID NO: 40 - protein, 16 aa, Artificial Sequence
  Synthetic
RKSGKIAAIVVKRPRK

SEQ ID NO: 41 - protein, 7 aa, Artificial Sequence
  Synthetic
PKKKRKV

SEQ ID NO: 42 - protein, 25 aa, Artificial Sequence
  Synthetic
MSRRRKANPTKLSENAKKLAKEVEN

SEQ ID NO: 43 - protein, 30 aa, Artificial Sequence
  Synthetic
MDSLLMNRRKFLYQFKNVRWAKGRRETYLC

SEQ ID NO: 44 - protein, 9 aa, Artificial Sequence
  Synthetic
KLKIKRPVK

SEQ ID NO: 45 - protein, 9 aa, Artificial Sequence
  Synthetic
PAAKRVKLD

SEQ ID NO: 46 - protein, 25 aa, Artificial Sequence
  Synthetic
MLSLRQSIRFFKPATRTLCSSRYLL

SEQ ID NO: 47 - protein, 55 aa, Artificial Sequence
  Synthetic
MVKLSKEAKQRLQQLFKGSQFAIRWGFIPLVIYLGFKRGADPGMPEPTVLSLLWG

SEQ ID NO: 48 - protein, 47 aa, Artificial Sequence
  Synthetic
KLIAKSAEDEKAKEEPGNHTTLILAMLAIGVFSLGAFIKIIQLRKNN

SEQ ID NO: 49 - protein, 9 aa, Artificial Sequence
  Synthetic
YPYDVPDYA

SEQ ID NO: 50 - protein, 8 aa, Artificial Sequence
  Synthetic
DYKDDDDK

SEQ ID NO: 51 - protein, 6 aa, Artificial Sequence
  Synthetic
HHHHHH

SEQ ID NO: 52 - protein, 10 aa, Artificial Sequence
  Synthetic
EQKLISEEDL

SEQ ID NO: 53 - protein, 14 aa, Artificial Sequence
  Synthetic
GKPIPNPLLGLDST

SEQ ID NO: 54 - protein, 15 aa, Artificial Sequence
  Synthetic
GLNDIFEAQKIEWHE

SEQ ID NO: 55 - DNA, 3639 bp, Artificial Sequence
  Synthetic
atgggccccaagaagaagagaaaggtggaggccagcatcgaaaaaaaaaagtccttcgccaagggcatgggcgtgaagtccacactcgtgtccggctccaaagtgtacatgacaaccttcgccgaaggcagcgacgccaggctggaaaagatcgtggagggcgacagcatcaggagcgtgaatgagggcgaggccttcagcgctgaaatggccgataaaaacgccggctataagatcggcaacgccaaattcagccatcctaagggctacgccgtggtggctaacaaccctctgtatacaggacccgtccagcaggatatgctcggcctgaaggaaactctggaaaagaggtacttcggcgagagcgctgatggcaatgacaatatttgtatccaggtgatccataacatcctggacattgaaaaaatcctcgccgaatacattaccaacgccgcctacgccgtcaacaatatctccggcctggataaggacattattggattcggcaagttctccacagtgtatacctacgacgaattcaaagaccccgagcaccatagggccgctttcaacaataacgataagctcatcaacgccatcaaggcccagtatgacgagttcgacaacttcctcgataaccccagactcggctatttcggccaggcctttttcagcaaggagggcagaaattacatcatcaattacggcaacgaatgctatgacattctggccctcctgagcggactgaggcactgggtggtccataacaacgaagaagagtccaggatctccaggacctggctctacaacctcgataagaacctcgacaacgaatacatctccaccctcaactacctctacgacaggatcaccaatgagctgaccaactccttctccaagaactccgccgccaacgtgaactatattgccgaaactctgggaatcaaccctgccgaattcgccgaacaatatttcagattcagcattatgaaagagcagaaaaacctcggattcaatatcaccaagctcagggaagtgatgctggacaggaaggatatgtccgagatcaggaaaaatcataaggtgttcgactccatcaggaccaaggtctacaccatgatggactttgtgatttataggtattacatcgaagaggatgccaaggtggctgccgccaataagtccctccccgataatgagaagtccctgagcgagaaggatatctttgtgattaacctgaggggctccttcaacgacgaccagaaggatgccctctactacgatgaagctaatagaatttggagaaagctcgaaaatatcatgcacaacatcaaggaatttaggggaaacaagacaagagagtataagaagaaggacgcccctagactgcccagaatcctgcccgctggccgtgatgtttccgccttcagcaaactcatgtatgccctgaccatgttcctggatggcaaggagatcaacgacctcctgaccaccctgattaataaattcgataacatccagagcttcctgaaggtgatgcctctcatcggagtcaacgctaagttcgtggaggaatacgcctttttcaaagactccgccaagatcgccgatgagctgaggctgatcaagtccttcgctagaatgggagaacctattgccgatgccaggagggccatgtatatcgacgccatccgtattttaggaaccaacctgtcctatgatgagctcaaggccctcgccgacaccttttccctggacgagaacggaaacaagctcaagaaaggcaagcacggcatgagaaatttcattattaataacgtgatcagcaataaaaggttccactacctgatcagatacggtgatcctgcccacctccatgagatcgccaaaaacgaggccgtggtgaagttcgtgctcggcaggatcgctgacatccagaaaaaacagggccagaacggcaagaaccagatcgacaggtactacgaaacttgtatcggaaaggataagggcaagagcgtgagcgaaaaggtggacgctctcacaaagatcatcaccggaatgaactacgaccaattcgacaagaaaaggagcgtcattgaggacaccggcagggaaaacgccgagagggagaagtttaaaaagatcatcagcctgtacctcaccgtgatctaccacatcctcaagaatattgtcaatatcaacgccaggtacgtcatcggattccattgcgtcgagcgtgatgctcaactgtacaaggagaaaggctacgacatcaatctcaagaaactggaagagaagggattcagctccgtcaccaagctctgcgctggcattgatgaaactgcccccgataagagaaaggacgtggaaaaggagatggctgaaagagccaaggagagcattgacagcctcgagagcgccaaccccaagctgtatgccaattacatcaaatacagcgacgagaagaaagccgaggagttcaccaggcagattaacagggagaaggccaaaaccgccctgaacgcctacctgaggaacaccaagtggaatgtgatcatcagggaggacctcctgagaattgacaacaagacatgtaccctgttcagaaacaaggccgtccacctggaagtggccaggtatgtccacgcctatatcaacgacattgccgaggtcaattcctacttccaactgtaccattacatcatgcagagaattatcatgaatgagaggtacgagaaaagcagcggaaaggtgtccgagtacttcgacgctgtgaatgacgagaagaagtacaacgataggctcctgaaactgctgtgtgtgcctttcggctactgtatccccaggtttaagaacctgagcatcgaggccctgttcgataggaacgaggccgccaagttcgacaaggagaaaaagaaggtgtccggcaattccggatccggacctaagaaaaagaggaaggtggcggccgcttacccatacgatgttccagattacgctggaggaggtggaagcggaggaggaggaagcggaggaggaggtagctctaggggagctggcgctggagcgggtgcaggggctggctctagattccagtgccggatctgcatgcggaacttcagcgaccggtccaacctgagcaggcacatcagaacccacaccggagaaaagcccttcgcctgcgacatttgcggccggaagttcgccatcagcagcaacctgaacagccacaccaagatccacactggcagccagaaacctttccagtgcagaatttgtatgagaaactttagcagaagcgacaacctggccagacacatccggacacatactggtgaaaaaccttttgcctgtgatatctgtggcagaaagtttgccacctccggcaatctgacccggcacacaaagattcacctgcggggcagccagctatcgattgtcgacgctcctgaacaacgtgaaggtgcttctcaagtttctgtttctgttacttttgaagatgttgctgttctttttactcgtgatgaatggaaaaaacttgatctttctcaacgttctctttatcgtgaagttatgcttgaaaattattctaatcttgcttctatggctggatcccccaagaaaaagcgcaaggtataa

SEQ ID NO: 56 - protein, 1212 aa, Artificial Sequence
  Synthetic
MGPKKKRKVEASIEKKKSFAKGMGVKSTLVSGSKVYMTTFAEGSDARLEKIVEGDSIRSVNEGEAFSAEMADKNAGYKIGNAKFSHPKGYAVVANNPLYTGPVQQDMLGLKETLEKRYFGESADGNDNICIQVIHNILDIEKILAEYITNAAYAVNNISGLDKDIIGFGKFSTVYTYDEFKDPEHHRAAFNNNDKLINAIKAQYDEFDNFLDNPRLGYFGQAFFSKEGRNYIINYGNECYDILALLSGLRHWVVHNNEEESRISRTWLYNLDKNLDNEYISTLNYLYDRITNELTNSFSKNSAANVNYIAETLGINPAEFAEQYFRFSIMKEQKNLGFNITKLREVMLDRKDMSEIRKNHKVFDSIRTKVYTMMDFVIYRYYIEEDAKVAAANKSLPDNEKSLSEKDIFVINLRGSFNDDQKDALYYDEANRIWRKLENIMHNIKEFRGNKTREYKKKDAPRLPRILPAGRDVSAFSKLMYALTMFLDGKEINDLLTTLINKFDNIQSFLKVMPLIGVNAKFVEEYAFFKDSAKIADELRLIKSFARMGEPIADARRAMYIDAIRILGTNLSYDELKALADTFSLDENGNKLKKGKHGMRNFIINNVISNKRFHYLIRYGDPAHLHEIAKNEAVVKFVLGRIADIQKKQGQNGKNQIDRYYETCIGKDKGKSVSEKVDALTKIITGMNYDQFDKKRSVIEDTGRENAEREKFKKIISLYLTVIYHILKNIVNINARYVIGFHCVERDAQLYKEKGYDINLKKLEEKGFSSVTKLCAGIDETAPDKRKDVEKEMAERAKESIDSLESANPKLYANYIKYSDEKKAEEFTRQINREKAKTALNAYLRNTKWNVIIREDLLRIDNKTCTLFRNKAVHLEVARYVHAYINDIAEVNSYFQLYHYIMQRIIMNERYEKSSGKVSEYFDAVNDEKKYNDRLLKLLCVPFGYCIPRFKNLSIEALFDRNEAAKFDKEKKKVSGNSGSGPKKKRKVAAAYPYDVPDYAGGGGSGGGGSGGGGSSRGAGAGAGAGAGSRFQCRICMRNFSDRSNLSRHIRTHTGEKPFACDICGRKFAISSNLNSHTKIHTGSQKPFQCRICMRNFSRSDNLARHIRTHTGEKPFACDICGRKFATSGNLTRHTKIHLRGSQLSIVDAPEQREGASQVSVSVTFEDVAVLFTRDEWKKLDLSQRSLYREVMLENYSNLASMAGSPKKKRKV

SEQ ID NO: 57 - DNA, 3253 bp, Artificial Sequence
  Synthetic
ctcctgggcaacgtgctggttattgtgctgtctcatcattttggcaaagaattcggtaccattgtacacggccgcataatcgaaattaatacgactcactatagggcaagtaaacccctaccaactggtcggggtttgaaacgcagcagcagcagcagcagcagcaagtaaacccctaccaactggtcggggtttgaaacccgctgagcaataactagcataacccggtaccgcgggcccgggcgccaccatgggccccaagaagaagagaaaggtggaggccagcatcgaaaaaaaaaagtccttcgccaagggcatgggcgtgaagtccacactcgtgtccggctccaaagtgtacatgacaaccttcgccgaaggcagcgacgccaggctggaaaagatcgtggagggcgacagcatcaggagcgtgaatgagggcgaggccttcagcgctgaaatggccgataaaaacgccggctataagatcggcaacgccaaattcagccatcctaagggctacgccgtggtggctaacaaccctctgtatacaggacccgtccagcaggatatgctcggcctgaaggaaactctggaaaagaggtacttcggcgagagcgctgatggcaatgacaatatttgtatccaggtgatccataacatcctggacattgaaaaaatcctcgccgaatacattaccaacgccgcctacgccgtcaacaatatctccggcctggataaggacattattggattcggcaagttctccacagtgtatacctacgacgaattcaaagaccccgagcaccatagggccgctttcaacaataacgataagctcatcaacgccatcaaggcccagtatgacgagttcgacaacttcctcgataaccccagactcggctatttcggccaggcctttttcagcaaggagggcagaaattacatcatcaattacggcaacgaatgctatgacattctggccctcctgagcggactgaggcactgggtggtccataacaacgaagaagagtccaggatctccaggacctggctctacaacctcgataagaacctcgacaacgaatacatctccaccctcaactacctctacgacaggatcaccaatgagctgaccaactccttctccaagaactccgccgccaacgtgaactatattgccgaaactctgggaatcaaccctgccgaattcgccgaacaatatttcagattcagcattatgaaagagcagaaaaacctcggattcaatatcaccaagctcagggaagtgatgctggacaggaaggatatgtccgagatcaggaaaaatcataaggtgttcgactccatcaggaccaaggtctacaccatgatggactttgtgatttataggtattacatcgaagaggatgccaaggtggctgccgccaataagtccctccccgataatgagaagtccctgagcgagaaggatatctttgtgattaacctgaggggctccttcaacgacgaccagaaggatgccctctactacgatgaagctaatagaatttggagaaagctcgaaaatatcatgcacaacatcaaggaatttaggggaaacaagacaagagagtataagaagaaggacgcccctagactgcccagaatcctgcccgctggccgtgatgtttccgccttcagcaaactcatgtatgccctgaccatgttcctggatggcaaggagatcaacgacctcctgaccaccctgattaataaattcgataacatccagagcttcctgaaggtgatgcctctcatcggagtcaacgctaagttcgtggaggaatacgcctttttcaaagactccgccaagatcgccgatgagctgaggctgatcaagtccttcgctagaatgggagaacctattgccgatgccaggagggccatgtatatcgacgccatccgtattttaggaaccaacctgtcctatgatgagctcaaggccctcgccgacaccttttccctggacgagaacggaaacaagctcaagaaaggcaagcacggcatgagaaatttcattattaataacgtgatcagcaataaaaggttccactacctgatcagatacggtgatcctgcccacctccatgagatcgccaaaaacgaggccgtggtgaagttcgtgctcggcaggatcgctgacatccagaaaaaacagggccagaacggcaagaaccagatcgacaggtactacgaaacttgtatcggaaaggataagggcaagagcgtgagcgaaaaggtggacgctctcacaaagatcatcaccggaatgaactacgaccaattcgacaagaaaaggagcgtcattgaggacaccggcagggaaaacgccgagagggagaagtttaaaaagatcatcagcctgtacctcaccgtgatctaccacatcctcaagaatattgtcaatatcaacgccaggtacgtcatcggattccattgcgtcgagcgtgatgctcaactgtacaaggagaaaggctacgacatcaatctcaagaaactggaagagaagggattcagctccgtcaccaagctctgcgctggcattgatgaaactgcccccgataagagaaaggacgtggaaaaggagatggctgaaagagccaaggagagcattgacagcctcgagagcgccaaccccaagctgtatgccaattacatcaaatacagcgacgagaagaaagccgaggagttcaccaggcagattaacagggagaaggccaaaaccgccctgaacgcctacctgaggaacaccaagtggaatgtgatcatcagggaggacctcctgagaattgacaacaagacatgtaccctgttcagaaacaaggccgtccacctggaagtggccaggtatgtccacgcctatatcaacgacattgccgaggtcaattcctacttccaactgtaccattacatcatgcagagaattatcatgaatgagaggtacgagaaaagcagcggaaaggtgtccgagtacttcgacgctgtgaatgacgagaagaagtacaacgataggctcctgaaactgctgtgtgtgcctttcggctactgtatccccaggtttaagaacctgagcatcgaggccctgttcgataggaacgaggccgccaagttcgacaaggagaaaaagaaggtgtccggcaattccggatccggacctaagaaaaagaggaaggtggcggccgcttacccatacgatgttccagattacgcttaa

SEQ ID NO: 58 - DNA, 22 bp, Artificial Sequence
  Synthetic
ctgctgctgctgctgctgctgc

SEQ ID NO: 59 - DNA, 22 bp, Artificial Sequence
  Synthetic
gctgctgctgctgctgctgctg

SEQ ID NO: 60 - DNA, 22 bp, Artificial Sequence
  Synthetic
tgctgctgctgctgctgctgct

SEQ ID NO: 61 - DNA, 22 bp, Artificial Sequence
  Synthetic
cagcagcagcagcagcagcagc

SEQ ID NO: 62 - DNA, 22 bp, Artificial Sequence
  Synthetic
gcagcagcagcagcagcagcag

SEQ ID NO: 63 - DNA, 22 bp, Artificial Sequence
  Synthetic
ggggccggggccggggccgggg

SEQ ID NO: 64 - DNA, 22 bp, Artificial Sequence
  Synthetic
ggccggggccggggccggggcc

SEQ ID NO: 65 - DNA, 22 bp, Artificial Sequence
  Synthetic
tgcctgcctgcctgcctgcctg

SEQ ID NO: 66 - DNA, 22 bp, Artificial Sequence
  Synthetic
cctgcctgcctgcctgcctgcc

SEQ ID NO: 67 - DNA, 22 bp, Artificial Sequence
  Synthetic
ctattctattctattctattct

SEQ ID NO: 68 - DNA, 22 bp, Artificial Sequence
  Synthetic
attctattctattctattctat

SEQ ID NO: 69 - DNA, 22 bp, Artificial Sequence
  Synthetic
gccagttcacaaccgctccgag

SEQ ID NO: 70 - DNA, 22 bp, Artificial Sequence
  Synthetic
ggctcgaagggtccttgtagcc

SEQ ID NO: 71 - DNA, 22 bp, Artificial Sequence
  Synthetic
actcgctgacaggctacaggac

SEQ ID NO: 72 - DNA, 22 bp, Artificial Sequence
  Synthetic
tcctcggtatttattgtctgtc

SEQ ID NO: 73 - DNA, 22 bp, Artificial Sequence
  Synthetic
cctagaactgtcttcgactccg

SEQ ID NO: 74 - DNA, 22 bp, Artificial Sequence
  Synthetic
cctatcgttggttcgcaaagtg

SEQ ID NO: 75 - DNA, 22 bp, Artificial Sequence
  Synthetic
agttgcaggagcggatggagtt

SEQ ID NO: 76 - skipped

SEQ ID NO: 77 - DNA, 22 bp, Artificial Sequence
  Synthetic
gcgtgtatagacacctggagga

SEQ ID NO: 78 - DNA, 2865 bp, Unknown
  Anaerobic digester metagenome 15706
atgaacaacaaacgcaaaaccaaagcgaaagcggcgggcctgaaaagcgtgttttttgatcagaaacaggcggtgctgaccacctttgcgaaaggcaacaacagccagattgaaaaaaaagtggtgaacagcgaagtgaaagatctgcgccagccgccggcgtttgatctggaactgaaagaaaaaaccttttatattagcggcaaaaacaacattaacaccagccgcgaaaacccgctggcgagcgcgagcctgccgctgagcaaacgccagcgcattcgcgcggaacgcattaaacgcgcgcgcgaagaaaaccgcccgtatcataacgtgaaacgcgtgggcgaagatgatctgcgcgcgaaagcggatctggaaaaacattattttggcaaagaatatagcgataacctgaaaattcagattatttataacattctggatattaacaaaattattagcccgtatattaacgatattgtgtatagcatgaacaacctggcgcgcaacgatgaatatattgatggcaaaattgatgtgattggcagcctgagcagcaccaccgattatagcagctttatgagcccgaacaaagatctggaaaaagaaaaaaaatttagctttcatcgcgaaaactataaaaaatttgtggaagcgagcaaaccgtatatgcgctattatggcaaagtgtttattcgcgatgtgaaaaaaagcaaactgagcaccggcaaaggcgaaaaaattgaagtgatgtatcgcagcgatgaagaaatttttaccatttttcagattctgagctatgtgcgccagagcattatgcataacgatattggcaacaaaagcagcattctggcgattgaaaaatatccggcgcgctttgtgggctttctgagcgatctgctgaaaaccaaaaccaacgatgtgaaccgcatgtttattgataacaacagccagaccaacttttgggtgctgtttagcatttttggcctgcaggatcataccagcggcgcggataaaatttgccgcaacttttatgattttgtgattaaagcggatagcaaaaacctgggctttagcctgaaaaaaattcgcgaactgatgctggatctgccgaacgcgaacatgctgcgcgatcatcagtttgataccgtgcgcagcaaattttataccctgctggattttattatttatcagcattatctggaagaaaaaagccgcattgataacatggtggaaaaactgcgcatgaccctgaaagaagaagaaaaagaagtgctgtatgcggcggaagcgaaaattgtgtggaacgcgattggcgcgaaagtgattaacaaactggtgccgatgatgaacggcgatgcgctgaaagaaattaaacgcaaaaaccgcgatcgcaaactgccgcagagcgtgattgcgaccgtgcaggtgaacagcgatgcgaacgtgtttagcggcctgatttattttctgaccctgtttctggatggcaaagaaattaacgaaatggtgagcaacctgattaccaaatttgaaaacattgatagcctgctgcatgtggatcgcgaaatttataaaagcgatgaaaaagatctggatctggaaattgaaaaactggcgctgttttttaaaggcgtggtgcgcccgaacgcgaaaaccgataccggcgcgggcgaaattagcaaaagctttagcatttttcagagcgcggaacgcattattgaagaactgaaatttattaaaaacgtgacccgcatggataacgaaatttttccgagcgaaggcgtgtttctggatgcggcgaacgtgctgggcgtgcgcggcgatgattttgattttagcaacgaatttgtgggcgatgatctgcatagcgatgcgaacaaaaaaattattaacaaaattaacggcaccaaagaagatcgcaacctgcgcaactttattattaacaacgtggtgaaaagccgccgctttcagtatattgcgcgccatatgaacacccattatgtgaaacagctggcgaacaacgaaaccctgaaccgctttgtgctgaacaaaatgggcgatgcgaaaattattaaccgctattatgaaagcattagcggcaacaccccgaacattgaagtgcgcagccagattgattatctggtgaaacgcctgcgcagctttagctttgaagatctgaacgatgtgaaacagaaagtgcgcccgggcaccaacgaaagcattgaaaaagaaaaaaaaaaagcgctggtgggcctgtgcctgaccattcagtatctggtgtataaaaacctggtgaacattaacgcgcgctataccaccgcgttttattgcctggaacgcgatagcaaactgaaaggctttggcgtggatgtgtggcgcgattttgaaagctataccgcgctgaccaaccattttattaaagaaggctatctgccggtgcgcaaagcggaaattctgcgcgcgaacctgaaacatctggattgcgaagatggctttaaatattatcgcaaccaggtgacccatctgaacgcgattcgcgtggcgtataaatatattaacgaaattaaaagcgtgcatagctattttgcgctgtatcattatattatgcagcgccatctgtatgatagcctgcaggcgaaagcgaaagatagcagcggctttgtgattgatgcgctgaaaaaaagctttgaacataaaatttatagcaaagatctgctgcatgtgctgcatagcccgtttggctataacaccgcgcgctataaaaacctgagcattgaagcgctgtttgataaaaacgaaagccgcccggaagtgaacccgctgagcaccaacgattatccgtatgatgtgccggattatgcg

SEQ ID NO: 79 - DNA, 2889 bp, Eubacterium siraeum
atgggcaaaaaaattcatgcgcgcgatctgcgcgaacagcgcaaaaccgatcgcaccgaaaaatttgcggatcagaacaaaaaacgcgaagcggaacgcgcggtgccgaaaaaagatgcggcggtgagcgtgaaaagcgtgagcagcgtgagcagcaaaaaagataacgtgaccaaaagcatggcgaaagcggcgggcgtgaaaagcgtgtttgcggtgggcaacaccgtgtatatgaccagctttggccgcggcaacgatgcggtgctggaacagaaaattgtggataccagccatgaaccgctgaacattgatgatccggcgtatcagctgaacgtggtgaccatgaacggctatagcgtgaccggccatcgcggcgaaaccgtgagcgcggtgaccgataacccgctgcgccgctttaacggccgcaaaaaagatgaaccggaacagagcgtgccgaccgatatgctgtgcctgaaaccgaccctggaaaaaaaattttttggcaaagaatttgatgataacattcatattcagctgatttataacattctggatattgaaaaaattctggcggtgtatagcaccaacgcgatttatgcgctgaacaacatgagcgcggatgaaaacattgaaaacagcgatttttttatgaaacgcaccaccgatgaaacctttgatgattttgaaaaaaaaaaagaaagcaccaacagccgcgaaaaagcggattttgatgcgtttgaaaaatttattggcaactatcgcctggcgtattttgcggatgcgttttatgtgaacaaaaaaaacccgaaaggcaaagcgaaaaacgtgctgcgcgaagataaagaactgtatagcgtgctgaccctgattggcaaactgcgccattggtgcgtgcatagcgaagaaggccgcgcggaattttggctgtataaactggatgaactgaaagatgattttaaaaacgtgctggatgtggtgtataaccgcccggtggaagaaattaacaaccgctttattgaaaacaacaaagtgaacattcagattctgggcagcgtgtataaaaacaccgatattgcggaactggtgcgcagctattatgaatttctgattaccaaaaaatataaaaacatgggctttagcattaaaaaactgcgcgaaagcatgctggaaggcaaaggctatgcggataaagaatatgatagcgtgcgcaacaaactgtatcagatgaccgattttattctgtataccggctatattaacgaagatagcgatcgcgcggatgatctggtgaacaccctgcgcagcagcctgaaagaagatgataaaaccaccgtgtattgcaaagaagcggattatctgtggaaaaaatatcgcgaaagcattcgcgaagtggcggatgcgctggatggcgataacattaaaaaactgagcaaaagcaacattgaaattcaggaagataaactgcgcaaatgctttattagctatgcggatagcgtgagcgaatttaccaaactgatttatctgctgacccgctttctgagcggcaaagaaattaacgatctggtgaccaccctgattaacaaatttgataacattcgcagctttctggaaattatggatgaactgggcctggatcgcacctttaccgcggaatatagcttttttgaaggcagcaccaaatatctggcggaactggtggaactgaacagctttgtgaaaagctgcagctttgatattaacgcgaaacgcaccatgtatcgcgatgcgctggatattctgggcattgaaagcgataaaaccgaagaagatattgaaaaaatgattgataacattctgcagattgatgcgaacggcgataaaaaactgaaaaaaaacaacggcctgcgcaactttattgcgagcaacgtgattgatagcaaccgctttaaatatctggtgcgctatggcaacccgaaaaaaattcgcgaaaccgcgaaatgcaaaccggcggtgcgctttgtgctgaacgaaattccggatgcgcagattgaacgctattatgaagcgtgctgcccgaaaaacaccgcgctgtgcagcgcgaacaaacgccgcgaaaaactggcggatatgattgcggaaattaaatttgaaaactttagcgatgcgggcaactatcagaaagcgaacgtgaccagccgcaccagcgaagcggaaattaaacgcaaaaaccaggcgattattcgcctgtatctgaccgtgatgtatattatgctgaaaaacctggtgaacgtgaacgcgcgctatgtgattgcgtttcattgcgtggaacgcgataccaaactgtatgcggaaagcggcctggaagtgggcaacattgaaaaaaacaaaaccaacctgaccatggcggtgatgggcgtgaaactggaaaacggcattattaaaaccgaatttgataaaagctttgcggaaaacgcggcgaaccgctatctgcgcaacgcgcgctggtataaactgattctggataacctgaaaaaaagcgaacgcgcggtggtgaacgaatttcgcaacaccgtgtgccatctgaacgcgattcgcaacattaacattaacattaaagaaattaaagaagtggaaaactattttgcgctgtatcattatctgattcagaaacatctggaaaaccgctttgcggataaaaaagtggaacgcgataccggcgattttattagcaaactggaagaacataaaacctattgcaaagattttgtgaaagcgtattgcaccccgtttggctataacctggtgcgctataaaaacctgaccattgatggcctgtttgataaaaactatccgggcaaagatgatagcgatgaacagaaatatccgtatgatgtgccggattatgcg

SEQ ID NO: 80 - DNA, 2745 bp, Unknown
  Gut metagenome assembly P1E0-k21
atggaacgcgaagtgaaaaaaccgccgaaaaaaagcctggcgaaagcggcgggcctgaaaagcacctttgtgattagcccgcaggaaaaagaactggcgatgaccgcgtttggccgcggcaacgatgcgctgctgcagaaacgcattgtggatggcgtggtgcgcgatgtggcgggcgaaaaacagcagtttcaggtgcagcgccaggatgaaagccgctttcgcctgcagaacagccgcctggcggatcgcaccgtgaccgcggatgatccgctgcatcgcgcggaaaccccgcgccgccagccgctgggcgcgggcatggatcagctgcgccgcaaagcgattctggaacagaaatattttggccgcacctttgatgataacattcatattcagctgatttataacattctggatattcataaaatgctggcggtgccggcgaaccatattgtgcataccctgaacctgctgggcggctatggcgaaaccgattttgtgggcatgctgccggcgggcctgccgtatgataaactgcgcgtggtgaaaaaaaaaaacggcgataccgtggatattaaagcggatattgcggcgtatgcgaaacgcccgcagctggcgtatctgggcgcggcgttttatgatgtgaccccgggcaaaagcaaacgcgatgcggcgcgcggccgcgtgaaacgcgaacaggatgtgtatgcgattctgagcctgatgagcctgctgcgccagttttgcgcgcatgatagcgtgcgcatttggggccagaacaccaccgcggcgctgtatcatctgcaggcgctgccgcaggatatgaaagatctgctggatgatggctggcgccgcgcgctgggcggcgtgaacgatcattttctggataccaacaaagtgaacctgctgaccctgtttgaatattatggcgcggaaaccaaacaggcgcgcgtggcgctgacccaggatttttatcgctttgtggtgctgaaagaacagaaaaacatgggctttagcctgcgccgcctgcgcgaagaactgctgaaactgccggatgcggcgtatctgaccggccaggaatatgatagcgtgcgccagaaactgtatatgctgctggattttctgctgtgccgcctgtatgcgcaggaacgcgcggatcgctgcgaagaactggtgagcgcgctgcgctgcgcgctgagcgatgaagaaaaagataccgtgtatcaggcggaagcggcggcgctgtggcaggcgctgggcgataccctgcgccgcaaactgctgccgctgctgaaaggcaaaaaactgcaggataaagataaaaaaaaaagcgatgaactgggcctgagccgcgatgtgctggatggcgtgctgtttcgcccggcgcagcagggcagccgcgcgaacgcggattatttttgccgcctgatgcatctgagcacctggtttatggatggcaaagaaattaacaccctgctgaccaccctgattagcaaactggaaaacattgatagcctgcgcagcgtgctggaaagcatgggcctggcgtatagctttgtgccggcgtatgcgatgtttgatcatagccgctatattgcgggccagctgcgcgtggtgaacaacattgcgcgcatgcgcaaaccggcgattggcgcgaaacgcgaaatgtatcgcgcggcggtggtgctgctgggcgtggatagcccggaagcggcggcggcgattaccgatgatctgctgcagattgatccggaaaccggcaaagtgcgcccgcgcagcgatagcgcgcgcgataccggcctgcgcaactttattgcgaacaacgtggtggaaagccgccgctttacctatctgctgcgctatatgaccccggaacaggcgcgcgtgctggcgcagaacgaaaaactgattgcgtttgtgctgagcaccgtgccggatacccagctggaacgctattgccgcacctgcggccgcgaagatattaccggccgcccggcgcagattcgctatctgaccgcgcagattatgggcgtgcgctatgaaagctttaccgatgtggaacagcgcggccgcggcgataacccgaaaaaagaacgctataaagcgctgattggcctgtatctgaccgtgctgtatctggcggtgaaaaacatggtgaactgcaacgcgcgctatgtgattgcgttttattgccgcgatcgcgataccgcgctgtatcagaaagaagtgtgctggtatgatctggaagaagataaaaaaagcggcaaacagcgccaggtggaagattataccgcgctgacccgctattttgtgagccagggctatctgaaccgccatgcgtgcggctatctgcgcagcaacatgaacggcattagcaacagcctgctgaccgcgtatcgcaacgcggtggatcatctgaacgcgattccgccgctgggcagcctgtgccgcgatattggccgcgtggatagctattttgcgctgtatcattatgcggtgcagcagtatctgaacggccgctattatcgcaaaaccccgcgcgaacaggaactgtttgcggcgatggcgcagcatcgcacctggtgcagcgatctggtgaaagcgctgaacaccccgtttggctataacctggcgcgctataaaaacctgagcattgatggcctgtttgatcgcgaaggcgatcatgtggtgcgcgaagatggcgaaaaaccggcggaatatccgtatgatgtgccggattatgcg

SEQ ID NO: 81 - DNA, 2859 bp, Ruminococcus albus
atggcgaaaaaaagcaaaggcatgagcctgcgcgaaaaacgcgaactggaaaaacagaaacgcattcagaaagcggcggtgaacagcgtgaacgataccccggaaaaaaccgaagaagcgaacgtggtgagcgtgaacgtgcgcaccagcgcggaaaacaaacatagcaaaaaaagcgcggcgaaagcgctgggcctgaaaagcggcctggtgattggcgatgaactgtatctgaccagctttggccgcggcaacgaagcgaaactggaaaaaaaaattagcggcgataccgtggaaaaactgggcattggcgcgtttgaagtggcggaacgcgatgaaagcaccctgaccctggaaagcggccgcattaaagataaaaccgcgcgcccgaaagatccgcgccatattaccgtggatacccagggcaaatttaaagaagatatgctgggcattcgcagcgtgctggaaaaaaaaatttttggcaaaacctttgatgataacattcatgtgcagctggcgtataacattctggatgtggaaaaaattatggcgcagtatgtgagcgatattgtgtatatgctgcataacaccgataaaaccgaacgcaacgataacctgatgggctatatgagcattcgcaacacctataaaaccttttgcgataccagcaacctgccggatgataccaaacagaaagtggaaaaccagaaacgcgaatttgataaaattattaaaagcggccgcctgggctattttggcgaagcgtttatggtgaacagcggcaacagcaccaaactgcgcccggaaaaagaaatttatcatatttttgcgctgatggcgagcctgcgccagagctattttcatggctatgtgaaagataccgattatcagggcaccacctgggcgtataccctggaagataaactgaaaggcccgagccatgaatttcgcgaaaccattgataaaatttttgatgaaggctttagcaaaattagcaaagattttggcaaaatgaacaaagtgaacctgcagattctggaacagatgattggcgaactgtatggcagcattgaacgccagaacctgacctgcgattattatgattttattcagctgaaaaaacataaatatctgggctttagcattaaacgcctgcgcgaaaccatgctggaaaccaccccggcggaatgctataaagcggaatgctataacagcgaacgccagaaactgtataaactgattgattttctgatttatgatctgtattataaccgcaaaccggcgcgcattgaagaaattgtggataaactgcgcgaaagcgtgaacgatgaagaaaaagaaagcatttatagcgtggaagcgaaatatgtgtatgaaagcctgagcaaagtgctggataaaagcctgaaaaacagcgtgagcggcgaaaccattaaagatctgcagaaacgctatgatgatgaaaccgcgaaccgcatttgggatattagccagcatagcattagcggcaacgtgaactgcttttgcaaactgatttatattatgaccctgatgctggatggcaaagaaattaacgatctgctgaccaccctggtgaacaaatttgataacattgcgagctttattgatgtgatggatgaactgggcctggaacatagctttaccgataactataaaatgtttgcggatagcaaagcgatttgcctggatctgcagtttattaacagctttgcgcgcatgagcaaaattgatgatgaaaaaagcaaacgccagctgtttcgcgatgcgctggtgattctggatattggcaacaaagatgaaacctggattaacaactatctggatagcgatatttttaaactggataaagaaggcaacaaactgaaaggcgcgcgccatgattttcgcaactttattgcgaacaacgtgattaaaagcagccgctttaaatatctggtgaaatatagcagcgcggatggcatgattaaactgaaaaccaacgaaaaactgattggctttgtgctggataaactgccggaaacccagattgatcgctattatgaaagctgcggcctggataacgcggtggtggataaaaaagtgcgcattgaaaaactgagcggcctgattcgcgatatgaaatttgatgattttagcggcgtgaaaaccagcaacaaagcgggcgataacgataaacaggataaagcgaaatatcaggcgattattagcctgtatctgatggtgctgtatcagattgtgaaaaacatgatttatgtgaacagccgctatgtgattgcgtttcattgcctggaacgcgattttggcatgtatggcaaagattttggcaaatattatcagggctgccgcaaactgaccgatcattttattgaagaaaaatatatgaaagaaggcaaactgggctgcaacaaaaaagtgggccgctatctgaaaaacaacattagctgctgcaccgatggcctgattaacacctatcgcaaccaggtggatcattttgcggtggtgcgcaaaattggcaactatgcggcgtatattaaaagcattggcagctggtttgaactgtatcattatgtgattcagcgcattgtgtttgatgaatatcgctttgcgctgaacaacaccgaaagcaactataaaaacagcattattaaacatcatacctattgcaaagatatggtgaaagcgctgaacaccccgtttggctatgatctgccgcgctataaaaacctgagcattggcgatctgtttgatcgcaacaactatctgaacaaaaccaaagaaagcattgatgcgaacagcagcattgatagccagtatccgtatgatgtgccggattatgcg

SEQ ID NO: 82 - DNA, 2826 bp, Ruminococcus flavefaciens
atgaaaaaaaaaatgagcctgcgcgaaaaacgcgaagcggaaaaacaggcgaaaaaagcggcgtatagcgcggcgagcaaaaacaccgatagcaaaccggcggaaaaaaaagcggaaaccccgaaaccggcggaaattattagcgataacagccgcaacaaaaccgcggtgaaagcggcgggcctgaaaagcaccattattagcggcgataaactgtatatgaccagctttggcaaaggcaacgcggcggtgattgaacagaaaattgatattaacgattatagctttagcgcgatgaaagataccccgagcctggaagtggataaagcggaaagcaaagaaattagctttagcagccatcatccgtttgtgaaaaacgataaactgaccacctataacccgctgtatggcggcaaagataacccggaaaaaccggtgggccgcgatatgctgggcctgaaagataaactggaagaacgctattttggctgcacctttaacgataacctgcatattcagattatttataacattctggatattgaaaaaattctggcggtgcatagcgcgaacattaccaccgcgctggatcatatggtggatgaagatgatgaaaaatatctgaacagcgattatattggctatatgaacaccattaacacctatgatgtgtttatggatccgagcaaaaacagcagcctgagcccgaaagatcgcaaaaacattgataacagccgcgcgaaatttgaaaaactgctgagcaccaaacgcctgggctattttggctttgattatgatgcgaacggcaaagataaaaaaaaaaacgaagaaattaaaaaacgcctgtatcatctgaccgcgtttgcgggccagctgcgccagtggagctttcatagcgcgggcaactatccgcgcacctggctgtataaactggatagcctggataaagaatatctggataccctggatcattattttgataaacgctttaacgatattaacgatgattttgtgaccaaaaacgcgaccaacctgtatattctgaaagaagtgtttccggaagcgaactttaaagatattgcggatctgtattatgattttattgtgattaaaagccataaaaacatgggctttagcattaaaaaactgcgcgaaaaaatgctggaatgcgatggcgcggatcgcattaaagaacaggatatggatagcgtgcgcagcaaactgtataaactgattgatttttgcatttttaaatattatcatgaatttccggaactgagcgaaaaaaacgtggatattctgcgcgcggcggtgagcgataccaaaaaagataacctgtatagcgatgaagcggcgcgcctgtggagcatttttaaagaaaaatttctgggcttttgcgataaaattgtggtgtgggtgaccggcgaacatgaaaaagatattaccagcgtgattgataaagatgcgtatcgcaaccgcagcaacgtgagctattttagcaaactgatgtatgcgatgtgcttttttctggatggcaaagaaattaacgatctgctgaccaccctgattaacaaatttgataacattgcgaaccagattaaaaccgcgaaagaactgggcattaacaccgcgtttgtgaaaaactatgatttttttaaccatagcgaaaaatatgtggatgaactgaacattgtgaaaaacattgcgcgcatgaaaaaaccgagcagcaacgcgaaaaaagcgatgtatcatgatgcgctgaccattctgggcattccggaagatatggatgaaaaagcgctggatgaagaactggatctgattctggaaaaaaaaaccgatccggtgaccggcaaaccgctgaaaggcaaaaacccgctgcgcaactttattgcgaacaacgtgattgaaaacagccgctttatttatctgattaaattttgcaacccggaaaacgtgcgcaaaattgtgaacaacaccaaagtgaccgaatttgtgctgaaacgcattccggatgcgcagattgaacgctattataaaagctgcaccgatagcgaaatgaacccgccgaccgaaaaaaaaattaccgaactggcgggcaaactgaaagatatgaactttggcaactttcgcaacgtgcgccagagcgcgaaagaaaacatggaaaaagaacgctttaaagcggtgattggcctgtatctgaccgtggtgtatcgcgtggtgaaaaacctggtggatgtgaacagccgctatattatggcgtttcatagcctggaacgcgatagccagctgtataacgtgagcgtggataacgattatctggcgctgaccgataccctggtgaaagaaggcgataacagccgcagccgctatctggcgggcaacaaacgcctgcgcgattgcgtgaaacaggatattgataacgcgaaaaaatggtttgtgagcgataaatataacagcattaccaaatatcgcaacaacgtggcgcatctgaccgcggtgcgcaactgcgcggaatttattggcgatattaccaaaattgatagctattttgcgctgtatcattatctgattcagcgccagctggcgaaaggcctggatcatgaacgcagcggctttgatcgcaactatccgcagtatgcgccgctgtttaaatggcatacctatgtgaaagatgtggtgaaagcgctgaacgcgccgtttggctataacattccgcgctttaaaaacctgagcattgatgcgctgtttgatcgcaacgaaattaaaaaaaacgatggcgaaaaaaaaagcgatgattatccgtatgatgtgccggattatgcg

SEQ ID NO: 83 - DNA, 2904 bp, Ruminococcus flavefaciens
atgatcgaaaaaaaaaagtccttcgccaagggcatgggcgtgaagtccacactcgtgtccggctccaaagtgtacatgacaaccttcgccgaaggcagcgacgccaggctggaaaagatcgtggagggcgacagcatcaggagcgtgaatgagggcgaggccttcagcgctgaaatggccgataaaaacgccggctataagatcggcaacgccaaattcagccatcctaagggctacgccgtggtggctaacaaccctctgtatacaggacccgtccagcaggatatgctcggcctgaaggaaactctggaaaagaggtacttcggcgagagcgctgatggcaatgacaatatttgtatccaggtgatccataacatcctggacattgaaaaaatcctcgccgaatacattaccaacgccgcctacgccgtcaacaatatctccggcctggataaggacattattggattcggcaagttctccacagtgtatacctacgacgaattcaaagaccccgagcaccatagggccgctttcaacaataacgataagctcatcaacgccatcaaggcccagtatgacgagttcgacaacttcctcgataaccccagactcggctatttcggccaggcctttttcagcaaggagggcagaaattacatcatcaattacggcaacgaatgctatgacattctggccctcctgagcggactgaggcactgggtggtccataacaacgaagaagagtccaggatctccaggacctggctctacaacctcgataagaacctcgacaacgaatacatctccaccctcaactacctctacgacaggatcaccaatgagctgaccaactccttctccaagaactccgccgccaacgtgaactatattgccgaaactctgggaatcaaccctgccgaattcgccgaacaatatttcagattcagcattatgaaagagcagaaaaacctcggattcaatatcaccaagctcagggaagtgatgctggacaggaaggatatgtccgagatcaggaaaaatcataaggtgttcgactccatcaggaccaaggtctacaccatgatggactttgtgatttataggtattacatcgaagaggatgccaaggtggctgccgccaataagtccctccccgataatgagaagtccctgagcgagaaggatatctttgtgattaacctgaggggctccttcaacgacgaccagaaggatgccctctactacgatgaagctaatagaatttggagaaagctcgaaaatatcatgcacaacatcaaggaatttaggggaaacaagacaagagagtataagaagaaggacgcccctagactgcccagaatcctgcccgctggccgtgatgtttccgccttcagcaaactcatgtatgccctgaccatgttcctggatggcaaggagatcaacgacctcctgaccaccctgattaataaattcgataacatccagagcttcctgaaggtgatgcctctcatcggagtcaacgctaagttcgtggaggaatacgcctttttcaaagactccgccaagatcgccgatgagctgaggctgatcaagtccttcgctagaatgggagaacctattgccgatgccaggagggccatgtatatcgacgccatccgtattttaggaaccaacctgtcctatgatgagctcaaggccctcgccgacaccttttccctggacgagaacggaaacaagctcaagaaaggcaagcacggcatgagaaatttcattattaataacgtgatcagcaataaaaggttccactacctgatcagatacggtgatcctgcccacctccatgagatcgccaaaaacgaggccgtggtgaagttcgtgctcggcaggatcgctgacatccagaaaaaacagggccagaacggcaagaaccagatcgacaggtactacgaaacttgtatcggaaaggataagggcaagagcgtgagcgaaaaggtggacgctctcacaaagatcatcaccggaatgaactacgaccaattcgacaagaaaaggagcgtcattgaggacaccggcagggaaaacgccgagagggagaagtttaaaaagatcatcagcctgtacctcaccgtgatctaccacatcctcaagaatattgtcaatatcaacgccaggtacgtcatcggattccattgcgtcgagcgtgatgctcaactgtacaaggagaaaggctacgacatcaatctcaagaaactggaagagaagggattcagctccgtcaccaagctctgcgctggcattgatgaaactgcccccgataagagaaaggacgtggaaaaggagatggctgaaagagccaaggagagcattgacagcctcgagagcgccaaccccaagctgtatgccaattacatcaaatacagcgacgagaagaaagccgaggagttcaccaggcagattaacagggagaaggccaaaaccgccctgaacgcctacctgaggaacaccaagtggaatgtgatcatcagggaggacctcctgagaattgacaacaagacatgtaccctgttcagaaacaaggccgtccacctggaagtggccaggtatgtccacgcctatatcaacgacattgccgaggtcaattcctacttccaactgtaccattacatcatgcagagaattatcatgaatgagaggtacgagaaaagcagcggaaaggtgtccgagtacttcgacgctgtgaatgacgagaagaagtacaacgataggctcctgaaactgctgtgtgtgcctttcggctactgtatccccaggtttaagaacctgagcatcgaggccctgttcgataggaacgaggccgccaagttcgacaaggagaaaaagaaggtgtccggcaattcctaa

SEQ ID NO: 84 - DNA, 2769 bp, Unknown
  Uncultured Ruminococcus species
atggcaaaaaaaaataaaatgaaaccgcgtgaactgcgtgaagcacagaaaaaagcacgtcagctgaaagcagcagaaattaataataatgcagcaccggcaattgcagcaatgccggcagcagaagttattgcaccggttgcagaaaaaaaaaaaagcagcgttaaagcagcaggtatgaaaagcattctggttagcgaaaataaaatgtatattaccagctttggtaaaggtaatagcgcagttctggaatatgaagttgataataatgattataataaaacccagctgagcagcaaagataatagcaatattgaactgggtgatgttaatgaagttaatattacctttagcagcaaacatggttttggtagcggtgttgaaattaataccagcaatccgacccatcgtagcggtgaaagcagcccggttcgtggtgatatgctgggtctgaaaagcgaactggaaaaacgtttttttggtaaaacctttgatgataatattcatattcagctgatttataatattctggatattgaaaaaattctggcagtttatgttaccaatattgtttatgcactgaataatatgctgggtattaaagatagcgaaagctatgatgattttatgggttatctgagcgcacgtaatacctatgaagtttttacccatccggataaaagcaatctgagcgataaagttaaaggtaatattaaaaaaagcctgagcaaatttaatgatctgctgaaaaccaaacgtctgggttattttggtctggaagaaccgaaaaccaaagatacccgtgcaagcgaagcatataaaaaacgtgtttatcacatgctggcaattgttggtcagattcgtcagtgtgtttttcatgataaaagcggtgcaaaacgttttgatctgtatagctttattaataatattgatccggaatatcgtgataccctggattatctggttgaagaacgtctgaaaagcattaataaagattttattgaaggtaataaagttaatattagcctgctgattgatatgatgaaaggttatgaagcagatgatattattcgtctgtattatgattttattgttctgaaaagccagaaaaatctgggttttagcattaaaaaactgcgtgaaaaaatgctggaagaatatggttttcgttttaaagataaacagtatgatagcgttcgtagcaaaatgtataaactgatggattttctgctgttttgtaattattatcgtaatgatgttgcagcaggtgaagcactggttcgtaaactgcgttttagcatgaccgatgatgaaaaagaaggtatttatgcagatgaagcagcaaaactgtggggtaaatttcgtaatgattttgaaaatattgcagatcacatgaatggtgatgttattaaagaactgggtaaagcagatatggattttgatgaaaaaattctggatagcgaaaaaaaaaatgcaagcgatctgctgtattttagcaaaatgatttatatgctgacctattttctggatggtaaagaaattaatgatctgctgaccaccctgattagcaaatttgataatattaaagaatttctgaaaattatgaaaagcagcgcagttgatgttgaatgtgaactgaccgcaggttataaactgtttaatgatagccagcgtattaccaatgaactgtttattgttaaaaatattgcaagcatgcgtaaaccggcagcaagcgcaaaactgaccatgtttcgtgatgcactgaccattctgggtattgatgataatattaccgatgatcgtattagcgaaattctgaaactgaaagaaaaaggtaaaggtattcatggtctgcgtaattttattaccaataatgttattgaaagcagccgttttgtttatctgattaaatatgcaaatgcacagaaaattcgtgaagttgcaaaaaatgaaaaagttgttatgtttgttctgggtggtattccggatacccagattgaacgttattataaaagctgtgttgaatttccggatatgaatagcagcctggaagcaaaacgtagcgaactggcacgtatgattaaaaatattagctttgatgattttaaaaatgttaaacagcaggcaaaaggtcgtgaaaatgttgcaaaagaacgtgcaaaagcagttattggtctgtatctgaccgttatgtatctgctggttaaaaatctggttaatgttaatgcacgttatgttattgcaattcattgtctggaacgtgattttggtctgtataaagaaattattccggaactggcaagcaaaaatctgaaaaatgattatcgtattctgagccagaccctgtgtgaactgtgtgatgatcgtaatgaaagcagcaatctgtttctgaaaaaaaataaacgtctgcgtaaatgtgttgaagttgatattaataatgcagatagcagcatgacccgtaaatatcgtaattgtattgcacatctgaccgttgttcgtgaactgaaagaatatattggtgatattcgtaccgttgatagctattttagcatttatcattatgttatgcagcgttgtattaccaaacgtggtgatgataccaaacaggaagaaaaaattaaatatgaagatgatctgctgaaaaatcatggttataccaaagattttgttaaagcactgaatagcccgtttggttataatattccgcgttttaaaaatctgagcattgaacagctgtttgatcgtaatgaatatctgaccgaaaaataa

SEQ ID NO: 85 - protein, 18 aa, Artificial Sequence
  Synthetic
KRTADGSEFESPKKKRKV

SEQ ID NO: 86 - DNA, 49 bp, Artificial Sequence
  Synthetic
taacaccgtgcgtgttgactattttacctctggcggtgataatggttgc

SEQ ID NO: 87 - DNA, 27 bp, Artificial Sequence
  Synthetic
tttaccggaggacagtactccgacgta

SEQ ID NO: 88 - DNA, 14 bp, Artificial Sequence
  Synthetic
  misc_feature (4)..(13): n is a, c, g, or t
taannnnnnnnnnt

SEQ ID NO: 89 - DNA, 14 bp, Artificial Sequence
  Synthetic
  misc_feature (2)..(3): n is a, c, g, or t
  misc_feature (6)..(13): n is a, c, g, or t
tnnaannnnnnnnt

SEQ ID NO: 90 - DNA, 14 bp, Artificial Sequence
  Synthetic
  misc_feature (2)..(5): n is a, c, g, or t
  misc_feature (8)..(13): n is a, c, g, or t
tnnnnaannnnnnt

SEQ ID NO: 91 - DNA, 14 bp, Artificial Sequence
  Synthetic
  misc_feature (2)..(7): n is a, c, g, or t
  misc_feature (10)..(13): n is a, c, g, or t
tnnnnnnaannnnt

SEQ ID NO: 92 - DNA, 14 bp, Artificial Sequence
  Synthetic
  misc_feature (2)..(9): n is a, c, g, or t
  misc_feature (12)..(13): n is a, c, g, or t
tnnnnnnnnaannt

SEQ ID NO: 93 - DNA, 14 bp, Artificial Sequence
  Synthetic
  misc_feature (2)..(11): n is a, c, g, or t
tnnnnnnnnnnaat

SEQ ID NO: 94 - DNA, 14 bp, Artificial Sequence
  Synthetic
  misc_feature (2)..(3): n is a, c, g, or t
  misc_feature (6)..(13): n is a, c, g, or t
tnnacnnnnnnnnt

SEQ ID NO: 95 - DNA, 14 bp, Artificial Sequence
  Synthetic
  misc_feature (2)..(5): n is a, c, g, or t
  misc_feature (8)..(13): n is a, c, g, or t
tnnnnacnnnnnnt

SEQ ID NO: 96 - DNA, 14 bp, Artificial Sequence
  Synthetic
  misc_feature (2)..(7): n is a, c, g, or t
  misc_feature (10)..(13): n is a, c, g, or t
tnnnnnnacnnnnt

SEQ ID NO: 97 - DNA, 14 bp, Artificial Sequence
  Synthetic
  misc_feature (2)..(9): n is a, c, g, or t
  misc_feature (12)..(13): n is a, c, g, or t
tnnnnnnnnacnnt

SEQ ID NO: 98 - DNA, 14 bp, Artificial Sequence
  Synthetic
  misc_feature (2)..(11): n is a, c, g, or t
tnnnnnnnnnnact

SEQ ID NO: 99 - DNA, 14 bp, Artificial Sequence
  Synthetic
  misc_feature (2)..(3): n is a, c, g, or t
  misc_feature (6)..(13): n is a, c, g, or t
tnnagnnnnnnnnt

SEQ ID NO: 100 - DNA, 14 bp, Artificial Sequence
  Synthetic
  misc_feature (2)..(5): n is a, c, g, or t
  misc_feature (8)..(13): n is a, c, g, or t
tnnnnagnnnnnnt

SEQ ID NO: 101 - DNA, 14 bp, Artificial Sequence
  Synthetic
  misc_feature (2)..(7): n is a, c, g, or t
  misc_feature (10)..(13): n is a, c, g, or t
tnnnnnnagnnnnt

SEQ ID NO: 102 - DNA, 14 bp, Artificial Sequence
  Synthetic
  misc_feature (2)..(9): n is a, c, g, or t
  misc_feature (12)..(13): n is a, c, g, or t
tnnnnnnnnagnnt

SEQ ID NO: 103 - DNA, 14 bp, Artificial Sequence
  Synthetic
  misc_feature (2)..(11): n is a, c, g, or t
tnnnnnnnnnnagt

SEQ ID NO: 104 - DNA, 14 bp, Artificial Sequence
  Synthetic
  misc_feature (2)..(3): n is a, c, g, or t
  misc_feature (6)..(13): n is a, c, g, or t
tnnatnnnnnnnnt

SEQ ID NO: 105 - DNA, 14 bp, Artificial Sequence
  Synthetic
  misc_feature (2)..(5): n is a, c, g, or t
  misc_feature (8)..(13): n is a, c, g, or t
tnnnnatnnnnnnt

SEQ ID NO: 106 - DNA, 14 bp, Artificial Sequence
  Synthetic
  misc_feature (2)..(7): n is a, c, g, or t
  misc_feature (10)..(13): n is a, c, g, or t
tnnnnnnatnnnnt

SEQ ID NO: 107 - DNA, 14 bp, Artificial Sequence
  Synthetic
  misc_feature (2)..(9): n is a, c, g, or t
  misc_feature (12)..(13): n is a, c, g, or t
tnnnnnnnnatnnt

SEQ ID NO: 108 - DNA, 14 bp, Artificial Sequence
  Synthetic
  misc_feature (2)..(11): n is a, c, g, or t
tnnnnnnnnnnatt

SEQ ID NO: 109 - DNA, 14 bp, Artificial Sequence
  Synthetic
  misc_feature (2)..(3): n is a, c, g, or t
  misc_feature (6)..(13): n is a, c, g, or t
tnncannnnnnnnt

SEQ ID NO: 110 - DNA, 14 bp, Artificial Sequence
  Synthetic
  misc_feature (2)..(9): n is a, c, g, or t
  misc_feature (12)..(13): n is a, c, g, or t
tnnnnnnnncannt

SEQ ID NO: 111 - DNA, 14 bp, Artificial Sequence
  Synthetic
  misc_feature (2)..(11): n is a, c, g, or t
tnnnnnnnnnncat

SEQ ID NO: 112 - DNA, 14 bp, Artificial Sequence
  Synthetic
  misc_feature (4)..(13): n is a, c, g, or t
tccnnnnnnnnnnt

SEQ ID NO: 113 - DNA, 14 bp, Artificial Sequence
  Synthetic
  misc_feature (2)..(3): n is a, c, g, or t
  misc_feature (6)..(13): n is a, c, g, or t
tnnccnnnnnnnnt

SEQ ID NO: 114 - DNA, 14 bp, Artificial Sequence
  Synthetic
  misc_feature (2)..(5): n is a, c, g, or t
  misc_feature (8)..(13): n is a, c, g, or t
tnnnnccnnnnnnt

SEQ ID NO: 115 - DNA, 14 bp, Artificial Sequence
  Synthetic
  misc_feature (2)..(7): n is a, c, g, or t
  misc_feature (10)..(13): n is a, c, g, or t
tnnnnnnccnnnnt

SEQ ID NO: 116 - DNA, 14 bp, Artificial Sequence
  Synthetic
  misc_feature (2)..(9): n is a, c, g, or t
  misc_feature (12)..(13): n is a, c, g, or t
tnnnnnnnnccnnt

SEQ ID NO: 117 - DNA, 14 bp, Artificial Sequence
  Synthetic
  misc_feature (2)..(11): n is a, c, g, or t
tnnnnnnnnnncct

SEQ ID NO: 118 - DNA, 14 bp, Artificial Sequence
  Synthetic
  misc_feature (4)..(13): n is a, c, g, or t
tcgnnnnnnnnnnt

SEQ ID NO: 119 - DNA, 14 bp, Artificial Sequence
  Synthetic
  misc_feature (2)..(3): n is a, c, g, or t
  misc_feature (6)..(13): n is a, c, g, or t
tnncgnnnnnnnnt

SEQ ID NO: 120 - DNA, 14 bp, Artificial Sequence
  Synthetic
  misc_feature (2)..(5): n is a, c, g, or t
  misc_feature (8)..(13): n is a, c, g, or t
tnnnncgnnnnnnt

SEQ ID NO: 121 - DNA, 14 bp, Artificial Sequence
  Synthetic
  misc_feature (2)..(7): n is a, c, g, or t
  misc_feature (10)..(13): n is a, c, g, or t
tnnnnnncgnnnnt

SEQ ID NO: 122 - DNA, 14 bp, Artificial Sequence
  Synthetic
  misc_feature (2)..(9): n is a, c, g, or t
  misc_feature (12)..(13): n is a, c, g, or t
tnnnnnnnncgnnt

SEQ ID NO: 123 - DNA, 14 bp, Artificial Sequence
  Synthetic
  misc_feature (2)..(11): n is a, c, g, or t
tnnnnnnnnnncgt

SEQ ID NO: 124 - DNA, 14 bp, Artificial Sequence
  Synthetic
  misc_feature (2)..(5): n is a, c, g, or t
  misc_feature (8)..(13): n is a, c, g, or t
tnnnnctnnnnnnt

SEQ ID NO: 125 - DNA, 14 bp, Artificial Sequence
  Synthetic
  misc_feature (2)..(7): n is a, c, g, or t
  misc_feature (10)..(13): n is a, c, g, or t
tnnnnnnctnnnnt

SEQ ID NO: 126 - DNA, 14 bp, Artificial Sequence
  Synthetic
  misc_feature (2)..(9): n is a, c, g, or t
  misc_feature (12)..(13): n is a, c, g, or t
tnnnnnnnnctnnt

SEQ ID NO: 127 - DNA, 14 bp, Artificial Sequence
  Synthetic
  misc_feature (2)..(11): n is a, c, g, or t
tnnnnnnnnnnctt

SEQ ID NO: 128 - DNA, 14 bp, Artificial Sequence
  Synthetic
  misc_feature (4)..(13): n is a, c, g, or t
tgannnnnnnnnnt

SEQ ID NO: 129 - DNA, 14 bp, Artificial Sequence
  Synthetic
  misc_feature (2)..(3): n is a, c, g, or t
  misc_feature (6)..(13): n is a, c, g, or t
tnngannnnnnnnt

SEQ ID NO: 130 - DNA, 14 bp, Artificial Sequence
  Synthetic
  misc_feature (2)..(5): n is a, c, g, or t
  misc_feature (8)..(13): n is a, c, g, or t
tnnnngannnnnnt

SEQ ID NO: 131 - DNA, 14 bp, Artificial Sequence
  Synthetic
  misc_feature (2)..(7): n is a, c, g, or t
  misc_feature (10)..(13): n is a, c, g, or t
tnnnnnngannnnt

SEQ ID NO: 132 - DNA, 14 bp, Artificial Sequence
  Synthetic
  misc_feature (2)..(9): n is a, c, g, or t
  misc_feature (12)..(13): n is a, c, g, or t
tnnnnnnnngannt

SEQ ID NO: 133 - DNA, 14 bp, Artificial Sequence
  Synthetic
  misc_feature (2)..(11): n is a, c, g, or t
tnnnnnnnnnngat

SEQ ID NO: 134 - DNA, 14 bp, Artificial Sequence
  Synthetic
  misc_feature (4)..(13): n is a, c, g, or t
tgcnnnnnnnnnnt

SEQ ID NO: 135 - DNA, 14 bp, Artificial Sequence
  Synthetic
  misc_feature (2)..(3): n is a, c, g, or t
  misc_feature (6)..(13): n is a, c, g, or t
tnngcnnnnnnnnt

SEQ ID NO: 136 - DNA, 14 bp, Artificial Sequence
  Synthetic
  misc_feature (2)..(5): n is a, c, g, or t
  misc_feature (8)..(13): n is a, c, g, or t
tnnnngcnnnnnnt

SEQ ID NO: 137 - DNA, 14 bp, Artificial Sequence
  Synthetic
  misc_feature (2)..(7): n is a, c, g, or t
  misc_feature (10)..(13): n is a, c, g, or t
tnnnnnngcnnnnt

SEQ ID NO: 138 - DNA, 14 bp, Artificial Sequence
  Synthetic
  misc_feature (2)..(9): n is a, c, g, or t
  misc_feature (12)..(13): n is a, c, g, or t
tnnnnnnnngcnnt

SEQ ID NO: 139 - DNA, 14 bp, Artificial Sequence
  Synthetic
  misc_feature (2)..(11): n is a, c, g, or t
tnnnnnnnnnngct

SEQ ID NO: 140 - DNA, 14 bp, Artificial Sequence
  Synthetic
  misc_feature (4)..(13): n is a, c, g, or t
tggnnnnnnnnnnt

SEQ ID NO: 141 - DNA, 14 bp, Artificial Sequence
  Synthetic
  misc_feature (2)..(3): n is a, c, g, or t
  misc_feature (6)..(13): n is a, c, g, or t
tnnggnnnnnnnnt

SEQ ID NO: 142 - DNA, 14 bp, Artificial Sequence
  Synthetic
  misc_feature (2)..(5): n is a, c, g, or t
  misc_feature (8)..(13): n is a, c, g, or t
tnnnnggnnnnnnt

SEQ ID NO: 143 - DNA, 14 bp, Artificial Sequence
  Synthetic
  misc_feature (2)..(7): n is a, c, g, or t
  misc_feature (10)..(13): n is a, c, g, or t
tnnnnnnggnnnnt

SEQ ID NO: 144 - DNA, 14 bp, Artificial Sequence
  Synthetic
  misc_feature (2)..(9): n is a, c, g, or t
  misc_feature (12)..(13): n is a, c, g, or t
tnnnnnnnnggnnt

SEQ ID NO: 145 - DNA, 14 bp, Artificial Sequence
  Synthetic
  misc_feature (2)..(11): n is a, c, g, or t
tnnnnnnnnnnggt

SEQ ID NO: 146 - DNA, 14 bp, Artificial Sequence
  Synthetic
  misc_feature (4)..(13): n is a, c, g, or t
tgtnnnnnnnnnnt

SEQ ID NO: 147 - DNA, 14 bp, Artificial Sequence
  Synthetic
  misc_feature (2)..(3): n is a, c, g, or t
  misc_feature (6)..(13): n is a, c, g, or t
tnngtnnnnnnnnt

SEQ ID NO: 148 - DNA, 14 bp, Artificial Sequence
  Synthetic
  misc_feature (2)..(5): n is a, c, g, or t
  misc_feature (8)..(13): n is a, c, g, or t
tnnnngtnnnnnnt

SEQ ID NO: 149 - DNA, 14 bp, Artificial Sequence
  Synthetic
  misc_feature (2)..(7): n is a, c, g, or t
  misc_feature (10)..(13): n is a, c, g, or t
tnnnnnngtnnnnt

SEQ ID NO: 150 - DNA, 14 bp, Artificial Sequence
  Synthetic
  misc_feature (2)..(9): n is a, c, g, or t
  misc_feature (12)..(13): n is a, c, g, or t
tnnnnnnnngtnnt

SEQ ID NO: 151 - DNA, 14 bp, Artificial Sequence
  Synthetic
  misc_feature (2)..(11): n is a, c, g, or t
tnnnnnnnnnngtt

SEQ ID NO: 152 - DNA, 14 bp, Artificial Sequence
  Synthetic
  misc_feature (4)..(13): n is a, c, g, or t
ttannnnnnnnnnt

SEQ ID NO: 153 - DNA, 14 bp, Artificial Sequence
  Synthetic
  misc_feature (2)..(3): n is a, c, g, or t
  misc_feature (6)..(13): n is a, c, g, or t
tnntannnnnnnnt

SEQ ID NO: 154 - DNA, 14 bp, Artificial Sequence
  Synthetic
  misc_feature (2)..(5): n is a, c, g, or t
  misc_feature (8)..(13): n is a, c, g, or t
tnnnntannnnnnt

SEQ ID NO: 155 - DNA, 14 bp, Artificial Sequence
  Synthetic
  misc_feature (2)..(7): n is a, c, g, or t
  misc_feature (10)..(13): n is a, c, g, or t
tnnnnnntannnnt

SEQ ID NO: 156 - DNA, 14 bp, Artificial Sequence
  Synthetic
  misc_feature (2)..(9): n is a, c, g, or t
  misc_feature (12)..(13): n is a, c, g, or t
tnnnnnnnntannt

SEQ ID NO: 157 - DNA, 14 bp, Artificial Sequence
  Synthetic
  misc_feature (2)..(11): n is a, c, g, or t
tnnnnnnnnnntat

SEQ ID NO: 158 - DNA, 14 bp, Artificial Sequence
  Synthetic
  misc_feature (4)..(13): n is a, c, g, or t
ttcnnnnnnnnnnt

SEQ ID NO: 159 - DNA, 14 bp, Artificial Sequence
  Synthetic
  misc_feature (2)..(3): n is a, c, g, or t
  misc_feature (6)..(13): n is a, c, g, or t
tnntcnnnnnnnnt

SEQ ID NO: 160 - DNA, 14 bp, Artificial Sequence
  Synthetic
  misc_feature (2)..(5): n is a, c, g, or t
  misc_feature (8)..(13): n is a, c, g, or t
tnnnntcnnnnnnt

SEQ ID NO: 161 - DNA, 14 bp, Artificial Sequence
  Synthetic
  misc_feature (2)..(7): n is a, c, g, or t
  misc_feature (10)..(13): n is a, c, g, or t
tnnnnnntcnnnnt

SEQ ID NO: 162 - DNA, 14 bp, Artificial Sequence
  Synthetic
  misc_feature (2)..(9): n is a, c, g, or t
  misc_feature (12)..(13): n is a, c, g, or t
tnnnnnnnntcnnt

SEQ ID NO: 163 - DNA, 14 bp, Artificial Sequence
  Synthetic
  misc_feature (2)..(11): n is a, c, g, or t
tnnnnnnnnnntct

SEQ ID NO: 164 - DNA, 14 bp, Artificial Sequence
  Synthetic
  misc_feature (4)..(13): n is a, c, g, or t
ttgnnnnnnnnnnt

SEQ ID NO: 165 - DNA, 14 bp, Artificial Sequence
  Synthetic
  misc_feature (2)..(3): n is a, c, g, or t
  misc_feature (6)..(13): n is a, c, g, or t
tnntgnnnnnnnnt

SEQ ID NO: 166 - DNA, 14 bp, Artificial Sequence
  Synthetic
  misc_feature (2)..(5): n is a, c, g, or t
  misc_feature (8)..(13): n is a, c, g, or t
tnnnntgnnnnnnt

SEQ ID NO: 167 - DNA, 14 bp, Artificial Sequence
  Synthetic
  misc_feature (2)..(7): n is a, c, g, or t
  misc_feature (10)..(13): n is a, c, g, or t
tnnnnnntgnnnnt

SEQ ID NO: 168 - DNA, 14 bp, Artificial Sequence
  Synthetic
  misc_feature (2)..(9): n is a, c, g, or t
  misc_feature (12)..(13): n is a, c, g, or t
tnnnnnnnntgnnt

SEQ ID NO: 169 - DNA, 14 bp, Artificial Sequence
  Synthetic
  misc_feature (2)..(11): n is a, c, g, or t
tnnnnnnnnnntgt

SEQ ID NO: 170 - DNA, 14 bp, Artificial Sequence
  Synthetic
  misc_feature (4)..(13): n is a, c, g, or t
tttnnnnnnnnnnt

SEQ ID NO: 171 - DNA, 14 bp, Artificial Sequence
  Synthetic
  misc_feature (2)..(3): n is a, c, g, or t
  misc_feature (6)..(13): n is a, c, g, or t
tnnttnnnnnnnnt

SEQ ID NO: 172 - DNA, 14 bp, Artificial Sequence
  Synthetic
  misc_feature (2)..(5): n is a, c, g, or t
  misc_feature (8)..(13): n is a, c, g, or t
tnnnnttnnnnnnt

SEQ ID NO: 173 - DNA, 14 bp, Artificial Sequence
  Synthetic
  misc_feature (2)..(7): n is a, c, g, or t
  misc_feature (10)..(13): n is a, c, g, or t
tnnnnnnttnnnnt

SEQ ID NO: 174 - DNA, 14 bp, Artificial Sequence
  Synthetic
  misc_feature (2)..(9): n is a, c, g, or t
  misc_feature (12)..(13): n is a, c, g, or t
tnnnnnnnnttnnt

SEQ ID NO: 175 - DNA, 14 bp, Artificial Sequence
  Synthetic
  misc_feature (2)..(11): n is a, c, g, or t
tnnnnnnnnnnttt

SEQ ID NO: 176 - DNA, 15 bp, Artificial Sequence
  Synthetic
gaccacccaagacga

SEQ ID NO: 177 - DNA, 54 bp, Artificial Sequence
  Synthetic
tccctatcagtgatagagattgacatccctatcagtgatagagatactgagcac

SEQ ID NO: 178 - DNA, 6680 bp, Artificial Sequence
  Synthetic
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgccctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcagccgtgttttagagctagaaatagcgtcatcctcatcggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtcgaaattaatacgactcactatagggcaagtaaacccctaccaactggtcggggtttgaaacgcagcagcagcagcagcagcagcaagtaaacccctaccaactggtcggggtttgaaacccgctgagcaataactagcataacccggtaccgcgggcccgggcgccaccatgggccccaagaagaagagaaaggtggaggccagcatcgaaaaaaaaaagtccttcgccaagggcatgggcgtgaagtccacactcgtgtccggctccaaagtgtacatgacaaccttcgccgaaggcagcgacgccaggctggaaaagatcgtggagggcgacagcatcaggagcgtgaatgagggcgaggccttcagcgctgaaatggccgataaaaacgccggctataagatcggcaacgccaaattcagccatcctaagggctacgccgtggtggctaacaaccctctgtatacaggacccgtccagcaggatatgctcggcctgaaggaaactctggaaaagaggtacttcggcgagagcgctgatggcaatgacaatatttgtatccaggtgatccataacatcctggacattgaaaaaatcctcgccgaatacattaccaacgccgcctacgccgtcaacaatatctccggcctggataaggacattattggattcggcaagttctccacagtgtatacctacgacgaattcaaagaccccgagcaccatagggccgctttcaacaataacgataagctcatcaacgccatcaaggcccagtatgacgagttcgacaacttcctcgataaccccagactcggctatttcggccaggcctttttcagcaaggagggcagaaattacatcatcaattacggcaacgaatgctatgacattctggccctcctgagcggactgaggcactgggtggtccataacaacgaagaagagtccaggatctccaggacctggctctacaacctcgataagaacctcgacaacgaatacatctccaccctcaactacctctacgacaggatcaccaatgagctgaccaactccttctccaagaactccgccgccaacgtgaactatattgccgaaactctgggaatcaaccctgccgaattcgccgaacaatatttcagattcagcattatgaaagagcagaaaaacctcggattcaatatcaccaagctcagggaagtgatgctggacaggaaggatatgtccgagatcaggaaaaatcataaggtgttcgactccatcaggaccaaggtctacaccatgatggactttgtgatttataggtattacatcgaagaggatgccaaggtggctgccgccaataagtccctccccgataatgagaagtccctgagcgagaaggatatctttgtgattaacctgaggggctccttcaacgacgaccagaaggatgccctctactacgatgaagctaatagaatttggagaaagctcgaaaatatcatgcacaacatcaaggaatttaggggaaacaagacaagagagtataagaagaaggacgcccctagactgcccagaatcctgcccgctggccgtgatgtttccgccttcagcaaactcatgtatgccctgaccatgttcctggatggcaaggagatcaacgacctcctgaccaccctgattaataaattcgataacatccagagcttcctgaaggtgatgcctctcatcggagtcaacgctaagttcgtggaggaatacgcctttttcaaagactccgccaagatcgccgatgagctgaggctgatcaagtccttcgctagaatgggagaacctattgccgatgccaggagggccatgtatatcgacgccatccgtattttaggaaccaacctgtcctatgatgagctcaaggccctcgccgacaccttttccctggacgagaacggaaacaagctcaagaaaggcaagcacggcatgagaaatttcattattaataacgtgatcagcaataaaaggttccactacctgatcagatacggtgatcctgcccacctccatgagatcgccaaaaacgaggccgtggtgaagttcgtgctcggcaggatcgctgacatccagaaaaaacagggccagaacggcaagaaccagatcgacaggtactacgaaacttgtatcggaaaggataagggcaagagcgtgagcgaaaaggtggacgctctcacaaagatcatcaccggaatgaactacgaccaattcgacaagaaaaggagcgtcattgaggacaccggcagggaaaacgccgagagggagaagtttaaaaagatcatcagcctgtacctcaccgtgatctaccacatcctcaagaatattgtcaatatcaacgccaggtacgtcatcggattccattgcgtcgagcgtgatgctcaactgtacaaggagaaaggctacgacatcaatctcaagaaactggaagagaagggattcagctccgtcaccaagctctgcgctggcattgatgaaactgcccccgataagagaaaggacgtggaaaaggagatggctgaaagagccaaggagagcattgacagcctcgagagcgccaaccccaagctgtatgccaattacatcaaatacagcgacgagaagaaagccgaggagttcaccaggcagattaacagggagaaggccaaaaccgccctgaacgcctacctgaggaacaccaagtggaatgtgatcatcagggaggacctcctgagaattgacaacaagacatgtaccctgttcagaaacaaggccgtccacctggaagtggccaggtatgtccacgcctatatcaacgacattgccgaggtcaattcctacttccaactgtaccattacatcatgcagagaattatcatgaatgagaggtacgagaaaagcagcggaaaggtgtccgagtacttcgacgctgtgaatgacgagaagaagtacaacgataggctcctgaaactgctgtgtgtgcctttcggctactgtatccccaggtttaagaacctgagcatcgaggccctgttcgataggaacgaggccgccaagttcgacaaggagaaaaagaaggtgtccggcaattccggatccggacctaagaaaaagaggaaggtggcggccgcttacccatacgatgttccagattacgcttaacttaagatacattgatgagtttggacaaaccacaactagaatgcagtgaaaaaaatgctttatttgtgaaatttgtgatgctattgctttatttgtaaccattataagctgcaataaacaagttctgattttgtagttaacacgtgcgaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 179 - DNA, 6680 bp, Artificial Sequence
  Synthetic
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgccctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcagccgtgttttagagctagaaatagcgtcatcctcatcggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtcgaaattaatacgactcactatagggcaagtaaacccctaccaactggtcggggtttgaaaccgagggcgacttaaccttaggtcaagtaaacccctaccaactggtcggggtttgaaacccgctgagcaataactagcataacccggtaccgcgggcccgggcgccaccatgggccccaagaagaagagaaaggtggaggccagcatcgaaaaaaaaaagtccttcgccaagggcatgggcgtgaagtccacactcgtgtccggctccaaagtgtacatgacaaccttcgccgaaggcagcgacgccaggctggaaaagatcgtggagggcgacagcatcaggagcgtgaatgagggcgaggccttcagcgctgaaatggccgataaaaacgccggctataagatcggcaacgccaaattcagccatcctaagggctacgccgtggtggctaacaaccctctgtatacaggacccgtccagcaggatatgctcggcctgaaggaaactctggaaaagaggtacttcggcgagagcgctgatggcaatgacaatatttgtatccaggtgatccataacatcctggacattgaaaaaatcctcgccgaatacattaccaacgccgcctacgccgtcaacaatatctccggcctggataaggacattattggattcggcaagttctccacagtgtatacctacgacgaattcaaagaccccgagcaccatagggccgctttcaacaataacgataagctcatcaacgccatcaaggcccagtatgacgagttcgacaacttcctcgataaccccagactcggctatttcggccaggcctttttcagcaaggagggcagaaattacatcatcaattacggcaacgaatgctatgacattctggccctcctgagcggactgaggcactgggtggtccataacaacgaagaagagtccaggatctccaggacctggctctacaacctcgataagaacctcgacaacgaatacatctccaccctcaactacctctacgacaggatcaccaatgagctgaccaactccttctccaagaactccgccgccaacgtgaactatattgccgaaactctgggaatcaaccctgccgaattcgccgaacaatatttcagattcagcattatgaaagagcagaaaaacctcggattcaatatcaccaagctcagggaagtgatgctggacaggaaggatatgtccgagatcaggaaaaatcataaggtgttcgactccatcaggaccaaggtctacaccatgatggactttgtgatttataggtattacatcgaagaggatgccaaggtggctgccgccaataagtccctccccgataatgagaagtccctgagcgagaaggatatctttgtgattaacctgaggggctccttcaacgacgaccagaaggatgccctctactacgatgaagctaatagaatttggagaaagctcgaaaatatcatgcacaacatcaaggaatttaggggaaacaagacaagagagtataagaagaaggacgcccctagactgcccagaatcctgcccgctggccgtgatgtttccgccttcagcaaactcatgtatgccctgaccatgttcctggatggcaaggagatcaacgacctcctgaccaccctgattaataaattcgataacatccagagcttcctgaaggtgatgcctctcatcggagtcaacgctaagttcgtggaggaatacgcctttttcaaagactccgccaagatcgccgatgagctgaggctgatcaagtccttcgctagaatgggagaacctattgccgatgccaggagggccatgtatatcgacgccatccgtattttaggaaccaacctgtcctatgatgagctcaaggccctcgccgacaccttttccctggacgagaacggaaacaagctcaagaaaggcaagcacggcatgagaaatttcattattaataacgtgatcagcaataaaaggttccactacctgatcagatacggtgatcctgcccacctccatgagatcgccaaaaacgaggccgtggtgaagttcgtgctcggcaggatcgctgacatccagaaaaaacagggccagaacggcaagaaccagatcgacaggtactacgaaacttgtatcggaaaggataagggcaagagcgtgagcgaaaaggtggacgctctcacaaagatcatcaccggaatgaactacgaccaattcgacaagaaaaggagcgtcattgaggacaccggcagggaaaacgccgagagggagaagtttaaaaagatcatcagcctgtacctcaccgtgatctaccacatcctcaagaatattgtcaatatcaacgccaggtacgtcatcggattccattgcgtcgagcgtgatgctcaactgtacaaggagaaaggctacgacatcaatctcaagaaactggaagagaagggattcagctccgtcaccaagctctgcgctggcattgatgaaactgcccccgataagagaaaggacgtggaaaaggagatggctgaaagagccaaggagagcattgacagcctcgagagcgccaaccccaagctgtatgccaattacatcaaatacagcgacgagaagaaagccgaggagttcaccaggcagattaacagggagaaggccaaaaccgccctgaacgcctacctgaggaacaccaagtggaatgtgatcatcagggaggacctcctgagaattgacaacaagacatgtaccctgttcagaaacaaggccgtccacctggaagtggccaggtatgtccacgcctatatcaacgacattgccgaggtcaattcctacttccaactgtaccattacatcatgcagagaattatcatgaatgagaggtacgagaaaagcagcggaaaggtgtccgagtacttcgacgctgtgaatgacgagaagaagtacaacgataggctcctgaaactgctgtgtgtgcctttcggctactgtatccccaggtttaagaacctgagcatcgaggccctgttcgataggaacgaggccgccaagttcgacaaggagaaaaagaaggtgtccggcaattccggatccggacctaagaaaaagaggaaggtggcggccgcttacccatacgatgttccagattacgcttaacttaagatacattgatgagtttggacaaaccacaactagaatgcagtgaaaaaaatgctttatttgtgaaatttgtgatgctattgctttatttgtaaccattataagctgcaataaacaagttctgattttgtagttaacacgtgcgaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 180 - DNA, 27 bp, Artificial Sequence
  Synthetic
tttactatcaccgccagaggtaacgta

SEQ ID NO: 181 - DNA, 29 bp, Artificial Sequence
  Synthetic
tttactccctatcagtgatagagaacgta

SEQ ID NO: 182 - RNA, 22 nt, Artificial Sequence
  Synthetic
gacgacgacgacgacgacgacg

SEQ ID NO: 183 - RNA, 22 nt, Artificial Sequence
  Synthetic
cgacgacgacgacgacgacgac

SEQ ID NO: 184 - RNA, 22 nt, Artificial Sequence
  Synthetic
acgacgacgacgacgacgacga

SEQ ID NO: 185 - RNA, 27 nt, Artificial Sequence
  Synthetic
cugcugcugcugcugcugcugcugcug

SEQ ID NO: 186 - DNA, 22 bp, Artificial Sequence
  Synthetic
tggattccaattcagcgggagc